SEQUENCE LISTING (18 sequences)

SEQ ID NO: 1 - DNA, 22 bp, synthetic construct
  source 1..22: mol_type = other DNA
gtgacggtgttgatggtaagat

SEQ ID NO: 2 - DNA, 21 bp, synthetic construct
  source 1..21: mol_type = other DNA
agctccacagagtgttccttg

SEQ ID NO: 3 - DNA, 20 bp, synthetic construct
  source 1..20: mol_type = other DNA
caggtggttgcattcttggc

SEQ ID NO: 4 - DNA, 20 bp, synthetic construct
  source 1..20: mol_type = other DNA
gagccggttcaagtctctgt

SEQ ID NO: 5 - DNA, 23 bp, synthetic construct
  source 1..23: mol_type = other DNA
atgttcgccatgaaaactaaggc

SEQ ID NO: 6 - DNA, 20 bp, synthetic construct
  source 1..20: mol_type = other DNA
gcgacgccacaatccttgta

SEQ ID NO: 7 - DNA, 19 bp, synthetic construct
  source 1..19: mol_type = other DNA
ggcgttgtccatgtgcaag

SEQ ID NO: 8 - DNA, 20 bp, synthetic construct
  source 1..20: mol_type = other DNA
ggactccggtagctgtagga

SEQ ID NO: 9 - DNA, 20 bp, synthetic construct
  source 1..20: mol_type = other DNA
gtaacccgttgaaccccatt

SEQ ID NO: 10 - DNA, 20 bp, synthetic construct
  source 1..20: mol_type = other DNA
ccatccaatcggtagtagcg

SEQ ID NO: 11 - DNA, 20 bp, synthetic construct
  source 1..20: mol_type = other DNA
ggcccacaatatcaaagaac

SEQ ID NO: 12 - DNA, 20 bp, synthetic construct
  source 1..20: mol_type = other DNA
gctacctagcaacaaggccc

SEQ ID NO: 13 - DNA, 20 bp, synthetic construct
  source 1..20: mol_type = other DNA
cgagtttgccaccttcccca

SEQ ID NO: 14 - DNA, 20 bp, synthetic construct
  source 1..20: mol_type = other DNA
acctgcgaaccaccagaatg

SEQ ID NO: 15 - DNA, 20 bp, synthetic construct
  source 1..20: mol_type = other DNA
ccagctattcggagtccaaa

SEQ ID NO: 16 - DNA, 20 bp, synthetic construct
  source 1..20: mol_type = other DNA
cacggagtcgcccaagaagc

SEQ ID NO: 17 - DNA, 20 bp, synthetic construct
  source 1..20: mol_type = other DNA
gagtacatcgccaagggtga

SEQ ID NO: 18 - DNA, 20 bp, synthetic construct
  source 1..20: mol_type = other DNA
aagtttgcaaaccacgtgag

(19) United States
(12) Patent Application Publication
(10) Pub. No.: US 2025/0090661 A1
(43) Pub. Date: Mar. 20, 2025

(54) EXPANSION OF HUMAN GROUP 2 INNATE LYMPHOID CELLS AND METHODS OF USE THEREOF

(71) Applicant: CITY OF HOPE, Duarte, CA (US)

(72) Inventors: Jianhua YU, Duarte, CA (US); Michael A. CALIGIURI, Duarte, CA (US)

(21) Appl. No.: 18/785,997

(22) Filed: Jul. 26, 2024

Related U.S. Application Data
(60) Provisional application No. 63/515,781, filed on Jul. 26, 2023.

(51) Int. Cl. A61K 39/00; C07K 14/725; C12N 5/0783

(52) U.S. Cl. CPC A61K 40/31; A61K 40/11; A61K 40/15; A61K 40/428; C07K 14/7051; C12N 5/0636; C12N 5/0646; C12N 2501/2302; C12N 2501/2307; C12N 2501/2315; C12N 2502/30

(57) ABSTRACT

Provided herein, inter alia, are methods for expanding a population of human group 2 innate lymphoid cells (ILC2), and methods of treating cancer in a subject including administering to the subject the population of expanded human ILC2. Further provided are genetically engineered human ILC2s including chimeric antigen receptors, and methods of treating cancer in a subject including administering to the subject the genetically engineered human ILC2.

### CROSS-REFERENCES TO RELATED APPLICATIONS

This application claims priority to U.S. Provisional Application No. 63/515,781, filed Jul. 26, 2023, which is hereby incorporated by reference in its entirety and for all purposes.

### REFERENCE TO AN ELECTRONIC SEQUENCE LISTING

The contents of the electronic sequence listing (048440-873001US_Sequence_Listing_ST26.xml; Size: 16,880 bytes; and Date of Creation: Jun. 26, 2024) is hereby incorporated by reference in its entirety.

### BACKGROUND

Immunotherapy, including adoptive cell therapy, has revolutionized the treatment of cancer but still with limited success in many tumor types. The potential for innate lymphoid cells (ILCs) has not been explored due to limited cell numbers and expansion challenges. Further, the anti-tumor effects of group 2 innate lymphoid cells (ILC2s) are poorly understood, particularly in humans. Therefore, eludication of mechanistic and therapeutic effects of human ILC2s and methods for effectively expanding human ILC2s cells are needed.

### BRIEF SUMMARY

In an aspect is provided a method of expanding a population of human group 2 innate lymphoid cells (ILC2), including contacting the population of human ILC2s with IL-2, IL-7, and IL-15, thereby forming a population of expanded human ILC2.

In another aspect is provided a population of expanded human group 2 innate lymphoid cells (ILC2), including at least 30% ILC2. In another aspect is provided a population of expanded human group 2 innate lymphoid cells (ILC2), including at least 50% ILC2. In another aspect is provided a population of expanded human group 2 innate lymphoid cells (ILC2), including at least 90% ILC2. In another aspect is provided a population of expanded human group 2 innate lymphoid cells (ILC2), including at least 95% ILC2.

In another aspect a method of treating cancer in a subject in need thereof is provided, the method including administering to the subject an effective amount of the population of expanded human ILC2s provided herein including embodiments thereof.

In an aspect is provided a genetically modified human group 2 innate lymphoid cell (ILC2) including a chimeric antigen receptor (CAR), wherein the CAR includes: i) an antibody region; and ii) a transmembrane domain.

In another aspect is provided a method of treating cancer in a subject in need thereof, including administering to the subject an effective amount of the genetically modified human ILC2s provided herein including embodiments thereof.

In an aspect is provided a population of expanded human ILC2, made by a method provided herein including embodiments thereof.

### BRIEF DESCRIPTION OF THE DRAWINGS

FIGS. 1A-1R. Expanded human ILC2s prevented the development of AML in vitro and in vivo. (FIG. 1A) Schematic of culture conditions. Total ILCs were isolated from PBMCs and cultured on OP9 stromal cells in the presence of IL-2, IL-7, and IL-15. Fourteen days later, ILC2s were sorted using flow cytometry and expanded in an ILC medium with cytokines. (FIG. 1B) Representative flow cytometry plots of the percentage of CD161⁺ cells (upper) and CRTH2⁺CD117⁺ cells (bottom) after sorting (n=4 individual donors). (FIG. 1C) Representative flow cytometry plots (left) and the bar graph of the expression of GATA3, EOMES, T-BET, and RORγt in expended ILC2s (Ex ILC2; n=4 individual donors). (FIG. 1D) The percentage of IL-4, IL-5, IL-9, and IL-13 produced by freshly isolated ILC2s and Ex ILC2s (n=4 individual donors). (FIG. 1E) The fold change of harvested Ex ILC2s vs. pre-seeded ILC2s isolated from PBMCs after 28 days (n=9 individual donors). (FIG. 1F) Representative images (5× magnification, scale bar, 200 μm, n=4 individual donors). (FIG. 1G) Ex ILC2s were cultured at the indicated ratios with MOML13, U937, or THP-1 for 48 h. Luciferase activity in the wells with tumor cells was measured with a luminescence microplate reader (n=7 individual donors). (FIG. 1H) Statistics of the percentages of dead cells identified by Annexin V and DAPI in MOLM13, U937, and THP1 cells (n=7 individual donors). (FIG. 1I) Design and procedures for FIG. 1J and FIG. 1K. (FIG. 1J) Images represent bioluminescence (BLI) at the indicated time points (n=4 individual mice in the No ILC2s+MOLM13 group individual mice; n=5 individual mice in the ILC2s+MOLM13 group). (FIG. 1K) Survival of mice injected with or without Ex ILC2s (n=4 individual mice in the No ILC2s+MOLM13 group individual mice; n=5 individual mice in the ILC2s+MOLM13 group). (FIG. 1L) Design and procedures for FIG. 1M and FIG. 1N. (FIG. 1M) Images represent BLI at the indicated time points (n=6 individual mice in the No ILC2s+U937 group individual mice; n=6 individual mice in the ILC2s+U937 group). (FIG. 1N) Survival of mice injected with or without Ex ILC2s (n=6 individual mice in the No ILC2s+U937 group individual mice; n=6 individual mice in the ILC2s+U937 group). (FIG. 1O) Design and procedures for FIG. 1P and FIG. 1Q. (FIG. 1P) Images represent BLI at the indicated time points (n=6 individual mice in the No ILC2s+MOLM13 group individual mice; n=6 individual mice in the ILC2s+MOLM13 group). (FIG. 1Q) Survival of mice injected with or without Ex ILC2s (n=6 individual mice in the No ILC2s+MOLM13 group individual mice; n=6 individual mice in the ILC2s+MOLM13 group). Data represent two (FIGS. 1B-1D) or three (FIGS. 1F, 1G, and 1H) independent experiments. Data are shown as mean±s.d. and were assessed by t test (FIGS. 1C, 1D, and 1E) and one-way ANOVA (FIG. 1H). Survival data are representative of two independent experiments and were analyzed by Kaplan-Meier survival analysis and log-rank test (FIGS. 1K, 1N, and 1Q). (FIG. 1R) ILC2s sorted from the lung of mice were cultured with IL-2 (100 IU), IL-7 (10 ng/ml), and IL-33 (10 ng/ml) for 5 days. The expanded ILC2s (ex ILC2) were stimulated with Leukocyte Activation Cocktail in the presence of BD GolgiPlug™ for 4 h. Intracellular staining for GzmB was performed using a Fix/Perm kit (BD Biosciences), followed by staining with PE-conjugated GzmB. Representative flow cytometry plots of the percentage of GzmB+ ex ILC2s. Fluorescence minus one (FMO) and T cells/NK cells served as negative and motive controls, respectively.

FIGS. 2A-2N. Ex ILC2s induce the pyroptosis and/or apoptosis of AML cells through granzyme B production. (FIG. 2A) Time-lapse microscopy images of co-cultures of Far red-labeled Ex ILC2s with CSFE-labeled AML cells cells in a medium containing DAPI. (FIG. 2B) Representative flow cytometry plots (left) and bar graphs (right) of the percentage of granzyme B (GZMB) and perforin in the freshly isolated ILC2s and Ex ILC2s (n=4 individual donors). (FIG. 2C) Confocal microscopy images of GZMB and perforin in Ex ILC2s (scale bar, 10 μm, n=3 individual donors). (FIG. 2D) Statistics of the percentage of GZMB in Ex ILC2s co-cultured with or without MOLM13, U937, or THP1 cells (effector cell/target cell (E/T) ratio=2:1; n=4 individual donors). (FIG. 2E) Supernatants from AML cells co-cultured with or without Ex ILC2s were collected and subjected to enzyme-linked immunosorbent assay (ELISA) to determine levels of GZMB (n=4 individual donors). (FIG. 2F) Left: Supernatants from AML cells co-cultured with or without wildtype (WT) Ex ILC2s or GZMB knockdown ILC2s (KD Ex ILC2) were collected and subjected to ELISA to determine levels of GZMB. Right: WT Ex ILC2s and GZMB KD Ex ILC2s were cultured at the 2:1 ratio with MOML13, U937, or THP-1 for 48 h. Luciferase activity in the wells with tumor cells was measured with a luminescence microplate reader (n=3 individual donors). (FIG. 2G) GSDME cleavage in MOLM13 incubated with or without Ex ILC2s at the 2:1 E/T ratios for 6 h, assessed by immunoblot. (FIG. 2H) Caspase 3 cleavage in MOLM13 incubated with or without Ex ILC2s was assessed by immunoblot. (FIG. 2I) GSDME and Caspase 3 cleavage in U937 incubated with or without Ex ILC2s was assessed by immunoblot. (FIG. 2J) GSDME and Caspase 3 cleavage in THP1 incubated with or without Ex ILC2s were assessed by immunoblot. (FIG. 2K) GSDME and Caspase, 3 cleavages in MOLM13, incubated with or without WT Ex ILC2s or GZMB KD Ex ILC2s, were assessed by immunoblot. (FIG. 2L) Caspase 3 cleavage in U937 incubated with or without WT Ex ILC2s or GZMB KD Ex ILC2s was assessed by immunoblot. (FIG. 2M) Expression of GSDME and caspase 3 in WT GSDME and KD GSDME MOLM13, assessed by immunoblot. (FIG. 2N) Representative flow cytometry plots (left) and statistics (right) of the percentage of dead cells (n=4 individual donors). Data represent two (FIGS. 2C, 2M, and 2N) or three (FIGS. 2A, 2B, 2D, 2E, 2F, 2G, 2H, 2I, 2J, 2K, and 2L) independent experiments. Data are shown as mean±s.d. and were assessed by t test (FIG. 2B) and one-way ANOVA (FIGS. 2D, 2E, 2F, and 2N). Survival data were analyzed by Kaplan-Meier survival analysis and log-rank test (FIGS. 2K, and 2N)

FIGS. 3A-3J. The cell-cell contact between Ex ILC2s and tumor cells through DNAM-1 is required for Ex ILC2-induced GZMB production. (FIG. 3A) Ex ILC2s were loaded into the bottom chambers of a 24-well Transwell plate. The top wells of the plate were loaded with or without MOLM13, U937, or THP1. Representative flow cytometry plots of the percentage of GzmB+Ex ILC2s. (FIG. 3B) Statistics of the percentage of GzmB+Ex ILC2s (n=8 individual donors). (FIG. 3C) Supernatants were collected from (FIG. 3A) and subjected to ELISA to determine levels of GZMB (n=8 individual donors). (FIG. 3D) Representative histograms showing the expression of NKD2D, DNAM-1, and NKp30 on Ex ILC2s (n=4 individual donors). (FIG. 3E) Ex ILC2s were co-cultured with or without MOLM13, U937, or THP1 in the presence or absence of an anti-NKG2D, anti-DNAM-1, or anti-NKp30 neutralizing antibody (10 μg/ml) or separately, for 48 h. Representative flow cytometry plots of the percentage of GzmB+Ex ILC2s (n=8 individual donors). (FIG. 3F) Statistics of the percentage of GzmB+Ex ILC2s (n=8 individual donors). (FIG. 3G) Supernatants were collected from (FIG. 3E) and subjected to ELISA to determine levels of GZMB (n=8 individual donors). (FIG. 3H) Ex ILC2s were co-cultured with or without WT, CD112-KO, CD155-KO, or CD112/CD155 double KO MOLM13, U937, or THP1, separately, for 48 h. Representative flow cytometry plots of the percentage of GzmB+Ex ILC2s (n=8 individual donors). (FIG. 3I) Statistics of the percentage of GzmB+Ex ILC2s (n=8 individual donors). (FIG. 3J) Supernatants were collected from (FIG. 3H) and subjected to ELISA to determine levels of GZMB (n=8 individual donors). Data are representative of two (FIG. 3D) or three (FIGS. 3A, 3B, 3C, 3E, 3F, 3G, 3H, 3I, and 3J) independent experiments. Data are shown as mean±s.d. and were assessed by one-way ANOVA (FIGS. 3B, 3C, 3D, 3F, 3G, 3I, and 3J).

FIGS. 4A-4D. ILC2s are dysregulated in human AML. (FIG. 4A) Representative histograms (left) and statistics (right) of the expression of DNAM-1 on the ILC2s from the blood of healthy donors and patients with AML (n=4 individual healthy donors and individual patients). (FIG. 4B) Representative flow cytometry plots and statistics of the percentage of GzmB+Ex ILC2s from the blood of healthy donors and patients with AML (n=4 individual healthy donors and individual patients). (FIG. 4C) Ex ILC2s were co-cultured with or without primary AML-derived blasts for 48 h. Representative histograms and statistics of the expression of DNAM-1 on the Ex ILC2s (n=4 individual healthy donors). Log-rank Mantel-Cox test. Data are representative of two (FIG. 4A and FIG. 4B) or three (FIG. 4C) independent experiments. Data are shown as mean±s.d. and were assessed by t test (FIGS. 4A, 4B, and 4C) one-way ANOVA. (FIG. 4D) Survival analyses based on ILC2s signature (CD117, PTGDR2, GATA3, IL9, IL13, HPGDS, S1PR1, TLE4, IL1RL1, IL17RB, and ICOS) in TCGA-LAML individual cohort (n=53 individual patients).

FIGS. 5A-5F. ILC2s have the ability to lyse solid tumor cells in vitro and exert anti-tumor function in vivo. (FIG. 5A) Ex ILC2s were cultured at the indicated effector (E)/target (T) ratio with A549, Capan-1, MIAPaCa-2, GLi36, LN229, U251, and GBM30 for 48 h. Statistics of the percentages of dead cells identified by Annexin V and DAPI (n=4 individual donors). (FIG. 5B) Representative real-time cell analysis (RTCA) data showing ILC2s lysis against A549, Capan-1, MIAPaCa-2, GLi36, LN229, and U251 at the indicated E/T ratios (n=4 individual donors). (FIG. 5C) Design and procedures for FIG. 5D and FIG. 5E. (FIG. 5D) Images represent BLI at the indicated time points (n=6 individual mice). Data represent two (FIGS. 5B-5D) or three (FIG. 5E) independent experiments. Data are shown as mean±s.d. Survival data are representative of one experiment and were analyzed by Kaplan-Meier survival analysis and log-rank test (FIG. 5D). (FIG. 5E) NK cells were first enriched from peripheral blood using the RosetteSep human enrichment kit to remove non-NK cells and red blood cells, and then total ILCs were enriched to remove non-ILCs from enriched NK cells using the pan-ILC isolation kit. Isolated ILCs were cultured on Notch ligand delta-like 1 (DL1)-transfected OP9 stromal cells with IL-2 and IL-7 in the presence or absence of IL-15. The kinetic growth curve of ILC2s (Lineage-CD161+CRTH2+CD117+) is presented. Donor 1; Donor 2. (FIG. 5F) The expression of CD122 was detected on ILC2s (Lineage-CD161+CRTH2+CD117+) on indicated days.

FIGS. 6A-6J. The gating strategy and phenotypes of human ILC2s and the frequency of ILC2s in the blood of patients with AML are decreased. (FIG. 6A) The gating strategy of human ILC1s, ILC2s, and ILC3s in the blood of healthy donors. (FIG. 6B) The gating strategy of expanded ILC2s. (FIG. 6C) Representative flow cytometry plots of the percentage of IFNγ⁺ and TNF⁺ ILC2s. (FIG. 6D) Representative flow cytometry plots of the percentage of IL-4, IL-5, IL-9, and IL-13 produced by freshly isolated or expanded ILC2s. (FIG. 6E) Representative histograms and statistics of the expression of IL-33R on the Ex ILC2s (n=3 individual donors). (FIG. 6F) Representative histograms and statistics of the expression of NKp30 on the Ex ILC2s (n=3 individual donors). (FIG. 6G) The gating strategy of human ILC2s in the blood of patients with AML. (FIG. 6H) Representative flow cytometry plots and statistics of the percentage of ILC2s among total ILCs in the blood of patients with AML (n=8 individual patients). (FIG. 6I) Statistics of the percentage of ILC2s among Lin negative cells in the blood of patients with AML (n=8 individual patients). (FIG. 6J) Correlation analyses on ILC2s signature (CD117, PTGDR2, GATA3, IL9, IL13, HPGDS, S1PR1, TLE4, IL1RL1, IL17RB, and ICOS) and leukemia cell (leukemia blasts and/or LSCs) signature (CD34, CD33, CD133, CD7, and CD13) in TCGA-LAML cohort. Two-tailed Pearson correlation test. Data are representative of two (FIG. 6E and FIG. 6F) or three (FIG. 6H and FIG. 6I) independent experiments. Data are shown as mean±s.d. and were assessed by t test (FIGS. 6E, 6F, 6H, and 6I).

FIGS. 7A-7G. ILC2s have an anti-AML function in vitro and in vivo. (FIG. 7A) Representative flow cytometry plots of the percentage of AML cell death when co-cultured with expanded ILC2s. (FIG. 7B) Graphic showing the percentage of AML cell death when co-cultured with freshly isolated ILC2s (n=4 individual healthy donors). (FIG. 7C) Statistics of the primary AML blast cell death percentage when co-cultured with expanded ILC2s (n=3 individual patients). (FIG. 7D) Representative flow cytometry plots and statistics of the percentage of ILC2s in the bone marrow, spleen, and liver of mice transplanted with MOLM13 and Ex ILC2s (n=3 individual mice). (FIG. 7E) Representative flow cytometry plots and statistics of the percentage of ILC2s in the bone marrow, spleen, and liver of mice transplanted with U937 and Ex ILC2s (n=3 individual mice). (FIG. 7F) Survival of mice injected with or without Ex ILC2s (n=6 individual mice in the No ILC2s+THP1 group individual mice; n=6 individual mice in the ILC2s+THP1 group). (FIG. 7G) Release assay of various cytokines in the sera from the mice implanted with the MOLM13 and Ex ILC2s, measured by a cytokine release array assay (n=4 individual mice). Data represent one (FIG. 7G), two (FIG. 7B and FIG. 7C), or three (FIG. 7A) independent experiments. Data are shown as mean±s.d. Survival data are representative of one experiment and were analyzed by Kaplan-Meier survival analysis and log-rank test (FIG. 7F).

FIGS. 8A-8D. ILC2s-induced AML cell death is neither due to the secretion of IL-4, IL-5, IL-9, and IL-13 nor conversion into cytotoxic NK cells/ILC1s, and ILC3s. (FIG. 8A) Representative images (5× magnification, scale bar, 200 μm) of AML cells (n=4 individual healthy donors). (FIG. 8B) Representative flow cytometry plots of TLC2s co-cultured with AML cells (n=4 individual healthy donors). (FIG. 8C) Representative flow cytometry plots of the percentage of GATA3 and RORγt expressed by Ex ILC2s. (FIG. 8D) RT-PCR analysis of human IL25, IL33, TSLP, PGD2, and I8S mRNA expression in MOLM13, U937, and THP1 cells.

FIGS. 9A-9I. ILC2-induced AML cell death through activating caspase 3. (FIG. 9A) Caspase 3 activity in the wells with MOLM13, U937, and THP1 cells was measured using a luminescence microplate reader (n=4 individual donors). Representative flow cytometry plots of (FIG. 9B) statistics of (FIG. 9C) the percentages of dead cells identified by Annexin V and DAPI in MOLM13, U937, and THP1 cells (n=4 individual donors). (FIG. 9D) Representative of microscopy images of GZMB and perforin in Ex ILC2s (scale bar, 200 μm, n=3 individual donors). (FIG. 9E) Representative flow cytometry plots of the percentage of GZMB in Ex ILC2s co-cultured with or without MOLM13, U937, and THP1 cells (n=4 individual healthy donors). (FIG. 9F) Representative flow cytometry plots and statistics of the percentage of perforin in Ex ILC2s co-cultured with or without MOLM13, U937, and THP1 cells (n=4 individual healthy donors). (FIG. 9G) Supernatants from AML cells co-cultured with or without Ex ILC2s were collected and subjected to ELISA to determine levels of perforin (n=4 individual healthy donors). (FIG. 9H) Immunoblotting measured the expression of GSDME in MOLM13, U937, and THP1 cells. (FIG. 91) Representative flow cytometry plots and statistics of the percentage of MOLM13 cell death (n=6 individual healthy donors). Data represent two (FIGS. 9A, 9B, 9C, and 9D) or three (FIGS. 9E, 9F, 9G, and 9I) independent experiments. Data are shown as mean±s.d. and were assessed by one-way ANOVA (FIGS. 9A, 9C, 9F, 9G, and 9I).

FIGS. 10A-10D. ILC2-induced AML cell death and the cleavage of caspase 3 and GSDME through the interaction between ILC2s and AML cells. (FIG. 10A) Representative flow cytometry plots and statistics of the percentage of AML cell death (n=4 individual healthy donors). (FIG. 10B) The percentage of AML cell death was measured by luminescence-based assay (n=4 individual healthy donors). (FIG. 10C) Immunoblotting measured the cleavage of GSDME and caspase 3 in MOLM13 cells (n=2 individual healthy donors). (FIG. 10D) Immunoblotting measured the cleavage of caspase 3 in U937 cells (n=2 individual healthy donors). Data represent two (FIGS. 10A, 10B, 10C, and 10D) independent experiments. Data are shown as mean±s.d. and were assessed by one-way ANOVA (FIGS. 10A and 10B).

FIGS. 11A-11E. Blockade of DNAM-1 and NKG2D decreased the lysis of AML cells by Ex ILC2s. (FIG. 11A) Representative histograms showing the expression of NKG2D ligands (MICA, MICB, ULBP1/2/5/6) in AML cells. (FIG. 11B) Representative histograms showing the expression of DNAM-1 ligands (CD112 and CD155) and NKp30 ligand (B7H6) in AML cells. (FIG. 11C) Representative flow cytometry plots and statistics of the percentage of perforin produced by ILC2s co-cultured with or without AML cells (n=4 individual healthy donors). (FIG. 11D) Representative images (5× magnification, scale bar, 200 m) of AML cells (n=4 individual healthy donors). (FIG. 11E) Statistics of the percentage of AML cell death co-cultured with or without Ex ILC2s in the absence or presence of NKG2D, DNAM-1, or NKp30 blockade antibodies (n=4 individual healthy donors). Data represent two (FIGS. 11A, 11B, and 11C) and three (FIGS. 11D and 11E) independent experiments. Data are shown as mean±s.d. and were assessed by one-way ANOVA (FIGS. 11C and 11E).

FIGS. 12A-12E. DNAM-1 knockout reduced the lysis of AML cells and the production of GZMB by Ex ILC2s. (FIG. 12A) Knockout of DNAM-1 on Ex ILC2s using CRISPR-Cas9 system. Representative flow cytometry plots and histograms showing the expression of DNAM-1 on WT ILC2s and DNAM-1 knockout ILC2s. (FIG. 12B) Representative flow cytometry plots and statistics of the percentage of AML cell death when co-cultured with or without WT ILC2s or DNAM KO ILC2s (n=4 individual healthy donors). (FIG. 12C) Supernatants from AML cells co-cultured with or without WT ILC2s or DNAM-1 KO ILC2s were collected and subjected to ELISA to determine levels of GZMB (n=4 individual healthy donors). (FIG. 12D) Representative flow cytometry plots and statistics of the percentage of GZMB production in Ex ILC2s when co-cultured with or without primary AML blasts in the absence or presence of DNAM-1 blockade antibody (n=4 individual healthy donors). (FIG. 12E) Supernatants were collected from (FIG. 12D) and subjected to ELISA to determine levels of GZMB (n=4 individual healthy donors). Data represent two (FIGS. 12D and 12E) and three (FIGS. 12A, 12B, and 12C) independent experiments. Data are shown as mean±s.d. and were assessed by one-way ANOVA (FIGS. 12C and 12E).

FIGS. 13A-13D. DNAM-1 ligands, CD112 and CD155, are required for ILC2-induced lysis of AML cells. (FIG. 13A) Knockout of CD112 and CD155 on AML cells using CRISPR-Cas9 system. Representative flow cytometry plots showing the expression of CD112 and CD155 on AML cells. (FIGS. 13B-13D) Ex ILC2s were co-cultured with or without WT, CD112-KO, CD155-KO, or CD112/CD155 double KO MOLM13, U937, or THP1, separately, for 48 h. Representative flow cytometry plots and statistics of the percentage of MOLM13 (FIG. 13B), U937 (FIG. 13C), and THP1 (FIG. 13D) cell death (n=8 individual healthy donors). Data represent three (FIGS. 13B, 13C, and 13D) independent experiments. Data are shown as mean±s.d. and were assessed by one-way ANOVA (FIGS. 13B, 13C, and 13D).

FIGS. 14A-14H. AML cells induce GZMB production by ILC2s via CD112/CD155-DNAM-1-AKT-FOXO1 axis. (FIG. 14A) Immunoblotting measured the phosphorylation of FOXO1 and AKT in ILC2s co-cultured with or without WT, CD112-KO, CD155-KO, or CD112/CD155 double KO U937 at various times. (FIG. 14B) Immunoblotting measured the phosphorylation of FOXO1 and AKT in ILC2s co-cultured with or without WT U937 in the absence or presence of DNAM-1 blockade antibodies. (FIG. 14C) Representative flow cytometry plots of the percentage of ILC2s cell death in the absence or presence of FOXO1 inhibitor or AKT inhibitor (n=3). (FIGS. 14D and 14E) Representative flow cytometry plots (FIG. 14D) and statistics (FIG. 14E) of the percentage of MOLM13, U937, and THP1 cell death when co-cultured with or without ILC2s in the absence or presence of FOXO1 inhibitor or AKT inhibitor (n=6 individual healthy donors). (FIGS. 14F and 14G) Representative flow cytometry plots (FIG. 14F) and statistics (FIG. 14G) of the percentage of GZMB production by ILC2s when co-cultured with or without AML cells in the absence or presence of FOXO1 inhibitor or AKT inhibitor (n=6 individual healthy donors). (FIG. 14H) Supernatants were collected from (FIGS. 14F and 14G) and subjected to ELISA to determine levels of GZMB (n=6 individual healthy donors). All data represent three independent experiments. Data are shown as mean±s.d. and were assessed by one-way ANOVA (FIGS. 14E, 14G, and 14H).

FIGS. 15A-15G. ILC2s have a comparable ability with NK cells to lyse AML cells and exert anti-tumor function in vitro and in vivo. (FIG. 15A) Statistics of the expression of PD-1, TIM3, and TIGIT on expanded ILC2s, expanded NK cells, or expanded T cells co-cultured with or without AML cells (n=4 individual healthy donors). Representative (FIG. 15B) histograms and (FIG. 15C) statistics of the expression of NKD2D, DNAM-1, and NKp30 on freshly isolated or expanded ILC2s, NK cells, or T cells (n=4 individual donors). (FIG. 15D) Statistics of the percentage of AML cell death when co-cultured with or without expanded ILC2s, expanded NK cells, or expanded T cells (n=4 individual healthy donors). (FIG. 15E) Design and procedures for FIG. 15F and FIG. 15G. (FIG. 15F) Images represent BLI at the indicated time points (n=5 individual mice in MOLM13 alone group and MOLM13+T cell group; n=6 individual mice in MOLM13+ILC2s group and MOLM13+NK cells group). (FIG. 15G) Survival of mice injected with or without Ex ILC2s, Ex NK cells, and Ex T cells (n=5 individual mice in MOLM13 alone group and MOLM13+T cell group; n=6 individual mice in MOLM13+ILC2s group and MOLM13+NK cells group). Data represent one (FIGS. 15E, 15F, and 15G) and two (FIGS. 15B, 15C, and 15D) or independent experiments. Data are shown as mean±s.d. Survival data were analyzed by Kaplan-Meier survival analysis and log-rank test (FIG. 15G).

FIGS. 16A-16G. ILC2s induce solid tumor cell death via apoptosis and/or pyroptosis. (FIGS. 16A-16C) Representative flow cytometry plots of the percentage of lung cancer cell (FIG. 16A), pancreatic cancer cell (FIG. 16B), and brain cancer cell (FIG. 16C) death when co-cultured with ILC2s (n=4 individual healthy donors). (FIG. 16D) Immunoblotting measured the expression of GSDME in multiple solid tumor cells. (FIG. 16E) Representative microscopy images showing the pyroptosis or apoptosis of A549, Capan-1, and MIAPaCa-2 co-cultured with or without ILC2s (n=4 individual healthy donors). (FIG. 16F) Representative microscopy image showing the pyroptosis or apoptosis of GLi36, LN229, U251, and GBM30 co-cultured with or without ILC2s (n=4 individual healthy donors). (FIG. 16G) Time-lapse microscopy images of the pyroptosis of A549, Capan-1, and GBM30 labeled with CSFE dye when co-cultured with co-cultures of Far Ex ILC2s in a medium containing DAPI.

### DETAILED DESCRIPTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non-limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of an adenoviral vector, which may be transduced into a cell. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, 7-carboxyglutamate, and O-phosphoserine. In embodiments, the amino acid side chain may be a non-natural amino acid side chain. In embodiments, the amino acid side chain is H,

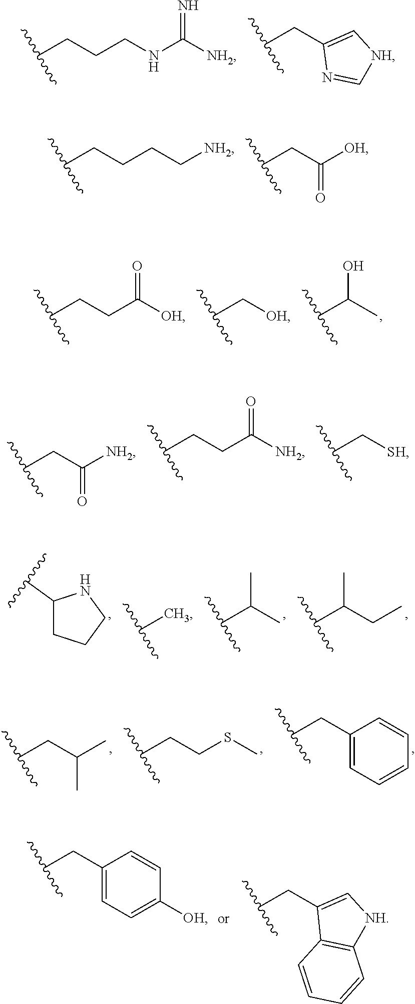

An amino acid residue in a protein “corresponds” to a given residue when it occupies the same essential structural position within the protein as the given residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein, the named protein includes any of the protein's naturally occurring forms, variants or homologs that maintain the protein activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “IL-2 protein” or “IL-2” as used herein includes any of the recombinant or naturally-occurring forms of interleukin-2 protein, also known as T-cell growth factor, TCGF, Aldesleukin, or variants or homologs thereof that maintain IL-2 activity (e.g. within at least 50%, 80%, 90%, 95%, 96%, 97%, 98%, 99% or 100% activity compared to IL-2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 protein. In embodiments, the IL-2 protein is substantially identical to the protein identified by the UniProt reference number P60568 or a variant or homolog having substantial identity thereto.

The term “IL-15 protein” or “IL-15” as used herein includes any of the recombinant or naturally-occurring forms of interleukin-15, or variants or homologs thereof that maintain IL-15 activity (e.g. within at least 50%, 80%, 90%, 95%, 96%, 97%, 98%, 99% or 100% activity compared to IL-15). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the UniProt reference number P40933 or a variant or homolog having substantial identity thereto.

The term “IL-7 protein” or “IL-7” as used herein includes any of the recombinant or naturally-occurring forms of interleukin-7, or variants or homologs thereof that maintain IL-7 activity (e.g. within at least 50%, 80%, 90%, 95%, 96%, 97%, 98%, 99% or 100% activity compared to IL-7). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-7 protein. In embodiments, the IL-7 protein is substantially identical to the protein identified by the UniProt reference number P13232 or a variant or homolog having substantial identity thereto.

The term “CD161 protein” or “CD161” as used herein includes any of the recombinant or naturally-occurring forms of CD161, also known as Killer cell lectin-like receptor subfamily B member 1, C-type lectin domain family 5 member B, HNKR-P1a (NKR-P1A), Natural killer cell surface protein P1A, or variants or homologs thereof that maintain CD161 activity (e.g. within at least 50%, 80%, 90%, 95%, 96%, 97%, 98%, 99% or 100% activity compared to CD161). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD161 protein. In embodiments, the CD161 protein is substantially identical to the protein identified by the UniProt reference number Q12918 or a variant or homolog having substantial identity thereto.

The term “CRTH2 protein” or “CRTH2” as used herein includes any of the recombinant or naturally-occurring forms of CRTH2, also known as Prostaglandin D2 receptor 2, Chemoattractant receptor-homologous molecule expressed on Th2 cells, G-protein coupled receptor 44, CD294, or variants or homologs thereof that maintain CRTH2 activity (e.g. within at least 50%, 80%, 90%, 95%, 96%, 97%, 98%, 99% or 100% activity compared to CRTH2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CRTH2 protein. In embodiments, the CRTH2 protein is substantially identical to the protein identified by the UniProt reference number Q9Y5Y4 or a variant or homolog having substantial identity thereto.

The term “CD117 protein” or “CD117” as used herein includes any of the recombinant or naturally-occurring forms of CD117, also known as Mast/stem cell growth factor receptor Kit, Piebald trait protein, Tyrosine-protein kinase Kit, Proto-oncogene c-Kit, or variants or homologs thereof that maintain CD117 activity (e.g. within at least 50%, 80%, 90%, 95%, 96%, 97%, 98%, 99% or 100% activity compared to CD117). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD117 protein. In embodiments, the CD117 protein is substantially identical to the protein identified by the UniProt reference number P10721 or a variant or homolog having substantial identity thereto.

The term “DNAX Accessory Molecule-1 protein” or “DNAX Accessory Molecule-1” as used herein includes any of the recombinant or naturally-occurring forms of DNAX Accessory Molecule-1 protein (DNAM-1), also known as CD226 antigen, or variants or homologs thereof that maintain DNAM-1 activity (e.g. within at least 50%, 80%, 90%, 95%, 96%, 97%, 98%, 99% or 100% activity compared to DNAM-1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring DNAM-1 protein. In embodiments, the DNAM-1 protein is substantially identical to the protein identified by the UniProt reference number Q15762 or a variant or homolog having substantial identity thereto.

The term “Perforin protein” or “Perforin” as used herein includes any of the recombinant or naturally-occurring forms of perforin (PRF), also known as P1, Cytolysin or variants or homologs thereof that maintain perforin activity (e.g. within at least 50%, 80%, 90%, 95%, 96%, 97%, 98%, 99% or 100% activity compared to perforin). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring perforin protein. In embodiments, the perforin protein is substantially identical to the protein identified by the UniProt reference number P14222 or a variant or homolog having substantial identity thereto.

The term “granzyme B protein” or “granzyme B” as used herein includes any of the recombinant or naturally-occurring forms of granzyme (GZMB), also known as P1, Cytolysin or variants or homologs thereof that maintain granzyme B activity (e.g. within at least 50%, 80%, 90%, 95%, 96%, 97%, 98%, 99% or 100% activity compared to granzyme B). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring granzyme B protein. In embodiments, the granzyme protein is substantially identical to the protein identified by the UniProt reference number P10144 or a variant or homolog having substantial identity thereto.

The term “IL-4 protein” or “IL-4” as used herein includes any of the recombinant or naturally-occurring forms of interleukin-4, also known as B-cell stimulatory factor 1, Binetrakin, Lymphocyte stimulatory factor 1, or variants or homologs thereof that maintain IL-4 activity (e.g. within at least 50%, 80%, 90%, 95%, 96%, 97%, 98%, 99% or 100% activity compared to IL-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-4 protein. In embodiments, the IL-4 protein is substantially identical to the protein identified by the UniProt reference number P05112 or a variant or homolog having substantial identity thereto.

The term “IL-5 protein” or “IL-5” as used herein includes any of the recombinant or naturally-occurring forms of interleukin-5, also known as B-cell differentiation factor I, Eosinophil differentiation factor, T-cell replacing factor, or variants or homologs thereof that maintain IL-5 activity (e.g. within at least 50%, 80%, 90%, 95%, 96%, 97%, 98%, 99% or 100% activity compared to IL-5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-5 protein. In embodiments, the IL-5 protein is substantially identical to the protein identified by the UniProt reference number P05113 or a variant or homolog having substantial identity thereto.

The term “IL-9 protein” or “IL-9” as used herein includes any of the recombinant or naturally-occurring forms of interleukin-9, also known as Cytokine P40, T-cell growth factor P40, or variants or homologs thereof that maintain IL-9 activity (e.g. within at least 50%, 80%, 90%, 95%, 96%, 97%, 98%, 99% or 100% activity compared to IL-9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-9 protein. In embodiments, the IL-9 protein is substantially identical to the protein identified by the UniProt reference number P15248 or a variant or homolog having substantial identity thereto.

The term “IL-13 protein” or “IL-13” as used herein includes any of the recombinant or naturally-occurring forms of interleukin-13, or variants or homologs thereof that maintain IL-13 activity (e.g. within at least 50%, 80%, 90%, 95%, 96%, 97%, 98%, 99% or 100% activity compared to IL-13). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-13 protein. In embodiments, the IL-13 protein is substantially identical to the protein identified by the UniProt reference number P35225 or a variant or homolog having substantial identity thereto.

The term “IL-33R protein” or “IL-33R” as used herein includes any of the recombinant or naturally-occurring forms of Interleukin-1 receptor-like 1, also known as ST2, or variants or homologs thereof that maintain IL-33R activity (e.g. within at least 50%, 80%, 90%, 95%, 96%, 97%, 98%, 99% or 100% activity compared to IL-13). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-33R protein. In embodiments, the IL-33R protein is substantially identical to the protein identified by the UniProt reference number Q01638 or a variant or homolog having substantial identity thereto.

The term “NKp30 protein” or “NKp30” as used herein includes any of the recombinant or naturally-occurring forms of Natural killer cell p30-related protein (NKp30), also known as Natural cytotoxicity triggering receptor 3, Activating natural killer receptor p30, CD337, or variants or homologs thereof that maintain NKp30 activity (e.g. within at least 50%, 80%, 90%, 95%, 96%, 97%, 98%, 99% or 100% activity compared to NKp30). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring NKp30 protein. In embodiments, the NKp30 protein is substantially identical to the protein identified by the UniProt reference number 014931 or a variant or homolog having substantial identity thereto

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa. In embodiments, the antibody is an scFv.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “recombinant” when used with reference, e.g., to a virus, cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, a recombinant virus is generated by combining portions of nucleic acids using recombinant nucleic acid technology. For example, a recombinant virus may be generated by replacing one or more viral genes with an exogenous gene. In instances, recombinant cells express genes that are not found within the native (non-recombinant) form of the cell or express native genes that are otherwise abnormally expressed, under expressed or not expressed at all. For example, a recombinant ILC2s may be generated by introducing a nucleic acid encoding a chimeric antigen receptor into the ILC2. A recombinant ILC2s may therefore express a chimeric antigen receptor not found within the native ILC2. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous protein” as referred to herein is a protein that does not originate from the cell or organism it is expressed by. Conversely, the term “endogenous” refers to a molecule or substance that is native to, or originates within, a given cell or organism.

As used herein, the terms “isolated”, “isolating” or the like when applied to a cell, denotes that the cell has been separated from its natural environment or from components of the environment in which it is produced. For example, a naturally occurring cell (e.g., a ILC2) present in a living animal, including humans, is not isolated. However, the same cell, when separated from some or all of the coexisting materials in the animal, is considered isolated. As a further example, a population of ILC2s that are present in a biological sample obtained from a human subject would be considered isolated. It should be appreciated that cells obtained from such a sample using further purification steps would also be referred to as isolated. For example, in embodiments, a population of ILC2s may be isolated from non-ILC2 cells (e.g. T cells, NK cells, etc.) in a population of cells obtained from a subject, wherein the population of cells includes ILC2s and non-ILC2. In embodiments, isolating a population of ILC2s refers to removing non-ILC2 cells (e.g. T cells, NK cells, etc.) from a population of cells including ILC2s and non-ILC2 cells. In embodiments, isolating a population of ILC2s refers to removing the population of ILC2s from the population of cells including ILC2s and non-ILC2 cells (e.g. T cells, NK cells, red blood cells, etc.).

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

“Biological sample” or “sample” refer to materials obtained from or derived from a subject or patient. In embodiments, the biological sample comprises periperal blood. In embodiments, the biological sample comprises peripheral blood mononuclear cells (PBMCs). In embodiments, the biological sample includes ILC2. In embodiments, the biological sample includes ILC2s and non-ILC2. In embodiments, the biological sample includes sections of tissues such as biopsy and autopsy samples, and frozen sections taken for histological purposes. In embodiments, such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the terms “ILC2” and “group 2 innate lymphoid cell”, also referred to as “type 2 innate lymphoid cell”, are used are used in accordance with their plain ordinary meaning and refer to a type of innate lymphoid cell that expresses type 2 cytokines, including IL-5, IL-5, IL-9, and IL-13. Native ILC2s (e.g. an ILC2s that has not been genetically modified) lack antigen specific B cell receptors and T cell receptors. ILC2s may be characterized by GATA3⁺ expression, and are typically activated by acute and chronic tissue damage and extracellular bacteria, parasites, and allergens.

As used herein, the terms “ILC1” and “group 1 innate lymphoid cell”, also referred to as “type 1 innate lymphoid cell”, are used are used in accordance with their plain ordinary meaning and refer to a heterogeneous population of innate lymphoid cells in the peripheral tissues that express IFNγ. A subset of ILC1 may include NK cells. ILC1 are typically characterized as T-bet⁺ expressing cells, and primarily respond to tissue inflammation and intracellular pathogens.

As used herein, the terms “ILC3” and “group 3 innate lymphoid cell”, also referred to as “type 3 innate lymphoid cell”, are used are used in accordance with their plain ordinary meaning and refer to a type of innate lymphoid cells that express RORγt⁺. ILC3 are activated in response to local inflammation and extracellular microbes, including commensal microbiota, pathogenic microbes, and fungi. ILC2s may express IL-17, IL-22, or both upon stimulation with IL-23 and IL-1β, and also IL-2 and granulocyte-macrophage colony-stimulating factor (GM-CSF) in the case of intestinal ILC3.

As used herein, the terms “natural killer cells” and “NK cells” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction. In embodiments, NK cells are identified by the presence of CD56 and the absence of CD3. NK cells may be capable of recognizing and killing stressed cells in the absence of antibodies and MHC.

“Allogeneic” is used in accordance with its plain and ordinary meaning and includes cells or tissues derived from different individuals of the same species. The term “allogeneic transplant” or “allogeneic transfusion” refers to the transfer of biological material to a recipient from a genetically non-identical donor of the same species. For example, an allogeneic transplant may include transfer of tissue, cells or an organ to a recipient that is genetically non-identical to the donor. In embodiments, the allogeneic cells are allogeneic human ILC2.

“Autologous” is used in accordance with its plain and ordinary meaning and includes cells or tissues derived from the same individual. In embodiments, the autologous cells are autologous human ILC2. An autolous ILC2s may be taken from an individual, expanded using a method provided herein including embodiments thereof, before being put back into the same individual.

“Marker” or “biomarker” is used in accordance with its plain ordinary meaning and refers to a measurable substance or compound in an biological sample that is indicative of a process or of a condition or a disease. A marker may be indicative of a cell subset (e.g. a ILC2). In embodiments, the marker is CD161, CRTH2, CD117, CD122, DNAM-1, GZMB, IL-33R, or NKp30, or a combination thereof.

The term “associated” or “associated with” in the context of a substance or substance activity or function associated with a disease (e.g., a protein associated disease, a cancer (e.g., leukemia)) means that the disease (e.g., cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

As used herein, the term “cancer” refers to all types of cancer, neoplasm or malignant tumors found in mammals (e.g., humans), including leukemia, lymphoma, carcinomas and sarcomas. Cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease (e.g. cancer) or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “donor” refers to a subject who provides an organ, tissue, or group of cells for transplantation to a recipient, wherein the donor is not genetically identical to the recipient. For example, a donor may provide a population of ILC2s cells to a subject in need thereof. In embodiments, a donor is healthy, e.g. does not suffer from cancer.

The term “healthy patient” or “healthy subject” as used herein refers to a subject that does not have cancer. In embodiments, the cancer is leukemia. As used herein, the healthy subject does not have leukemia. In embodiments, the healthy subject does not have AML.

A “effective amount” or “therapeutically effective amount” are used interchangeably and refer to an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). For any composition (e.g. ILC2s cell composition) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning in the art and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g., mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g., mice) and from humans.

### Methods of Expanding

Provided herein, inter alia, are methods for expanding a population of human type 2 innate lymphoid cells (ILC2s). As described herein, Applicant has discovered methods of expanding a population of human ILC2s in the presence of one or more compounds (e.g. IL-2, IL-7, IL-15) that promote proliferation and/or survival of the a population of human ILC2s. In embodiments, the methods provided herein are useful for generating a population of expanded human ILC2s, wherein the population of expanded human ILC2s include ILC2s expressing proteins (e.g. granzyme B, etc) effective for killing cancer cells (e.g. leukemia cells). In embodiments, the proteins (e.g. granzyme B) expressed by the expanded human ILC2s produced by the methods provided herein are typically found in lower levels in populations of ILC2s generated by other methods previously known in the art, or in populations of ILC2s derived from non-human mammals (e.g. mouse ILC2). Thus, in an aspect is provided a method of expanding a population of human group 2 innate lymphoid cells (ILC2s), including contacting the population of human ILC2s with IL-2, IL-7, and IL-15, thereby forming a population of expanded human ILC2s. The term “expand” as used herein, refers to increasing or proliferating the number of cells (e.g. human ILC2s) in a cell culture. The culture medium may include growth factors, serum, cytokines or and other additives to promote cell growth and/or differentiation. Thus, “expanding human ILC2s” or “expanding a population of human ILC2s” refers to the process of proliferating human ILC2s in a cell culture, thereby forming a population of expanded human ILC2s.

In embodiments, the population of human ILC2s is obtained from a subject having cancer. In embodiments, the cancer is lymphoma, leukemia, lung cancer pancreatic cancer, or brain cancer. In embodiments, the population of human ILC2s is obtained from a donor. In embodiments, the donor is a healthy subject (e.g. a subject who does not have cancer).

In embodiments, the population of human ILC2s obtained from the subject or the donor includes ILC2s and non-ILC2 cells (e.g. NK cells, T cells, etc). For example, for the method provided herein, in embodiments, the method includes obtaining a population of cells from a subject or a donor, wherein the population of cells includes a population of human ILC2s and a population of non-ILC2 cells. Thus, in embodiments, expanding a population of human ILC2s includes contacting a population of cells with IL-2, IL-7, and IL-15, wherein the population of cells includes a population of human ILC2s and a population of non-ILC2 cells. In embodiments, expanding a population of human ILC2s includes contacting a population of human ILC2s with IL-2, IL-7, and IL-15, wherein the population of human ILC2s includes a population of non-ILC2 cells.

In embodiments, the population of human ILC2s is contacted with about 10 IU to about 10000 IU of the IL-2, about 0.1 ng/mL to about 500 ng/mL of the IL-7, and about 0.1 ng/mL to about 500 ng/mL of the IL-15. In embodiments, the population of human ILC2s is contacted with about 100 IU to about 10000 IU of the IL-2. In embodiments, the population of human ILC2s is contacted with about 200 IU to about 10000 IU of the IL-2. In embodiments, the population of human ILC2s is contacted with about 300 IU to about 10000 IU of the IL-2. In embodiments, the population of human ILC2s is contacted with about 400 IU to about 10000 IU of the IL-2. In embodiments, the population of human ILC2s is contacted with about 500 IU to about 10000 IU of the IL-2. In embodiments, the population of human ILC2s is contacted with about 600 IU to about 10000 IU of the IL-2. In embodiments, the population of human ILC2s is contacted with about 700 IU to about 10000 IU of the IL-2. In embodiments, the population of human ILC2s is contacted with about 800 IU to about 10000 IU of the IL-2. In embodiments, the population of human ILC2s is contacted with about 900 IU to about 10000 IU of the IL-2. In embodiments, the population of human ILC2s is contacted with about 1000 IU to about 10000 IU of the IL-2. In embodiments, the population of human ILC2s is contacted with about 1100 IU to about 10000 IU of the IL-2. In embodiments, the population of human ILC2s is contacted with about 1200 IU to about 10000 IU of the IL-2. In embodiments, the population of human ILC2s is contacted with about 1300 IU to about 10000 IU of the IL-2. In embodiments, the population of human ILC2s is contacted with about 1400 IU to about 10000 IU of the IL-2. In embodiments, the population of human ILC2s is contacted with about 1500 IU to about 10000 IU of the IL-2. In embodiments, the population of human ILC2s is contacted with about 1600 IU to about 10000 IU of the IL-2. In embodiments, the population of human ILC2s is contacted with about 1700 IU to about 10000 IU of the IL-2. In embodiments, the population of human ILC2s is contacted with about 1800 IU to about 10000 IU of the IL-2. In embodiments, the population of human ILC2s is contacted with about 1900 IU to about 10000 IU of the IL-2. In embodiments, the population of human ILC2s is contacted with about 2000 IU to about 10000 IU of the IL-2. In embodiments, the population of human ILC2s is contacted with about 2100 IU to about 10000 IU of the IL-2. In embodiments, the population of human ILC2s is contacted with about 2200 IU to about 10000 IU of the IL-2. In embodiments, the population of human ILC2s is contacted with about 2300 IU to about 10000 IU of the IL-2. In embodiments, the population of human ILC2s is contacted with about 2400 IU to about 10000 IU of the IL-2. In embodiments, the population of human ILC2s is contacted with about 2500 IU to about 10000 IU of the IL-2. In embodiments, the population of human ILC2s is contacted with about 2600 IU to about 10000 IU of the IL-2. In embodiments, the population of human ILC2s is contacted with about 2700 IU to about 10000 IU of the IL-2. In embodiments, the population of human ILC2s is contacted with about 2800 IU to about 10000 IU of the IL-2. In embodiments, the population of human ILC2s is contacted with about 2900 IU to about 10000 IU of the IL-2. In embodiments, the population of human ILC2s is contacted with about 3000 IU to about 10000 IU of the IL-2. In embodiments, the population of human ILC2s is contacted with about 3100 IU to about 10000 IU of the IL-2. In embodiments, the population of human ILC2s is contacted with about 3200 IU to about 10000 IU of the IL-2. In embodiments, the population of human ILC2s is contacted with about 3300 IU to about 10000 IU of the IL-2. In embodiments, the population of human ILC2s is contacted with about 3400 IU to about 10000 IU of the IL-2. In embodiments, the population of human ILC2s is contacted with about 3500 IU to about 10000 IU of the IL-2. In embodiments, the population of human ILC2s is contacted with about 3600 IU to about 10000 IU of the IL-2. In embodiments, the population of human ILC2s is contacted with about 3700 IU to about 10000 IU of the IL-2. In embodiments, the population of human ILC2s is contacted with about 3800 IU to about 10000 IU of the IL-2. In embodiments, the population of human ILC2s is contacted with about 3900 IU to about 10000 IU of the IL-2. In embodiments, the population of human ILC2s is contacted with about 4000 IU to about 10000 IU of the IL-2. In embodiments, the population of human ILC2s is contacted with about 4100 IU to about 10000 IU of the IL-2. In embodiments, the population of human ILC2s is contacted with about 4200 IU to about 10000 IU of the IL-2. In embodiments, the population of human ILC2s is contacted with about 4300 IU to about 10000 IU of the IL-2. In embodiments, the population of human ILC2s is contacted with about 4400 IU to about 10000 IU of the IL-2. In embodiments, the population of human ILC2s is contacted with about 4500 IU to about 10000 IU of the IL-2. In embodiments, the population of human ILC2s is contacted with about 4600 IU to about 10000 IU of the IL-2. In embodiments, the population of human ILC2s is contacted with about 4700 IU to about 10000 IU of the IL-2. In embodiments, the population of human ILC2s is contacted with about 4800 IU to about 10000 IU of the IL-2. In embodiments, the population of human ILC2s is contacted with about 4900 IU to about 10000 IU of the IL-2. In embodiments, the population of human ILC2s is contacted with about 5000 IU to about 10000 IU of the IL-2. In embodiments, the population of human ILC2s is contacted with about 5100 IU to about 10000 IU of the IL-2. In embodiments, the population of human ILC2s is contacted with about 5200 IU to about 10000 IU of the IL-2. In embodiments, the population of human ILC2s is contacted with about 5300 IU to about 10000 IU of the IL-2. In embodiments, the population of human ILC2s is contacted with about 5400 IU to about 10000 IU of the IL-2. In embodiments, the population of human ILC2s is contacted with about 5500 IU to about 10000 IU of the IL-2. In embodiments, the population of human ILC2s is contacted with about 5600 IU to about 10000 IU of the IL-2. In embodiments, the population of human ILC2s is contacted with about 5700 IU to about 10000 IU of the IL-2. In embodiments, the population of human ILC2s is contacted with about 5800 IU to about 10000 IU of the IL-2. In embodiments, the population of human ILC2s is contacted with about 5900 IU to about 10000 IU of the IL-2. In embodiments, the population of human ILC2s is contacted with about 6000 IU to about 10000 IU of the IL-2. In embodiments, the population of human ILC2s is contacted with about 6100 IU to about 10000 IU of the IL-2. In embodiments, the population of human ILC2s is contacted with about 6200 IU to about 10000 IU of the IL-2. In embodiments, the population of human ILC2s is contacted with about 6300 IU to about 10000 IU of the IL-2. In embodiments, the population of human ILC2s is contacted with about 6400 IU to about 10000 IU of the IL-2. In embodiments, the population of human ILC2s is contacted with about 6500 IU to about 10000 IU of the IL-2. In embodiments, the population of human ILC2s is contacted with about 6600 IU to about 10000 IU of the IL-2. In embodiments, the population of human ILC2s is contacted with about 6700 IU to about 10000 IU of the IL-2. In embodiments, the population of human ILC2s is contacted with about 6800 IU to about 10000 IU of the IL-2. In embodiments, the population of human ILC2s is contacted with about 6900 IU to about 10000 IU of the IL-2. In embodiments, the population of human ILC2s is contacted with about 7000 IU to about 10000 IU of the IL-2. In embodiments, the population of human ILC2s is contacted with about 7100 IU to about 10000 IU of the IL-2. In embodiments, the population of human ILC2s is contacted with about 7200 IU to about 10000 IU of the IL-2. In embodiments, the population of human ILC2s is contacted with about 7300 IU to about 10000 IU of the IL-2. In embodiments, the population of human ILC2s is contacted with about 7400 IU to about 10000 IU of the IL-2. In embodiments, the population of human ILC2s is contacted with about 7500 IU to about 10000 IU of the IL-2. In embodiments, the population of human ILC2s is contacted with about 7600 IU to about 10000 IU of the IL-2. In embodiments, the population of human ILC2s is contacted with about 7700 IU to about 10000 IU of the IL-2. In embodiments, the population of human ILC2s is contacted with about 7800 IU to about 10000 IU of the IL-2. In embodiments, the population of human ILC2s is contacted with about 7900 IU to about 10000 IU of the IL-2. In embodiments, the population of human ILC2s is contacted with about 8000 IU to about 10000 IU of the IL-2. In embodiments, the population of human ILC2s is contacted with about 8100 IU to about 10000 IU of the IL-2. In embodiments, the population of human ILC2s is contacted with about 8200 IU to about 10000 IU of the IL-2. In embodiments, the population of human ILC2s is contacted with about 8300 IU to about 10000 IU of the IL-2. In embodiments, the population of human ILC2s is contacted with about 8400 IU to about 10000 IU of the IL-2. In embodiments, the population of human ILC2s is contacted with about 8500 IU to about 10000 IU of the IL-2. In embodiments, the population of human ILC2s is contacted with about 8600 IU to about 10000 IU of the IL-2. In embodiments, the population of human ILC2s is contacted with about 8700 IU to about 10000 IU of the IL-2. In embodiments, the population of human ILC2s is contacted with about 8800 IU to about 10000 IU of the IL-2. In embodiments, the population of human ILC2s is contacted with about 8900 IU to about 10000 IU of the IL-2. In embodiments, the population of human ILC2s is contacted with about 9000 IU to about 10000 IU of the IL-2. In embodiments, the population of human ILC2s is contacted with about 9100 IU to about 10000 IU of the IL-2. In embodiments, the population of human ILC2s is contacted with about 9200 IU to about 10000 IU of the IL-2. In embodiments, the population of human ILC2s is contacted with about 9300 IU to about 10000 IU of the IL-2. In embodiments, the population of human ILC2s is contacted with about 9400 IU to about 10000 IU of the IL-2. In embodiments, the population of human ILC2s is contacted with about 9500 IU to about 10000 IU of the IL-2. In embodiments, the population of human ILC2s is contacted with about 9600 IU to about 10000 IU of the IL-2. In embodiments, the population of human ILC2s is contacted with about 9700 IU to about 10000 IU of the IL-2. In embodiments, the population of human ILC2s is contacted with about 9800 IU to about 10000 IU of the IL-2. In embodiments, the population of human ILC2s is contacted with about 9900 IU to about 10000 IU of the IL-2.

In embodiments, the population of human ILC2s is contacted with about 100 IU to about 9900 IU of the IL-2. In embodiments, the population of human ILC2s is contacted with about 100 IU to about 9800 IU of the IL-2. In embodiments, the population of human ILC2s is contacted with about 100 IU to about 9700 IU of the IL-2. In embodiments, the population of human ILC2s is contacted with about 100 IU to about 9600 IU of the IL-2. In embodiments, the population of human ILC2s is contacted with about 100 IU to about 9500 IU of the IL-2. In embodiments, the population of human ILC2s is contacted with about 100 IU to about 9400 IU of the IL-2. In embodiments, the population of human ILC2s is contacted with about 100 IU to about 9300 IU of the IL-2. In embodiments, the population of human ILC2s is contacted with about 100 IU to about 9200 IU of the IL-2. In embodiments, the population of human ILC2s is contacted with about 100 IU to about 9100 IU of the IL-2. In embodiments, the population of human ILC2s is contacted with about 100 IU to about 9000 IU of the IL-2. In embodiments, the population of human ILC2s is contacted with about 100 IU to about 8900 IU of the IL-2. In embodiments, the population of human ILC2s is contacted with about 100 IU to about 8800 IU of the IL-2. In embodiments, the population of human ILC2s is contacted with about 100 IU to about 8700 IU of the IL-2. In embodiments, the population of human ILC2s is contacted with about 100 IU to about 8600 IU of the IL-2. In embodiments, the population of human ILC2s is contacted with about 100 IU to about 8500 IU of the IL-2. In embodiments, the population of human ILC2s is contacted with about 100 IU to about 8400 IU of the IL-2. In embodiments, the population of human ILC2s is contacted with about 100 IU to about 8300 IU of the IL-2. In embodiments, the population of human ILC2s is contacted with about 100 IU to about 8200 IU of the IL-2. In embodiments, the population of human ILC2s is contacted with about 100 IU to about 8100 IU of the IL-2. In embodiments, the population of human ILC2s is contacted with about 100 IU to about 8000 IU of the IL-2. In embodiments, the population of human ILC2s is contacted with about 100 IU to about 7900 IU of the IL-2. In embodiments, the population of human ILC2s is contacted with about 100 IU to about 7800 IU of the IL-2. In embodiments, the population of human ILC2s is contacted with about 100 IU to about 7700 IU of the IL-2. In embodiments, the population of human ILC2s is contacted with about 100 IU to about 7600 IU of the IL-2. In embodiments, the population of human ILC2s is contacted with about 100 IU to about 7500 IU of the IL-2. In embodiments, the population of human ILC2s is contacted with about 100 IU to about 7400 IU of the IL-2. In embodiments, the population of human ILC2s is contacted with about 100 IU to about 7300 IU of the IL-2. In embodiments, the population of human ILC2s is contacted with about 100 IU to about 7200 IU of the IL-2. In embodiments, the population of human ILC2s is contacted with about 100 IU to about 7100 IU of the IL-2. In embodiments, the population of human ILC2s is contacted with about 100 IU to about 7000 IU of the IL-2. In embodiments, the population of human ILC2s is contacted with about 100 IU to about 6900 IU of the IL-2. In embodiments, the population of human ILC2s is contacted with about 100 IU to about 6800 IU of the IL-2. In embodiments, the population of human ILC2s is contacted with about 100 IU to about 6700 IU of the IL-2. In embodiments, the population of human ILC2s is contacted with about 100 IU to about 6600 IU of the IL-2. In embodiments, the population of human ILC2s is contacted with about 100 IU to about 6500 IU of the IL-2. In embodiments, the population of human ILC2s is contacted with about 100 IU to about 6400 IU of the IL-2. In embodiments, the population of human ILC2s is contacted with about 100 IU to about 6300 IU of the IL-2. In embodiments, the population of human ILC2s is contacted with about 100 IU to about 6200 IU of the IL-2. In embodiments, the population of human ILC2s is contacted with about 100 IU to about 6100 IU of the IL-2. In embodiments, the population of human ILC2s is contacted with about 100 IU to about 6000 IU of the IL-2. In embodiments, the population of human ILC2s is contacted with about 100 IU to about 5900 IU of the IL-2. In embodiments, the population of human ILC2s is contacted with about 100 IU to about 5800 IU of the IL-2. In embodiments, the population of human ILC2s is contacted with about 100 IU to about 5700 IU of the IL-2. In embodiments, the population of human ILC2s is contacted with about 100 IU to about 5600 IU of the IL-2. In embodiments, the population of human ILC2s is contacted with about 100 IU to about 5500 IU of the IL-2. In embodiments, the population of human ILC2s is contacted with about 100 IU to about 5400 IU of the IL-2. In embodiments, the population of human ILC2s is contacted with about 100 IU to about 5300 IU of the IL-2. In embodiments, the population of human ILC2s is contacted with about 100 IU to about 5200 IU of the IL-2. In embodiments, the population of human ILC2s is contacted with about 100 IU to about 5100 IU of the IL-2. In embodiments, the population of human ILC2s is contacted with about 100 IU to about 5000 IU of the IL-2. In embodiments, the population of human ILC2s is contacted with about 100 IU to about 4900 IU of the IL-2. In embodiments, the population of human ILC2s is contacted with about 100 IU to about 4800 IU of the IL-2. In embodiments, the population of human ILC2s is contacted with about 100 IU to about 4700 IU of the IL-2. In embodiments, the population of human ILC2s is contacted with about 100 IU to about 4600 IU of the IL-2. In embodiments, the population of human ILC2s is contacted with about 100 IU to about 4500 IU of the IL-2. In embodiments, the population of human ILC2s is contacted with about 100 IU to about 4400 IU of the IL-2. In embodiments, the population of human ILC2s is contacted with about 100 IU to about 4300 IU of the IL-2. In embodiments, the population of human ILC2s is contacted with about 100 IU to about 4200 IU of the IL-2. In embodiments, the population of human ILC2s is contacted with about 100 IU to about 4100 IU of the IL-2. In embodiments, the population of human ILC2s is contacted with about 100 IU to about 4000 IU of the IL-2. In embodiments, the population of human ILC2s is contacted with about 100 IU to about 3900 IU of the IL-2. In embodiments, the population of human ILC2s is contacted with about 100 IU to about 3800 IU of the IL-2. In embodiments, the population of human ILC2s is contacted with about 100 IU to about 3700 IU of the IL-2. In embodiments, the population of human ILC2s is contacted with about 100 IU to about 3600 IU of the IL-2. In embodiments, the population of human ILC2s is contacted with about 100 IU to about 3500 IU of the IL-2. In embodiments, the population of human ILC2s is contacted with about 100 IU to about 3400 IU of the IL-2. In embodiments, the population of human ILC2s is contacted with about 100 IU to about 3300 IU of the IL-2. In embodiments, the population of human ILC2s is contacted with about 100 IU to about 3200 IU of the IL-2. In embodiments, the population of human ILC2s is contacted with about 100 IU to about 3100 IU of the IL-2. In embodiments, the population of human ILC2s is contacted with about 100 IU to about 3000 IU of the IL-2. In embodiments, the population of human ILC2s is contacted with about 100 IU to about 2900 IU of the IL-2. In embodiments, the population of human ILC2s is contacted with about 100 IU to about 2800 IU of the IL-2. In embodiments, the population of human ILC2s is contacted with about 100 IU to about 2700 IU of the IL-2. In embodiments, the population of human ILC2s is contacted with about 100 IU to about 2600 IU of the IL-2. In embodiments, the population of human ILC2s is contacted with about 100 IU to about 2500 IU of the IL-2. In embodiments, the population of human ILC2s is contacted with about 100 IU to about 2400 IU of the IL-2. In embodiments, the population of human ILC2s is contacted with about 100 IU to about 2300 IU of the IL-2. In embodiments, the population of human ILC2s is contacted with about 100 IU to about 2200 IU of the IL-2. In embodiments, the population of human ILC2s is contacted with about 100 IU to about 2100 IU of the IL-2. In embodiments, the population of human ILC2s is contacted with about 100 IU to about 2000 IU of the IL-2. In embodiments, the population of human ILC2s is contacted with about 100 IU to about 1900 IU of the IL-2. In embodiments, the population of human ILC2s is contacted with about 100 IU to about 1800 IU of the IL-2. In embodiments, the population of human ILC2s is contacted with about 100 IU to about 1700 IU of the IL-2. In embodiments, the population of human ILC2s is contacted with about 100 IU to about 1600 IU of the IL-2. In embodiments, the population of human ILC2s is contacted with about 100 IU to about 1500 IU of the IL-2. In embodiments, the population of human ILC2s is contacted with about 100 IU to about 1400 IU of the IL-2. In embodiments, the population of human ILC2s is contacted with about 100 IU to about 1300 IU of the IL-2. In embodiments, the population of human ILC2s is contacted with about 100 IU to about 1200 IU of the IL-2. In embodiments, the population of human ILC2s is contacted with about 100 IU to about 1100 IU of the IL-2. In embodiments, the population of human ILC2s is contacted with about 100 IU to about 1000 IU of the IL-2. In embodiments, the population of human ILC2s is contacted with about 100 IU to about 900 IU of the IL-2. In embodiments, the population of human ILC2s is contacted with about 100 IU to about 800 IU of the IL-2. In embodiments, the population of human ILC2s is contacted with about 100 IU to about 700 IU of the IL-2. In embodiments, the population of human ILC2s is contacted with about 100 IU to about 600 IU of the IL-2. In embodiments, the population of human ILC2s is contacted with about 100 IU to about 500 IU of the IL-2. In embodiments, the population of human ILC2s is contacted with about 100 IU to about 400 IU of the IL-2. In embodiments, the population of human ILC2s is contacted with about 100 IU to about 300 IU of the IL-2. In embodiments, the population of human ILC2s is contacted with about 100 IU to about 200 IU of the IL-2. In embodiments, the population of human ILC2s is contacted with about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 IU of the IL-2. In embodiments, the population of human ILC2s is contacted with about about 500 IU of the IL-2.

In embodiments, the population of human ILC2s is contacted with about 0.1 ng/mL to about 500 ng/mL of the IL-7. In embodiments, the population of human ILC2s is contacted with about 10 ng/mL to about 500 ng/mL of the IL-7. In embodiments, the population of human ILC2s is contacted with about 20 ng/mL to about 500 ng/mL of the IL-7. In embodiments, the population of human ILC2s is contacted with about 30 ng/mL to about 500 ng/mL of the IL-7. In embodiments, the population of human ILC2s is contacted with about 40 ng/mL to about 500 ng/mL of the IL-7. In embodiments, the population of human ILC2s is contacted with about 50 ng/mL to about 500 ng/mL of the IL-7. In embodiments, the population of human ILC2s is contacted with about 60 ng/mL to about 500 ng/mL of the IL-7. In embodiments, the population of human ILC2s is contacted with about 70 ng/mL to about 500 ng/mL of the IL-7. In embodiments, the population of human ILC2s is contacted with about 80 ng/mL to about 500 ng/mL of the IL-7. In embodiments, the population of human ILC2s is contacted with about 90 ng/mL to about 500 ng/mL of the IL-7. In embodiments, the population of human ILC2s is contacted with about 100 ng/mL to about 500 ng/mL of the IL-7. In embodiments, the population of human ILC2s is contacted with about 110 ng/mL to about 500 ng/mL of the IL-7. In embodiments, the population of human ILC2s is contacted with about 120 ng/mL to about 500 ng/mL of the IL-7. In embodiments, the population of human ILC2s is contacted with about 130 ng/mL to about 500 ng/mL of the IL-7. In embodiments, the population of human ILC2s is contacted with about 140 ng/mL to about 500 ng/mL of the IL-7. In embodiments, the population of human ILC2s is contacted with about 150 ng/mL to about 500 ng/mL of the IL-7. In embodiments, the population of human ILC2s is contacted with about 160 ng/mL to about 500 ng/mL of the IL-7. In embodiments, the population of human ILC2s is contacted with about 170 ng/mL to about 500 ng/mL of the IL-7. In embodiments, the population of human ILC2s is contacted with about 180 ng/mL to about 500 ng/mL of the IL-7. In embodiments, the population of human ILC2s is contacted with about 190 ng/mL to about 500 ng/mL of the IL-7. In embodiments, the population of human ILC2s is contacted with about 200 ng/mL to about 500 ng/mL of the IL-7. In embodiments, the population of human ILC2s is contacted with about 210 ng/mL to about 500 ng/mL of the IL-7. In embodiments, the population of human ILC2s is contacted with about 220 ng/mL to about 500 ng/mL of the IL-7. In embodiments, the population of human ILC2s is contacted with about 230 ng/mL to about 500 ng/mL of the IL-7. In embodiments, the population of human ILC2s is contacted with about 240 ng/mL to about 500 ng/mL of the IL-7. In embodiments, the population of human ILC2s is contacted with about 250 ng/mL to about 500 ng/mL of the IL-7. In embodiments, the population of human ILC2s is contacted with about 260 ng/mL to about 500 ng/mL of the IL-7. In embodiments, the population of human ILC2s is contacted with about 270 ng/mL to about 500 ng/mL of the IL-7. In embodiments, the population of human ILC2s is contacted with about 280 ng/mL to about 500 ng/mL of the IL-7. In embodiments, the population of human ILC2s is contacted with about 290 ng/mL to about 500 ng/mL of the IL-7. In embodiments, the population of human ILC2s is contacted with about 300 ng/mL to about 500 ng/mL of the IL-7. In embodiments, the population of human ILC2s is contacted with about 310 ng/mL to about 500 ng/mL of the IL-7. In embodiments, the population of human ILC2s is contacted with about 320 ng/mL to about 500 ng/mL of the IL-7. In embodiments, the population of human ILC2s is contacted with about 330 ng/mL to about 500 ng/mL of the IL-7. In embodiments, the population of human ILC2s is contacted with about 340 ng/mL to about 500 ng/mL of the IL-7. In embodiments, the population of human ILC2s is contacted with about 350 ng/mL to about 500 ng/mL of the IL-7. In embodiments, the population of human ILC2s is contacted with about 360 ng/mL to about 500 ng/mL of the IL-7. In embodiments, the population of human ILC2s is contacted with about 370 ng/mL to about 500 ng/mL of the IL-7. In embodiments, the population of human ILC2s is contacted with about 380 ng/mL to about 500 ng/mL of the IL-7. In embodiments, the population of human ILC2s is contacted with about 390 ng/mL to about 500 ng/mL of the IL-7. In embodiments, the population of human ILC2s is contacted with about 400 ng/mL to about 500 ng/mL of the IL-7. In embodiments, the population of human ILC2s is contacted with about 410 ng/mL to about 500 ng/mL of the IL-7. In embodiments, the population of human ILC2s is contacted with about 420 ng/mL to about 500 ng/mL of the IL-7. In embodiments, the population of human ILC2s is contacted with about 430 ng/mL to about 500 ng/mL of the IL-7. In embodiments, the population of human ILC2s is contacted with about 440 ng/mL to about 500 ng/mL of the IL-7. In embodiments, the population of human ILC2s is contacted with about 450 ng/mL to about 500 ng/mL of the IL-7. In embodiments, the population of human ILC2s is contacted with about 460 ng/mL to about 500 ng/mL of the IL-7. In embodiments, the population of human ILC2s is contacted with about 470 ng/mL to about 500 ng/mL of the IL-7. In embodiments, the population of human ILC2s is contacted with about 480 ng/mL to about 500 ng/mL of the IL-7. In embodiments, the population of human ILC2s is contacted with about 490 ng/mL to about 500 ng/mL of the IL-7.

In embodiments, the population of human ILC2s is contacted with about 0.1 ng/mL to about 490 ng/mL of the IL-7. In embodiments, the population of human ILC2s is contacted with about 0.1 ng/mL to about 480 ng/mL of the IL-7. In embodiments, the population of human ILC2s is contacted with about 0.1 ng/mL to about 470 ng/mL of the IL-7. In embodiments, the population of human ILC2s is contacted with about 0.1 ng/mL to about 460 ng/mL of the IL-7. In embodiments, the population of human ILC2s is contacted with about 0.1 ng/mL to about 450 ng/mL of the IL-7. In embodiments, the population of human ILC2s is contacted with about 0.1 ng/mL to about 440 ng/mL of the IL-7. In embodiments, the population of human ILC2s is contacted with about 0.1 ng/mL to about 430 ng/mL of the IL-7. In embodiments, the population of human ILC2s is contacted with about 0.1 ng/mL to about 420 ng/mL of the IL-7. In embodiments, the population of human ILC2s is contacted with about 0.1 ng/mL to about 410 ng/mL of the IL-7. In embodiments, the population of human ILC2s is contacted with about 0.1 ng/mL to about 400 ng/mL of the IL-7. In embodiments, the population of human ILC2s is contacted with about 0.1 ng/mL to about 390 ng/mL of the IL-7. In embodiments, the population of human ILC2s is contacted with about 0.1 ng/mL to about 380 ng/mL of the IL-7. In embodiments, the population of human ILC2s is contacted with about 0.1 ng/mL to about 370 ng/mL of the IL-7. In embodiments, the population of human ILC2s is contacted with about 0.1 ng/mL to about 360 ng/mL of the IL-7. In embodiments, the population of human ILC2s is contacted with about 0.1 ng/mL to about 350 ng/mL of the IL-7. In embodiments, the population of human ILC2s is contacted with about 0.1 ng/mL to about 340 ng/mL of the IL-7. In embodiments, the population of human ILC2s is contacted with about 0.1 ng/mL to about 330 ng/mL of the IL-7. In embodiments, the population of human ILC2s is contacted with about 0.1 ng/mL to about 320 ng/mL of the IL-7. In embodiments, the population of human ILC2s is contacted with about 0.1 ng/mL to about 310 ng/mL of the IL-7. In embodiments, the population of human ILC2s is contacted with about 0.1 ng/mL to about 300 ng/mL of the IL-7. In embodiments, the population of human ILC2s is contacted with about 0.1 ng/mL to about 290 ng/mL of the IL-7. In embodiments, the population of human ILC2s is contacted with about 0.1 ng/mL to about 280 ng/mL of the IL-7. In embodiments, the population of human ILC2s is contacted with about 0.1 ng/mL to about 270 ng/mL of the IL-7. In embodiments, the population of human ILC2s is contacted with about 0.1 ng/mL to about 260 ng/mL of the IL-7. In embodiments, the population of human ILC2s is contacted with about 0.1 ng/mL to about 250 ng/mL of the IL-7. In embodiments, the population of human ILC2s is contacted with about 0.1 ng/mL to about 240 ng/mL of the IL-7. In embodiments, the population of human ILC2s is contacted with about 0.1 ng/mL to about 230 ng/mL of the IL-7. In embodiments, the population of human ILC2s is contacted with about 0.1 ng/mL to about 220 ng/mL of the IL-7. In embodiments, the population of human ILC2s is contacted with about 0.1 ng/mL to about 210 ng/mL of the IL-7. In embodiments, the population of human ILC2s is contacted with about 0.1 ng/mL to about 200 ng/mL of the IL-7. In embodiments, the population of human ILC2s is contacted with about 0.1 ng/mL to about 190 ng/mL of the IL-7. In embodiments, the population of human ILC2s is contacted with about 0.1 ng/mL to about 180 ng/mL of the IL-7. In embodiments, the population of human ILC2s is contacted with about 0.1 ng/mL to about 170 ng/mL of the IL-7. In embodiments, the population of human ILC2s is contacted with about 0.1 ng/mL to about 160 ng/mL of the IL-7. In embodiments, the population of human ILC2s is contacted with about 0.1 ng/mL to about 150 ng/mL of the IL-7. In embodiments, the population of human ILC2s is contacted with about 0.1 ng/mL to about 140 ng/mL of the IL-7. In embodiments, the population of human ILC2s is contacted with about 0.1 ng/mL to about 130 ng/mL of the IL-7. In embodiments, the population of human ILC2s is contacted with about 0.1 ng/mL to about 120 ng/mL of the IL-7. In embodiments, the population of human ILC2s is contacted with about 0.1 ng/mL to about 110 ng/mL of the IL-7. In embodiments, the population of human ILC2s is contacted with about 0.1 ng/mL to about 100 ng/mL of the IL-7. In embodiments, the population of human ILC2s is contacted with about 0.1 ng/mL to about 90 ng/mL of the IL-7. In embodiments, the population of human ILC2s is contacted with about 0.1 ng/mL to about 80 ng/mL of the IL-7. In embodiments, the population of human ILC2s is contacted with about 0.1 ng/mL to about 70 ng/mL of the IL-7. In embodiments, the population of human ILC2s is contacted with about 0.1 ng/mL to about 60 ng/mL of the IL-7. In embodiments, the population of human ILC2s is contacted with about 0.1 ng/mL to about 50 ng/mL of the IL-7. In embodiments, the population of human ILC2s is contacted with about 0.1 ng/mL to about 40 ng/mL of the IL-7. In embodiments, the population of human ILC2s is contacted with about 0.1 ng/mL to about 30 ng/mL of the IL-7. In embodiments, the population of human ILC2s is contacted with about 0.1 ng/mL to about 20 ng/mL of the IL-7. In embodiments, the population of human ILC2s is contacted with about 0.1 ng/mL to about 10 ng/mL of the IL-7. In embodiments, the population of human ILC2s is contacted with about 0.1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL of the IL-7. In embodiments, the population of human ILC2s is contacted with about 20 ng/mL of the IL-7.

In embodiments, the population of human ILC2s is contacted with about 0.1 ng/mL to about 500 ng/mL of the IL-15. In embodiments, the population of human ILC2s is contacted with about 10 ng/mL to about 500 ng/mL of the IL-15. In embodiments, the population of human ILC2s is contacted with about 20 ng/mL to about 500 ng/mL of the IL-15. In embodiments, the population of human ILC2s is contacted with about 30 ng/mL to about 500 ng/mL of the IL-15. In embodiments, the population of human ILC2s is contacted with about 40 ng/mL to about 500 ng/mL of the IL-15. In embodiments, the population of human ILC2s is contacted with about 50 ng/mL to about 500 ng/mL of the IL-15. In embodiments, the population of human ILC2s is contacted with about 60 ng/mL to about 500 ng/mL of the IL-15. In embodiments, the population of human ILC2s is contacted with about 70 ng/mL to about 500 ng/mL of the IL-15. In embodiments, the population of human ILC2s is contacted with about 80 ng/mL to about 500 ng/mL of the IL-15. In embodiments, the population of human ILC2s is contacted with about 90 ng/mL to about 500 ng/mL of the IL-15. In embodiments, the population of human ILC2s is contacted with about 100 ng/mL to about 500 ng/mL of the IL-15. In embodiments, the population of human ILC2s is contacted with about 110 ng/mL to about 500 ng/mL of the IL-15. In embodiments, the population of human ILC2s is contacted with about 120 ng/mL to about 500 ng/mL of the IL-15. In embodiments, the population of human ILC2s is contacted with about 130 ng/mL to about 500 ng/mL of the IL-15. In embodiments, the population of human ILC2s is contacted with about 140 ng/mL to about 500 ng/mL of the IL-15. In embodiments, the population of human ILC2s is contacted with about 150 ng/mL to about 500 ng/mL of the IL-15. In embodiments, the population of human ILC2s is contacted with about 160 ng/mL to about 500 ng/mL of the IL-15. In embodiments, the population of human ILC2s is contacted with about 170 ng/mL to about 500 ng/mL of the IL-15. In embodiments, the population of human ILC2s is contacted with about 180 ng/mL to about 500 ng/mL of the IL-15. In embodiments, the population of human ILC2s is contacted with about 190 ng/mL to about 500 ng/mL of the IL-15. In embodiments, the population of human ILC2s is contacted with about 200 ng/mL to about 500 ng/mL of the IL-15. In embodiments, the population of human ILC2s is contacted with about 210 ng/mL to about 500 ng/mL of the IL-15. In embodiments, the population of human ILC2s is contacted with about 220 ng/mL to about 500 ng/mL of the IL-15. In embodiments, the population of human ILC2s is contacted with about 230 ng/mL to about 500 ng/mL of the IL-15. In embodiments, the population of human ILC2s is contacted with about 240 ng/mL to about 500 ng/mL of the IL-15. In embodiments, the population of human ILC2s is contacted with about 250 ng/mL to about 500 ng/mL of the IL-15. In embodiments, the population of human ILC2s is contacted with about 260 ng/mL to about 500 ng/mL of the IL-15. In embodiments, the population of human ILC2s is contacted with about 270 ng/mL to about 500 ng/mL of the IL-15. In embodiments, the population of human ILC2s is contacted with about 280 ng/mL to about 500 ng/mL of the IL-15. In embodiments, the population of human ILC2s is contacted with about 290 ng/mL to about 500 ng/mL of the IL-15. In embodiments, the population of human ILC2s is contacted with about 300 ng/mL to about 500 ng/mL of the IL-15. In embodiments, the population of human ILC2s is contacted with about 310 ng/mL to about 500 ng/mL of the IL-15. In embodiments, the population of human ILC2s is contacted with about 320 ng/mL to about 500 ng/mL of the IL-15. In embodiments, the population of human ILC2s is contacted with about 330 ng/mL to about 500 ng/mL of the IL-15. In embodiments, the population of human ILC2s is contacted with about 340 ng/mL to about 500 ng/mL of the IL-15. In embodiments, the population of human ILC2s is contacted with about 350 ng/mL to about 500 ng/mL of the IL-15. In embodiments, the population of human ILC2s is contacted with about 360 ng/mL to about 500 ng/mL of the IL-15. In embodiments, the population of human ILC2s is contacted with about 370 ng/mL to about 500 ng/mL of the IL-15. In embodiments, the population of human ILC2s is contacted with about 380 ng/mL to about 500 ng/mL of the IL-15. In embodiments, the population of human ILC2s is contacted with about 390 ng/mL to about 500 ng/mL of the IL-15. In embodiments, the population of human ILC2s is contacted with about 400 ng/mL to about 500 ng/mL of the IL-15. In embodiments, the population of human ILC2s is contacted with about 410 ng/mL to about 500 ng/mL of the IL-15. In embodiments, the population of human ILC2s is contacted with about 420 ng/mL to about 500 ng/mL of the IL-15. In embodiments, the population of human ILC2s is contacted with about 430 ng/mL to about 500 ng/mL of the IL-15. In embodiments, the population of human ILC2s is contacted with about 440 ng/mL to about 500 ng/mL of the IL-15. In embodiments, the population of human ILC2s is contacted with about 450 ng/mL to about 500 ng/mL of the IL-15. In embodiments, the population of human ILC2s is contacted with about 460 ng/mL to about 500 ng/mL of the IL-15. In embodiments, the population of human ILC2s is contacted with about 470 ng/mL to about 500 ng/mL of the IL-15. In embodiments, the population of human ILC2s is contacted with about 480 ng/mL to about 500 ng/mL of the IL-15. In embodiments, the population of human ILC2s is contacted with about 490 ng/mL to about 500 ng/mL of the IL-15.

In embodiments, the population of human ILC2s is contacted with about 0.1 ng/mL to about 490 ng/mL of the IL-15. In embodiments, the population of human ILC2s is contacted with about 0.1 ng/mL to about 480 ng/mL of the IL-15. In embodiments, the population of human ILC2s is contacted with about 0.1 ng/mL to about 470 ng/mL of the IL-15. In embodiments, the population of human ILC2s is contacted with about 0.1 ng/mL to about 460 ng/mL of the IL-15. In embodiments, the population of human ILC2s is contacted with about 0.1 ng/mL to about 450 ng/mL of the IL-15. In embodiments, the population of human ILC2s is contacted with about 0.1 ng/mL to about 440 ng/mL of the IL-15. In embodiments, the population of human ILC2s is contacted with about 0.1 ng/mL to about 430 ng/mL of the IL-15. In embodiments, the population of human ILC2s is contacted with about 0.1 ng/mL to about 420 ng/mL of the IL-15. In embodiments, the population of human ILC2s is contacted with about 0.1 ng/mL to about 410 ng/mL of the IL-15. In embodiments, the population of human ILC2s is contacted with about 0.1 ng/mL to about 400 ng/mL of the IL-15. In embodiments, the population of human ILC2s is contacted with about 0.1 ng/mL to about 390 ng/mL of the IL-15. In embodiments, the population of human ILC2s is contacted with about 0.1 ng/mL to about 380 ng/mL of the IL-15. In embodiments, the population of human ILC2s is contacted with about 0.1 ng/mL to about 370 ng/mL of the IL-15. In embodiments, the population of human ILC2s is contacted with about 0.1 ng/mL to about 360 ng/mL of the IL-15. In embodiments, the population of human ILC2s is contacted with about 0.1 ng/mL to about 350 ng/mL of the IL-15. In embodiments, the population of human ILC2s is contacted with about 0.1 ng/mL to about 340 ng/mL of the IL-15. In embodiments, the population of human ILC2s is contacted with about 0.1 ng/mL to about 330 ng/mL of the IL-15. In embodiments, the population of human ILC2s is contacted with about 0.1 ng/mL to about 320 ng/mL of the IL-15. In embodiments, the population of human ILC2s is contacted with about 0.1 ng/mL to about 310 ng/mL of the IL-15. In embodiments, the population of human ILC2s is contacted with about 0.1 ng/mL to about 300 ng/mL of the IL-15. In embodiments, the population of human ILC2s is contacted with about 0.1 ng/mL to about 290 ng/mL of the IL-15. In embodiments, the population of human ILC2s is contacted with about 0.1 ng/mL to about 280 ng/mL of the IL-15. In embodiments, the population of human ILC2s is contacted with about 0.1 ng/mL to about 270 ng/mL of the IL-15. In embodiments, the population of human ILC2s is contacted with about 0.1 ng/mL to about 260 ng/mL of the IL-15. In embodiments, the population of human ILC2s is contacted with about 0.1 ng/mL to about 250 ng/mL of the IL-15. In embodiments, the population of human ILC2s is contacted with about 0.1 ng/mL to about 240 ng/mL of the IL-15. In embodiments, the population of human ILC2s is contacted with about 0.1 ng/mL to about 230 ng/mL of the IL-15. In embodiments, the population of human ILC2s is contacted with about 0.1 ng/mL to about 220 ng/mL of the IL-15. In embodiments, the population of human ILC2s is contacted with about 0.1 ng/mL to about 210 ng/mL of the IL-15. In embodiments, the population of human ILC2s is contacted with about 0.1 ng/mL to about 200 ng/mL of the IL-15. In embodiments, the population of human ILC2s is contacted with about 0.1 ng/mL to about 190 ng/mL of the IL-15. In embodiments, the population of human ILC2s is contacted with about 0.1 ng/mL to about 180 ng/mL of the IL-15. In embodiments, the population of human ILC2s is contacted with about 0.1 ng/mL to about 170 ng/mL of the IL-15. In embodiments, the population of human ILC2s is contacted with about 0.1 ng/mL to about 160 ng/mL of the IL-15. In embodiments, the population of human ILC2s is contacted with about 0.1 ng/mL to about 150 ng/mL of the IL-15. In embodiments, the population of human ILC2s is contacted with about 0.1 ng/mL to about 140 ng/mL of the IL-15. In embodiments, the population of human ILC2s is contacted with about 0.1 ng/mL to about 130 ng/mL of the IL-15. In embodiments, the population of human ILC2s is contacted with about 0.1 ng/mL to about 120 ng/mL of the IL-15. In embodiments, the population of human ILC2s is contacted with about 0.1 ng/mL to about 110 ng/mL of the IL-15. In embodiments, the population of human ILC2s is contacted with about 0.1 ng/mL to about 100 ng/mL of the IL-15. In embodiments, the population of human ILC2s is contacted with about 0.1 ng/mL to about 90 ng/mL of the IL-15. In embodiments, the population of human ILC2s is contacted with about 0.1 ng/mL to about 80 ng/mL of the IL-15. In embodiments, the population of human ILC2s is contacted with about 0.1 ng/mL to about 70 ng/mL of the IL-15. In embodiments, the population of human ILC2s is contacted with about 0.1 ng/mL to about 60 ng/mL of the IL-15. In embodiments, the population of human ILC2s is contacted with about 0.1 ng/mL to about 50 ng/mL of the IL-15. In embodiments, the population of human ILC2s is contacted with about 0.1 ng/mL to about 40 ng/mL of the IL-15. In embodiments, the population of human ILC2s is contacted with about 0.1 ng/mL to about 30 ng/mL of the IL-15. In embodiments, the population of human ILC2s is contacted with about 0.1 ng/mL to about 20 ng/mL of the IL-15. In embodiments, the population of human ILC2s is contacted with about 0.1 ng/mL to about 10 ng/mL of the IL-15. In embodiments, the population of human ILC2s is contacted with about 0.1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL of the IL-15. In embodiments, the population of human ILC2s is contacted with about 20 ng/mL of the IL-15.

In embodiments, the population of human ILC2s is contacted with about 100 IU to about 1000 IU of the IL-2, about 1 ng/mL to about 50 ng/mL of the IL-7, and about 1 ng/mL to about 50 ng/mL of the IL-15. In embodiments, the population of human ILC2s is contacted with about 100 IU to about 1000 IU of the IL-2. In embodiments, the population of human ILC2s is contacted with about 150 IU to about 1000 IU of the IL-2. In embodiments, the population of human ILC2s is contacted with about 200 IU to about 1000 IU of the IL-2. In embodiments, the population of human ILC2s is contacted with about 250 IU to about 1000 IU of the IL-2. In embodiments, the population of human ILC2s is contacted with about 300 IU to about 1000 IU of the IL-2. In embodiments, the population of human ILC2s is contacted with about 350 IU to about 1000 IU of the IL-2. In embodiments, the population of human ILC2s is contacted with about 400 IU to about 1000 IU of the IL-2. In embodiments, the population of human ILC2s is contacted with about 450 IU to about 1000 IU of the IL-2. In embodiments, the population of human ILC2s is contacted with about 500 IU to about 1000 IU of the IL-2. In embodiments, the population of human ILC2s is contacted with about 550 IU to about 1000 IU of the IL-2. In embodiments, the population of human ILC2s is contacted with about 600 IU to about 1000 IU of the IL-2. In embodiments, the population of human ILC2s is contacted with about 650 IU to about 1000 IU of the IL-2. In embodiments, the population of human ILC2s is contacted with about 700 IU to about 1000 IU of the IL-2. In embodiments, the population of human ILC2s is contacted with about 750 IU to about 1000 IU of the IL-2. In embodiments, the population of human ILC2s is contacted with about 800 IU to about 1000 IU of the IL-2. In embodiments, the population of human ILC2s is contacted with about 850 IU to about 1000 IU of the IL-2. In embodiments, the population of human ILC2s is contacted with about 900 IU to about 1000 IU of the IL-2. In embodiments, the population of human ILC2s is contacted with about 950 IU to about 1000 IU of the IL-2.

In embodiments, the population of human ILC2s is contacted with about 100 IU to about 950 IU of the IL-2. In embodiments, the population of human ILC2s is contacted with about 100 IU to about 900 IU of the IL-2. In embodiments, the population of human ILC2s is contacted with about 100 IU to about 850 IU of the IL-2. In embodiments, the population of human ILC2s is contacted with about 100 IU to about 800 IU of the IL-2. In embodiments, the population of human ILC2s is contacted with about 100 IU to about 750 IU of the IL-2. In embodiments, the population of human ILC2s is contacted with about 100 IU to about 700 IU of the IL-2. In embodiments, the population of human ILC2s is contacted with about 100 IU to about 650 IU of the IL-2. In embodiments, the population of human ILC2s is contacted with about 100 IU to about 600 IU of the IL-2. In embodiments, the population of human ILC2s is contacted with about 100 IU to about 550 IU of the IL-2. In embodiments, the population of human ILC2s is contacted with about 100 IU to about 500 IU of the IL-2. In embodiments, the population of human ILC2s is contacted with about 100 IU to about 450 IU of the IL-2. In embodiments, the population of human ILC2s is contacted with about 100 IU to about 400 IU of the IL-2. In embodiments, the population of human ILC2s is contacted with about 100 IU to about 250 IU of the IL-2. In embodiments, the population of human ILC2s is contacted with about 100 IU to about 200 IU of the IL-2. In embodiments, the population of human ILC2s is contacted with about 100 IU to about 150 IU of the IL-2.

In embodiments, the population of human ILC2s is contacted with about 1 ng/mL to about 50 ng/mL of the IL-7. In embodiments, the population of human ILC2s is contacted with about 5 ng/mL to about 50 ng/mL of the IL-7. In embodiments, the population of human ILC2s is contacted with about 10 ng/mL to about 50 ng/mL of the IL-7. In embodiments, the population of human ILC2s is contacted with about 15 ng/mL to about 50 ng/mL of the IL-7. In embodiments, the population of human ILC2s is contacted with about 20 ng/mL to about 50 ng/mL of the IL-7. In embodiments, the population of human ILC2s is contacted with about 25 ng/mL to about 50 ng/mL of the IL-7. In embodiments, the population of human ILC2s is contacted with about 30 ng/mL to about 50 ng/mL of the IL-7. In embodiments, the population of human ILC2s is contacted with about 35 ng/mL to about 50 ng/mL of the IL-7. In embodiments, the population of human ILC2s is contacted with about 40 ng/mL to about 50 ng/mL of the IL-7. In embodiments, the population of human ILC2s is contacted with about 45 ng/mL to about 50 ng/mL of the IL-7.

In embodiments, the population of human ILC2s is contacted with about 1 ng/mL to about 45 ng/mL of the IL-7. In embodiments, the population of human ILC2s is contacted with about 1 ng/mL to about 40 ng/mL of the IL-7. In embodiments, the population of human ILC2s is contacted with about 1 ng/mL to about 35 ng/mL of the IL-7. In embodiments, the population of human ILC2s is contacted with about 1 ng/mL to about 30 ng/mL of the IL-7. In embodiments, the population of human ILC2s is contacted with about 1 ng/mL to about 25 ng/mL of the IL-7. In embodiments, the population of human ILC2s is contacted with about 1 ng/mL to about 20 ng/mL of the IL-7. In embodiments, the population of human ILC2s is contacted with about 1 ng/mL to about ng/mL of the IL-7. In embodiments, the population of human ILC2s is contacted with about 1 ng/mL to about 10 ng/mL of the IL-7. In embodiments, the population of human ILC2s is contacted with about 1 ng/mL to about 5 ng/mL of the IL-7.

In embodiments, the population of human ILC2s is contacted with about 1 ng/mL to about 50 ng/mL of the IL-15. In embodiments, the population of human ILC2s is contacted with about 5 ng/mL to about 50 ng/mL of the IL-15. In embodiments, the population of human ILC2s is contacted with about 10 ng/mL to about 50 ng/mL of the IL-15. In embodiments, the population of human ILC2s is contacted with about 15 ng/mL to about 50 ng/mL of the IL-15. In embodiments, the population of human ILC2s is contacted with about 20 ng/mL to about 50 ng/mL of the IL-15. In embodiments, the population of human ILC2s is contacted with about 25 ng/mL to about 50 ng/mL of the IL-15. In embodiments, the population of human ILC2s is contacted with about 30 ng/mL to about 50 ng/mL of the IL-15. In embodiments, the population of human ILC2s is contacted with about 35 ng/mL to about 50 ng/mL of the IL-15. In embodiments, the population of human ILC2s is contacted with about 40 ng/mL to about 50 ng/mL of the IL-15. In embodiments, the population of human ILC2s is contacted with about 45 ng/mL to about 50 ng/mL of the IL-15.

In embodiments, the population of human ILC2s is contacted with about 1 ng/mL to about 45 ng/mL of the IL-15. In embodiments, the population of human ILC2s is contacted with about 1 ng/mL to about 40 ng/mL of the IL-15. In embodiments, the population of human ILC2s is contacted with about 1 ng/mL to about 35 ng/mL of the IL-15. In embodiments, the population of human ILC2s is contacted with about 1 ng/mL to about 30 ng/mL of the IL-15. In embodiments, the population of human ILC2s is contacted with about 1 ng/mL to about 25 ng/mL of the IL-15. In embodiments, the population of human ILC2s is contacted with about 1 ng/mL to about 20 ng/mL of the IL-15. In embodiments, the population of human ILC2s is contacted with about 1 ng/mL to about 15 ng/mL of the IL-15. In embodiments, the population of human ILC2s is contacted with about 1 ng/mL to about 10 ng/mL of the IL-15. In embodiments, the population of human ILC2s is contacted with about 1 ng/mL to about 5 ng/mL of the IL-15.

In embodiments, the population of human ILC2s is contacted with about 500 IU of the IL-2. In embodiments, the population of human ILC2s is contacted with about 20 ng/mL of the IL-7. In embodiments, the population of human ILC2s is contacted with about 20 ng/mL of the IL-15. In embodiments, the population of human ILC2s is contacted with 500 IU of the IL-2. In embodiments, the population of human ILC2s is contacted with 20 ng/mL of the IL-7. In embodiments, the population of human ILC2s is contacted with 20 ng/mL of the IL-15. In embodiments, the population of human ILC2s is contacted with about 500 IU of the IL-2, about 20 ng/mL of the IL-7, and about 20 ng/mL of the IL-15. In embodiments, the population of human ILC2s is contacted with 500 IU of the IL-2, 20 ng/mL of the IL-7, and 20 ng/mL of the IL-15.

For the methods provided herein, in embodiments, the population of human ILC2s are obtained from a subject with cancer. In embodiments, the population of human ILC2s are obtained from a healthy subject (e.g. a subject who does not have cancer). In embodiments, the population of human ILC2s are derived from peripheral blood obtained from the subject.

In embodiments, the population of human ILC2s are isolated from non-ILC2 cells. In embodiments, the population of human ILC2s are isolated from non-ILC2 cells using fluorescence-activated cell sorting (FACS), magnetic cell sorting, adhesion-based cell soring, complement depletion, or density gradient centrifugation. In embodiments, the population of human ILC2s are isolated from non-ILC2 cells using magnetic beads or density gradient centrifugation. In embodiments, isolating the population of human ILC2s refers to removing the population of human ILC2s from a population of cells including ILC2s and non-ILC2 cells. In embodiments, isolating the population of human ILC2s refers to removing non-ILC2 cells from a population of cells obtained from the subject or donor, wherein the population of cells includes the population of ILC2s and non-ILC2 cells. In embodiments, the non-ILC2 cells include NK cells. In embodiments, the non-ILC2 cells include T cells. In embodiments, the non-ILC2 cells include ILC1s. In embodiments, the non-ILC2 cells include ILC3s. In embodiments, the population of human ILC2s are isolated from non-ILC2 cells prior to contacting the population of human ILC2s with the IL-2, the IL-7, and the IL-15.

In embodiments, the population of human ILC2s is contacted with the IL-2, the IL-7, and the IL-15 in a cell culture. “Contacting” is used in accordance with its plain ordinary meaning and refers to the process of allowing at least two distinct species (e.g. a protein and a cell) to become sufficiently proximal to react, interact or physically touch. The two species may be, for example, IL-2 and a human ILC2. In embodiments contacting includes, for example, allowing a protein described herein (e.g. IL-2, IL-7, IL-15, etc.) to physically touch a ILC2. In embodiments, the contacting may result in delivery of a compound (e.g. a protein, DNA, a biomolecule, etc.) into a cell. For example, the contacting may result in delivery of protein into a cell (e.g. a ILC2). In embodiments, the contacting may result in binding of a protein to receptor expressed on the surface of a cell (e.g. a ILC2). In embodiments, the contacting may result in delivery of a nucleic acid into the cell. In embodiments, “contacting” or “contacted” includes culturing a population of human ILC2s in the presence of a species, e.g., a protein as provided herein. Thus, in embodiments, the contacting occurs in a cell culture.

In embodiments, the population of human ILC2s is contacted with the IL-2, the IL-7, and the IL-15 for about 5 to about 60 days. In embodiments, the population of human ILC2s is contacted with the IL-2, the IL-7, and the IL-15 for about 10 to about 60 days. In embodiments, the population of human ILC2s is contacted with the IL-2, the IL-7, and the IL-15 for about 15 to about 60 days. In embodiments, the population of human ILC2s is contacted with the IL-2, the IL-7, and the IL-15 for about 20 to about 60 days. In embodiments, the population of human ILC2s is contacted with the IL-2, the IL-7, and the IL-15 for about 25 to about 60 days. In embodiments, the population of human ILC2s is contacted with the IL-2, the IL-7, and the IL-15 for about 30 to about 60 days. In embodiments, the population of human ILC2s is contacted with the IL-2, the IL-7, and the IL-15 for about 35 to about 60 days. In embodiments, the population of human ILC2s is contacted with the IL-2, the IL-7, and the IL-15 for about 40 to about 60 days. In embodiments, the population of human ILC2s is contacted with the IL-2, the IL-7, and the IL-15 for about 45 to about 60 days. In embodiments, the population of human ILC2s is contacted with the IL-2, the IL-7, and the IL-15 for about 50 to about 60 days. In embodiments, the population of human ILC2s is contacted with the IL-2, the IL-7, and the IL-15 for about 55 to about 60 days.

In embodiments, the population of human ILC2s is contacted with the IL-2, the IL-7, and the IL-15 for about 5 to about 55 days. In embodiments, the population of human ILC2s is contacted with the IL-2, the IL-7, and the IL-15 for about 5 to about 50 days. In embodiments, the population of human ILC2s is contacted with the IL-2, the IL-7, and the IL-15 for about 5 to about 45 days. In embodiments, the population of human ILC2s is contacted with the IL-2, the IL-7, and the IL-15 for about 5 to about 40 days. In embodiments, the population of human ILC2s is contacted with the IL-2, the IL-7, and the IL-15 for about 5 to about 35 days. In embodiments, the population of human ILC2s is contacted with the IL-2, the IL-7, and the IL-15 for about 5 to about 30 days. In embodiments, the population of human ILC2s is contacted with the IL-2, the IL-7, and the IL-15 for about 5 to about 25 days. In embodiments, the population of human ILC2s is contacted with the IL-2, the IL-7, and the IL-15 for about 5 to about 20 days. In embodiments, the population of human ILC2s is contacted with the IL-2, the IL-7, and the IL-15 for about 5 to about 15 days. In embodiments, the population of human ILC2s is contacted with the IL-2, the IL-7, and the IL-15 for about 5 to about 10 days. In embodiments, the population of human ILC2s is contacted with the IL-2, the IL-7, and the IL-15 for about 5, 10, 15, 20, 25, 30, 35, 40, 45, 50, 55, or 60 days. In embodiments, the population of human ILC2s is contacted with the IL-2, the IL-7, and the IL-15 for about 28 days. In embodiments, the population of human ILC2s is contacted with the IL-2, the IL-7, and the IL-15 for 28 days.

For the methods provided herein, in embodiments, the population of human ILC2s is contacted with the IL-2, the IL-7, and the IL-15 for a first time period, the population of human ILC2s is isolated from non-ILC2 cells, then the population of expanded human ILC2s is contacted with the IL-2, the IL-7, and the IL-15 for a second (e.g. subsequent) time period, thereby producing the population of expanded human ILC2s cells. In embodiments, the contacting comprises: a) contacting the population of human ILC2s with the IL-2, the IL-7, and the IL-15 for to 20 days; b) isolating the population of human ILC2s from non-ILC2 cells; and c) contacting the population of human ILC2s with the IL-2, the IL-7, and the IL-15 for a subsequent 10 to 20 days. In embodiments, step a) comprises contacting the population of human ILC2s with the IL-2, the IL-7, and the IL-15 for 10 to 20 days. In embodiments, step a) comprises contacting the population of human ILC2s with the IL-2, the IL-7, and the IL-15 for 12 to 20 days. In embodiments, step a) comprises contacting the population of human ILC2s with the IL-2, the IL-7, and the IL-15 for 14 to 20 days. In embodiments, step a) comprises contacting the population of human ILC2s with the IL-2, the IL-7, and the IL-15 for 16 to 20 days. In embodiments, step a) comprises contacting the population of human ILC2s with the IL-2, the IL-7, and the IL-15 for 18 to 20 days.

In embodiments, step a) comprises contacting the population of human ILC2s with the IL-2, the IL-7, and the IL-15 for 10 to 18 days. In embodiments, step a) comprises contacting the population of human ILC2s with the IL-2, the IL-7, and the IL-15 for 10 to 16 days. In embodiments, step a) comprises contacting the population of human ILC2s with the IL-2, the IL-7, and the IL-15 for 10 to 14 days. In embodiments, step a) comprises contacting the population of human ILC2s with the IL-2, the IL-7, and the IL-15 for 10 to 12 days. In embodiments, step a) comprises contacting the population of human ILC2s with the IL-2, the IL-7, and the IL-15 for 1, 2, 3, 4, 5, 6, 7, 8, 9, 10, 11, 12, 13, 14, 15, 16, 17, 18, 19, or 20 days. In embodiments, step a) comprises contacting the population of human ILC2s with the IL-2, the IL-7, and the IL-15 for 10, 12, 14, 16, 18, or 20 days. In embodiments, step a) comprises contacting the population of human ILC2s with the IL-2, the IL-7, and the IL-15 for about 14 days.

In embodiments, step c) comprises contacting the population of human ILC2s with the IL-2, the IL-7, and the IL-15 for a subsequent 10 to 20 days. In embodiments, step c) comprises contacting the population of human ILC2s with the IL-2, the IL-7, and the IL-15 for a subsequent 12 to 20 days. In embodiments, step c) comprises contacting the population of human ILC2s with the IL-2, the IL-7, and the IL-15 for a subsequent 14 to 20 days. In embodiments, step c) comprises contacting the population of human ILC2s with the IL-2, the IL-7, and the IL-15 for a subsequent 16 to 20 days. In embodiments, step c) comprises contacting the population of human ILC2s with the IL-2, the IL-7, and the IL-15 for a subsequent 18 to 20 days.

In embodiments, step c) comprises contacting the population of human ILC2s with the IL-2, the IL-7, and the IL-15 for a subsequent 10 to 18 days. In embodiments, step c) comprises contacting the population of human ILC2s with the IL-2, the IL-7, and the IL-15 for a subsequent 10 to 16 days. In embodiments, step c) comprises contacting the population of human ILC2s with the IL-2, the IL-7, and the IL-15 for a subsequent 10 to 14 days. In embodiments, step c) comprises contacting the population of human ILC2s with the IL-2, the IL-7, and the IL-15 for a subsequent 10 to 12 days. In embodiments, step c) comprises contacting the population of human ILC2s with the IL-2, the IL-7, and the IL-15 for a subsequent 1, 2, 3, 4, 5, 6, 7, 8, 9, 10, 11, 12, 13, 14, 15, 16, 17, 18, 19, or 20 days. In embodiments, step c) comprises contacting the population of human ILC2s with the IL-2, the IL-7, and the IL-15 for a subsequent 10, 12, 14, 16, 18, or 20 days. In embodiments, step c) comprises contacting the population of human ILC2s with the IL-2, the IL-7, and the IL-15 for about a subsequent 14 days.

In embodiments, the contacting comprises: a) contacting the population of human ILC2s with the IL-2, the IL-7, and the IL-15 for 14 days; b) isolating the population of human ILC2s from non-ILC2 cells; and c) contacting the population of human ILC2s with the IL-2, the IL-7, and the IL-15 for a subsequent 14 days. In embodiments, the population of human ILC2s is isolated from non-ILC2 cells using flow cytometry. For example, the population of human ILC2s may be isolated from non-ILC2 cells by isolating CD161 expressing ILC2s cells. In embodiments, the population of human ILC2s may include at least 90%, 91% 92%, 93%, 94%, 95%, 96%, 97%, 98%, 99%, or 100% human ILC2s prior to step c).

In embodiments, the population of human ILC2s includes at least 90% human ILC2s prior to step c). In embodiments, the population of human ILC2s includes at least 91% human ILC2s prior to step c). In embodiments, the population of human ILC2s includes at least 92% human ILC2s prior to step c). In embodiments, the population of human ILC2s includes at least 93% human ILC2s prior to step c). In embodiments, the population of human ILC2s includes at least 94% human ILC2s prior to step c). In embodiments, the population of human ILC2s includes at least 95% human ILC2s prior to step c). In embodiments, the population of human ILC2s includes at least 96% human ILC2s prior to step c). In embodiments, the population of human ILC2s includes at least 97% human ILC2s prior to step c). In embodiments, the population of human ILC2s includes at least 98% human ILC2s prior to step c). In embodiments, the population of human ILC2s includes at least 99% human ILC2s prior to step c). In embodiments, the population of human ILC2s includes 100% human ILC2s prior to step c).

In embodiments, the population of human ILC2s is contacted with the IL-2, the IL-7, and the IL-15 in the presence of a stromal cell. In embodiments, the stromal cell includes an exogenous nucleic acid encoding a Notch ligand delta-like 1 (DL1) protein. In embodiments, the stromal cell stabily expresses a DL1 protein. In embodiments, the stromal cell is a DL1-transfected OP9 cell, also referred to as OP9-DL1 or a DL1 cell. In embodiments, the stromal cell includes an exogenous nucleic acid encoding a Notch ligand delta-like 4 (DL4) protein. In embodiments, the stromal cell stably expresses DL4 protein. In embodiments, the stromal cell is a DL4-transfected OP9, also referred to as OP9-DL4 or a DL4 cell. The term “stably transfected cell” is used in accordance with its plain ordinary meaning in the art and refers to a cell transfected with a nucleic acid wherein the nucleic acid is incorporated into genome of the cell.

For the methods provided herein, in embodiments, the population of expanded human ILC2s includes CD161 expressing ILC2s (e.g. CD161⁺ ILC2). For example, the population of expanded human ILC2s may include at least 30%, 50%, 80%, 90%, 95%, 98% or 99% CD161⁺ expressing ILC2s. In embodiments, the population of expanded human ILC2s includes at least 30% CD161⁺ ILC2s. In embodiments, the population of expanded human ILC2s includes at least 40% CD161⁺ ILC2s. In embodiments, the population of expanded human ILC2s includes at least 50% CD161⁺ ILC2s. In embodiments, the population of expanded human ILC2s includes at least 60% CD161⁺ ILC2s. In embodiments, the population of expanded human ILC2s includes at least 70% CD161⁺ ILC2s. In embodiments, the population of expanded human ILC2s includes at least 80% CD161⁺ ILC2s. In embodiments, the population of expanded human ILC2s includes at least 85% CD161⁺ ILC2s. In embodiments, the population of expanded human ILC2s includes at least 90% CD161⁺ ILC2s. In embodiments, the population of expanded human ILC2s includes at least 91% CD161⁺ ILC2s. In embodiments, the population of expanded human ILC2s includes at least 92% CD161⁺ ILC2s. In embodiments, the population of expanded human ILC2s includes at least 93% CD161⁺ ILC2s. In embodiments, the population of expanded human ILC2s includes at least 94% CD161⁺ ILC2s. In embodiments, the population of expanded human ILC2s includes at least 95% CD161⁺ ILC2s. In embodiments, the population of expanded human ILC2s includes at least 96% CD161⁺ ILC2s. In embodiments, the population of expanded human ILC2s includes at least 97% CD161⁺ ILC2s. In embodiments, the population of expanded human ILC2s includes at least 98% CD161⁺ ILC2s. In embodiments, the population of expanded human ILC2s includes at least 99% CD161⁺ ILC2s. In embodiments, the population of expanded human ILC2s includes about 100% CD161⁺ ILC2s.

In embodiments, the population of expanded human ILC2s includes about 30% CD161⁺ ILC2s. In embodiments, the population of expanded human ILC2s includes about 40% CD161⁺ ILC2s. In embodiments, the population of expanded human ILC2s includes about 50% CD161⁺ ILC2s. In embodiments, the population of expanded human ILC2s includes about 60% CD161⁺ ILC2s. In embodiments, the population of expanded human ILC2s includes about 70% CD161⁺ ILC2s. In embodiments, the population of expanded human ILC2s includes about 80% CD161⁺ ILC2s. In embodiments, the population of expanded human ILC2s includes about 85% CD161⁺ ILC2s. In embodiments, the population of expanded human ILC2s includes about 90% CD161⁺ ILC2s. In embodiments, the population of expanded human ILC2s includes about 91% CD161⁺ ILC2s. In embodiments, the population of expanded human ILC2s includes about 92% CD161⁺ ILC2s. In embodiments, the population of expanded human ILC2s includes about 93% CD161⁺ ILC2s. In embodiments, the population of expanded human ILC2s includes about 94% CD161⁺ ILC2s. In embodiments, the population of expanded human ILC2s includes about 95% CD161⁺ ILC2s. In embodiments, the population of expanded human ILC2s includes about 96% CD161⁺ ILC2s. In embodiments, the population of expanded human ILC2s includes about 97% CD161⁺ ILC2s. In embodiments, the population of expanded human ILC2s includes about 98% CD161⁺ ILC2s. In embodiments, the population of expanded human ILC2s includes about 99% CD161⁺ ILC2s. In embodiments, the population of expanded human ILC2s includes about 100% CD161⁺ ILC2s.

For the methods provided herein, in embodiments, the population of expanded human ILC2s includes CRTH2 and CD117 expressing ILC2s (e.g. CRTH2⁺CD117+ILC2). For example, the population of expanded human ILC2s may include at least 30%, 50%, 80%, 90%, 95%, 98% or 99% CRTH2⁺CD117⁺ ILC2s. In embodiments, the population of expanded human ILC2s includes at least 30% CRTH2⁺CD117⁺ ILC2s. In embodiments, the population of expanded human ILC2s includes at least 40% CRTH2⁺CD117⁺ ILC2s. In embodiments, the population of expanded human ILC2s includes at least 50% CRTH2⁺CD117⁺ ILC2s. In embodiments, the population of expanded human ILC2s includes at least 60% CRTH2⁺CD117+ILC2s. In embodiments, the population of expanded human ILC2s includes at least 70% CRTH2⁺CD117⁺ ILC2s. In embodiments, the population of expanded human ILC2s includes at least 80% CRTH2⁺CD117⁺ ILC2s. In embodiments, the population of expanded human ILC2s includes at least 85% CRTH2⁺CD117⁺ ILC2s. In embodiments, the population of expanded human ILC2s includes at least 90% CRTH2⁺CD117⁺ ILC2s. In embodiments, the population of expanded human ILC2s includes at least 91% CRTH2⁺CD117⁺ ILC2s. In embodiments, the population of expanded human ILC2s includes at least 92% CRTH2⁺CD117⁺ ILC2s. In embodiments, the population of expanded human ILC2s includes at least 93% CRTH2⁺CD117⁺ ILC2s. In embodiments, the population of expanded human ILC2s includes at least 94% CRTH2⁺CD117⁺ ILC2s. In embodiments, the population of expanded human ILC2s includes at least 95% CRTH2⁺CD117+ILC2s. In embodiments, the population of expanded human ILC2s includes at least 96% CRTH2⁺CD117⁺ ILC2s. In embodiments, the population of expanded human ILC2s includes at least 97% CRTH2⁺CD117⁺ ILC2s. In embodiments, the population of expanded human ILC2s includes at least 98% CRTH2⁺CD117⁺ ILC2s. In embodiments, the population of expanded human ILC2s includes at least 99% CRTH2⁺CD117⁺ ILC2s.

In embodiments, the population of expanded human ILC2s includes about 30% CRTH2⁺CD117⁺ ILC2s. In embodiments, the population of expanded human ILC2s includes about 40% CRTH2⁺CD117⁺ ILC2s. In embodiments, the population of expanded human ILC2s includes about 50% CRTH2⁺CD117⁺ ILC2s. In embodiments, the population of expanded human ILC2s includes about 60% CRTH2⁺CD117⁺ ILC2s. In embodiments, the population of expanded human ILC2s includes about 70% CRTH2⁺CD117⁺ ILC2s. In embodiments, the population of expanded human ILC2s includes about 80% CRTH2⁺CD117⁺ ILC2s. In embodiments, the population of expanded human ILC2s includes about 85% CRTH2⁺CD117⁺ ILC2s. In embodiments, the population of expanded human ILC2s includes about 90% CRTH2⁺CD117⁺ ILC2s. In embodiments, the population of expanded human ILC2s includes about 91% CRTH2⁺CD117+ILC2s. In embodiments, the population of expanded human ILC2s includes about 92% CRTH2⁺CD117⁺ ILC2s. In embodiments, the population of expanded human ILC2s includes about 93% CRTH2⁺CD117⁺ ILC2s. In embodiments, the population of expanded human ILC2s includes about 94% CRTH2⁺CD117⁺ ILC2s. In embodiments, the population of expanded human ILC2s includes about 95% CRTH2⁺CD117⁺ ILC2s. In embodiments, the population of expanded human ILC2s includes about 96% CRTH2⁺CD117⁺ ILC2s. In embodiments, the population of expanded human ILC2s includes about 97% CRTH2⁺CD117⁺ ILC2s. In embodiments, the population of expanded human ILC2s includes about 98% CRTH2⁺CD117⁺ ILC2s. In embodiments, the population of expanded human ILC2s includes about 99% CRTH2⁺CD117⁺ ILC2s. In embodiments, the population of expanded human ILC2s includes about 100% CRTH2⁺CD117⁺ ILC2s.

In embodiments, the CD161⁺ ILC2s further express CRTH2 and CD117 (e.g. CD161⁺CRTH2⁺CD117⁺ ILC2). For example, the CD161⁺ ILC2s may include at least 30%, 50%, 80%, 90%, 95%, 98% or 99% CRTH2⁺CD117⁺ ILC2s (e.g. CD161⁺CRTH2⁺CD117⁺ ILC2). In embodiments, the CD161⁺ ILC2s include at least 30% CRTH2⁺CD117⁺ ILC2. In embodiments, the CD161⁺ ILC2s include at least 40% CRTH2⁺CD117⁺ ILC2. In embodiments, the CD161⁺ ILC2s include at least 50% CRTH2⁺CD117⁺ ILC2. In embodiments, the CD161⁺ ILC2s include at least 60% CRTH2⁺CD117⁺ ILC2. In embodiments, the CD161⁺ ILC2s include at least 70% CRTH2⁺CD117⁺ ILC2. In embodiments, the CD161⁺ ILC2s include at least 80% CRTH2⁺CD117⁺ ILC2. In embodiments, the CD161⁺ ILC2s include at least 85% CRTH2⁺CD117⁺ ILC2. In embodiments, the CD161⁺ ILC2s include at least 90% CRTH2⁺CD117⁺ ILC2. In embodiments, the CD161⁺ ILC2s include at least 91% CRTH2⁺CD117⁺ ILC2. In embodiments, the CD161⁺ ILC2s include at least 92% CRTH2⁺CD117⁺ ILC2. In embodiments, the CD161⁺ ILC2s include at least 93% CRTH2⁺CD117⁺ ILC2. In embodiments, the CD161⁺ ILC2s include at least 94% CRTH2⁺CD117⁺ ILC2. In embodiments, the CD161⁺ ILC2s include at least 95% CRTH2⁺CD117⁺ ILC2. In embodiments, the CD161⁺ ILC2s include at least 96% CRTH2⁺CD117⁺ ILC2. In embodiments, the CD161⁺ ILC2s include at least 97% CRTH2⁺CD117⁺ ILC2. In embodiments, the CD161⁺ ILC2s include at least 98% CRTH2⁺CD117⁺ ILC2. In embodiments, the CD161⁺ ILC2s include at least 99% CRTH2⁺CD117⁺ ILC2.

In embodiments, the CD161⁺ ILC2s include about 30% CRTH2⁺CD117⁺ ILC2. In embodiments, the CD161⁺ ILC2s include about 40% CRTH2⁺CD117⁺ ILC2. In embodiments, the CD161⁺ ILC2s include about 50% CRTH2⁺CD117⁺ ILC2. In embodiments, the CD161⁺ ILC2s include about 60% CRTH2⁺CD117⁺ ILC2. In embodiments, the CD161⁺ ILC2s include about 70% CRTH2⁺CD117⁺ ILC2. In embodiments, the CD161⁺ ILC2s include about 80% CRTH2⁺CD117⁺ ILC2. In embodiments, the CD161⁺ ILC2s include about 85% CRTH2⁺CD117⁺ ILC2. In embodiments, the CD161⁺ ILC2s include about 90% CRTH2⁺CD117⁺ ILC2. In embodiments, the CD161⁺ ILC2s include about 91% CRTH2⁺CD117⁺ ILC2. In embodiments, the CD161⁺ ILC2s include about 92% CRTH2⁺CD117⁺ ILC2. In embodiments, the CD161⁺ ILC2s include about 93% CRTH2⁺CD117⁺ ILC2. In embodiments, the CD161⁺ ILC2s include about 94% CRTH2⁺CD117⁺ ILC2. In embodiments, the CD161⁺ ILC2s include about 95% CRTH2⁺CD117⁺ ILC2. In embodiments, the CD161⁺ ILC2s include about 96% CRTH2⁺CD117⁺ ILC2. In embodiments, the CD161⁺ ILC2s include about 97% CRTH2⁺CD117⁺ ILC2. In embodiments, the CD161⁺ ILC2s include about 98% CRTH2⁺CD117⁺ ILC2. In embodiments, the CD161⁺ ILC2s include about 99% CRTH2⁺CD117⁺ ILC2. In embodiments, the CD161⁺ ILC2s include about 100% CRTH2⁺CD117⁺ ILC2.

For the methods provided herein, in embodiments, the population of expanded human ILC2s includes DNAM-1 expressing ILC2s (e.g. DNAM-1⁺ ILC2). For example, the population of expanded human ILC2s may include at least 30%, 50%, 80%, 90%, 95%, 98% or 99% DNAM-1⁺ ILC2. In embodiments, the population of expanded human ILC2s includes DNAX Accessory Molecule-1 (DNAM-1) expressing ILC2. In embodiments, the population of expanded human ILC2s includes at least 30% DNAM-1 expressing ILC2. In embodiments, the population of expanded human ILC2s includes at least 40% DNAM-1 expressing ILC2. In embodiments, the population of expanded human ILC2s includes at least 50% DNAM-1 expressing ILC2. In embodiments, the population of expanded human ILC2s includes at least 60% DNAM-1 expressing ILC2. In embodiments, the population of expanded human ILC2s includes at least 70% DNAM-1 expressing ILC2. In embodiments, the population of expanded human ILC2s includes at least 80% DNAM-1 expressing ILC2. In embodiments, the population of expanded human ILC2s includes at least 85% DNAM-1 expressing ILC2. In embodiments, the population of expanded human ILC2s includes at least 90% DNAM-1 expressing ILC2. In embodiments, the population of expanded human ILC2s includes at least 91% DNAM-1 expressing ILC2. In embodiments, the population of expanded human ILC2s includes at least 92% DNAM-1 expressing ILC2. In embodiments, the population of expanded human ILC2s includes at least 93% DNAM-1 expressing ILC2. In embodiments, the population of expanded human ILC2s includes at least 94% DNAM-1 expressing ILC2. In embodiments, the population of expanded human ILC2s includes at least 95% DNAM-1 expressing ILC2. In embodiments, the population of expanded human ILC2s includes at least 96% DNAM-1 expressing ILC2. In embodiments, the population of expanded human ILC2s includes at least 97% DNAM-1 expressing ILC2. In embodiments, the population of expanded human ILC2s includes at least 99% DNAM-1 expressing ILC2. In embodiments, the population of expanded human ILC2s includes at least 99% DNAM-1 expressing ILC2.

In embodiments, the population of expanded human ILC2s includes about 30% DNAM-1 expressing ILC2. In embodiments, the population of expanded human ILC2s includes about 40% DNAM-1 expressing ILC2. In embodiments, the population of expanded human ILC2s includes about 50% DNAM-1 expressing ILC2. In embodiments, the population of expanded human ILC2s includes about 60% DNAM-1 expressing ILC2. In embodiments, the population of expanded human ILC2s includes about 70% DNAM-1 expressing ILC2. In embodiments, the population of expanded human ILC2s includes about 80% DNAM-1 expressing ILC2. In embodiments, the population of expanded human ILC2s includes about 85% DNAM-1 expressing ILC2. In embodiments, the population of expanded human ILC2s includes about 90% DNAM-1 expressing ILC2. In embodiments, the population of expanded human ILC2s includes about 91% DNAM-1 expressing ILC2. In embodiments, the population of expanded human ILC2s includes about 92% DNAM-1 expressing ILC2. In embodiments, the population of expanded human ILC2s includes about 93% DNAM-1 expressing ILC2. In embodiments, the population of expanded human ILC2s includes about 94% DNAM-1 expressing ILC2. In embodiments, the population of expanded human ILC2s includes about 95% DNAM-1 expressing ILC2. In embodiments, the population of expanded human ILC2s includes about 96% DNAM-1 expressing ILC2. In embodiments, the population of expanded human ILC2s includes about 97% DNAM-1 expressing ILC2. In embodiments, the population of expanded human ILC2s includes about 99% DNAM-1 expressing ILC2. In embodiments, the population of expanded human ILC2s includes about 99% DNAM-1 expressing ILC2. In embodiments, the population of expanded human ILC2s includes about 100% DNAM-1 expressing ILC2.

For the methods provided herein, in embodiments, the population of expanded human ILC2s includes GZMB expressing ILC2s (e.g. GZMB⁺ ILC2). For example, the population of expanded human ILC2s may include at least 30%, 50%, 80%, 90%, 95%, 98% or 99% GZMB⁺ ILC2. In embodiments, the population of expanded human ILC2s includes granzyme B (GZMB) expressing ILC2. In embodiments, the population of expanded human ILC2s includes at least 30% GZMB expressing ILC2. In embodiments, the population of expanded human ILC2s includes at least 40% GZMB expressing ILC2. In embodiments, the population of expanded human ILC2s includes at least 50% GZMB expressing ILC2. In embodiments, the population of expanded human ILC2s includes at least 60% GZMB expressing ILC2. In embodiments, the population of expanded human ILC2s includes at least 70% GZMB expressing ILC2. In embodiments, the population of expanded human ILC2s includes at least 80% GZMB expressing ILC2. In embodiments, the population of expanded human ILC2s includes at least 85% GZMB expressing ILC2. In embodiments, the population of expanded human ILC2s includes at least 90% GZMB expressing ILC2. In embodiments, the population of expanded human ILC2s includes at least 91% GZMB expressing ILC2. In embodiments, the population of expanded human ILC2s includes at least 92% GZMB expressing ILC2. In embodiments, the population of expanded human ILC2s includes at least 93% GZMB expressing ILC2. In embodiments, the population of expanded human ILC2s includes at least 94% GZMB expressing ILC2. In embodiments, the population of expanded human ILC2s includes at least 95% GZMB expressing ILC2. In embodiments, the population of expanded human ILC2s includes at least 96% GZMB expressing ILC2. In embodiments, the population of expanded human ILC2s includes at least 97% GZMB expressing ILC2. In embodiments, the population of expanded human ILC2s includes at least 98% GZMB expressing ILC2. In embodiments, the population of expanded human ILC2s includes at least 99% GZMB expressing ILC2.

In embodiments, the population of expanded human ILC2s includes about 30% GZMB expressing ILC2. In embodiments, the population of expanded human ILC2s includes about 40% GZMB expressing ILC2. In embodiments, the population of expanded human ILC2s includes about 50% GZMB expressing ILC2. In embodiments, the population of expanded human ILC2s includes about 60% GZMB expressing ILC2. In embodiments, the population of expanded human ILC2s includes about 70% GZMB expressing ILC2. In embodiments, the population of expanded human ILC2s includes about 80% GZMB expressing ILC2. In embodiments, the population of expanded human ILC2s includes about 85% GZMB expressing ILC2. In embodiments, the population of expanded human ILC2s includes about 90% GZMB expressing ILC2. In embodiments, the population of expanded human ILC2s includes about 91% GZMB expressing ILC2. In embodiments, the population of expanded human ILC2s includes about 92% GZMB expressing ILC2. In embodiments, the population of expanded human ILC2s includes about 93% GZMB expressing ILC2. In embodiments, the population of expanded human ILC2s includes about 94% GZMB expressing ILC2. In embodiments, the population of expanded human ILC2s includes about 95% GZMB expressing ILC2. In embodiments, the population of expanded human ILC2s includes about 96% GZMB expressing ILC2. In embodiments, the population of expanded human ILC2s includes about 97% GZMB expressing ILC2. In embodiments, the population of expanded human ILC2s includes about 99% GZMB expressing ILC2. In embodiments, the population of expanded human ILC2s includes about 99% GZMB expressing ILC2. In embodiments, the population of expanded human ILC2s includes about 100% GZMB expressing ILC2.

For the methods provided herein, in embodiments, the population of expanded human ILC2s includes human ILC2s expressing IL-4, IL-5, IL-9, IL-13, or a combination thereof. In embodiments, the population of expanded human ILC2s expresses IL-4, IL-5, IL-9, IL-13, or a combination thereof. In embodiments, the population of expanded human ILC2s expresses IL-4. In embodiments, the population of expanded human ILC2s expresses IL-5. In embodiments, the population of expanded human ILC2s expresses IL-9. In embodiments, the population of expanded human ILC2s expresses IL-13.

In embodiments, at least 10% of the population of expanded human ILC2s expresses IL-4. In embodiments, at least 20% of the population of expanded human ILC2s expresses IL-4. In embodiments, at least 30% of the population of expanded human ILC2s expresses IL-4. In embodiments, at least 40% of the population of expanded human ILC2s expresses IL-4. In embodiments, at least 50% of the population of expanded human ILC2s expresses IL-4. In embodiments, at least 60% of the population of expanded human ILC2s expresses IL-4. In embodiments, at least 70% of the population of expanded human ILC2s expresses IL-4. In embodiments, at least 80% of the population of expanded human ILC2s expresses IL-4. In embodiments, at least 85% of the population of expanded human ILC2s expresses IL-4. In embodiments, at least 90% of the population of expanded human ILC2s expresses IL-4. In embodiments, at least 91% of the population of expanded human ILC2s expresses IL-4. In embodiments, at least 92% of the population of expanded human ILC2s expresses IL-4. In embodiments, at least 93% of the population of expanded human ILC2s expresses IL-4. In embodiments, at least 94% of the population of expanded human ILC2s expresses IL-4. In embodiments, at least 95% of the population of expanded human ILC2s expresses IL-4. In embodiments, at least 96% of the population of expanded human ILC2s expresses IL-4. In embodiments, at least 97% of the population of expanded human ILC2s expresses IL-4. In embodiments, at least 98% of the population of expanded human ILC2s expresses IL-4. In embodiments, at least 99% of the population of expanded human ILC2s expresses IL-4.

In embodiments, about 10% of the population of expanded human ILC2s expresses IL-4. In embodiments, about 20% of the population of expanded human ILC2s expresses IL-4. In embodiments, about 30% of the population of expanded human ILC2s expresses IL-4. In embodiments, about 40% of the population of expanded human ILC2s expresses IL-4. In embodiments, about 50% of the population of expanded human ILC2s expresses IL-4. In embodiments, about 60% of the population of expanded human ILC2s expresses IL-4. In embodiments, about 70% of the population of expanded human ILC2s expresses IL-4. In embodiments, about 80% of the population of expanded human ILC2s expresses IL-4. In embodiments, about 85% of the population of expanded human ILC2s expresses IL-4. In embodiments, about 90% of the population of expanded human ILC2s expresses IL-4. In embodiments, about 91% of the population of expanded human ILC2s expresses IL-4. In embodiments, about 92% of the population of expanded human ILC2s expresses IL-4. In embodiments, about 93% of the population of expanded human ILC2s expresses IL-4. In embodiments, about 94% of the population of expanded human ILC2s expresses IL-4. In embodiments, about 95% of the population of expanded human ILC2s expresses IL-4. In embodiments, about 96% of the population of expanded human ILC2s expresses IL-4. In embodiments, about 97% of the population of expanded human ILC2s expresses IL-4. In embodiments, about 98% of the population of expanded human ILC2s expresses IL-4. In embodiments, about 99% of the population of expanded human ILC2s expresses IL-4. In embodiments, about 100% of the population of expanded human ILC2s expresses IL-4.

In embodiments, the population of expanded human ILC2s includes at least 30% IL-4 expressing ILC2. In embodiments, the population of expanded human ILC2s includes at least 40% IL-4 expressing ILC2. In embodiments, the population of expanded human ILC2s includes at least 50% IL-4 expressing ILC2. In embodiments, the population of expanded human ILC2s includes at least 60% IL-4 expressing ILC2. In embodiments, the population of expanded human ILC2s includes at least 70% IL-4 expressing ILC2. In embodiments, the population of expanded human ILC2s includes at least 80% IL-4 expressing ILC2. In embodiments, the population of expanded human ILC2s includes at least 85% IL-4 expressing ILC2. In embodiments, the population of expanded human ILC2s includes at least 90% IL-4 expressing ILC2. In embodiments, the population of expanded human ILC2s includes at least 91% IL-4 expressing ILC2. In embodiments, the population of expanded human ILC2s includes at least 92% IL-4 expressing ILC2. In embodiments, the population of expanded human ILC2s includes at least 93% IL-4 expressing ILC2. In embodiments, the population of expanded human ILC2s includes at least 94% IL-4 expressing ILC2. In embodiments, the population of expanded human ILC2s includes at least 95% IL-4 expressing ILC2. In embodiments, the population of expanded human ILC2s includes at least 96% IL-4 expressing ILC2. In embodiments, the population of expanded human ILC2s includes at least 97% IL-4 expressing ILC2. In embodiments, the population of expanded human ILC2s includes at least 98% IL-4 expressing ILC2. In embodiments, the population of expanded human ILC2s includes at least 99% IL-4 expressing ILC2.

In embodiments, the population of expanded human ILC2s includes about 30% IL-4 expressing ILC2. In embodiments, the population of expanded human ILC2s includes about 40% IL-4 expressing ILC2. In embodiments, the population of expanded human ILC2s includes about 50% IL-4 expressing ILC2. In embodiments, the population of expanded human ILC2s includes about 60% IL-4 expressing ILC2. In embodiments, the population of expanded human ILC2s includes about 70% IL-4 expressing ILC2. In embodiments, the population of expanded human ILC2s includes about 80% IL-4 expressing ILC2. In embodiments, the population of expanded human ILC2s includes about 85% IL-4 expressing ILC2. In embodiments, the population of expanded human ILC2s includes about 90% IL-4 expressing ILC2. In embodiments, the population of expanded human ILC2s includes about 91% IL-4 expressing ILC2. In embodiments, the population of expanded human ILC2s includes about 92% IL-4 expressing ILC2. In embodiments, the population of expanded human ILC2s includes about 93% IL-4 expressing ILC2. In embodiments, the population of expanded human ILC2s includes about 94% IL-4 expressing ILC2. In embodiments, the population of expanded human ILC2s includes about 95% IL-4 expressing ILC2. In embodiments, the population of expanded human ILC2s includes about 96% IL-4 expressing ILC2. In embodiments, the population of expanded human ILC2s includes about 97% IL-4 expressing ILC2. In embodiments, the population of expanded human ILC2s includes about 99% IL-4 expressing ILC2. In embodiments, the population of expanded human ILC2s includes about 99% IL-4 expressing ILC2. In embodiments, the population of expanded human ILC2s includes about 100% IL-4 expressing ILC2.

In embodiments, at least 10% of the population of expanded human ILC2s expresses IL-5. In embodiments, at least 20% of the population of expanded human ILC2s expresses IL-5. In embodiments, at least 30% of the population of expanded human ILC2s expresses IL-5. In embodiments, at least 40% of the population of expanded human ILC2s expresses IL-5. In embodiments, at least 50% of the population of expanded human ILC2s expresses IL-5. In embodiments, at least 60% of the population of expanded human ILC2s expresses IL-5. In embodiments, at least 70% of the population of expanded human ILC2s expresses IL-5. In embodiments, at least 80% of the population of expanded human ILC2s expresses IL-5. In embodiments, at least 85% of the population of expanded human ILC2s expresses IL-5. In embodiments, at least 90% of the population of expanded human ILC2s expresses IL-5. In embodiments, at least 91% of the population of expanded human ILC2s expresses IL-5. In embodiments, at least 92% of the population of expanded human ILC2s expresses IL-5. In embodiments, at least 93% of the population of expanded human ILC2s expresses IL-5. In embodiments, at least 94% of the population of expanded human ILC2s expresses IL-5. In embodiments, at least 95% of the population of expanded human ILC2s expresses IL-5. In embodiments, at least 96% of the population of expanded human ILC2s expresses IL-5. In embodiments, at least 97% of the population of expanded human ILC2s expresses IL-5. In embodiments, at least 98% of the population of expanded human ILC2s expresses IL-5. In embodiments, at least 99% of the population of expanded human ILC2s expresses IL-5.

In embodiments, about 10% of the population of expanded human ILC2s expresses IL-5. In embodiments, about 20% of the population of expanded human ILC2s expresses IL-5. In embodiments, about 30% of the population of expanded human ILC2s expresses IL-5. In embodiments, about 40% of the population of expanded human ILC2s expresses IL-5. In embodiments, about 50% of the population of expanded human ILC2s expresses IL-5. In embodiments, about 60% of the population of expanded human ILC2s expresses IL-5. In embodiments, about 70% of the population of expanded human ILC2s expresses IL-5. In embodiments, about 80% of the population of expanded human ILC2s expresses IL-5. In embodiments, about 85% of the population of expanded human ILC2s expresses IL-5. In embodiments, about 90% of the population of expanded human ILC2s expresses IL-5. In embodiments, about 91% of the population of expanded human ILC2s expresses IL-5. In embodiments, about 92% of the population of expanded human ILC2s expresses IL-5. In embodiments, about 93% of the population of expanded human ILC2s expresses IL-5. In embodiments, about 94% of the population of expanded human ILC2s expresses IL-5. In embodiments, about 95% of the population of expanded human ILC2s expresses IL-5. In embodiments, about 96% of the population of expanded human ILC2s expresses IL-5. In embodiments, about 97% of the population of expanded human ILC2s expresses IL-5. In embodiments, about 98% of the population of expanded human ILC2s expresses IL-5. In embodiments, about 99% of the population of expanded human ILC2s expresses IL-5. In embodiments, about 100% of the population of expanded human ILC2s expresses IL-5.

In embodiments, the population of expanded human ILC2s includes at least 30% IL-5 expressing ILC2. In embodiments, the population of expanded human ILC2s includes at least 40% IL-5 expressing ILC2. In embodiments, the population of expanded human ILC2s includes at least 50% IL-5 expressing ILC2. In embodiments, the population of expanded human ILC2s includes at least 60% IL-5 expressing ILC2. In embodiments, the population of expanded human ILC2s includes at least 70% IL-5 expressing ILC2. In embodiments, the population of expanded human ILC2s includes at least 80% IL-5 expressing ILC2. In embodiments, the population of expanded human ILC2s includes at least 85% IL-5 expressing ILC2. In embodiments, the population of expanded human ILC2s includes at least 90% IL-5 expressing ILC2. In embodiments, the population of expanded human ILC2s includes at least 91% IL-5 expressing ILC2. In embodiments, the population of expanded human ILC2s includes at least 92% IL-5 expressing ILC2. In embodiments, the population of expanded human ILC2s includes at least 93% IL-5 expressing ILC2. In embodiments, the population of expanded human ILC2s includes at least 94% IL-5 expressing ILC2. In embodiments, the population of expanded human ILC2s includes at least 95% IL-5 expressing ILC2. In embodiments, the population of expanded human ILC2s includes at least 96% IL-5 expressing ILC2. In embodiments, the population of expanded human ILC2s includes at least 97% IL-5 expressing ILC2. In embodiments, the population of expanded human ILC2s includes at least 98% IL-5 expressing ILC2. In embodiments, the population of expanded human ILC2s includes at least 99% IL-5 expressing ILC2.

In embodiments, the population of expanded human ILC2s includes about 30% IL-5 expressing ILC2. In embodiments, the population of expanded human ILC2s includes about 40% IL-5 expressing ILC2. In embodiments, the population of expanded human ILC2s includes about 50% IL-5 expressing ILC2. In embodiments, the population of expanded human ILC2s includes about 60% IL-5 expressing ILC2. In embodiments, the population of expanded human ILC2s includes about 70% IL-5 expressing ILC2. In embodiments, the population of expanded human ILC2s includes about 80% IL-5 expressing ILC2. In embodiments, the population of expanded human ILC2s includes about 85% IL-5 expressing ILC2. In embodiments, the population of expanded human ILC2s includes about 90% IL-5 expressing ILC2. In embodiments, the population of expanded human ILC2s includes about 91% IL-5 expressing ILC2. In embodiments, the population of expanded human ILC2s includes about 92% IL-5 expressing ILC2. In embodiments, the population of expanded human ILC2s includes about 93% IL-5 expressing ILC2. In embodiments, the population of expanded human ILC2s includes about 94% IL-5 expressing ILC2. In embodiments, the population of expanded human ILC2s includes about 95% IL-5 expressing ILC2. In embodiments, the population of expanded human ILC2s includes about 96% IL-5 expressing ILC2. In embodiments, the population of expanded human ILC2s includes about 97% IL-5 expressing ILC2. In embodiments, the population of expanded human ILC2s includes about 99% IL-5 expressing ILC2. In embodiments, the population of expanded human ILC2s includes about 99% IL-5 expressing ILC2. In embodiments, the population of expanded human ILC2s includes about 100% IL-5 expressing ILC2.

In embodiments, at least 10% of the population of expanded human ILC2s expresses IL-9. In embodiments, at least 20% of the population of expanded human ILC2s expresses IL-9. In embodiments, at least 30% of the population of expanded human ILC2s expresses IL-9. In embodiments, at least 40% of the population of expanded human ILC2s expresses IL-9. In embodiments, at least 50% of the population of expanded human ILC2s expresses IL-9. In embodiments, at least 60% of the population of expanded human ILC2s expresses IL-9. In embodiments, at least 70% of the population of expanded human ILC2s expresses IL-9. In embodiments, at least 80% of the population of expanded human ILC2s expresses IL-9. In embodiments, at least 85% of the population of expanded human ILC2s expresses IL-9. In embodiments, at least 90% of the population of expanded human ILC2s expresses IL-9. In embodiments, at least 91% of the population of expanded human ILC2s expresses IL-9. In embodiments, at least 92% of the population of expanded human ILC2s expresses IL-9. In embodiments, at least 93% of the population of expanded human ILC2s expresses IL-9. In embodiments, at least 94% of the population of expanded human ILC2s expresses IL-9. In embodiments, at least 95% of the population of expanded human ILC2s expresses IL-9. In embodiments, at least 96% of the population of expanded human ILC2s expresses IL-9. In embodiments, at least 97% of the population of expanded human ILC2s expresses IL-9. In embodiments, at least 98% of the population of expanded human ILC2s expresses IL-9. In embodiments, at least 99% of the population of expanded human ILC2s expresses IL-9.

In embodiments, about 10% of the population of expanded human ILC2s expresses IL-9. In embodiments, about 20% of the population of expanded human ILC2s expresses IL-9. In embodiments, about 30% of the population of expanded human ILC2s expresses IL-9. In embodiments, about 40% of the population of expanded human ILC2s expresses IL-9. In embodiments, about 50% of the population of expanded human ILC2s expresses IL-9. In embodiments, about 60% of the population of expanded human ILC2s expresses IL-9. In embodiments, about 70% of the population of expanded human ILC2s expresses IL-9. In embodiments, about 80% of the population of expanded human ILC2s expresses IL-9. In embodiments, about 85% of the population of expanded human ILC2s expresses IL-9. In embodiments, about 90% of the population of expanded human ILC2s expresses IL-9. In embodiments, about 91% of the population of expanded human ILC2s expresses IL-9. In embodiments, about 92% of the population of expanded human ILC2s expresses IL-9. In embodiments, about 93% of the population of expanded human ILC2s expresses IL-9. In embodiments, about 94% of the population of expanded human ILC2s expresses IL-9. In embodiments, about 95% of the population of expanded human ILC2s expresses IL-9. In embodiments, about 96% of the population of expanded human ILC2s expresses IL-9. In embodiments, about 97% of the population of expanded human ILC2s expresses IL-9. In embodiments, about 98% of the population of expanded human ILC2s expresses IL-9. In embodiments, about 99% of the population of expanded human ILC2s expresses IL-9. In embodiments, about 100% of the population of expanded human ILC2s expresses IL-9.

In embodiments, the population of expanded human ILC2s includes at least 30% IL-9 expressing ILC2. In embodiments, the population of expanded human ILC2s includes at least 40% IL-9 expressing ILC2. In embodiments, the population of expanded human ILC2s includes at least 50% IL-9 expressing ILC2. In embodiments, the population of expanded human ILC2s includes at least 60% IL-9 expressing ILC2. In embodiments, the population of expanded human ILC2s includes at least 70% IL-9 expressing ILC2. In embodiments, the population of expanded human ILC2s includes at least 80% IL-9 expressing ILC2. In embodiments, the population of expanded human ILC2s includes at least 85% IL-9 expressing ILC2. In embodiments, the population of expanded human ILC2s includes at least 90% IL-9 expressing ILC2. In embodiments, the population of expanded human ILC2s includes at least 91% IL-9 expressing ILC2. In embodiments, the population of expanded human ILC2s includes at least 92% IL-9 expressing ILC2. In embodiments, the population of expanded human ILC2s includes at least 93% IL-9 expressing ILC2. In embodiments, the population of expanded human ILC2s includes at least 94% IL-9 expressing ILC2. In embodiments, the population of expanded human ILC2s includes at least 95% IL-9 expressing ILC2. In embodiments, the population of expanded human ILC2s includes at least 96% IL-9 expressing ILC2. In embodiments, the population of expanded human ILC2s includes at least 97% IL-9 expressing ILC2. In embodiments, the population of expanded human ILC2s includes at least 98% IL-9 expressing ILC2. In embodiments, the population of expanded human ILC2s includes at least 99% IL-9 expressing ILC2.

In embodiments, the population of expanded human ILC2s includes about 30% IL-9 expressing ILC2. In embodiments, the population of expanded human ILC2s includes about 40% IL-9 expressing ILC2. In embodiments, the population of expanded human ILC2s includes about 50% IL-9 expressing ILC2. In embodiments, the population of expanded human ILC2s includes about 60% IL-9 expressing ILC2. In embodiments, the population of expanded human ILC2s includes about 70% IL-9 expressing ILC2. In embodiments, the population of expanded human ILC2s includes about 80% IL-9 expressing ILC2. In embodiments, the population of expanded human ILC2s includes about 85% IL-9 expressing ILC2. In embodiments, the population of expanded human ILC2s includes about 90% IL-9 expressing ILC2. In embodiments, the population of expanded human ILC2s includes about 91% IL-9 expressing ILC2. In embodiments, the population of expanded human ILC2s includes about 92% IL-9 expressing ILC2. In embodiments, the population of expanded human ILC2s includes about 93% IL-9 expressing ILC2. In embodiments, the population of expanded human ILC2s includes about 94% IL-9 expressing ILC2. In embodiments, the population of expanded human ILC2s includes about 95% IL-9 expressing ILC2. In embodiments, the population of expanded human ILC2s includes about 96% IL-9 expressing ILC2. In embodiments, the population of expanded human ILC2s includes about 97% IL-9 expressing ILC2. In embodiments, the population of expanded human ILC2s includes about 99% IL-9 expressing ILC2. In embodiments, the population of expanded human ILC2s includes about 99% IL-9 expressing ILC2. In embodiments, the population of expanded human ILC2s includes about 100% IL-9 expressing ILC2.

In embodiments, at least 10% of the population of expanded human ILC2s expresses IL-13. In embodiments, at least 20% of the population of expanded human ILC2s expresses IL-13. In embodiments, at least 30% of the population of expanded human ILC2s expresses IL-13. In embodiments, at least 40% of the population of expanded human ILC2s expresses IL-13. In embodiments, at least 50% of the population of expanded human ILC2s expresses IL-13. In embodiments, at least 60% of the population of expanded human ILC2s expresses IL-13. In embodiments, at least 70% of the population of expanded human ILC2s expresses IL-13. In embodiments, at least 80% of the population of expanded human ILC2s expresses IL-13. In embodiments, at least 85% of the population of expanded human ILC2s expresses IL-13. In embodiments, at least 90% of the population of expanded human ILC2s expresses IL-13. In embodiments, at least 91% of the population of expanded human ILC2s expresses IL-13. In embodiments, at least 92% of the population of expanded human ILC2s expresses IL-13. In embodiments, at least 93% of the population of expanded human ILC2s expresses IL-13. In embodiments, at least 94% of the population of expanded human ILC2s expresses IL-13. In embodiments, at least 95% of the population of expanded human ILC2s expresses IL-13. In embodiments, at least 96% of the population of expanded human ILC2s expresses IL-13. In embodiments, at least 97% of the population of expanded human ILC2s expresses IL-13. In embodiments, at least 98% of the population of expanded human ILC2s expresses IL-13. In embodiments, at least 99% of the population of expanded human ILC2s expresses IL-13.

In embodiments, about 10% of the population of expanded human ILC2s expresses IL-13. In embodiments, about 20% of the population of expanded human ILC2s expresses IL-13. In embodiments, about 30% of the population of expanded human ILC2s expresses IL-13. In embodiments, about 40% of the population of expanded human ILC2s expresses IL-13. In embodiments, about 50% of the population of expanded human ILC2s expresses IL-13. In embodiments, about 60% of the population of expanded human ILC2s expresses IL-13. In embodiments, about 70% of the population of expanded human ILC2s expresses IL-13. In embodiments, about 80% of the population of expanded human ILC2s expresses IL-13. In embodiments, about 85% of the population of expanded human ILC2s expresses IL-13. In embodiments, about 90% of the population of expanded human ILC2s expresses IL-13. In embodiments, about 91% of the population of expanded human ILC2s expresses IL-13. In embodiments, about 92% of the population of expanded human ILC2s expresses IL-13. In embodiments, about 93% of the population of expanded human ILC2s expresses IL-13. In embodiments, about 94% of the population of expanded human ILC2s expresses IL-13. In embodiments, about 95% of the population of expanded human ILC2s expresses IL-13. In embodiments, about 96% of the population of expanded human ILC2s expresses IL-13. In embodiments, about 97% of the population of expanded human ILC2s expresses IL-13. In embodiments, about 98% of the population of expanded human ILC2s expresses IL-13. In embodiments, about 99% of the population of expanded human ILC2s expresses IL-13. In embodiments, about 100% of the population of expanded human ILC2s expresses IL-13.

In embodiments, the population of expanded human ILC2s includes at least 30% IL-13 expressing ILC2. In embodiments, the population of expanded human ILC2s includes at least 40% IL-13 expressing ILC2. In embodiments, the population of expanded human ILC2s includes at least 50% IL-13 expressing ILC2. In embodiments, the population of expanded human ILC2s includes at least 60% IL-13 expressing ILC2. In embodiments, the population of expanded human ILC2s includes at least 70% IL-13 expressing ILC2. In embodiments, the population of expanded human ILC2s includes at least 80% IL-13 expressing ILC2. In embodiments, the population of expanded human ILC2s includes at least 85% IL-13 expressing ILC2. In embodiments, the population of expanded human ILC2s includes at least 90% IL-13 expressing ILC2. In embodiments, the population of expanded human ILC2s includes at least 91% IL-13 expressing ILC2. In embodiments, the population of expanded human ILC2s includes at least 92% IL-13 expressing ILC2. In embodiments, the population of expanded human ILC2s includes at least 93% IL-13 expressing ILC2. In embodiments, the population of expanded human ILC2s includes at least 94% IL-13 expressing ILC2. In embodiments, the population of expanded human ILC2s includes at least 95% IL-13 expressing ILC2. In embodiments, the population of expanded human ILC2s includes at least 96% IL-13 expressing ILC2. In embodiments, the population of expanded human ILC2s includes at least 97% IL-13 expressing ILC2. In embodiments, the population of expanded human ILC2s includes at least 98% IL-13 expressing ILC2. In embodiments, the population of expanded human ILC2s includes at least 99% IL-13 expressing ILC2.

In embodiments, the population of expanded human ILC2s includes about 30% IL-13 expressing ILC2. In embodiments, the population of expanded human ILC2s includes about 40% IL-13 expressing ILC2. In embodiments, the population of expanded human ILC2s includes about 50% IL-13 expressing ILC2. In embodiments, the population of expanded human ILC2s includes about 60% IL-13 expressing ILC2. In embodiments, the population of expanded human ILC2s includes about 70% IL-13 expressing ILC2. In embodiments, the population of expanded human ILC2s includes about 80% IL-13 expressing ILC2. In embodiments, the population of expanded human ILC2s includes about 85% IL-13 expressing ILC2. In embodiments, the population of expanded human ILC2s includes about 90% IL-13 expressing ILC2. In embodiments, the population of expanded human ILC2s includes about 91% IL-13 expressing ILC2. In embodiments, the population of expanded human ILC2s includes about 92% IL-13 expressing ILC2. In embodiments, the population of expanded human ILC2s includes about 93% IL-13 expressing ILC2. In embodiments, the population of expanded human ILC2s includes about 94% IL-13 expressing ILC2. In embodiments, the population of expanded human ILC2s includes about 95% IL-13 expressing ILC2. In embodiments, the population of expanded human ILC2s includes about 96% IL-13 expressing ILC2. In embodiments, the population of expanded human ILC2s includes about 97% IL-13 expressing ILC2. In embodiments, the population of expanded human ILC2s includes about 99% IL-13 expressing ILC2. In embodiments, the population of expanded human ILC2s includes about 99% IL-13 expressing ILC2. In embodiments, the population of expanded human ILC2s includes about 100% IL-13 expressing ILC2.

In embodiments, the population of expanded human ILC2s includes IL-33R expressing ILC2, NKp30 expressing ILC2, or a combination thereof. In embodiments, the population of expanded human ILC2s includes IL-33R expressing ILC2. In embodiments, the population of expanded human ILC2s includes NKp30 expressing ILC2.

In embodiments, at least 10% of the population of expanded human ILC2s expresses IL-33R. In embodiments, at least 20% of the population of expanded human ILC2s expresses IL-33R. In embodiments, at least 30% of the population of expanded human ILC2s expresses IL-33R. In embodiments, at least 40% of the population of expanded human ILC2s expresses IL-33R. In embodiments, at least 50% of the population of expanded human ILC2s expresses IL-33R. In embodiments, at least 60% of the population of expanded human ILC2s expresses IL-33R. In embodiments, at least 70% of the population of expanded human ILC2s expresses IL-33R. In embodiments, at least 80% of the population of expanded human ILC2s expresses IL-33R. In embodiments, at least 85% of the population of expanded human ILC2s expresses IL-33R. In embodiments, at least 90% of the population of expanded human ILC2s expresses IL-33R. In embodiments, at least 91% of the population of expanded human ILC2s expresses IL-33R. In embodiments, at least 92% of the population of expanded human ILC2s expresses IL-33R. In embodiments, at least 93% of the population of expanded human ILC2s expresses IL-33R. In embodiments, at least 94% of the population of expanded human ILC2s expresses IL-33R. In embodiments, at least 95% of the population of expanded human ILC2s expresses IL-33R. In embodiments, at least 96% of the population of expanded human ILC2s expresses IL-33R. In embodiments, at least 97% of the population of expanded human ILC2s expresses IL-33R. In embodiments, at least 98% of the population of expanded human ILC2s expresses IL-33R. In embodiments, at least 99% of the population of expanded human ILC2s expresses IL-33R.

In embodiments, about 10% of the population of expanded human ILC2s expresses IL-33R. In embodiments, about 20% of the population of expanded human ILC2s expresses IL-33R. In embodiments, about 30% of the population of expanded human ILC2s expresses IL-33R. In embodiments, about 40% of the population of expanded human ILC2s expresses IL-33R. In embodiments, about 50% of the population of expanded human ILC2s expresses IL-33R. In embodiments, about 60% of the population of expanded human ILC2s expresses IL-33R. In embodiments, about 70% of the population of expanded human ILC2s expresses IL-33R. In embodiments, about 80% of the population of expanded human ILC2s expresses IL-33R. In embodiments, about 85% of the population of expanded human ILC2s expresses IL-33R. In embodiments, about 90% of the population of expanded human ILC2s expresses IL-33R. In embodiments, about 91% of the population of expanded human ILC2s expresses IL-33R. In embodiments, about 92% of the population of expanded human ILC2s expresses IL-33R. In embodiments, about 93% of the population of expanded human ILC2s expresses IL-33R. In embodiments, about 94% of the population of expanded human ILC2s expresses IL-33R. In embodiments, about 95% of the population of expanded human ILC2s expresses IL-33R. In embodiments, about 96% of the population of expanded human ILC2s expresses IL-33R. In embodiments, about 97% of the population of expanded human ILC2s expresses IL-33R. In embodiments, about 98% of the population of expanded human ILC2s expresses IL-33R. In embodiments, about 99% of the population of expanded human ILC2s expresses IL-33R. In embodiments, about 100% of the population of expanded human ILC2s expresses IL-33R.

In embodiments, the population of expanded human ILC2s includes at least 30% IL-33R expressing ILC2. In embodiments, the population of expanded human ILC2s includes at least 40% IL-33R expressing ILC2. In embodiments, the population of expanded human ILC2s includes at least 50% IL-33R expressing ILC2. In embodiments, the population of expanded human ILC2s includes at least 60% IL-33R expressing ILC2. In embodiments, the population of expanded human ILC2s includes at least 70% IL-33R expressing ILC2. In embodiments, the population of expanded human ILC2s includes at least 80% IL-33R expressing ILC2. In embodiments, the population of expanded human ILC2s includes at least 85% IL-33R expressing ILC2. In embodiments, the population of expanded human ILC2s includes at least 90% IL-33R expressing ILC2. In embodiments, the population of expanded human ILC2s includes at least 91% IL-33R expressing ILC2. In embodiments, the population of expanded human ILC2s includes at least 92% IL-33R expressing ILC2. In embodiments, the population of expanded human ILC2s includes at least 93% IL-33R expressing ILC2. In embodiments, the population of expanded human ILC2s includes at least 94% IL-33R expressing ILC2. In embodiments, the population of expanded human ILC2s includes at least 95% IL-33R expressing ILC2. In embodiments, the population of expanded human ILC2s includes at least 96% IL-33R expressing ILC2. In embodiments, the population of expanded human ILC2s includes at least 97% IL-33R expressing ILC2. In embodiments, the population of expanded human ILC2s includes at least 98% IL-33R expressing ILC2. In embodiments, the population of expanded human ILC2s includes at least 99% IL-33R expressing ILC2.

In embodiments, the population of expanded human ILC2s includes about 30% IL-33R expressing ILC2. In embodiments, the population of expanded human ILC2s includes about 40% IL-33R expressing ILC2. In embodiments, the population of expanded human ILC2s includes about 50% IL-33R expressing ILC2. In embodiments, the population of expanded human ILC2s includes about 60% IL-33R expressing ILC2. In embodiments, the population of expanded human ILC2s includes about 70% IL-33R expressing ILC2. In embodiments, the population of expanded human ILC2s includes about 80% IL-33R expressing ILC2. In embodiments, the population of expanded human ILC2s includes about 85% IL-33R expressing ILC2. In embodiments, the population of expanded human ILC2s includes about 90% IL-33R expressing ILC2. In embodiments, the population of expanded human ILC2s includes about 91% IL-33R expressing ILC2. In embodiments, the population of expanded human ILC2s includes about 92% IL-33R expressing ILC2. In embodiments, the population of expanded human ILC2s includes about 93% IL-33R expressing ILC2. In embodiments, the population of expanded human ILC2s includes about 94% IL-33R expressing ILC2. In embodiments, the population of expanded human ILC2s includes about 95% IL-33R expressing ILC2. In embodiments, the population of expanded human ILC2s includes about 96% IL-33R expressing ILC2. In embodiments, the population of expanded human ILC2s includes about 97% IL-33R expressing ILC2. In embodiments, the population of expanded human ILC2s includes about 99% IL-33R expressing ILC2. In embodiments, the population of expanded human ILC2s includes about 99% IL-33R expressing ILC2. In embodiments, the population of expanded human ILC2s includes about 100% IL-33R expressing ILC2.

In embodiments, at least 10% of the population of expanded human ILC2s expresses NKp30. In embodiments, at least 20% of the population of expanded human ILC2s expresses NKp30. In embodiments, at least 30% of the population of expanded human ILC2s expresses NKp30. In embodiments, at least 40% of the population of expanded human ILC2s expresses NKp30. In embodiments, at least 50% of the population of expanded human ILC2s expresses NKp30. In embodiments, at least 60% of the population of expanded human ILC2s expresses NKp30. In embodiments, at least 70% of the population of expanded human ILC2s expresses NKp30. In embodiments, at least 80% of the population of expanded human ILC2s expresses NKp30. In embodiments, at least 85% of the population of expanded human ILC2s expresses NKp30. In embodiments, at least 90% of the population of expanded human ILC2s expresses NKp30. In embodiments, at least 91% of the population of expanded human ILC2s expresses NKp30. In embodiments, at least 92% of the population of expanded human ILC2s expresses NKp30. In embodiments, at least 93% of the population of expanded human ILC2s expresses NKp30. In embodiments, at least 94% of the population of expanded human ILC2s expresses NKp30. In embodiments, at least 95% of the population of expanded human ILC2s expresses NKp30. In embodiments, at least 96% of the population of expanded human ILC2s expresses NKp30. In embodiments, at least 97% of the population of expanded human ILC2s expresses NKp30. In embodiments, at least 98% of the population of expanded human ILC2s expresses NKp30. In embodiments, at least 99% of the population of expanded human ILC2s expresses NKp30.

In embodiments, about 10% of the population of expanded human ILC2s expresses NKp30. In embodiments, about 20% of the population of expanded human ILC2s expresses NKp30. In embodiments, about 30% of the population of expanded human ILC2s expresses NKp30. In embodiments, about 40% of the population of expanded human ILC2s expresses NKp30. In embodiments, about 50% of the population of expanded human ILC2s expresses NKp30. In embodiments, about 60% of the population of expanded human ILC2s expresses NKp30. In embodiments, about 70% of the population of expanded human ILC2s expresses NKp30. In embodiments, about 80% of the population of expanded human ILC2s expresses NKp30. In embodiments, about 85% of the population of expanded human ILC2s expresses NKp30. In embodiments, about 90% of the population of expanded human ILC2s expresses NKp30. In embodiments, about 91% of the population of expanded human ILC2s expresses NKp30. In embodiments, about 92% of the population of expanded human ILC2s expresses NKp30. In embodiments, about 93% of the population of expanded human ILC2s expresses NKp30. In embodiments, about 94% of the population of expanded human ILC2s expresses NKp30. In embodiments, about 95% of the population of expanded human ILC2s expresses NKp30. In embodiments, about 96% of the population of expanded human ILC2s expresses NKp30. In embodiments, about 97% of the population of expanded human ILC2s expresses NKp30. In embodiments, about 98% of the population of expanded human ILC2s expresses NKp30. In embodiments, about 99% of the population of expanded human ILC2s expresses NKp30. In embodiments, about 100% of the population of expanded human ILC2s expresses NKp30.

In embodiments, the population of expanded human ILC2s includes at least 30% NKp30 expressing ILC2. In embodiments, the population of expanded human ILC2s includes at least 40% NKp30 expressing ILC2. In embodiments, the population of expanded human ILC2s includes at least 50% NKp30 expressing ILC2. In embodiments, the population of expanded human ILC2s includes at least 60% NKp30 expressing ILC2. In embodiments, the population of expanded human ILC2s includes at least 70% NKp30 expressing ILC2. In embodiments, the population of expanded human ILC2s includes at least 80% NKp30 expressing ILC2. In embodiments, the population of expanded human ILC2s includes at least 85% NKp30 expressing ILC2. In embodiments, the population of expanded human ILC2s includes at least 90% NKp30 expressing ILC2. In embodiments, the population of expanded human ILC2s includes at least 91% NKp30 expressing ILC2. In embodiments, the population of expanded human ILC2s includes at least 92% NKp30 expressing ILC2. In embodiments, the population of expanded human ILC2s includes at least 93% NKp30 expressing ILC2. In embodiments, the population of expanded human ILC2s includes at least 94% NKp30 expressing ILC2. In embodiments, the population of expanded human ILC2s includes at least 95% NKp30 expressing ILC2. In embodiments, the population of expanded human ILC2s includes at least 96% NKp30 expressing ILC2. In embodiments, the population of expanded human ILC2s includes at least 97% NKp30 expressing ILC2. In embodiments, the population of expanded human ILC2s includes at least 98% NKp30 expressing ILC2. In embodiments, the population of expanded human ILC2s includes at least 99% NKp30 expressing ILC2.

In embodiments, the population of expanded human ILC2s includes about 30% NKp30 expressing ILC2. In embodiments, the population of expanded human ILC2s includes about 40% NKp30 expressing ILC2. In embodiments, the population of expanded human ILC2s includes about 50% NKp30 expressing ILC2. In embodiments, the population of expanded human ILC2s includes about 60% NKp30 expressing ILC2. In embodiments, the population of expanded human ILC2s includes about 70% NKp30 expressing ILC2. In embodiments, the population of expanded human ILC2s includes about 80% NKp30 expressing ILC2. In embodiments, the population of expanded human ILC2s includes about 85% NKp30 expressing ILC2. In embodiments, the population of expanded human ILC2s includes about 90% NKp30 expressing ILC2. In embodiments, the population of expanded human ILC2s includes about 91% NKp30 expressing ILC2. In embodiments, the population of expanded human ILC2s includes about 92% NKp30 expressing ILC2. In embodiments, the population of expanded human ILC2s includes about 93% NKp30 expressing ILC2. In embodiments, the population of expanded human ILC2s includes about 94% NKp30 expressing ILC2. In embodiments, the population of expanded human ILC2s includes about 95% NKp30 expressing ILC2. In embodiments, the population of expanded human ILC2s includes about 96% NKp30 expressing ILC2. In embodiments, the population of expanded human ILC2s includes about 97% NKp30 expressing ILC2. In embodiments, the population of expanded human ILC2s includes about 99% NKp30 expressing ILC2. In embodiments, the population of expanded human ILC2s includes about 99% NKp30 expressing ILC2. In embodiments, the population of expanded human ILC2s includes about 100% NKp30 expressing ILC2.

In embodiments, the population of human ILC2s is expanded about 600-fold to about 3000-fold. In embodiments, the population of human ILC2s is expanded about 700-fold to about 3000-fold. In embodiments, the population of human ILC2s is expanded about 800-fold to about 3000-fold. In embodiments, the population of human ILC2s is expanded about 900-fold to about 3000-fold. In embodiments, the population of human ILC2s is expanded about 1000-fold to about 3000-fold. In embodiments, the population of human ILC2s is expanded about 1100-fold to about 3000-fold. In embodiments, the population of human ILC2s is expanded about 1200-fold to about 3000-fold. In embodiments, the population of human ILC2s is expanded about 1300-fold to about 3000-fold. In embodiments, the population of human ILC2s is expanded about 1400-fold to about 3000-fold. In embodiments, the population of human ILC2s is expanded about 1500-fold to about 3000-fold. In embodiments, the population of human ILC2s is expanded about 1600-fold to about 3000-fold. In embodiments, the population of human ILC2s is expanded about 1700-fold to about 3000-fold. In embodiments, the population of human ILC2s is expanded about 1800-fold to about 3000-fold. In embodiments, the population of human ILC2s is expanded about 1900-fold to about 3000-fold. In embodiments, the population of human ILC2s is expanded about 2000-fold to about 3000-fold. In embodiments, the population of human ILC2s is expanded about 2100-fold to about 3000-fold. In embodiments, the population of human ILC2s is expanded about 2200-fold to about 3000-fold. In embodiments, the population of human ILC2s is expanded about 2300-fold to about 3000-fold. In embodiments, the population of human ILC2s is expanded about 2400-fold to about 3000-fold. In embodiments, the population of human ILC2s is expanded about 2500-fold to about 3000-fold. In embodiments, the population of human ILC2s is expanded about 2600-fold to about 3000-fold. In embodiments, the population of human ILC2s is expanded about 2700-fold to about 3000-fold. In embodiments, the population of human ILC2s is expanded about 2800-fold to about 3000-fold. In embodiments, the population of human ILC2s is expanded about 2900-fold to about 3000-fold.

In embodiments, the population of human ILC2s is expanded about 600-fold to about 2900-fold. In embodiments, the population of human ILC2s is expanded about 600-fold to about 2800-fold. In embodiments, the population of human ILC2s is expanded about 600-fold to about 2700-fold. In embodiments, the population of human ILC2s is expanded about 600-fold to about 2600-fold. In embodiments, the population of human ILC2s is expanded about 600-fold to about 2500-fold. In embodiments, the population of human ILC2s is expanded about 600-fold to about 2400-fold. In embodiments, the population of human ILC2s is expanded about 600-fold to about 2300-fold. In embodiments, the population of human ILC2s is expanded about 600-fold to about 2200-fold. In embodiments, the population of human ILC2s is expanded about 600-fold to about 2100-fold. In embodiments, the population of human ILC2s is expanded about 600-fold to about 2000-fold. In embodiments, the population of human ILC2s is expanded about 600-fold to about 1900-fold. In embodiments, the population of human ILC2s is expanded about 600-fold to about 1800-fold. In embodiments, the population of human ILC2s is expanded about 600-fold to about 1700-fold. In embodiments, the population of human ILC2s is expanded about 600-fold to about 1600-fold. In embodiments, the population of human ILC2s is expanded about 600-fold to about 1500-fold. In embodiments, the population of human ILC2s is expanded about 600-fold to about 1400-fold. In embodiments, the population of human ILC2s is expanded about 600-fold to about 1300-fold. In embodiments, the population of human ILC2s is expanded about 600-fold to about 1200-fold. In embodiments, the population of human ILC2s is expanded about 600-fold to about 1100-fold. In embodiments, the population of human ILC2s is expanded about 600-fold to about 1000-fold. In embodiments, the population of human ILC2s is expanded about 600-fold to about 900-fold. In embodiments, the population of human ILC2s is expanded about 600-fold to about 800-fold. In embodiments, the population of human ILC2s is expanded about 600-fold to about 700-fold. In embodiments, the population of human ILC2s is expanded about 600-, 700-, 800-, 900, 1000-, 1100-, 1200-, 1300-, 1400-, 1500-1600-1700-, 1800-, 1900-, 2000-, 2100-, 2200-, 2300-, 2400-, 2500-2600-2700-, 2800-, 2900-, or 3000-fold. In embodiments, the population of human ILC2s is expanded at least 600-, 700-, 800-, 900, 1000-, 1100-, 1200-, 1300-, 1400-, 1500-1600-1700-, 1800-, 1900-, 2000-, 2100-, 2200-, 2300-, 2400-, 2500-2600-2700-, 2800-, 2900-, or 3000-fold.

For the methods provided herein, in embodiments, the population of human ILC2s is expanded about 1200-fold. In embodiments, the population of human ILC2s is expanded 1200-fold. In embodiments, the population of human ILC2s is expanded about 2500-fold. In embodiments, the population of human ILC2s is expanded 2500-fold.

The methods provided herein are contemplated to be useful for generating a population of expanded human ILC2s with high purity. In embodiments, the population of expanded ILC2s generated by the method provided herein including embodiments thereof includes cells that are not ILC2 cells (e.g. non-ILC2 cells). In embodiments, the population of expanded human ILC2s generated by the method provided herein does not include a substantial number of non-ILC2 cells. In embodiments, the population of expanded human ILC2s does not comprise a substantial number of human group 1 innate lymphoid cells (ILC1s), cytotoxic natural killer (NK) cells, or human group 3 innate lymphoid cells (ILC3s). In embodiments, a substantial number of cells refers to greater than about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, or 0.05% of a specific cell type within a population of cells. Thus, in embodiments, the population of expanded human ILC2s provided herein including embodiments thereof comprises less than about 30% cells that are not ILC2s (e.g. non-ILC2 cells). In embodiments, the population of expanded human ILC2s provided herein including embodiments thereof comprises less than about 25% cells that are not ILC2s. In embodiments, the population of expanded human ILC2s provided herein including embodiments thereof comprises less than about 20% cells that are not ILC2s. In embodiments, the population of expanded human ILC2s provided herein including embodiments thereof comprises less than about 19% cells that are not ILC2. In embodiments, the population of expanded human ILC2s provided herein including embodiments thereof comprises less than about 18% cells that are not ILC2s. In embodiments, the population of expanded human ILC2s provided herein including embodiments thereof comprises less than about 17% cells that are not ILC2s. In embodiments, the population of expanded human ILC2s provided herein including embodiments thereof comprises less than about 16% cells that are not ILC2. In embodiments, the population of expanded human ILC2s provided herein including embodiments thereof comprises less than about 15% cells that are not ILC2s. In embodiments, the population of expanded human ILC2s provided herein including embodiments thereof comprises less than about 14% cells that are not ILC2s. In embodiments, the population of expanded human ILC2s provided herein including embodiments thereof comprises less than about 13% cells that are not ILC2s. In embodiments, the population of expanded human ILC2s provided herein including embodiments thereof comprises less than about 12% cells that are not ILC2. In embodiments, the population of expanded human ILC2s provided herein including embodiments thereof comprises less than about 11% cells that are not ILC2. In embodiments, the population of expanded human ILC2s provided herein including embodiments thereof comprises less than about 10% cells that are not ILC2. In embodiments, the population of expanded human ILC2s provided herein including embodiments thereof comprises less than about 9% cells that are not ILC2. In embodiments, the population of expanded human ILC2s provided herein including embodiments thereof comprises less than about 8% cells that are not ILC2. In embodiments, the population of expanded human ILC2s provided herein including embodiments thereof comprises less than about 7% cells that are not ILC2. In embodiments, the population of expanded human ILC2s provided herein including embodiments thereof comprises less than about 6% cells that are not ILC2s. In embodiments, the population of expanded human ILC2s provided herein including embodiments thereof comprises less than about 5% cells that are not ILC2. In embodiments, the population of expanded human ILC2s provided herein including embodiments thereof comprises less than about 4% cells that are not ILC2s. In embodiments, the population of expanded human ILC2s provided herein including embodiments thereof comprises less than about 3% cells that are not ILC2s. In embodiments, the population of expanded human ILC2s provided herein including embodiments thereof comprises less than about 2% cells that are not ILC2s. In embodiments, the population of expanded human ILC2s provided herein including embodiments thereof comprises less than about 1% cells that are not ILC2s. In embodiments, the population of expanded human ILC2s provided herein including embodiments thereof comprises less than about 0.5% cells that are not ILC2s. In embodiments, the population of expanded human ILC2s provided herein including embodiments thereof comprises less than about 0.4% cells that are not ILC2s. In embodiments, the population of expanded human ILC2s provided herein including embodiments thereof comprises less than about 0.3% cells that are not ILC2s. In embodiments, the population of expanded human ILC2s provided herein including embodiments thereof comprises less than about 0.2% cells that are not ILC2s. In embodiments, the population of expanded human ILC2s provided herein including embodiments thereof comprises less than about 0.1% cells that are not ILC2s. In embodiments, the population of expanded human ILC2s provided herein including embodiments thereof comprises less than about 0.05% cells that are not ILC2s.

In embodiments, the population of expanded human ILC2s provided herein including embodiments thereof comprises less than about 30% ILC1s, cytotoxic NK cells or ILC3s. In embodiments, the population of expanded human ILC2s provided herein including embodiments thereof comprises less than about 25% ILC1s, cytotoxic NK cells or ILC3s. In embodiments, the population of expanded human ILC2s provided herein including embodiments thereof comprises less than about 20% ILC1s, cytotoxic NK cells or ILC3s. In embodiments, the population of expanded human ILC2s provided herein including embodiments thereof comprises less than about 19% ILC1s, cytotoxic NK cells or ILC3s. In embodiments, the population of expanded human ILC2s provided herein including embodiments thereof comprises less than about 18% ILC1, cytotoxic NK cells or ILC3s. In embodiments, the population of expanded human ILC2s provided herein including embodiments thereof comprises less than about 17% ILC1s, cytotoxic NK cells or ILC3s. In embodiments, the population of expanded human ILC2s provided herein including embodiments thereof comprises less than about 16% ILC1s, cytotoxic NK cells or ILC3s. In embodiments, the population of expanded human ILC2s provided herein including embodiments thereof comprises less than about 15% ILC1s, cytotoxic NK cells or ILC3s. In embodiments, the population of expanded human ILC2s provided herein including embodiments thereof comprises less than about 14% ILC1s, cytotoxic NK cells or ILC3s. In embodiments, the population of expanded human ILC2s provided herein including embodiments thereof comprises less than about 13% ILC1s, cytotoxic NK cells or ILC3s. In embodiments, the population of expanded human ILC2s provided herein including embodiments thereof comprises less than about 12% ILC1s, cytotoxic NK cells or ILC3s. In embodiments, the population of expanded human ILC2s provided herein including embodiments thereof comprises less than about 11% ILC1s, cytotoxic NK cells or ILC3s. In embodiments, the population of expanded human ILC2s comprises less than about 10% ILC1s, cytotoxic NK cells or ILC3s. In embodiments, the population of expanded human ILC2s provided herein including embodiments thereof comprises less than about 9% ILC1s, cytotoxic NK cells or ILC3s. In embodiments, the population of expanded human ILC2s provided herein including embodiments thereof comprises less than about 8% ILC1s, cytotoxic NK cells or ILC3s. In embodiments, the population of expanded human ILC2s provided herein including embodiments thereof comprises less than about 7% ILC1s, cytotoxic NK cells or ILC3s. In embodiments, the population of expanded human ILC2s provided herein including embodiments thereof comprises less than about 6% ILC1s, cytotoxic NK cells or ILC3s. In embodiments, the population of expanded human ILC2s comprises less than about 5% ILC1s, cytotoxic NK cells or ILC3s. In embodiments, the population of expanded human ILC2s provided herein including embodiments thereof comprises less than about 4% ILC1s, cytotoxic NK cells or ILC3s. In embodiments, the population of expanded human ILC2s provided herein including embodiments thereof comprises less than about 3% ILC1s, cytotoxic NK cells or ILC3s. In embodiments, the population of expanded human ILC2s comprises less than about 2% ILC1s, cytotoxic NK cells or ILC3s. In embodiments, the population of expanded human ILC2s comprises less than about 1% ILC1s, cytotoxic NK cells or ILC3s. In embodiments, the population of expanded human ILC2s provided herein including embodiments thereof comprises less than about 0.9% ILC1, cytotoxic NK cells or ILC3s. In embodiments, the population of expanded human ILC2s provided herein including embodiments thereof comprises less than about 0.8% ILC1s, cytotoxic NK cells or ILC3s. In embodiments, the population of expanded human ILC2s provided herein including embodiments thereof comprises less than about 0.7% ILC1s, cytotoxic NK cells or ILC3s. In embodiments, the population of expanded human ILC2s provided herein including embodiments thereof comprises less than about 0.6% ILC1s, cytotoxic NK cells or ILC3s. In embodiments, the population of expanded human ILC2s provided herein including embodiments thereof comprises less than about 0.5% ILC1, cytotoxic NK cells or ILC3s. In embodiments, the population of expanded human ILC2s comprises less than about 0.4% ILC1s, cytotoxic NK cells or ILC3s. In embodiments, the population of expanded human ILC2s comprises less than about 0.3% ILC1s, cytotoxic NK cells or ILC3s. In embodiments, the population of expanded human ILC2s comprises less than about 0.2% ILC1, cytotoxic NK cells or ILC3s. In embodiments, the population of expanded human ILC2s comprises less than about 0.1% ILC1s, cytotoxic NK cells or ILC3s. In embodiments, the population of expanded human ILC2s provided herein including embodiments thereof comprises less than about 0.05% ILC1s, cytotoxic NK cells or ILC3s.

For the methods provided herein, in embodiments, the population of expanded human ILC2s does not include a substantial number of cells expressing T-BET or EOMES. In embodiments, the population of of expanded human ILC2s includes less than about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, or 0.05% cells expressing T-BET or EOMES. In embodiments, the population of expanded human ILC2s does not include a substantial number of cells expressing T-BET. In embodiments, the population of expanded human ILC does not include a substantial number of cells expressing EOMES.

In embodiments, the population of expanded human ILC does not include a substantial number of cells expressing IFNγ or TNF. In embodiments, the population of of expanded human ILC2s includes less than about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, or 0.05% cells expressing IFNγ or TNF. In embodiments, the population of expanded human ILC2s does not include a substantial number of cells expressing IFNγ. In embodiments, the population of expanded human ILC does not include a substantial number of cells expressing TNF.

In embodiments, the population of expanded human ILC2s does not include a substantial number of cells expressing RORγ. In embodiments, the population of of expanded human ILC2s includes less than about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, or 0.05% cells expressing RORγ.

### Human Ilc2 Compositions

Provided herein, inter alia, is a population of expanded human group 2 innate lymphoid cells (ILC2). The population of expanded human ILC2s provided herein including embodiments thereof include ILC2s expressing one or more proteins (e.g. granzyme B) involved in killing cancer cells. As described herein, the population of expanded human ILC2s provided herein including embodiments thereof does not include a substantial amount of non-ILC2 cells. For example, in embodiments, the population of expanded human ILC2s includes less than about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, or 0.05% non-ILC2 cells. Thus, in an aspect is provided a population of expanded human ILC2s made by the method provided herein including embodiments thereof.

In another aspect is provided a population of expanded human group 2 innate lymphoid cells (ILC2), wherein the population of expanded ILC2s includes at least 30% ILC2s. In embodiments, the population of expanded human ILC2s includes less than about 70% non-ILC2 cells (e.g. ILC1, cytotoxic NK cells or ILC3s). In embodiments, the population of expanded human ILC2s includes at least 40% ILC2s. In embodiments, the population of expanded human ILC2s includes at least 50% ILC2s. In embodiments, the population of expanded human ILC2s includes at least 60% ILC2s. In embodiments, the population of expanded human ILC2s includes at least 70% ILC2s. In embodiments, the population of expanded human ILC2s includes at least 80% ILC2s. In embodiments, the population of expanded human ILC2s includes at least 85% ILC2s. In embodiments, the population of expanded human ILC2s includes at least 90% ILC2s. In embodiments, the population of expanded human ILC2s includes at least 91% ILC2s. In embodiments, the population of expanded human ILC2s includes at least 92% ILC2s. In embodiments, the population of expanded human ILC2s includes at least 93% ILC2s. In embodiments, the population of expanded human ILC2s includes at least 94% ILC2s. In embodiments, the population of expanded human ILC2s includes at least 95% ILC2s. In embodiments, the population of expanded human ILC2s includes at least 96% ILC2s. In embodiments, the population of expanded human ILC2s includes at least 97% ILC2s. In embodiments, the population of expanded human ILC2s includes at least 98% ILC2s. In embodiments, the population of expanded human ILC2s includes at least 99% ILC2s.

In embodiments, the population of expanded human ILC2s includes about 30% ILC2s. In embodiments, the population of expanded human ILC2s includes about 40% ILC2s. In embodiments, the population of expanded human ILC2s includes about 50% ILC2s. In embodiments, the population of expanded human ILC2s includes about 60% ILC2s. In embodiments, the population of expanded human ILC2s includes about 70% ILC2s. In embodiments, the population of expanded human ILC2s includes about 80% ILC2s. In embodiments, the population of expanded human ILC2s includes about 85% ILC2s. In embodiments, the population of expanded human ILC2s includes about 90% ILC2s. In embodiments, the population of expanded human ILC2s includes about 91% ILC2s. In embodiments, the population of expanded human ILC2s includes about 92% ILC2s. In embodiments, the population of expanded human ILC2s includes about 93% ILC2s. In embodiments, the population of expanded human ILC2s includes about 94% ILC2s. In embodiments, the population of expanded human ILC2s includes about 95% ILC2s. In embodiments, the population of expanded human ILC2s includes about 96% ILC2s. In embodiments, the population of expanded human ILC2s includes about 97% ILC2s. In embodiments, the population of expanded human ILC2s includes about 98% ILC2s. In embodiments, the population of expanded human ILC2s includes about 99% ILC2s. In embodiments, the population of expanded human ILC2s includes about 100% ILC2s.

For the population of expanded human ILC2s provided herein, in embodiments, the ILC2s include CD161⁺ ILC2s. In embodiments, at least 30% of the ILC2s in the population of expanded ILC2s are CD161⁺ ILC2s. In embodiments, at least 40% of the ILC2s in the population of expanded ILC2s are CD161⁺ ILC2s. In embodiments, at least 50% of the ILC2s in the population of expanded ILC2s are CD161⁺ ILC2s. In embodiments, at least 60% of the ILC2s in the population of expanded ILC2s are CD161⁺ ILC2s. In embodiments, at least 70% of the ILC2s in the population of expanded ILC2s are CD161⁺ ILC2s. In embodiments, at least 80% of the ILC2s in the population of expanded ILC2s are CD161⁺ ILC2s. In embodiments, at least 85% of the ILC2s in the population of expanded ILC2s are CD161⁺ ILC2s. In embodiments, at least 90% of the ILC2s in the population of expanded ILC2s are CD161⁺ ILC2s. In embodiments, at least 91% of the ILC2s in the population of expanded ILC2s are CD161⁺ ILC2s. In embodiments, at least 92% of the ILC2s in the population of expanded ILC2s are CD161⁺ ILC2s. In embodiments, at least 93% of the ILC2s in the population of expanded ILC2s are CD161⁺ ILC2s. In embodiments, at least 94% of the ILC2s in the population of expanded ILC2s are CD161⁺ ILC2s. In embodiments, at least 95% of the ILC2s in the population of expanded ILC2s are CD161⁺ ILC2s. In embodiments, at least 96% of the ILC2s in the population of expanded ILC2s are CD161⁺ ILC2s. In embodiments, at least 97% of the ILC2s in the population of expanded ILC2s are CD161⁺ ILC2s. In embodiments, at least 98% of the ILC2s in the population of expanded ILC2s are CD161⁺ ILC2s. In embodiments, at least 99% of the ILC2s in the population of expanded ILC2s are CD161⁺ ILC2s.

In embodiments, about 30% of the ILC2s in the population of expanded ILC2s are CD161⁺ ILC2s. In embodiments, about 40% of the ILC2s in the population of expanded ILC2s are CD161⁺ ILC2s. In embodiments, about 50% of the ILC2s in the population of expanded ILC2s are CD161⁺ ILC2s. In embodiments, about 60% of the ILC2s in the population of expanded ILC2s are CD161⁺ ILC2s. In embodiments, about 70% of the ILC2s in the population of expanded ILC2s are CD161⁺ ILC2s. In embodiments, about 80% of the ILC2s in the population of expanded ILC2s are CD161⁺ ILC2s. In embodiments, about 85% of the ILC2s in the population of expanded ILC2s are CD161⁺ ILC2s. In embodiments, about 90% of the ILC2s in the population of expanded ILC2s are CD161⁺ ILC2s. In embodiments, about 91% of the ILC2s in the population of expanded ILC2s are CD161⁺ ILC2s. In embodiments, about 92% of the ILC2s in the population of expanded ILC2s are CD161⁺ ILC2s. In embodiments, about 93% of the ILC2s in the population of expanded ILC2s are CD161⁺ ILC2s. In embodiments, about 94% of the ILC2s in the population of expanded ILC2s are CD161⁺ ILC2s. In embodiments, about 95% of the ILC2s in the population of expanded ILC2s are CD161⁺ ILC2s. In embodiments, about 96% of the ILC2s in the population of expanded ILC2s are CD161⁺ ILC2s. In embodiments, about 97% of the ILC2s in the population of expanded ILC2s are CD161⁺ ILC2s. In embodiments, about 98% of the ILC2s in the population of expanded ILC2s are CD161⁺ ILC2s. In embodiments, about 99% of the ILC2s in the population of expanded ILC2s are CD161⁺ ILC2s. In embodiments, about 100% of the ILC2s in the population of expanded ILC2s are CD161⁺ ILC2s.

For the population of expanded human ILC2s provided herein, in embodiments, the ILC2s include CRTH2+CD117+ ILC2s. In embodiments, at least 30% of the ILC2s in the population of expanded ILC2s are CRTH2+CD117+ ILC2s. In embodiments, at least 40% of the ILC2s in the population of expanded ILC2s are CRTH2+CD117+ ILC2s. In embodiments, at least 50% of the ILC2s in the population of expanded ILC2s are CRTH2+CD117+ ILC2s. In embodiments, at least 60% of the ILC2s in the population of expanded ILC2s are CRTH2+CD117+ ILC2s. In embodiments, at least 70% of the ILC2s in the population of expanded ILC2s are CRTH2+CD117+ ILC2s. In embodiments, at least 80% of the ILC2s in the population of expanded ILC2s are CRTH2+CD117+ ILC2s. In embodiments, at least 85% of the ILC2s in the population of expanded ILC2s are CRTH2+CD117+ ILC2s. In embodiments, at least 90% of the ILC2s in the population of expanded ILC2s are CRTH2+CD117+ ILC2s. In embodiments, at least 91% of the ILC2s in the population of expanded ILC2s are CRTH2+CD117+ ILC2s. In embodiments, at least 92% of the ILC2s in the population of expanded ILC2s are CRTH2+CD117+ ILC2s. In embodiments, at least 93% of the ILC2s in the population of expanded ILC2s are CRTH2+CD117+ ILC2s. In embodiments, at least 94% of the ILC2s in the population of expanded ILC2s are CRTH2+CD117+ ILC2s. In embodiments, at least 95% of the ILC2s in the population of expanded ILC2s are CRTH2+CD117+ ILC2s. In embodiments, at least 96% of the ILC2s in the population of expanded ILC2s are CRTH2+CD117+ ILC2s. In embodiments, at least 97% of the ILC2s in the population of expanded ILC2s are CRTH2+CD117+ ILC2s. In embodiments, at least 98% of the ILC2s in the population of expanded ILC2s are CRTH2+CD117+ ILC2s. In embodiments, at least 99% of the ILC2s in the population of expanded ILC2s are CRTH2+CD117+ ILC2s.

In embodiments, about 30% of the ILC2s in the population of expanded ILC2s are CRTH2+CD117+ ILC2s. In embodiments, about 40% of the ILC2s in the population of expanded ILC2s are CRTH2+CD117+ ILC2s. In embodiments, about 50% of the ILC2s in the population of expanded ILC2s are CRTH2+CD117+ ILC2s. In embodiments, about 60% of the ILC2s in the population of expanded ILC2s are CRTH2+CD117+ ILC2s. In embodiments, about 70% of the ILC2s in the population of expanded ILC2s are CRTH2+CD117+ ILC2s. In embodiments, about 80% of the ILC2s in the population of expanded ILC2s are CRTH2+CD117+ILC2s. In embodiments, about 85% of the ILC2s in the population of expanded ILC2s are CRTH2+CD117+ ILC2s. In embodiments, about 90% of the ILC2s in the population of expanded ILC2s are CRTH2+CD117+ ILC2s. In embodiments, about 91% of the ILC2s in the population of expanded ILC2s are CRTH2+CD117+ ILC2s. In embodiments, about 92% of the ILC2s in the population of expanded ILC2s are CRTH2+CD117+ ILC2s. In embodiments, about 93% of the ILC2s in the population of expanded ILC2s are CRTH2+CD117+ ILC2s. In embodiments, about 94% of the ILC2s in the population of expanded ILC2s are CRTH2+CD117+ ILC2s. In embodiments, about 95% of the ILC2s in the population of expanded ILC2s are CRTH2+CD117+ILC2s. In embodiments, about 96% of the ILC2s in the population of expanded ILC2s are CRTH2+CD117+ ILC2s. In embodiments, about 97% of the ILC2s in the population of expanded ILC2s are CRTH2+CD117+ ILC2s. In embodiments, about 98% of the ILC2s in the population of expanded ILC2s are CRTH2+CD117+ ILC2s. In embodiments, about 99% of the ILC2s in the population of expanded ILC2s are CRTH2+CD117+ ILC2s. In embodiments, about 100% of the ILC2s in the population of expanded ILC2s are CRTH2+CD117+ ILC2s.

For the population of expanded human ILC2s provided herein, in embodiments, the ILC2s include CD122+ ILC2s. In embodiments, at least 30% of the ILC2s in the population of expanded ILC2s are CD122+ ILC2s. In embodiments, at least 40% of the ILC2s in the population of expanded ILC2s are CD122+ ILC2s. In embodiments, at least 50% of the ILC2s in the population of expanded ILC2s are CD122+ ILC2s. In embodiments, at least 60% of the ILC2s in the population of expanded ILC2s are CD122+ ILC2s. In embodiments, at least 70% of the ILC2s in the population of expanded ILC2s are CD122+ ILC2s. In embodiments, at least 80% of the ILC2s in the population of expanded ILC2s are CD122+ ILC2s. In embodiments, at least 85% of the ILC2s in the population of expanded ILC2s are CD122+ ILC2s. In embodiments, at least 90% of the ILC2s in the population of expanded ILC2s are CD122+ ILC2s. In embodiments, at least 91% of the ILC2s in the population of expanded ILC2s are CD122+ ILC2s. In embodiments, at least 92% of the ILC2s in the population of expanded ILC2s are CD122+ ILC2s. In embodiments, at least 93% of the ILC2s in the population of expanded ILC2s are CD122+ ILC2s. In embodiments, at least 94% of the ILC2s in the population of expanded ILC2s are CD122+ ILC2s. In embodiments, at least 95% of the ILC2s in the population of expanded ILC2s are CD122+ ILC2s. In embodiments, at least 96% of the ILC2s in the population of expanded ILC2s are CD122+ ILC2s. In embodiments, at least 97% of the ILC2s in the population of expanded ILC2s are CD122+ ILC2s. In embodiments, at least 98% of the ILC2s in the population of expanded ILC2s are CD122+ ILC2s. In embodiments, at least 99% of the ILC2s in the population of expanded ILC2s are CD122+ ILC2s.

In embodiments, about 30% of the ILC2s in the population of expanded ILC2s are CD122+ ILC2s. In embodiments, about 40% of the ILC2s in the population of expanded ILC2s are CD122+ ILC2s. In embodiments, about 50% of the ILC2s in the population of expanded ILC2s are CD122+ ILC2s. In embodiments, about 60% of the ILC2s in the population of expanded ILC2s are CD122+ ILC2s. In embodiments, about 70% of the ILC2s in the population of expanded ILC2s are CD122+ ILC2s. In embodiments, about 80% of the ILC2s in the population of expanded ILC2s are CD122+ ILC2s. In embodiments, about 85% of the ILC2s in the population of expanded ILC2s are CD122+ ILC2s. In embodiments, about 90% of the ILC2s in the population of expanded ILC2s are CD122+ ILC2s. In embodiments, about 91% of the ILC2s in the population of expanded ILC2s are CD122+ ILC2s. In embodiments, about 92% of the ILC2s in the population of expanded ILC2s are CD122+ ILC2s. In embodiments, about 93% of the ILC2s in the population of expanded ILC2s are CD122+ ILC2s. In embodiments, about 94% of the ILC2s in the population of expanded ILC2s are CD122+ ILC2s. In embodiments, about 95% of the ILC2s in the population of expanded ILC2s are CD122+ ILC2s. In embodiments, about 96% of the ILC2s in the population of expanded ILC2s are CD122+ ILC2s. In embodiments, about 97% of the ILC2s in the population of expanded ILC2s are CD122+ ILC2s. In embodiments, about 98% of the ILC2s in the population of expanded ILC2s are CD122+ ILC2s. In embodiments, about 99% of the ILC2s in the population of expanded ILC2s are CD122+ ILC2s. In embodiments, about 100% of the ILC2s in the population of expanded ILC2s are CD122+ ILC2s.

For the population of expanded human ILC2s provided herein, in embodiments, the ILC2s express one or more proteins involved in killing cancer cells. For example, the population of expanded ILC2s may include ILC2s expressing granzyme B (GZMB) and/or DNAM-1. Thus, for the population of expanded human ILC2s provided herein, in embodiments, the ILC2s include DNAX Accessory Molecule-1 (DNAM-1) expressing ILC2s. In embodiments, at least 30% of the ILC2s in the population of expanded ILC2s are DNAM-1 expressing ILC2s. In embodiments, at least 40% of the ILC2s in the population of expanded ILC2s are DNAM-1 expressing ILC2s. In embodiments, at least 50% of the ILC2s in the population of expanded ILC2s are DNAM-1 expressing ILC2s. In embodiments, at least 60% of the ILC2s in the population of expanded ILC2s are DNAM-1 expressing ILC2s. In embodiments, at least 70% of the ILC2s in the population of expanded ILC2s are DNAM-1 expressing ILC2s. In embodiments, at least 80% of the ILC2s in the population of expanded ILC2s are DNAM-1 expressing ILC2s. In embodiments, at least 85% of the ILC2s in the population of expanded ILC2s are DNAM-1 expressing ILC2s. In embodiments, at least 90% of the ILC2s in the population of expanded ILC2s are DNAM-1 expressing ILC2s. In embodiments, at least 91% of the ILC2s in the population of expanded ILC2s are DNAM-1 expressing ILC2s. In embodiments, at least 92% of the ILC2s in the population of expanded ILC2s are DNAM-1 expressing ILC2s. In embodiments, at least 93% of the ILC2s in the population of expanded ILC2s are DNAM-1 expressing ILC2s. In embodiments, at least 94% of the ILC2s in the population of expanded ILC2s are DNAM-1 expressing ILC2s. In embodiments, at least 95% of the ILC2s in the population of expanded ILC2s are DNAM-1 expressing ILC2s. In embodiments, at least 96% of the ILC2s in the population of expanded ILC2s are DNAM-1 expressing ILC2s. In embodiments, at least 97% of the ILC2s in the population of expanded ILC2s are DNAM-1 expressing ILC2s. In embodiments, at least 98% of the ILC2s in the population of expanded ILC2s are DNAM-1 expressing ILC2s. In embodiments, at least 99% of the ILC2s in the population of expanded ILC2s are DNAM-1 expressing ILC2s.

In embodiments, about 30% of the ILC2s in the population of expanded ILC2s are DNAM-1 expressing ILC2s. In embodiments, about 40% of the ILC2s in the population of expanded ILC2s are DNAM-1 expressing ILC2s. In embodiments, about 50% of the ILC2s in the population of expanded ILC2s are DNAM-1 expressing ILC2s. In embodiments, about 60% of the ILC2s in the population of expanded ILC2s are DNAM-1 expressing ILC2s. In embodiments, about 70% of the ILC2s in the population of expanded ILC2s are DNAM-1 expressing ILC2s. In embodiments, about 80% of the ILC2s in the population of expanded ILC2s are DNAM-1 expressing ILC2s. In embodiments, about 85% of the ILC2s in the population of expanded ILC2s are DNAM-1 expressing ILC2s. In embodiments, about 90% of the ILC2s in the population of expanded ILC2s are DNAM-1 expressing ILC2s. In embodiments, about 91% of the ILC2s in the population of expanded ILC2s are DNAM-1 expressing ILC2s. In embodiments, about 92% of the ILC2s in the population of expanded ILC2s are DNAM-1 expressing ILC2s. In embodiments, about 93% of the ILC2s in the population of expanded ILC2s are DNAM-1 expressing ILC2s. In embodiments, about 94% of the ILC2s in the population of expanded ILC2s are DNAM-1 expressing ILC2s. In embodiments, about 95% of the ILC2s in the population of expanded ILC2s are DNAM-1 expressing ILC2s. In embodiments, about 96% of the ILC2s in the population of expanded ILC2s are DNAM-1 expressing ILC2s. In embodiments, about 97% of the ILC2s in the population of expanded ILC2s are DNAM-1 expressing ILC2s. In embodiments, about 98% of the ILC2s in the population of expanded ILC2s are DNAM-1 expressing ILC2s. In embodiments, about 99% of the ILC2s in the population of expanded ILC2s are DNAM-1 expressing ILC2s. In embodiments, about 100% of the ILC2s in the population of expanded ILC2s are DNAM-1 expressing ILC2s.

For the population of expanded human ILC2s provided herein, in embodiments, the ILC2s includes granzyme B (GZMB) expressing ILC2s (e.g. GZMB⁺ ILC2). In embodiments, at least 30% of the ILC2s in the population of expanded ILC2s are GZMB expressing ILC2s. In embodiments, at least 40% of the ILC2s in the population of expanded ILC2s are GZMB expressing ILC2s. In embodiments, at least 50% of the ILC2s in the population of expanded ILC2s are GZMB expressing ILC2s. In embodiments, at least 60% of the ILC2s in the population of expanded ILC2s are GZMB expressing ILC2s. In embodiments, at least 70% of the ILC2s in the population of expanded ILC2s are GZMB expressing ILC2s. In embodiments, at least 80% of the ILC2s in the population of expanded ILC2s are GZMB expressing ILC2s. In embodiments, at least 85% of the ILC2s in the population of expanded ILC2s are GZMB expressing ILC2s. In embodiments, at least 90% of the ILC2s in the population of expanded ILC2s are GZMB expressing ILC2s. In embodiments, at least 91% of the ILC2s in the population of expanded ILC2s are GZMB expressing ILC2s. In embodiments, at least 92% of the ILC2s in the population of expanded ILC2s are GZMB expressing ILC2s. In embodiments, at least 93% of the ILC2s in the population of expanded ILC2s are GZMB expressing ILC2s. In embodiments, at least 94% of the ILC2s in the population of expanded ILC2s are GZMB expressing ILC2s. In embodiments, at least 95% of the ILC2s in the population of expanded ILC2s are GZMB expressing ILC2s. In embodiments, at least 96% of the ILC2s in the population of expanded ILC2s are GZMB expressing ILC2s. In embodiments, at least 97% of the ILC2s in the population of expanded ILC2s are GZMB expressing ILC2s. In embodiments, at least 98% of the ILC2s in the population of expanded ILC2s are GZMB expressing ILC2s. In embodiments, at least 99% of the ILC2s in the population of expanded ILC2s are GZMB expressing ILC2s.

In embodiments, about 30% of the ILC2s in the population of expanded ILC2s are GZMB expressing ILC2s. In embodiments, about 40% of the ILC2s in the population of expanded ILC2s are GZMB expressing ILC2s. In embodiments, about 50% of the ILC2s in the population of expanded ILC2s are GZMB expressing ILC2s. In embodiments, about 60% of the ILC2s in the population of expanded ILC2s are GZMB expressing ILC2s. In embodiments, about 70% of the ILC2s in the population of expanded ILC2s are GZMB expressing ILC2s. In embodiments, about 80% of the ILC2s in the population of expanded ILC2s are GZMB expressing ILC2s. In embodiments, about 85% of the ILC2s in the population of expanded ILC2s are GZMB expressing ILC2s. In embodiments, about 90% of the ILC2s in the population of expanded ILC2s are GZMB expressing ILC2s. In embodiments, about 91% of the ILC2s in the population of expanded ILC2s are GZMB expressing ILC2s. In embodiments, about 92% of the ILC2s in the population of expanded ILC2s are GZMB expressing ILC2s. In embodiments, about 93% of the ILC2s in the population of expanded ILC2s are GZMB expressing ILC2s. In embodiments, about 94% of the ILC2s in the population of expanded ILC2s are GZMB expressing ILC2s. In embodiments, about 95% of the ILC2s in the population of expanded ILC2s are GZMB expressing ILC2s. In embodiments, about 96% of the ILC2s in the population of expanded ILC2s are GZMB expressing ILC2s. In embodiments, about 97% of the ILC2s in the population of expanded ILC2s are GZMB expressing ILC2s. In embodiments, about 98% of the ILC2s in the population of expanded ILC2s are GZMB expressing ILC2s. In embodiments, about 99% of the ILC2s in the population of expanded ILC2s are GZMB expressing ILC2s. In embodiments, about 100% of the ILC2s in the population of expanded ILC2s are GZMB expressing ILC2s.

In embodiments, the population of expanded human ILC2s includes ILC2s expressing IL-4, IL-5, IL-9, IL-13, or a combination thereof. For the population of expanded human ILC2s provided herein, in embodiments, the ILC2s includes IL-4 expressing ILC2s (e.g. IL-4⁺ ILC2). In embodiments, at least 30% of the ILC2s in the population of expanded ILC2s are IL-4 expressing ILC2s. In embodiments, at least 40% of the ILC2s in the population of expanded ILC2s are IL-4 expressing ILC2s. In embodiments, at least 50% of the ILC2s in the population of expanded ILC2s are IL-4 expressing ILC2s. In embodiments, at least 60% of the ILC2s in the population of expanded ILC2s are IL-4 expressing ILC2s. In embodiments, at least 70% of the ILC2s in the population of expanded ILC2s are IL-4 expressing ILC2s. In embodiments, at least 80% of the ILC2s in the population of expanded ILC2s are IL-4 expressing ILC2s. In embodiments, at least 85% of the ILC2s in the population of expanded ILC2s are IL-4 expressing ILC2s. In embodiments, at least 90% of the ILC2s in the population of expanded ILC2s are IL-4 expressing ILC2s. In embodiments, at least 91% of the ILC2s in the population of expanded ILC2s are IL-4 expressing ILC2s. In embodiments, at least 92% of the ILC2s in the population of expanded ILC2s are IL-4 expressing ILC2s. In embodiments, at least 93% of the ILC2s in the population of expanded ILC2s are IL-4 expressing ILC2s. In embodiments, at least 94% of the ILC2s in the population of expanded ILC2s are IL-4 expressing ILC2s. In embodiments, at least 95% of the ILC2s in the population of expanded ILC2s are IL-4 expressing ILC2s. In embodiments, at least 96% of the ILC2s in the population of expanded ILC2s are IL-4 expressing ILC2s. In embodiments, at least 97% of the ILC2s in the population of expanded ILC2s are IL-4 expressing ILC2s. In embodiments, at least 98% of the ILC2s in the population of expanded ILC2s are IL-4 expressing ILC2s. In embodiments, at least 99% of the ILC2s in the population of expanded ILC2s are IL-4 expressing ILC2s.

In embodiments, about 30% of the ILC2s in the population of expanded ILC2s are IL-4 expressing ILC2s. In embodiments, about 40% of the ILC2s in the population of expanded ILC2s are IL-4 expressing ILC2s. In embodiments, about 50% of the ILC2s in the population of expanded ILC2s are IL-4 expressing ILC2s. In embodiments, about 60% of the ILC2s in the population of expanded ILC2s are IL-4 expressing ILC2s. In embodiments, about 70% of the ILC2s in the population of expanded ILC2s are IL-4 expressing ILC2s. In embodiments, about 80% of the ILC2s in the population of expanded ILC2s are IL-4 expressing ILC2s. In embodiments, about 85% of the ILC2s in the population of expanded ILC2s are IL-4 expressing ILC2s. In embodiments, about 90% of the ILC2s in the population of expanded ILC2s are IL-4 expressing ILC2s. In embodiments, about 91% of the ILC2s in the population of expanded ILC2s are IL-4 expressing ILC2s. In embodiments, about 92% of the ILC2s in the population of expanded ILC2s are IL-4 expressing ILC2s. In embodiments, about 93% of the ILC2s in the population of expanded ILC2s are IL-4 expressing ILC2s. In embodiments, about 94% of the ILC2s in the population of expanded ILC2s are IL-4 expressing ILC2s. In embodiments, about 95% of the ILC2s in the population of expanded ILC2s are IL-4 expressing ILC2s. In embodiments, about 96% of the ILC2s in the population of expanded ILC2s are IL-4 expressing ILC2s. In embodiments, about 97% of the ILC2s in the population of expanded ILC2s are IL-4 expressing ILC2s. In embodiments, about 98% of the ILC2s in the population of expanded ILC2s are IL-4 expressing ILC2s. In embodiments, about 99% of the ILC2s in the population of expanded ILC2s are IL-4 expressing ILC2s. In embodiments, about 100% of the ILC2s in the population of expanded ILC2s are IL-4 expressing ILC2s.

For the population of expanded human ILC2s provided herein, in embodiments, the ILC2s includes IL-5 expressing ILC2s (e.g. IL-5⁺ ILC2). In embodiments, at least 30% of the ILC2s in the population of expanded ILC2s are IL-5 expressing ILC2s. In embodiments, at least 40% of the ILC2s in the population of expanded ILC2s are IL-5 expressing ILC2s. In embodiments, at least 50% of the ILC2s in the population of expanded ILC2s are IL-5 expressing ILC2s. In embodiments, at least 60% of the ILC2s in the population of expanded ILC2s are IL-5 expressing ILC2s. In embodiments, at least 70% of the ILC2s in the population of expanded ILC2s are IL-5 expressing ILC2s. In embodiments, at least 80% of the ILC2s in the population of expanded ILC2s are IL-5 expressing ILC2s. In embodiments, at least 85% of the ILC2s in the population of expanded ILC2s are IL-5 expressing ILC2s. In embodiments, at least 90% of the ILC2s in the population of expanded ILC2s are IL-5 expressing ILC2s. In embodiments, at least 91% of the ILC2s in the population of expanded ILC2s are IL-5 expressing ILC2s. In embodiments, at least 92% of the ILC2s in the population of expanded ILC2s are IL-5 expressing ILC2s. In embodiments, at least 93% of the ILC2s in the population of expanded ILC2s are IL-5 expressing ILC2s. In embodiments, at least 94% of the ILC2s in the population of expanded ILC2s are IL-5 expressing ILC2s. In embodiments, at least 95% of the ILC2s in the population of expanded ILC2s are IL-5 expressing ILC2s. In embodiments, at least 96% of the ILC2s in the population of expanded ILC2s are IL-5 expressing ILC2s. In embodiments, at least 97% of the ILC2s in the population of expanded ILC2s are IL-5 expressing ILC2s. In embodiments, at least 98% of the ILC2s in the population of expanded ILC2s are IL-5 expressing ILC2s. In embodiments, at least 99% of the ILC2s in the population of expanded ILC2s are IL-5 expressing ILC2s.

In embodiments, about 30% of the ILC2s in the population of expanded ILC2s are IL-5 expressing ILC2s. In embodiments, about 40% of the ILC2s in the population of expanded ILC2s are IL-5 expressing ILC2s. In embodiments, about 50% of the ILC2s in the population of expanded ILC2s are IL-5 expressing ILC2s. In embodiments, about 60% of the ILC2s in the population of expanded ILC2s are IL-5 expressing ILC2s. In embodiments, about 70% of the ILC2s in the population of expanded ILC2s are IL-5 expressing ILC2s. In embodiments, about 80% of the ILC2s in the population of expanded ILC2s are IL-5 expressing ILC2s. In embodiments, about 85% of the ILC2s in the population of expanded ILC2s are IL-5 expressing ILC2s. In embodiments, about 90% of the ILC2s in the population of expanded ILC2s are IL-5 expressing ILC2s. In embodiments, about 91% of the ILC2s in the population of expanded ILC2s are IL-5 expressing ILC2s. In embodiments, about 92% of the ILC2s in the population of expanded ILC2s are IL-5 expressing ILC2s. In embodiments, about 93% of the ILC2s in the population of expanded ILC2s are IL-5 expressing ILC2s. In embodiments, about 94% of the ILC2s in the population of expanded ILC2s are IL-5 expressing ILC2s. In embodiments, about 95% of the ILC2s in the population of expanded ILC2s are IL-5 expressing ILC2s. In embodiments, about 96% of the ILC2s in the population of expanded ILC2s are IL-5 expressing ILC2s. In embodiments, about 97% of the ILC2s in the population of expanded ILC2s are IL-5 expressing ILC2s. In embodiments, about 98% of the ILC2s in the population of expanded ILC2s are IL-5 expressing ILC2s. In embodiments, about 99% of the ILC2s in the population of expanded ILC2s are IL-5 expressing ILC2s. In embodiments, about 100% of the ILC2s in the population of expanded ILC2s are IL-5 expressing ILC2s.

For the population of expanded human ILC2s provided herein, in embodiments, the ILC2s includes IL-9 expressing ILC2s (e.g. IL-9⁺ ILC2). In embodiments, at least 30% of the ILC2s in the population of expanded ILC2s are IL-9 expressing ILC2s. In embodiments, at least 40% of the ILC2s in the population of expanded ILC2s are IL-9 expressing ILC2s. In embodiments, at least 50% of the ILC2s in the population of expanded ILC2s are IL-9 expressing ILC2s. In embodiments, at least 60% of the ILC2s in the population of expanded ILC2s are IL-9 expressing ILC2s. In embodiments, at least 70% of the ILC2s in the population of expanded ILC2s are IL-9 expressing ILC2s. In embodiments, at least 80% of the ILC2s in the population of expanded ILC2s are IL-9 expressing ILC2s. In embodiments, at least 85% of the ILC2s in the population of expanded ILC2s are IL-9 expressing ILC2s. In embodiments, at least 90% of the ILC2s in the population of expanded ILC2s are IL-9 expressing ILC2s. In embodiments, at least 91% of the ILC2s in the population of expanded ILC2s are IL-9 expressing ILC2s. In embodiments, at least 92% of the ILC2s in the population of expanded ILC2s are IL-9 expressing ILC2s. In embodiments, at least 93% of the ILC2s in the population of expanded ILC2s are IL-9 expressing ILC2s. In embodiments, at least 94% of the ILC2s in the population of expanded ILC2s are IL-9 expressing ILC2s. In embodiments, at least 95% of the ILC2s in the population of expanded ILC2s are IL-9 expressing ILC2s. In embodiments, at least 96% of the ILC2s in the population of expanded ILC2s are IL-9 expressing ILC2s. In embodiments, at least 97% of the ILC2s in the population of expanded ILC2s are IL-9 expressing ILC2s. In embodiments, at least 98% of the ILC2s in the population of expanded ILC2s are IL-9 expressing ILC2s. In embodiments, at least 99% of the ILC2s in the population of expanded ILC2s are IL-9 expressing ILC2s.

In embodiments, about 30% of the ILC2s in the population of expanded ILC2s are IL-9 expressing ILC2s. In embodiments, about 40% of the ILC2s in the population of expanded ILC2s are IL-9 expressing ILC2s. In embodiments, about 50% of the ILC2s in the population of expanded ILC2s are IL-9 expressing ILC2s. In embodiments, about 60% of the ILC2s in the population of expanded ILC2s are IL-9 expressing ILC2s. In embodiments, about 70% of the ILC2s in the population of expanded ILC2s are IL-9 expressing ILC2s. In embodiments, about 80% of the ILC2s in the population of expanded ILC2s are IL-9 expressing ILC2s. In embodiments, about 85% of the ILC2s in the population of expanded ILC2s are IL-9 expressing ILC2s. In embodiments, about 90% of the ILC2s in the population of expanded ILC2s are IL-9 expressing ILC2s. In embodiments, about 91% of the ILC2s in the population of expanded ILC2s are IL-9 expressing ILC2s. In embodiments, about 92% of the ILC2s in the population of expanded ILC2s are IL-9 expressing ILC2s. In embodiments, about 93% of the ILC2s in the population of expanded ILC2s are IL-9 expressing ILC2s. In embodiments, about 94% of the ILC2s in the population of expanded ILC2s are IL-9 expressing ILC2s. In embodiments, about 95% of the ILC2s in the population of expanded ILC2s are IL-9 expressing ILC2s. In embodiments, about 96% of the ILC2s in the population of expanded ILC2s are IL-9 expressing ILC2s. In embodiments, about 97% of the ILC2s in the population of expanded ILC2s are IL-9 expressing ILC2s. In embodiments, about 98% of the ILC2s in the population of expanded ILC2s are IL-9 expressing ILC2s. In embodiments, about 99% of the ILC2s in the population of expanded ILC2s are IL-9 expressing ILC2s. In embodiments, about 100% of the ILC2s in the population of expanded ILC2s are IL-9 expressing ILC2s.

For the population of expanded human ILC2s provided herein, in embodiments, the ILC2s includes IL-13 expressing ILC2s (e.g. IL-13⁺ ILC2). In embodiments, at least 30% of the ILC2s in the population of expanded ILC2s are IL-13 expressing ILC2s. In embodiments, at least 40% of the ILC2s in the population of expanded ILC2s are IL-13 expressing ILC2s. In embodiments, at least 50% of the ILC2s in the population of expanded ILC2s are IL-13 expressing ILC2s. In embodiments, at least 60% of the ILC2s in the population of expanded ILC2s are IL-13 expressing ILC2s. In embodiments, at least 70% of the ILC2s in the population of expanded ILC2s are IL-13 expressing ILC2s. In embodiments, at least 80% of the ILC2s in the population of expanded ILC2s are IL-13 expressing ILC2s. In embodiments, at least 85% of the ILC2s in the population of expanded ILC2s are IL-13 expressing ILC2s. In embodiments, at least 90% of the ILC2s in the population of expanded ILC2s are IL-13 expressing ILC2s. In embodiments, at least 91% of the ILC2s in the population of expanded ILC2s are IL-13 expressing ILC2s. In embodiments, at least 92% of the ILC2s in the population of expanded ILC2s are IL-13 expressing ILC2s. In embodiments, at least 93% of the ILC2s in the population of expanded ILC2s are IL-13 expressing ILC2s. In embodiments, at least 94% of the ILC2s in the population of expanded ILC2s are IL-13 expressing ILC2s. In embodiments, at least 95% of the ILC2s in the population of expanded ILC2s are IL-13 expressing ILC2s. In embodiments, at least 96% of the ILC2s in the population of expanded ILC2s are IL-13 expressing ILC2s. In embodiments, at least 97% of the ILC2s in the population of expanded ILC2s are IL-13 expressing ILC2s. In embodiments, at least 98% of the ILC2s in the population of expanded ILC2s are IL-13 expressing ILC2s. In embodiments, at least 99% of the ILC2s in the population of expanded ILC2s are IL-13 expressing ILC2s.

In embodiments, about 30% of the ILC2s in the population of expanded ILC2s are IL-13 expressing ILC2s. In embodiments, about 40% of the ILC2s in the population of expanded ILC2s are IL-13 expressing ILC2s. In embodiments, about 50% of the ILC2s in the population of expanded ILC2s are IL-13 expressing ILC2s. In embodiments, about 60% of the ILC2s in the population of expanded ILC2s are IL-13 expressing ILC2s. In embodiments, about 70% of the ILC2s in the population of expanded ILC2s are IL-13 expressing ILC2s. In embodiments, about 80% of the ILC2s in the population of expanded ILC2s are IL-13 expressing ILC2s. In embodiments, about 85% of the ILC2s in the population of expanded ILC2s are IL-13 expressing ILC2s. In embodiments, about 90% of the ILC2s in the population of expanded ILC2s are IL-13 expressing ILC2s. In embodiments, about 91% of the ILC2s in the population of expanded ILC2s are IL-13 expressing ILC2s. In embodiments, about 92% of the ILC2s in the population of expanded ILC2s are IL-13 expressing ILC2s. In embodiments, about 93% of the ILC2s in the population of expanded ILC2s are IL-13 expressing ILC2s. In embodiments, about 94% of the ILC2s in the population of expanded ILC2s are IL-13 expressing ILC2s. In embodiments, about 95% of the ILC2s in the population of expanded ILC2s are IL-13 expressing ILC2s. In embodiments, about 96% of the ILC2s in the population of expanded ILC2s are IL-13 expressing ILC2s. In embodiments, about 97% of the ILC2s in the population of expanded ILC2s are IL-13 expressing ILC2s. In embodiments, about 98% of the ILC2s in the population of expanded ILC2s are IL-13 expressing ILC2s. In embodiments, about 99% of the ILC2s in the population of expanded ILC2s are IL-13 expressing ILC2s. In embodiments, about 100% of the ILC2s in the population of expanded ILC2s are IL-13 expressing ILC2s.

For the population of expanded human ILC2s provided herein, in embodiments, the population of expanded human ILC2s includes ILC2s expressing IL-33R, NKp30, or a combination thereof. In embodiments, the ILC2s includes IL-33R expressing ILC2s (e.g. IL-33R+ILC2). In embodiments, at least 30% of the ILC2s in the population of expanded ILC2s are IL-33R expressing ILC2s. In embodiments, at least 40% of the ILC2s in the population of expanded ILC2s are IL-33R expressing ILC2s. In embodiments, at least 50% of the ILC2s in the population of expanded ILC2s are IL-33R expressing ILC2s. In embodiments, at least 60% of the ILC2s in the population of expanded ILC2s are IL-33R expressing ILC2s. In embodiments, at least 70% of the ILC2s in the population of expanded ILC2s are IL-33R expressing ILC2s. In embodiments, at least 80% of the ILC2s in the population of expanded ILC2s are IL-33R expressing ILC2s. In embodiments, at least 85% of the ILC2s in the population of expanded ILC2s are IL-33R expressing ILC2s. In embodiments, at least 90% of the ILC2s in the population of expanded ILC2s are IL-33R expressing ILC2s. In embodiments, at least 91% of the ILC2s in the population of expanded ILC2s are IL-33R expressing ILC2s. In embodiments, at least 92% of the ILC2s in the population of expanded ILC2s are IL-33R expressing ILC2s. In embodiments, at least 93% of the ILC2s in the population of expanded ILC2s are IL-33R expressing ILC2s. In embodiments, at least 94% of the ILC2s in the population of expanded ILC2s are IL-33R expressing ILC2s. In embodiments, at least 95% of the ILC2s in the population of expanded ILC2s are IL-33R expressing ILC2s. In embodiments, at least 96% of the ILC2s in the population of expanded ILC2s are IL-33R expressing ILC2s. In embodiments, at least 97% of the ILC2s in the population of expanded ILC2s are IL-33R expressing ILC2s. In embodiments, at least 98% of the ILC2s in the population of expanded ILC2s are IL-33R expressing ILC2s. In embodiments, at least 99% of the ILC2s in the population of expanded ILC2s are IL-33R expressing ILC2s.

In embodiments, about 30% of the ILC2s in the population of expanded ILC2s are IL-33R expressing ILC2s. In embodiments, about 40% of the ILC2s in the population of expanded ILC2s are IL-33R expressing ILC2s. In embodiments, about 50% of the ILC2s in the population of expanded ILC2s are IL-33R expressing ILC2s. In embodiments, about 60% of the ILC2s in the population of expanded ILC2s are IL-33R expressing ILC2s. In embodiments, about 70% of the ILC2s in the population of expanded ILC2s are IL-33R expressing ILC2s. In embodiments, about 80% of the ILC2s in the population of expanded ILC2s are IL-33R expressing ILC2s. In embodiments, about 85% of the ILC2s in the population of expanded ILC2s are IL-33R expressing ILC2s. In embodiments, about 90% of the ILC2s in the population of expanded ILC2s are IL-33R expressing ILC2s. In embodiments, about 91% of the ILC2s in the population of expanded ILC2s are IL-33R expressing ILC2s. In embodiments, about 92% of the ILC2s in the population of expanded ILC2s are IL-33R expressing ILC2s. In embodiments, about 93% of the ILC2s in the population of expanded ILC2s are IL-33R expressing ILC2s. In embodiments, about 94% of the ILC2s in the population of expanded ILC2s are IL-33R expressing ILC2s. In embodiments, about 95% of the ILC2s in the population of expanded ILC2s are IL-33R expressing ILC2s. In embodiments, about 96% of the ILC2s in the population of expanded ILC2s are IL-33R expressing ILC2s. In embodiments, about 97% of the ILC2s in the population of expanded ILC2s are IL-33R expressing ILC2s. In embodiments, about 98% of the ILC2s in the population of expanded ILC2s are IL-33R expressing ILC2s. In embodiments, about 99% of the ILC2s in the population of expanded ILC2s are IL-33R expressing ILC2s. In embodiments, about 100% of the ILC2s in the population of expanded ILC2s are IL-33R expressing ILC2s.

In embodiments, the ILC2s includes NKp30 expressing ILC2s (e.g. NKp30⁺ ILC2). In embodiments, at least 30% of the ILC2s in the population of expanded ILC2s are NKp30 expressing ILC2s. In embodiments, at least 40% of the ILC2s in the population of expanded ILC2s are NKp30 expressing ILC2s. In embodiments, at least 50% of the ILC2s in the population of expanded ILC2s are NKp30 expressing ILC2s. In embodiments, at least 60% of the ILC2s in the population of expanded ILC2s are NKp30 expressing ILC2s. In embodiments, at least 70% of the ILC2s in the population of expanded ILC2s are NKp30 expressing ILC2s. In embodiments, at least 80% of the ILC2s in the population of expanded ILC2s are NKp30 expressing ILC2s. In embodiments, at least 85% of the ILC2s in the population of expanded ILC2s are NKp30 expressing ILC2s. In embodiments, at least 90% of the ILC2s in the population of expanded ILC2s are NKp30 expressing ILC2s. In embodiments, at least 91% of the ILC2s in the population of expanded ILC2s are NKp30 expressing ILC2s. In embodiments, at least 92% of the ILC2s in the population of expanded ILC2s are NKp30 expressing ILC2s. In embodiments, at least 93% of the ILC2s in the population of expanded ILC2s are NKp30 expressing ILC2s. In embodiments, at least 94% of the ILC2s in the population of expanded ILC2s are NKp30 expressing ILC2s. In embodiments, at least 95% of the ILC2s in the population of expanded ILC2s are NKp30 expressing ILC2s. In embodiments, at least 96% of the ILC2s in the population of expanded ILC2s are NKp30 expressing ILC2s. In embodiments, at least 97% of the ILC2s in the population of expanded ILC2s are NKp30 expressing ILC2s. In embodiments, at least 98% of the ILC2s in the population of expanded ILC2s are NKp30 expressing ILC2s. In embodiments, at least 99% of the ILC2s in the population of expanded ILC2s are NKp30 expressing ILC2s.

In embodiments, about 30% of the ILC2s in the population of expanded ILC2s are NKp30 expressing ILC2s. In embodiments, about 40% of the ILC2s in the population of expanded ILC2s are NKp30 expressing ILC2s. In embodiments, about 50% of the ILC2s in the population of expanded ILC2s are NKp30 expressing ILC2s. In embodiments, about 60% of the ILC2s in the population of expanded ILC2s are NKp30 expressing ILC2s. In embodiments, about 70% of the ILC2s in the population of expanded ILC2s are NKp30 expressing ILC2s. In embodiments, about 80% of the ILC2s in the population of expanded ILC2s are NKp30 expressing ILC2s. In embodiments, about 85% of the ILC2s in the population of expanded ILC2s are NKp30 expressing ILC2s. In embodiments, about 90% of the ILC2s in the population of expanded ILC2s are NKp30 expressing ILC2s. In embodiments, about 91% of the ILC2s in the population of expanded ILC2s are NKp30 expressing ILC2s. In embodiments, about 92% of the ILC2s in the population of expanded ILC2s are NKp30 expressing ILC2s. In embodiments, about 93% of the ILC2s in the population of expanded ILC2s are NKp30 expressing ILC2s. In embodiments, about 94% of the ILC2s in the population of expanded ILC2s are NKp30 expressing ILC2s. In embodiments, about 95% of the ILC2s in the population of expanded ILC2s are NKp30 expressing ILC2s. In embodiments, about 96% of the ILC2s in the population of expanded ILC2s are NKp30 expressing ILC2s. In embodiments, about 97% of the ILC2s in the population of expanded ILC2s are NKp30 expressing ILC2s. In embodiments, about 98% of the ILC2s in the population of expanded ILC2s are NKp30 expressing ILC2s. In embodiments, about 99% of the ILC2s in the population of expanded ILC2s are NKp30 expressing ILC2s. In embodiments, about 100% of the ILC2s in the population of expanded ILC2s are NKp30 expressing ILC2s.

### Methods of Treatment

The population of expanded human ILC2s provided herein including embodiments thereof is contemplated to be effective for treating cancer. As described throughout the specification, in embodiments, the population of expanded human ILC2s provided herein including embodiments thereof has been shown to express proteins (e.g. GZMB, DNAM-1), the expression of which are impaired in ILC2s from patients with cancer. Expression of these proteins, including GZMB, by the population of expanded human ILC2s provided herein is demonstrated to induce apoptosis and pyroptosis in cancer cells. Applicant has thereby demonstrated that the population of expanded human ILC2s provided herein including embodiments thereof is able to exert potent anti-tumor activity. Thus, in an aspect is provided a method of treating cancer in a subject in need thereof, including administering to the subject an effective amount of a population of expanded human ILC2s provided herein including embodiments thereof. The population of expanded human ILC2s provided herein are contemplated to be effective for treatment of solid tumors and liquid tumors, also referred to as blood cancers. In embodiments, the cancer is a blood cancer (e.g. lymphoma, leukemia etc.). In embodiments, the cancer is a solid tumor (e.g. a tumor derived from the lung, pancreas, brain, etc.).

In embodiments, the population of expanded human ILC2s are derived from the subject. For example, the population of expanded human ILC2s may be autologous to the subject. In embodiments, the population of expanded human ILC2s are derived from a donor. In embodiments, the donor is a healthy donor. A healthy donor may be, for example, a subject who does not have cancer.

In embodiments, the cancer is leukemia, lung cancer pancreatic cancer, or brain cancer. In embodiments, the cancer is leukemia. In embodiments, the cancer is lung cancer. In embodiments, the cancer is pancreatic cancer. In embodiments, the cancer is brain cancer. In embodiments, the leukemia is acute myeloid leukemia (AML). In embodiments, the cancer is lymphoma.

Genetically modified human ILC2s including antibody regions that bind cancer antigens are contemplated to be useful for targeting specific cancer cells. Thus, in an aspect is provided a method of treating cancer in a subject in need thereof, including administering to the subject an effective amount of the genetically modified human ILC2s provided herein including embodiments thereof. In embodiments, the genetically modified human ILC2s is derived from the subject. In embodiments, the genetically modified human ILC2s is derived from a donor. In embodiments, the cancer is leukemia, lung cancer pancreatic cancer, or brain cancer. In embodiments, the leukemia is acute myeloid leukemia (AML). In embodiments, the cancer is lymphoma.

### Genetically Modified ILC2

The human ILC2s (e.g. expanded human ILC2) provided herein may be genetically modified to express a recombinant protein. For example, the human ILC2s may be genetically modified to express a chimeric antigen receptor (CAR) capable of targeting a a cancer cell. As used herein, “genetically modified” refers to introduction of a non-native nucleic acid or protein into a cell, or that the cell is derived from a cell so modified. For example, the ILC2s provided herein including embodiments thereof may be genetically modified by introducing a nucleic acid encoding a recombinant protein into the ILC2. Introduction of the nucleic acid may include use of any molecular biology technique known by a person having ordinary skill in the biological arts. Thus, in an aspect is provided a genetically modified human group 2 innate lymphoid cell (ILC2) including a chimeric antigen receptor (CAR), wherein the CAR includes: i) an antibody region; and ii) a transmembrane domain.

An “antibody region” as provided herein refers to a monovalent or multivalent protein moiety that forms part of the CAR (e.g. recombinant protein) provided herein including embodiments thereof. A person of ordinary skill in the art will therefore immediately recognize that the antibody region is a protein moiety capable of binding an antigen (epitope). Thus, the antibody region provided herein may include a domain of an antibody (e.g., a light chain variable (VL) domain, a heavy chain variable (VH) domain) or a fragment of an antibody (e.g., Fab). In embodiments, the antibody region is a protein conjugate. A “protein conjugate” as provided herein refers to a construct consisting of more than one polypeptide, wherein the polypeptides are bound together covalently or non-covalently. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker. In embodiments, the antibody region is an scFv. The antibody region may include a light chain variable (VL) domain and/or a heavy chain variable (VH) domain. In embodiments, the antibody region includes a light chain variable (VL) domain. In embodiments, the antibody region includes a heavy chain variable (VH) domain.

In embodiments, the antibody region is capable of binding a cancer antigen. In embodiments, the cancer antigen is a leukemia antigen, lung cancer antigen, pancreatic cancer antigen, or brain cancer antigen. In embodiments, the cancer antigen is a leukemia antigen. In embodiments, the cancer antigen is a lung cancer antigen. In embodiments, the cancer antigen is a pancreatic cancer antigen. In embodiments, the cancer antigen is a brain cancer antigen. In embodiments, the leukemia antigen is a acute myeloid leukemia antigen. In embodiments, the acute myeloid leukemia antigen is CD123, FLT3, CD70, CD33, or IL1RAP. In embodiments, the acute myeloid leukemia antigen is CD123. In embodiments, the acute myeloid leukemia antigen is FLT3. In embodiments, the acute myeloid leukemia antigen is CD70. In embodiments, the acute myeloid leukemia antigen is CD33. In embodiments, the acute myeloid leukemia antigen is IL1RAP. In embodiments, the lung cancer antigen is EGFR, EGFRvIII, HER2, PSCA, or PSMA. In embodiments, the lung cancer antigen is EGFR. In embodiments, the lung cancer antigen is EGFRvIII. In embodiments, the lung cancer antigen is HER2. In embodiments, the lung cancer antigen is PSCA. In embodiments, the lung cancer antigen is PSMA. In embodiments, the pancreatic cancer antigen is EGFR, EGFRvIII, HER2, PSCA, or PSMA. In embodiments, the pancreatic cancer antigen is EGFR. In embodiments, the pancreatic cancer antigen is EGFRvIII. In embodiments, the pancreatic cancer antigen is HER2. In embodiments, the pancreatic cancer antigen is PSCA. In embodiments, the pancreatic cancer antigen is PSMA. In embodiments, the brain cancer antigen is IL13Ra2, EGFR, or GD2. In embodiments, the brain cancer antigen is IL13Ra2. In embodiments, the brain cancer antigen is EGFR. In embodiments, the brain cancer antigen is GD2. In embodiments, the cancer antigen is TNFRSF17, IL3RA, SDC1, CD19, MS4A1, TNFRSF8, CD22, CD33, CD38, CD5, NCAM1, CD70, ULBP1, ULBP2, CEACAM5, MET, EPCAM, EPHA2, ERBB2, GPC3, MSLN, Muc1, PDCD1, CD274, KDR, IL13RA2, FOLH1, FAP, CA9, FOLR1, L1CAM, ROR1, CD23, CD44, CD174, SLAMF7, GD2, PSCA, GPNMB, CD276, CSPG4, CD133, or TEM1.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4 or CD3-zeta. In embodiments, the transmembrane domain is a CD4 transmembrane domain.

In embodiments, the transmembrane domain is a CD28 transmembrane domain. The term “CD28 transmembrane domain” as provided herein includes any of the recombinant or naturally-occurring forms of the transmembrane domain of CD28, or variants or homologs thereof that maintain CD28 transmembrane domain activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, CD28 is the protein as identified by the NCBI sequence reference GI:340545506, homolog or functional fragment thereof.

In embodiments, the transmembrane domain is a CD8 transmembrane domain. The term “CD8 transmembrane domain” as provided herein includes any of the recombinant or naturally-occurring forms of the transmembrane domain of CD8, or variants or homologs thereof that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, CD8 is the protein as identified by the NCBI sequence reference GI:225007534, homolog or functional fragment thereof.

In embodiments, the transmembrane domain is a CD4 transmembrane domain. The term “CD4 transmembrane domain” as provided herein includes any of the recombinant or naturally-occurring forms of the transmembrane domain of CD4, or variants or homologs thereof that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, CD4 is the protein as identified by the NCBI sequence reference GI:303522473, homolog or functional fragment thereof.

In embodiments, the transmembrane domain is a CD3-zeta (also known as CD247) transmembrane domain. The term “CD3-zeta transmembrane domain” as provided herein includes any of the recombinant or naturally-occurring forms of the transmembrane domain of CD3-zeta, or variants or homologs thereof that maintain CD3-zeta transmembrane domain activity (e.g. within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In embodiments, CD3-zeta is the protein as identified by the NCBI sequence reference GI:166362721, homolog or functional fragment thereof.

In embodiments, the chimeric antigen receptor further includes an intracellular signaling domain. An “intracellular signaling domain” as provided herein includes amino acid sequences capable of providing primary signaling in response to binding of an antigen to the antibody region provided herein including embodiments thereof. In embodiments, the signaling of the intracellular signaling domain results in activation of the cell expressing the same. In embodiments, the signaling of the intracellular signaling domain results in proliferation (cell division) of the cell expressing the same. In embodiments, the signaling of the intracellular signaling domain results in expression by said cell of proteins known in the art to be characteristic of activated innate lymphoid cells (e.g., CTLA-4, PD-1, CD28, CD69). In embodiments, the intracellular signaling domain is a CD3 ζ intracellular T-cell signaling domain.

In embodiments, the chimeric antigen receptor further includes an intracellular co-stimulatory signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the cell expressing the same. In embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a 4-1BB intracellular co-stimulatory signaling domain. In embodiments, the intracellular co-stimulatory signaling domain is an ICOS intracellular co-stimulatory signaling domain. In embodiments, the intracellular co-stimulatory signaling domain is an OX-40 intracellular co-stimulatory signaling domain.

In embodiments, the antibody region includes an Fc domain. In embodiments, the antibody region includes a spacer region. In embodiments, the spacer region is between the transmembrane domain and the antibody region. A “spacer region” as provided herein is a polypeptide connecting the antibody region with the transmembrane domain. In embodiments, the spacer region connects the heavy chain constant region with the transmembrane domain. In embodiments, the spacer region includes an Fc region. In embodiments, the spacer region is an Fc region. Examples of spacer regions contemplated for the compositions provided herein include without limitation, immunoglobulin molecules or fragments thereof (e.g., IgG1, IgG2, IgG3, IgG4) and immunoglobulin molecules or fragments thereof (e.g., IgG1, IgG2, IgG3, IgG4) including mutations affecting Fc receptor binding. In embodiments, the spacer region is a hinge region.

The term “CTLA-4” as referred to herein includes any of the recombinant or naturally-occurring forms of the cytotoxic T-lymphocyte-associated protein 4 protein, also known as CD152 (cluster of differentiation 152), or variants or homologs thereof that maintain CTLA-4 activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, the CTLA-4 protein is substantially identical to the protein identified by the UniProt reference number P16410 or a variant or homolog having substantial identity thereto.

The term “CD28” as referred to herein includes any of the recombinant or naturally-occurring forms of the Cluster of Differentiation 28 protein, or variants or homologs thereof that maintain CD28 activity (e.g. within at least 50%, 80%, 90%, 95%, 96%, 97%, 98%, 99% or 100% activity compared to CD28). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD69” as referred to herein includes any of the recombinant or naturally-occurring forms of the Cluster of Differentiation 69 protein, or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “4-1BB” as referred to herein includes any of the recombinant or naturally-occurring forms of the 4-1BB protein, also known as tumor necrosis factor receptor superfamily member 9 (TNFRSF9), Cluster of Differentiation 137 (CD137) and induced by lymphocyte activation (ILA), or variants or homologs thereof that maintain 4-1BB activity (e.g. within at least 50%, 80%, 90%, 95%, 96%, 97%, 98%, 99% or 100% activity compared to 4-1BB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the 4-1BB protein is substantially identical to the protein identified by the UniProt reference number Q07011 or a variant or homolog having substantial identity thereto.

For the genetically modified human ILC2s provided herein, in embodiments, the genetically modified ILC2s is a CD161⁺ ILC2. In embodiments, the genetically modified ILC2s is a CRTH2⁺CD117⁺ ILC2. In embodiments, the genetically modified ILC2s is a CD122⁺ ILC2. In embodiments, the genetically modified ILC2s is a DNAX Accessory Molecule-1 (DNAM-1) expressing ILC2. In embodiments, the genetically modified ILC2s is a granzyme B (GZMB) expressing ILC2.

In embodiments, the genetically modified human ILC2s expresses IL-4, IL-5, IL-9, IL-13, or a combination thereof. In embodiments, the genetically modified human ILC2s expresses IL-4 In embodiments, the genetically modified human ILC2s expresses IL-5. In embodiments, the genetically modified human ILC2s expresses IL-9. In embodiments, the genetically modified human ILC2s expresses IL-13.

In embodiments, the genetically modified human ILC2s expresses IL-33R, NKp30, or a combination thereof. In embodiments, the genetically modified human ILC2s expresses IL-33R. In embodiments, the genetically modified human ILC2s expresses NKp30.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Introduction to Exemplary Studies

Herein, we describe a reliable platform for up to 1200-fold ex vivo expansion of ILC2s from healthy donors in 4 weeks. We use both liquid and solid tumor models to demonstrate that human ILC2s can directly lyse tumor cells and exert antitumor function in vitro and in vivo, similar as NK cells. Mechanistically, granzyme B (GZMB) released by ILC2s induces pyroptosis and/or apoptosis of tumor cells. This process is mediated by DNAM-1-CD112/CD155 receptor-ligand direct interaction resulting in downstream phosphorylation and degradation of the inhibitor FOXO1. ILC2s obtained from acute myeloid leukemia patients but not from healthy donors have impaired DNAM-1 and GZMB expression. These data define a protective role for ILC2s in cancer with a tumor evasion mechanism. ILC2s are therefore a new member of cytotoxic effector cells and allogeneic cell therapy.

### Example 2: Introduction to Exemplary Studies

Cancer immunotherapy, including checkpoint inhibitors and adoptive cell therapy, is an evolving and promising modality of cancer treatment. Cell-based immunotherapy such as chimeric antigen receptor T cells (CAR-T)¹, natural killer (NK) cells or CAR-NK cells², and CAR-macrophages³ has revolutionized the treatment of multiple cancers. Although adoptive immunotherapy has successfully treated B-cell lymphoma and acute lymphoblastic leukemia and achieved remarkably effective and durable responses in many patients⁴⁻⁶, these treatments can also result in high levels of inflammation and subsequent negative consequences such as severe and at times lethal cytokine release syndrome (CRS)¹. Its efficacy in treating other hematological (e.g., myeloid malignancies) and solid tumors has been limited, highlighting the need for further innovation in cell-based therapy.

Group 2 innate lymphoid cells, also known as ILC2s, are characterized by the expression of GATA3 and the production of Th₂ cell-associated cytokines including IL-4, IL-5, IL-9, and IL-13 as well as amphiregulin (AREG) in response to stimulation with the cytokines IL-25, IL-33, and thymic stromal lymphopoietin (TSLP)⁷. In mice, two main subgroups of ILC2s have been identified: the IL-33-induced steady-state natural ILC2, and the IL-25-elicited inflammatory ILC2s^{8,9}. In humans, ILC2s have been characterized as CD127⁺CRTH2⁺c-Kit^{-/-} and expressing the IL-33 receptor ST2 and a subunit of the IL-25 receptor IL-17RB¹⁰⁻¹². Functionally, ILC2s have been implicated in regulating inflammation and as a crucial bridge between innate and adaptive type 2 immunity¹³. ILC2s can promote the expansion and activity of myeloid-derived suppressor cells (MDSCs) via producing type-2 cytokines¹⁸⁻²⁰ further, studies suggest that ILC2s can reduce metastatic dissemination in pancreatic²², lung^{23,24}, and colorectal cancer²⁵ models by exerting antitumor effects. However, the role of ILC2s in different tumor types is unclear and justify the need for a better understanding of this unique cell type among the oncology and hematology communities. Additionally, studies with human ILC2s are limited, leading us to the present characterization of human ILC2s in both normal and disease settings.

We herein report a novel strategy to study ILC2s function and their potential use in adoptive immunotherapy. We demonstrate that human ILC2s isolated from the peripheral blood of healthy donors can be reliably expanded ex vivo. We also utilize models for both hematologic malignancy (acute myeloid leukemia, AML) and three solid tumors (lung, pancreatic, and brain cancer) to investigate the function of ILC2s in antitumor immunity. Experiments with these cell lines (three AML cell lines, and seven solid tumor cell lines) reveal that ILC2s can directly lyse tumor cells and exert antitumor function in vitro and/or in vivo and ILC2s are shown to have comparable antitumor activity with NK cells but more potent than T cells in vivo in AML. Mechanistically, we use a model of AML to demonstrate that lysis of tumor cells by ILC2s occurs via GZMB that once released, induces pyroptosis and/or apoptosis. Cell-cell contact-mediated interaction between DNAM-1 (expressed on ILC2s) with their ligands CD112/CD155 (expressed on target cells) enhanced GZMB production and the lytic function of ILC2s. In healthy donors, GZMB regulation is controlled by DNAM-1-induced phosphorylation and degradation of the inhibitor FOXO1. However, in patients with AML, ILC2s exhibit decreased expression of DNAM-1 and subsequent limited production of GZMB thus suggesting a mechanism for tumor escape. Collectively, our work demonstrates that ILC2s, with up to a 1200-fold ex vivo expansion in 4 weeks, is a potent antitumor agent in pre-clinical models and presents a new promising adoptive allogeneic cell therapy for cancer.

### Results

ILC2s Isolated from Human Peripheral Blood can be Reliably Expanded Ex Vivo

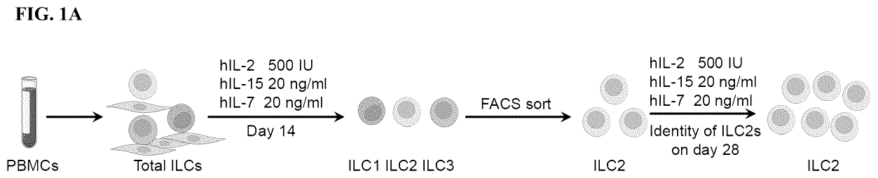

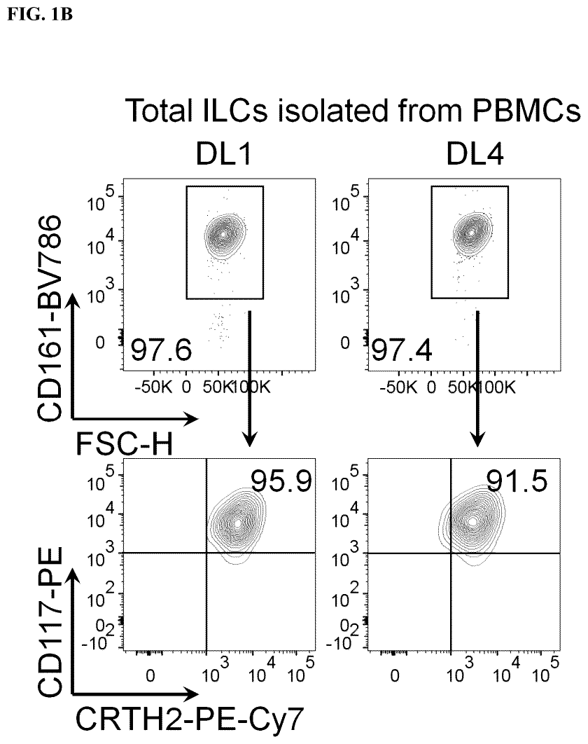

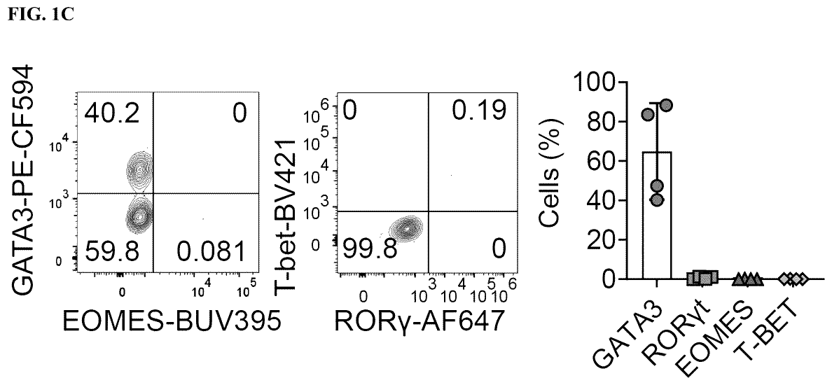

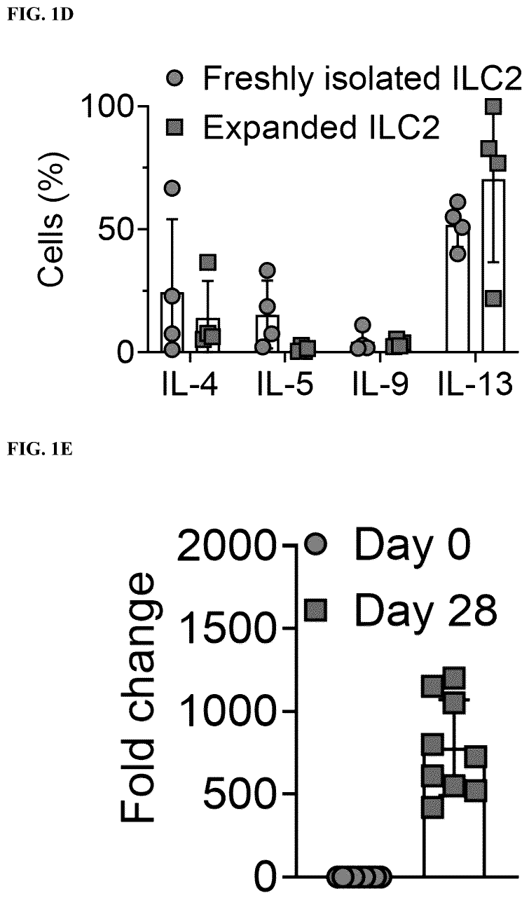

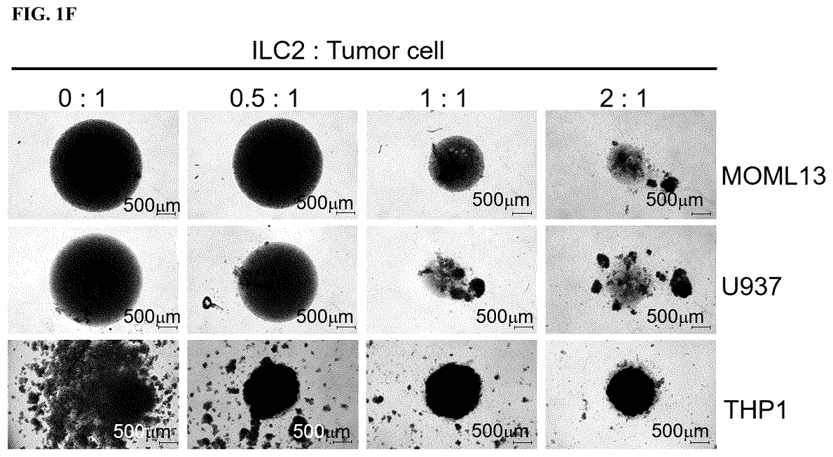

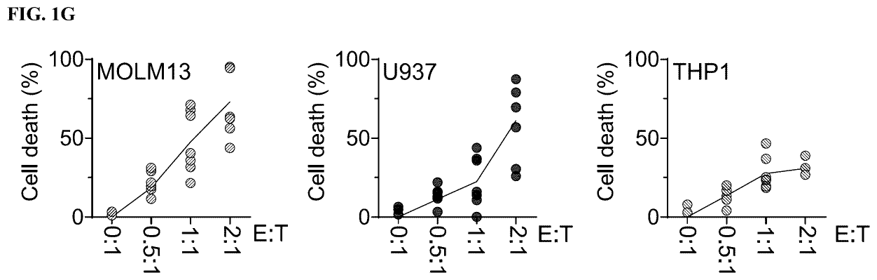

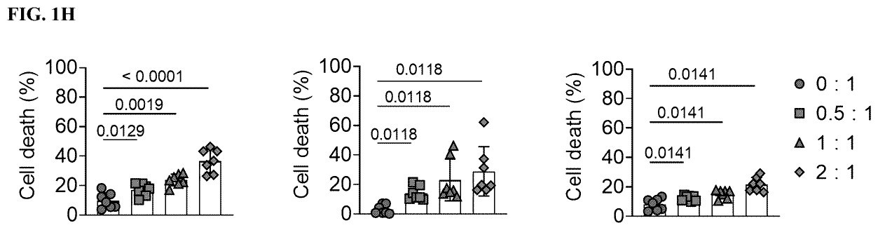

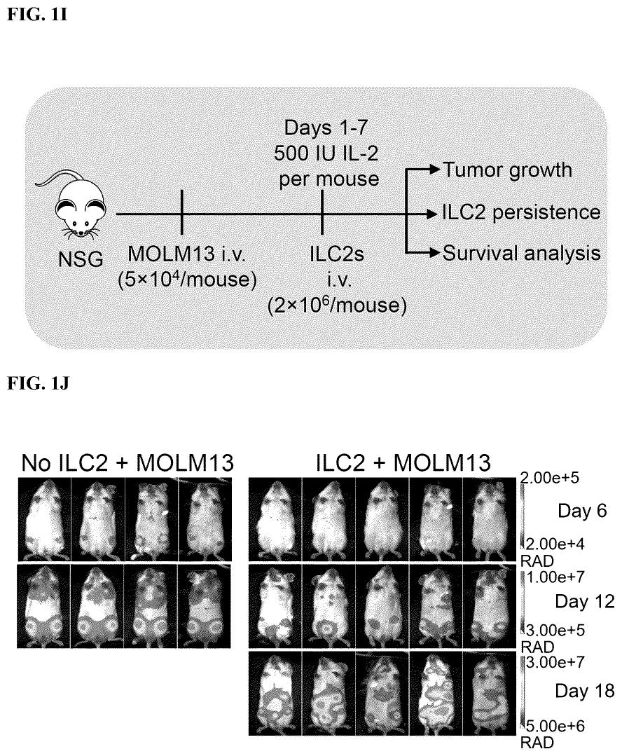

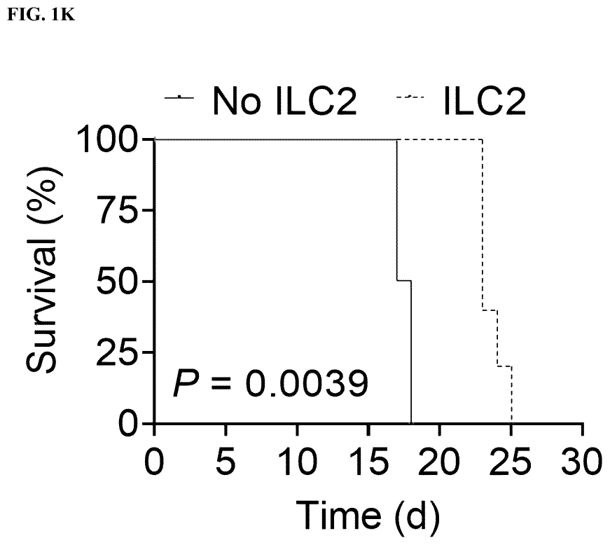

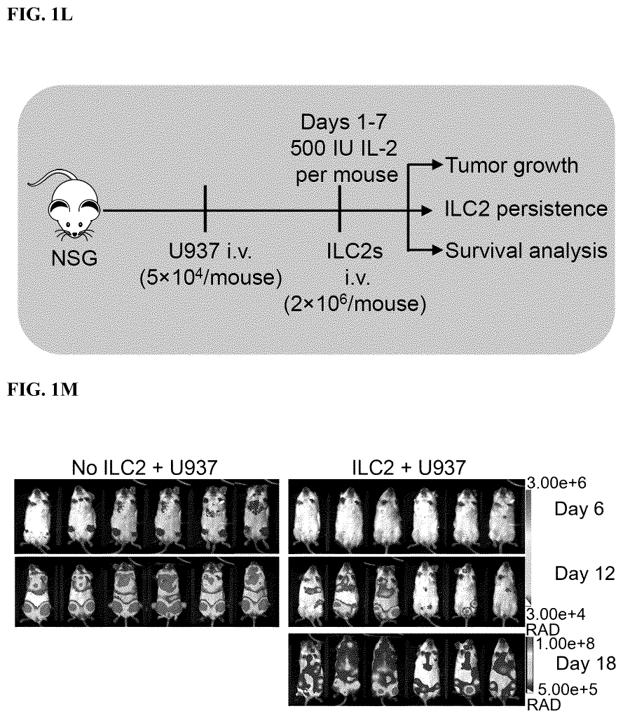

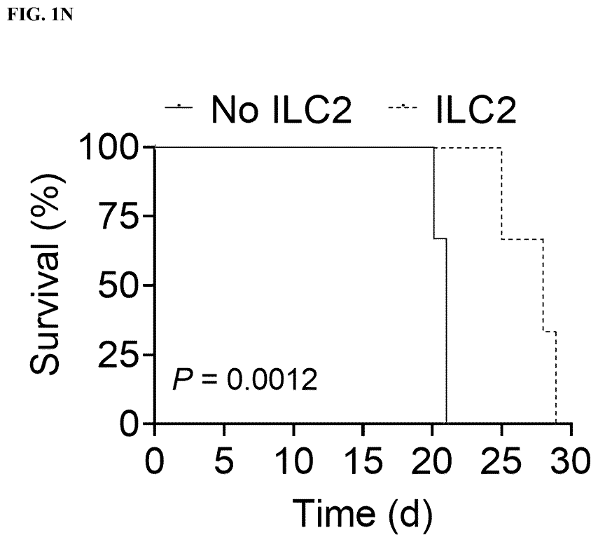

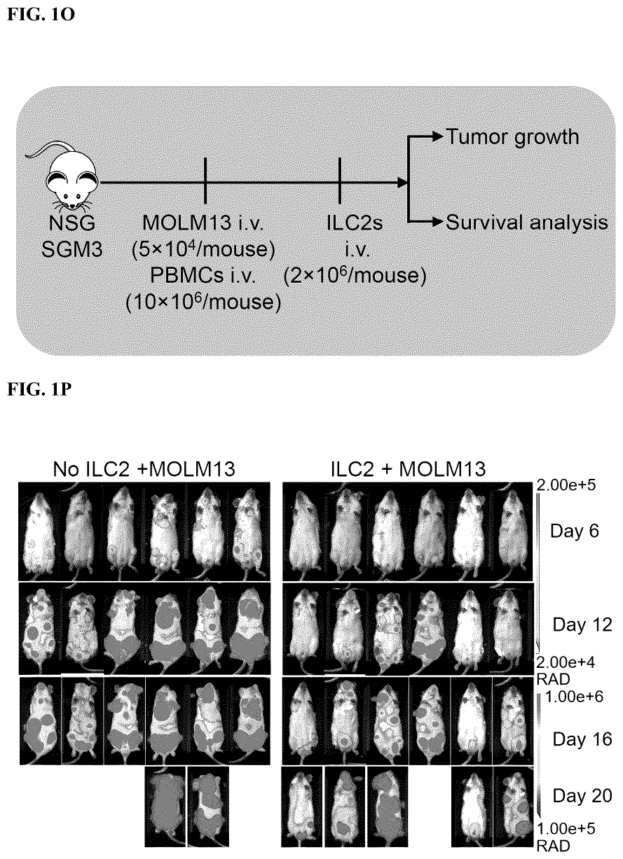

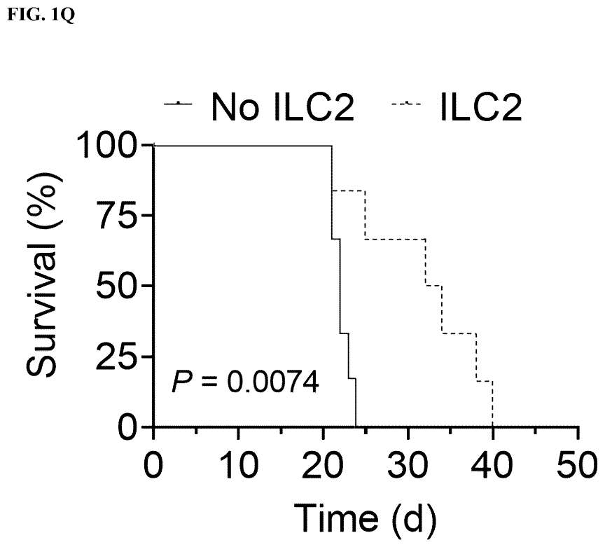

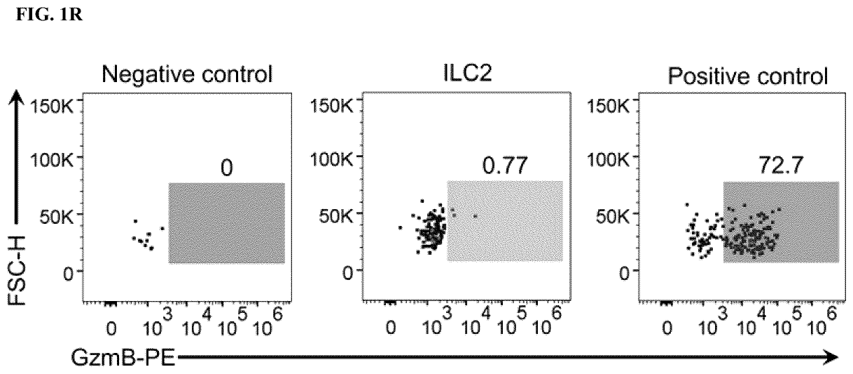

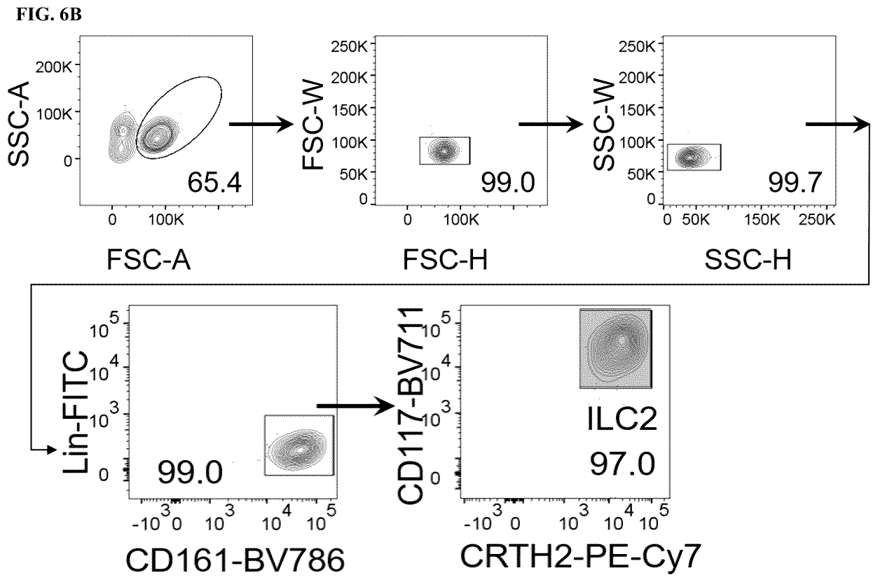

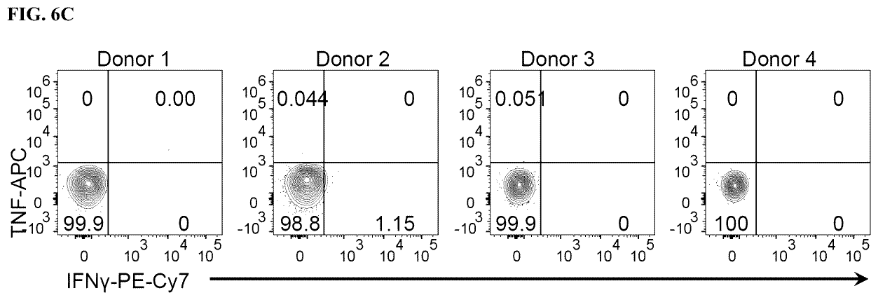

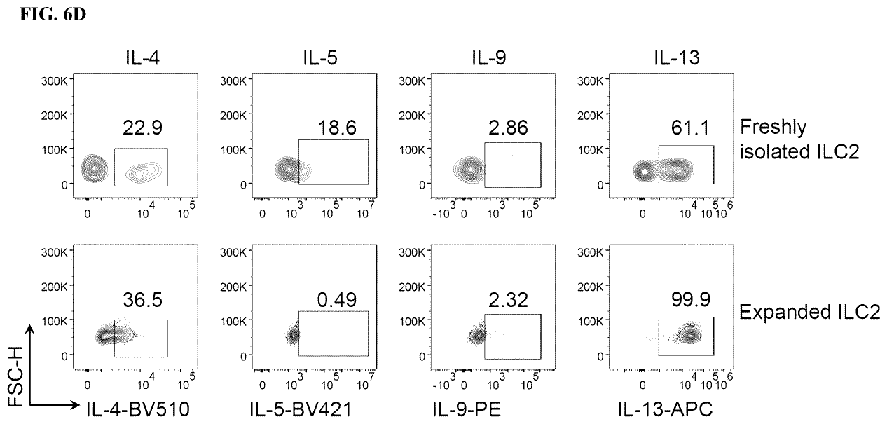

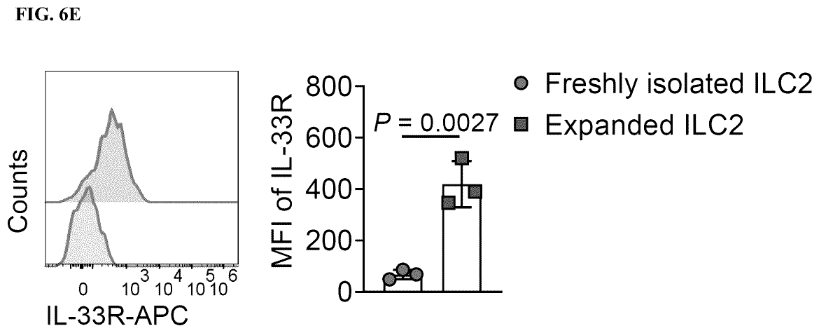

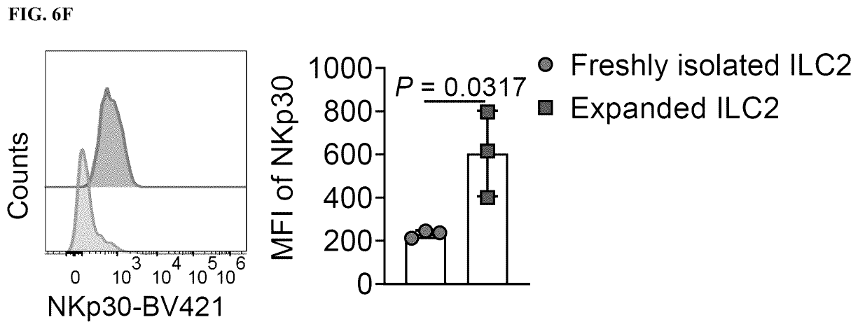

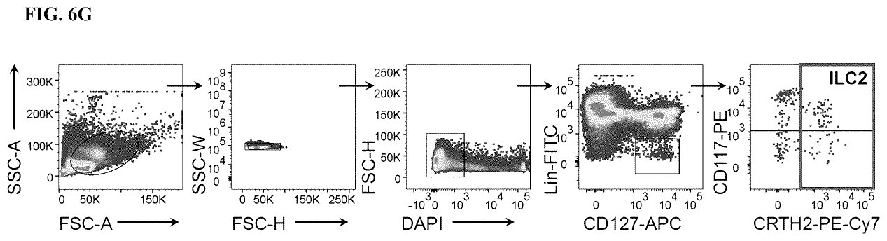

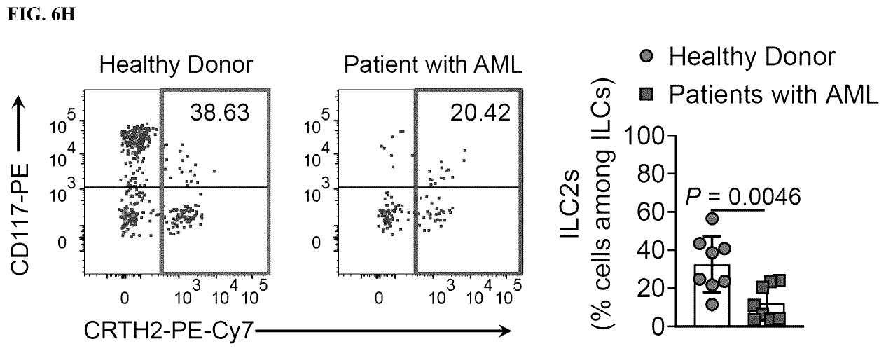

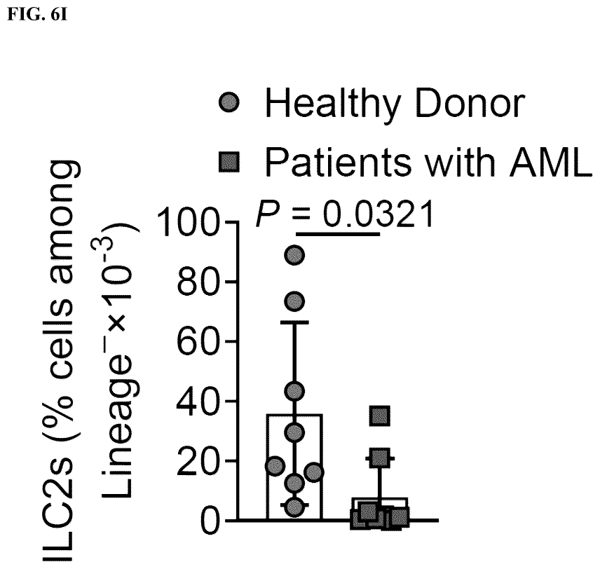

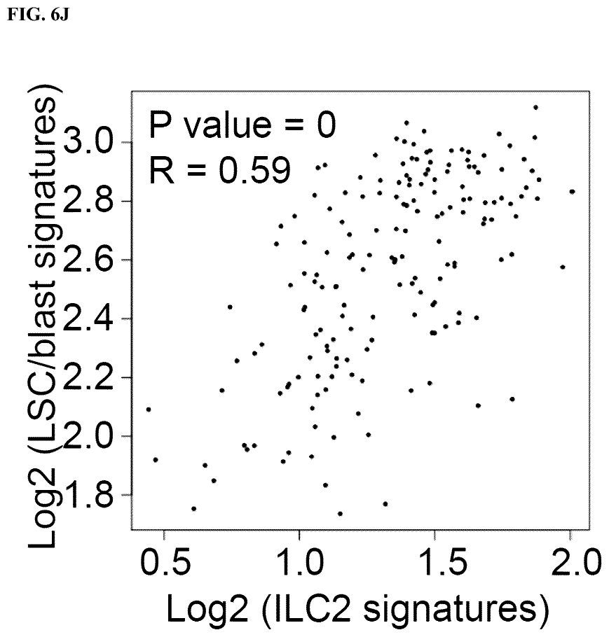

ILC2s have been mainly described as tissue-resident cells, but they can also be detected at low levels in human peripheral blood (PB). However, unlike mouse ILC2s, there is still no consistent methodology for purifying and expanding human ILC2s to enable in-depth analysis. To establish a system where human ILC2s could be reliably isolated and expanded with high purity from PB, we explored a variety of lineage depletion techniques using RosetteSep and EasySep magnetic systems. NK cells were first enriched using the RosetteSep human enrichment kit to remove non-NK cells and red blood cells, and then total ILCs were enriched to remove non-ILCs using the pan-ILC isolation kit. Isolated ILCs were cultured on either DL1-transfected OP9 (hereafter referred to as DL1) or DL4-transfected OP9 (hereafter referred to as DL4) stromal cells in the presence of IL-2, IL-7, and IL-15. After 14 days, we sorted for ILC2s using the surface marker CD161, a human ILC marker, and a combination of CD56, chemoattractant receptor-homologous molecule expressed on TH2 lymphocytes (CRTH2), and CD117. As surface staining of CD127 (IL-7 receptor-α), a human ILC marker, is affected by high IL-2 and IL-7 concentrations²⁶⁻²⁸, we instead used CD161. The gating of freshly isolated ILC2s and expanded ILC2s is described in FIG. 6A and FIG. 6B. Next, we co-cultured and expanded isolated ILC2s on OP9 stromal cells in the presence of IL-2, IL-7, and IL-15. The culture conditions used throughout the study are schematically depicted in FIG. 1A. The purity of CD161⁺ cells was approximately 97% after flow cytometry sorting on day 14 (FIG. 6B). Regardless of co-culture on either DL1 or DL4 stromal cells, more than 91% of fluorescence-activated cell sorting (FACS)-sorted ILC2s CD161⁺ cells on day 28 were CRTH2⁺CD117⁺ cells (FIG. 1B). Among ILC2s, we observed the presence of GATA3 transcription factor (FIG. 1C), which was previously described in ILC2s²⁹. We did not observe T-BET/EOMES (FIG. 1C) nor did they produce IFNγ or TNF (FIG. 6C), as previously described for NK cells/ILC1s¹³, nor did they express RORγt as described for ILC3s under these conditions¹³ (FIG. 6C). Compared to freshly FACS-sorted ILC2s from peripheral blood mononuclear cells (PBMCs), ex. vivo expanded ILC2s can produce comparable ILC2-associated cytokines such as IL-4, IL-5, IL-9, and IL-13 following phorbol-12-myristate-13-acetate/ionomycin stimulation (FIG. 1D, FIG. 6D). These cells also express IL-33 receptor and NKp30 (FIG. 6E and FIG. 6F), as demonstrated in freshly isolated PB ILC2s³⁰. Culture conditions resulted in up to a 1200-fold increase in cell numbers from freshly isolated ILC2s on day 28 (FIG. 1E), referred to as Ex ILC2s.

### ILC2s Induce AML Cell Death In Vitro and Prevent Tumor Growth In Vivo

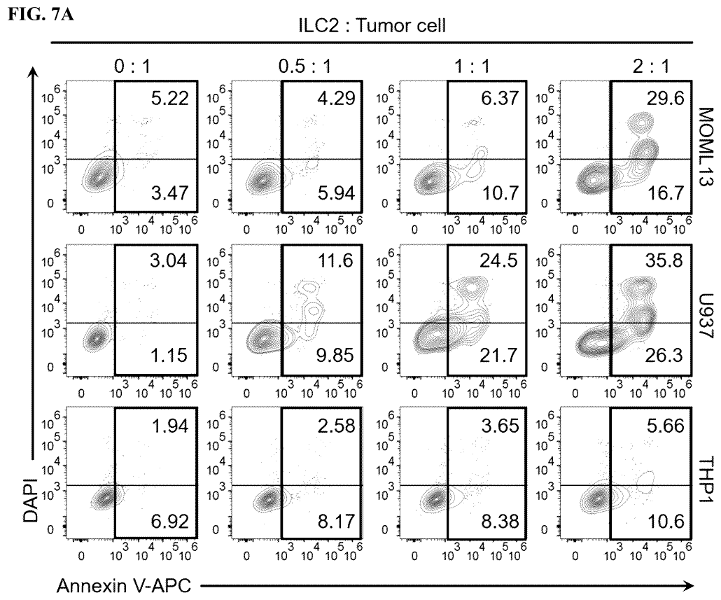

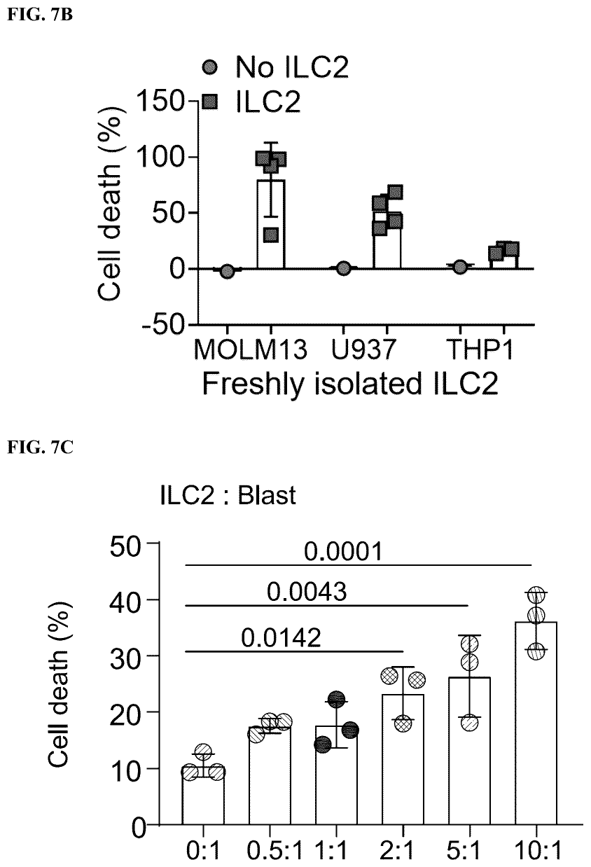

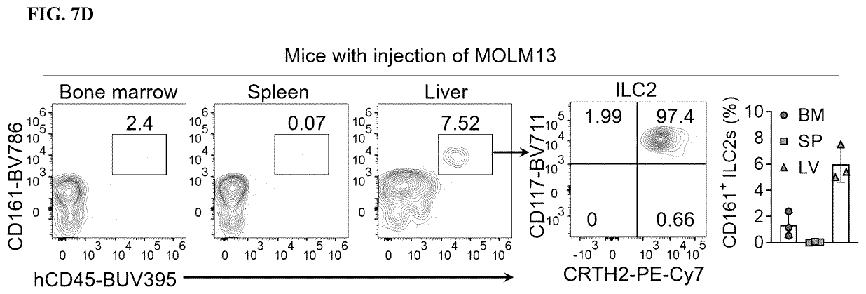

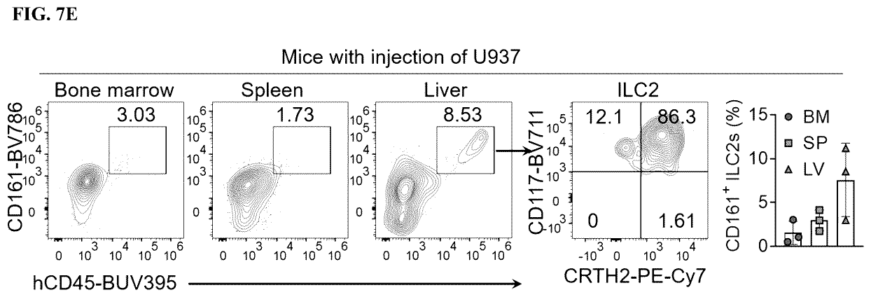

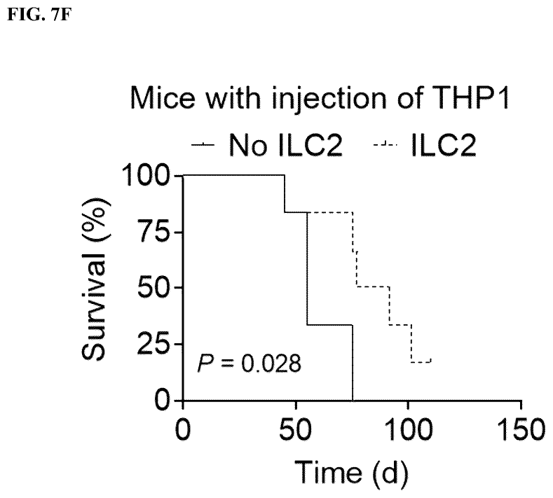

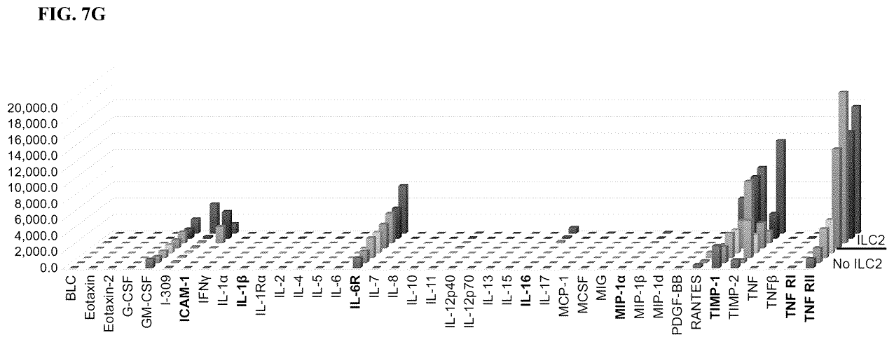

To further define the function of ILC2s in cancer immunity, we explored their interaction with AML, a disease with an overall poor prognosis³². Among eight patients with AML, we noted a highly significant reduction in total peripheral blood ILC2s among both lineage-negative (Lin) cells and ILC subsets (defined as Lin-CD127⁺) at disease onset relative to eight healthy donors (FIGS. 6G-6I). Using the Cancer Genome Atlas (TCGA) database, we identified a significant correlation between the ILC2s and leukemia cell (blast/LSC) signatures (FIG. 6J). Given that human ILC2s become reduced in the context of AML, we asked if ILC2s have an adverse effect on the genesis of AML. To address this, we expanded ILC2s ex vivo from the blood of healthy donors using our established culture system described above and co-cultured them with different types of AML cell lines (MOLM13, U937, and THP1) at various ratios for two days. Ex ILC2s lysed AML cell lines at the effector to target cell ratio of 2:1, 1:1, or 0.5:1 in a dose-dependent manner, as evidenced by a visually appreciable size reduction in the pellet of AML cells under microscopy (FIG. 1F), and an increase in the fraction of dead AML cells using luminescence-based (FIG. 1G) and flow cytometry-based assays (FIG. 1H and FIG. 7A) compared to co-culture without Ex ILC2s. Ex ILC2s lysed all three cells lines tested; however, the ability of Ex ILC2s to lyse MOLM13 and U937 is more potent than THP1 cells. We achieved similar results using freshly isolated ILC2s from the blood of healthy donors when co-cultured with these three AML cell lines (FIG. 7B). Likewise, we also observed that Ex ILC2s were able to lyse primary AML blast in a dose-dependent manner (FIG. 7C).

Next, we hypothesized that ILC2s from healthy donors could suppress AML growth in vivo. To test this, we employed an AML model of intravenously (i.v.) injected firefly luciferase (FFLuc)-labeled MOML13 cells into NSG mice, followed by i.v. infusion of Ex ILC2s. On days 1-7, the mice received a daily intraperitoneal (i.p.) injection of human IL-2 to maintain ILC2s (FIG. 1I). Tumor growth was monitored by measuring changes in tumor bioluminescence over time (FIG. 1J). Single dose infusion of Ex ILC2s improved tumor control and significantly prolonged mouse survival compared to the control group without Ex ILC2s treatment (FIG. 1J and FIG. 1K). Using flow cytometry, we observed adoptively transferred human Ex ILC2s mainly existed in mouse liver at the endpoint of the experiment (FIG. 7D). We achieved similar results using FFLuc-labeled U937 AML (FIGS. 1L-1N) and THP1 AML cells, (FIG. 7E and FIG. 7F). Next, to better mimic the physiologic environment of the human body, we added i.v. infusion of human PBMCs into NSG-SGM3 mice prior to i.v. transplantation of FFLuc-labeled MOLM13 cells and followed by injection with Ex ILC2s. Similarly, tumor growth was monitored by measuring changes in tumor bioluminescence over time. We observed that single dose infusion of Ex ILC2s in the presence of PBMCs also prevented tumor growth and significantly prolonged mouse survival compared to the control group without Ex ILC2s treatment in the presence of PBMCs (FIGS. 1O-1Q). We additionally evaluated the levels of pro-inflammatory proteins in the serum of these mice using an antibody-based array. We showed that infusion of ILC2s increased the levels of pro-inflammatory cytokines including ICAM1, IL-1β, TL-6R, IL-16, TIMP-1, MIP-1α, TNF RI, and TNF RII compared to infusion of MOLM13 alone (FIG. 7G and Table 1). Histopathologic analyses after Ex ILC2s infusion did not reveal any evidence of graft-versus-host disease (GVHD), tissue destruction, or toxicity, indicating the safety and effectiveness of Ex ILC2s in response to tumors in vivo. Taken together, our in vitro and in vivo data demonstrate that Ex ILC2s have the potential to delay progression of AML with relatively low toxicity.

### ILC2-Secreted Granzyme B Induces Pyroptosis or Apoptosis in AML Cells

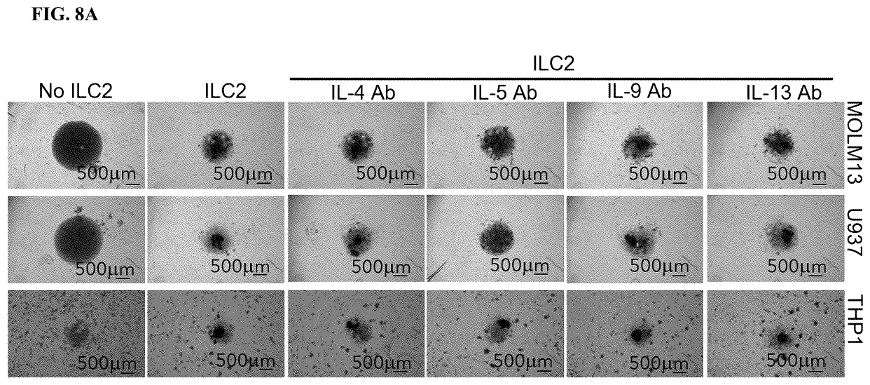

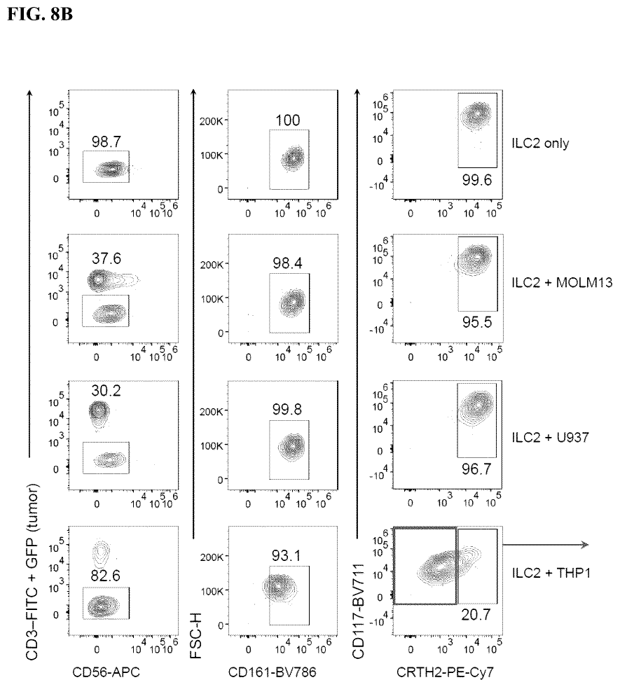

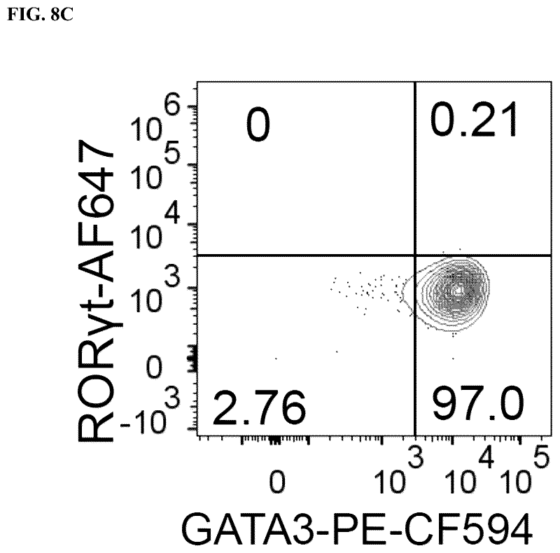

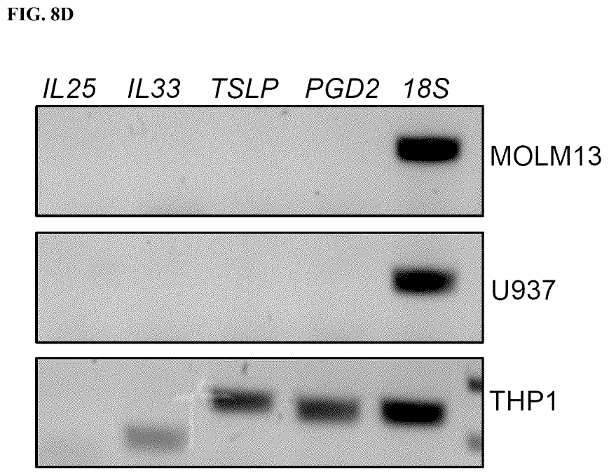

We found that Ex ILC2-induced AML death was not suppressed in the presence of neutralizing antibodies against IL-4, IL-5, IL-9, or IL-13 (the cytokines produced by ILC2s) when co-cultured with MOLM13, U937, or THP1 cells (FIG. 8A). We also found that Ex ILC2s did not convert into cytotoxic NK cells (CD3-CD56⁺)/ILC1s (CD56-CD161⁺CRTH2⁻CD117⁻) or cytotoxic ILC3s (CD56⁺CD161⁺CRTH2-CD117⁺) under co-culture conditions with MOLM13, U937, and THP1 (FIG. 8B) as defined by the surface markers for ILC2s (CD161⁺CRTH2⁺CD117⁺), although this conversion has been previously reported in response to tissue inflammation³³. Additionally, if there was conversion of ILC2s to ILC3s we would expect to see the disappearance of CRTH2, yet this was not observed for co-culture of ILC2s with MOLM13 and U937 cells. Although we did observe downregulation of CRTH2 in co-culture with THP1, ILC2s with downregulated CRTH2 exhibited high expression of the transcription factor GATA3 rather than RORγt (FIG. 8C), indicating that ILC2s co-cultured with THP1 maintain an identity of ILC2s and do not convert to ILC3s. Our results were consistent with a previous report that IL-33³⁴ and prostaglandin D2 (PGD2; a ligand of CRTH2)³⁵, expressed by THP1 cells but not MOLM13 and U937 cells (FIG. 8D) can suppress expression of CRTH2 on ILC2s. Collectively, these results suggest that ILC2-induced AML cell death is neither due to the secretion of IL-4, IL-5, IL-9, and IL-13 nor conversion into cytotoxic NK cells/ILC1s, and ILC3s.

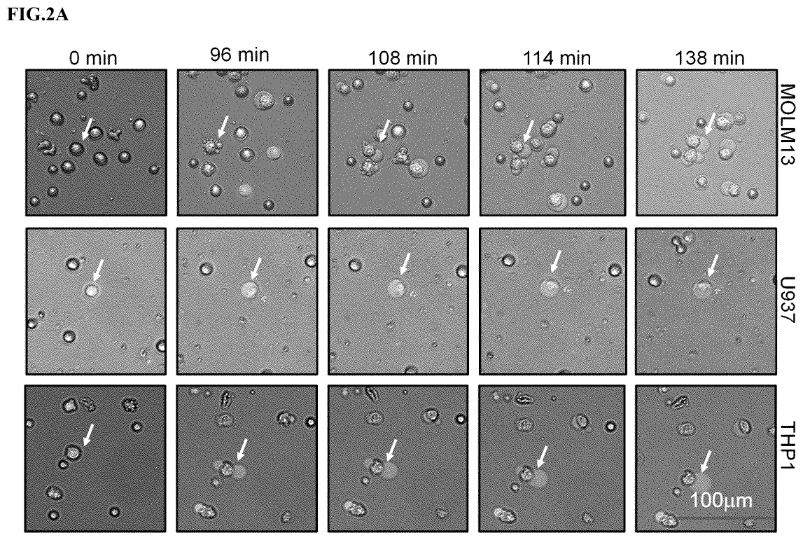

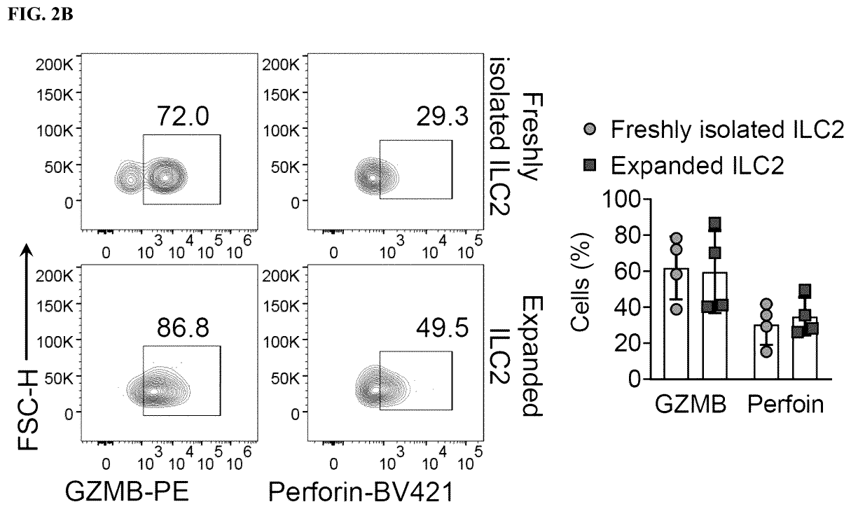

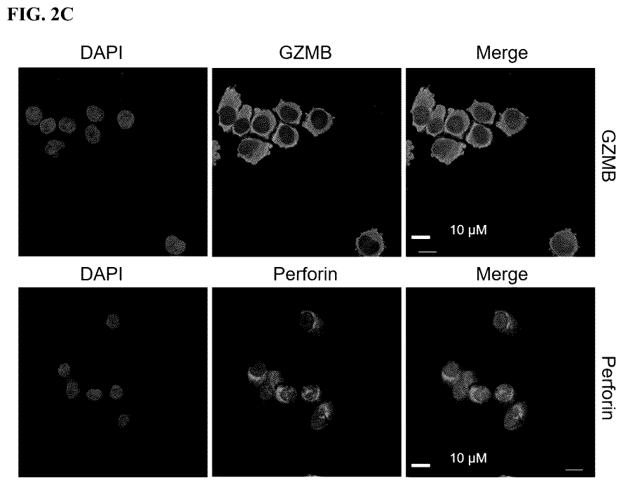

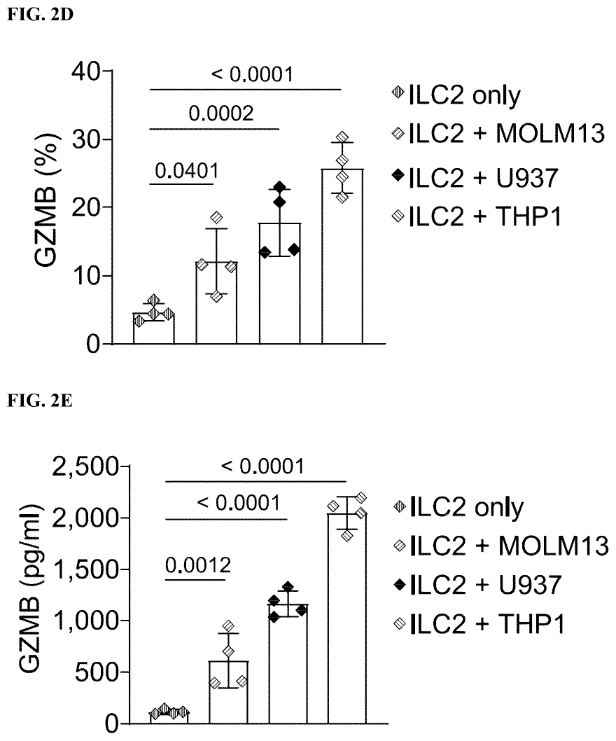

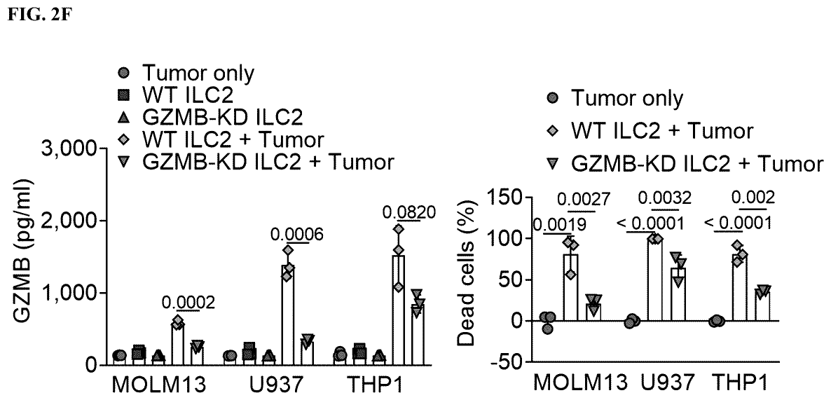

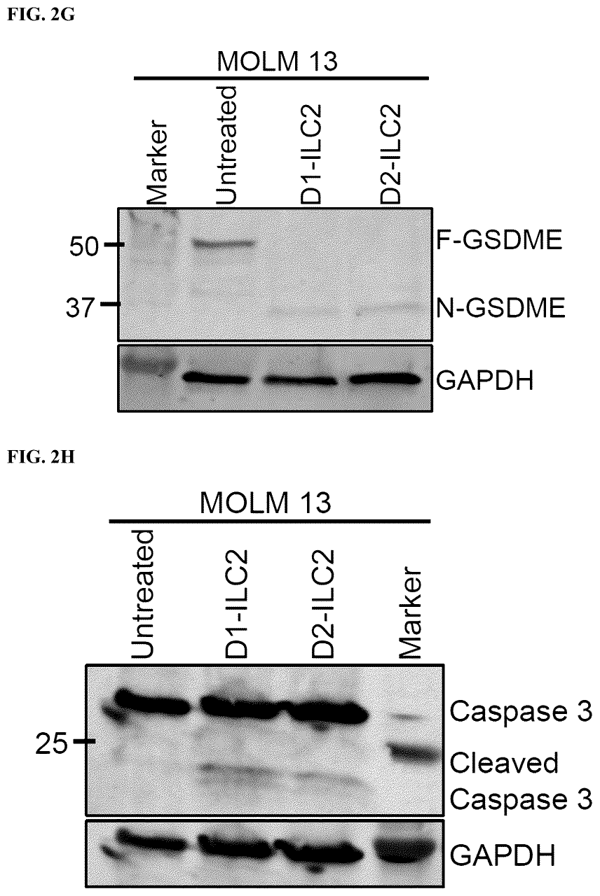

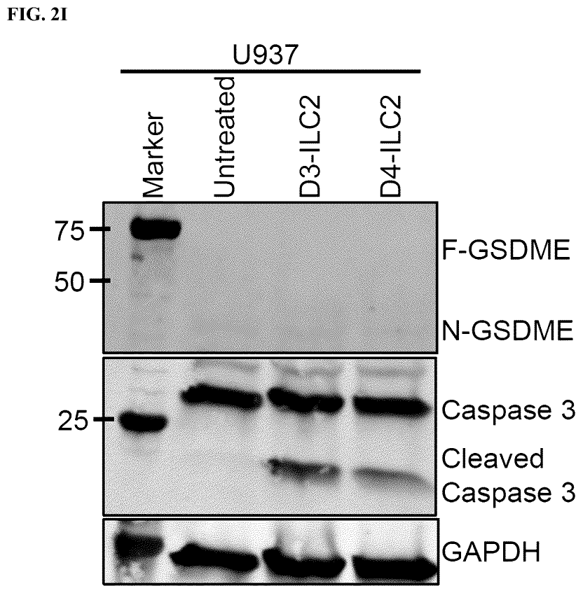

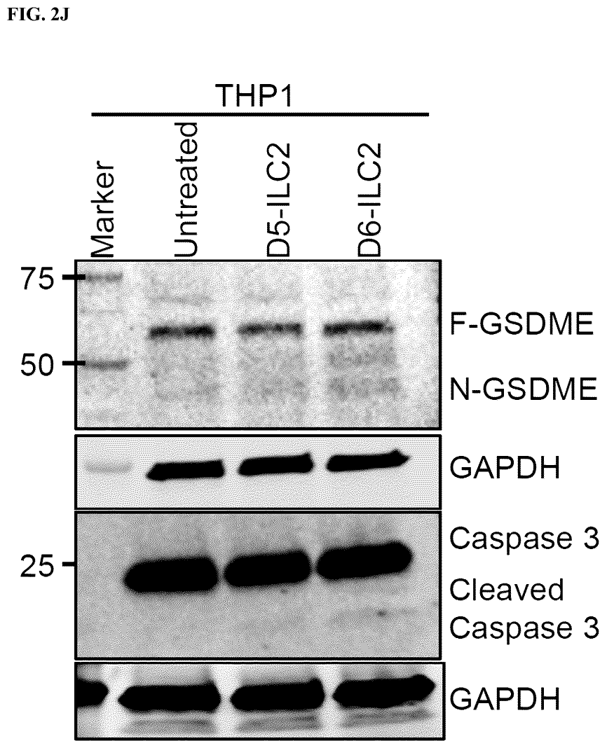

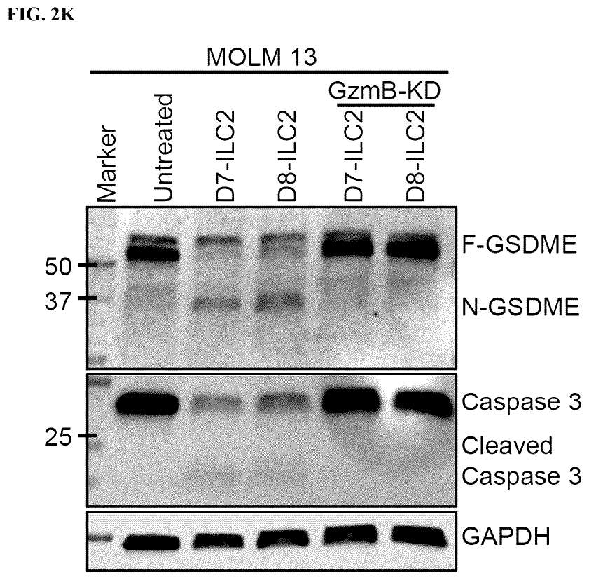

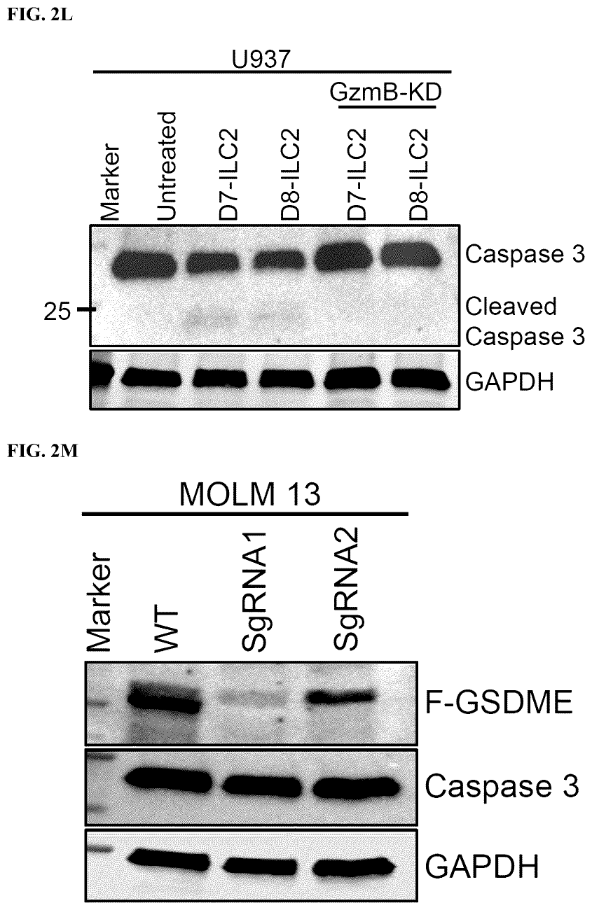

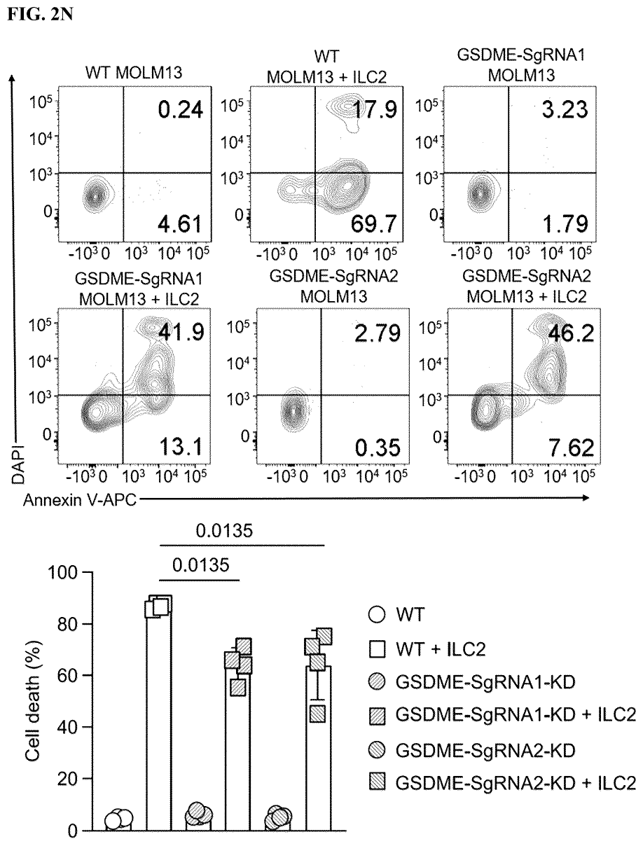

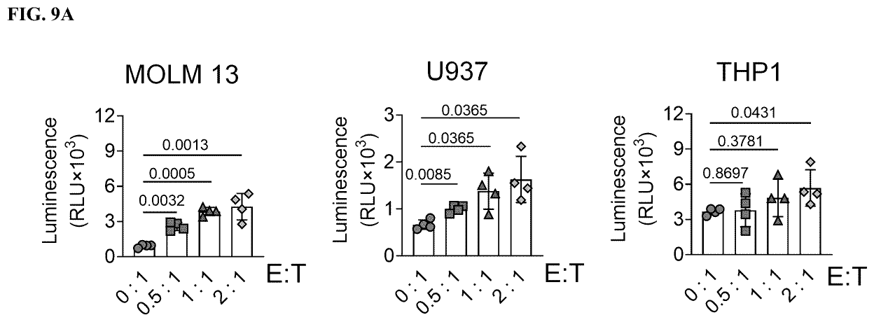

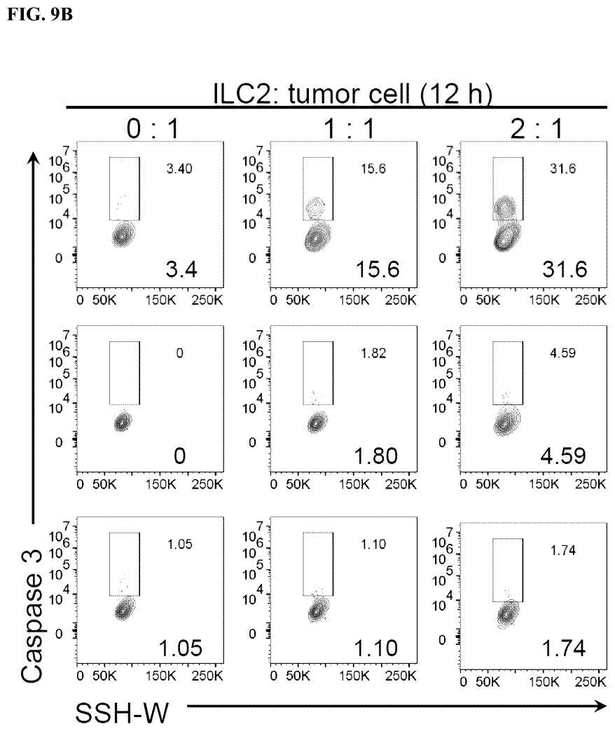

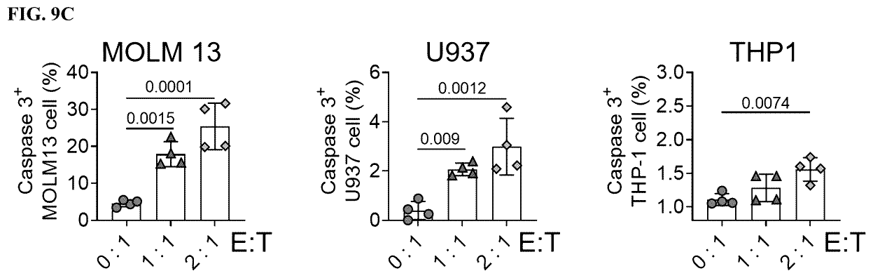

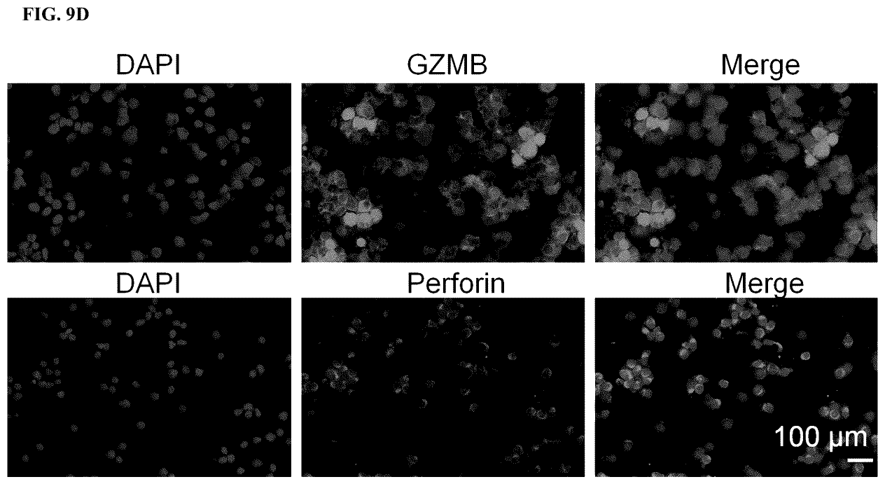

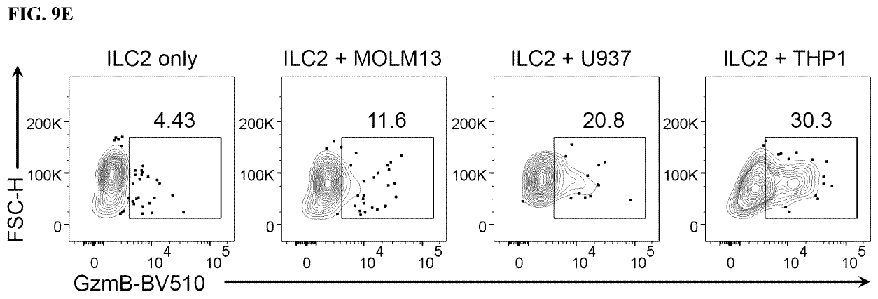

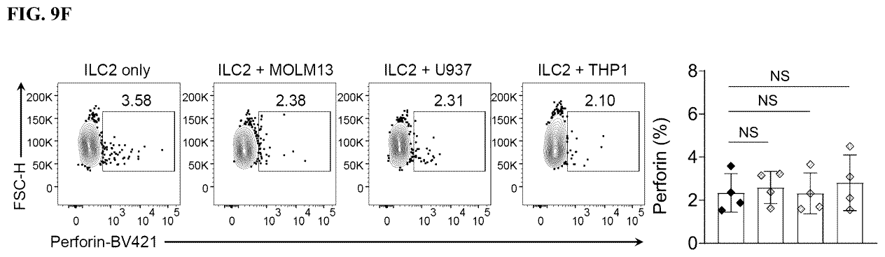

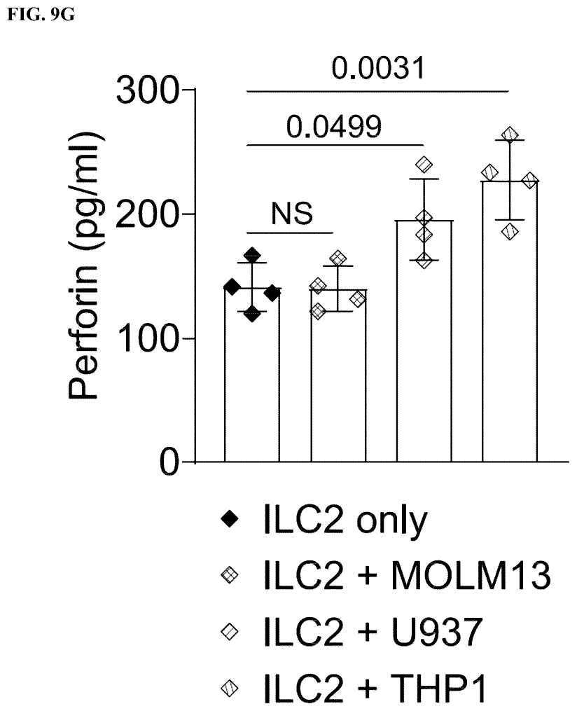

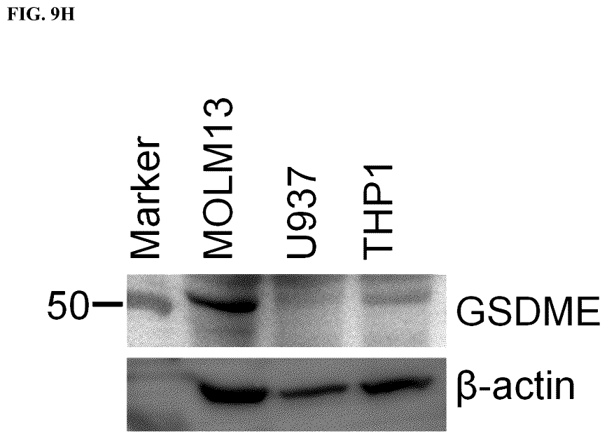

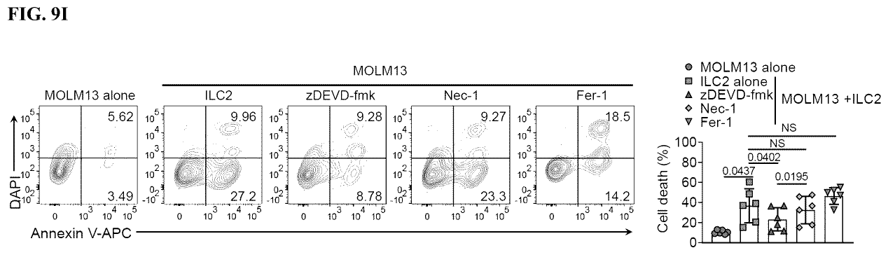

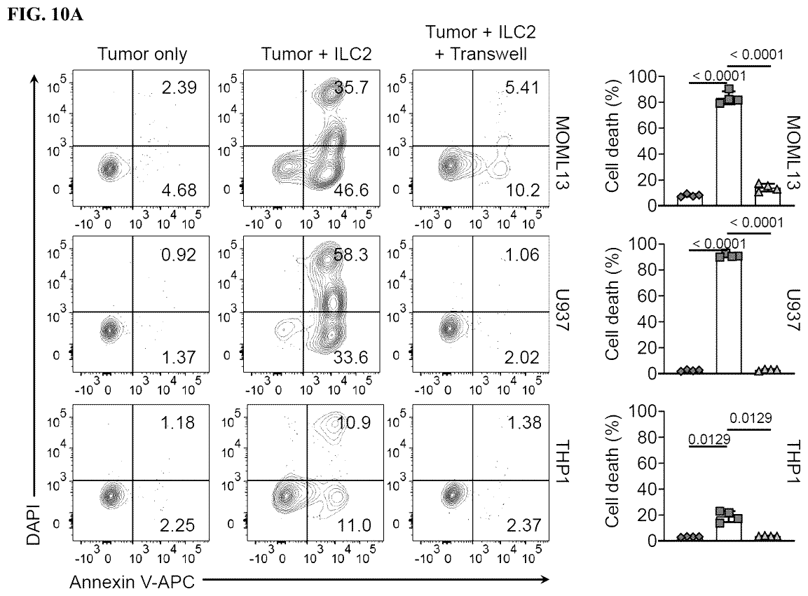

These results encouraged us to search for an alternative mechanism of ILC2-mediated AML cell lysis. Using time-lapse microscopy, we found that MOLM13 and THP1 but not U937 cells showed progressive cell death morphology with cellular swelling and bubble-like protrusions appearing on the surface of cellular membranes before their subsequent rupture (FIG. 2A). We also observed that activation of caspase 3 in AML cell lines was increased when co-cultured with Ex ILC2s at various ratios compared to co-culture without Ex ILC2s using luminescence-based and flow cytometry-based assays (FIGS. 9A-9C). These observations are unique characteristics of pyroptosis as mediated by granzyme B (GZMB)³⁶. Our results suggested that ILC2s have phenotypic properties similar to cytotoxic lymphocytes (e.g., NK cells) that can produce GZMB and perforin to lyse tumor cells through pyroptosis³⁶. Indeed, using flow cytometry we observed freshly isolated ILC2s and Ex ILC2s had the ability to produce GZMB and perforin upon stimulation with phorbol 12-myristate 13-acetate (PMA)/ionomycin (FIG. 2B). Similar results were obtained using immunofluorescence staining (FIG. 2C and FIG. 9D). The production of GZMB rather than perforin in Ex ILC2s was significantly induced when co-cultured with AML cells (FIG. 2D and FIG. 2E; FIGS. 9E-9G). Knock down (KD) of GZMB in Ex ILC2s using the CRISPR-Cas9 system decreased the ability of Ex ILC2s to lyse target AML cells (FIG. 2F), indicating that Ex ILC2s-mediated AML cell death depends on cytotoxic granule release.

GZMB can activate caspase 3-dependent pyroptosis in target cells by cleaving gasdermin E (GSDME), a feature typical for pyroptosis³⁶. To determine whether ILC2s induce pyroptosis by cleaving GSDME, we incubated the Ex ILC2s with MOLM13, U937 or THP1 for 6 h, and then sorted AML cells for immunoblotting. We observed that MOLM13 and THP1 but not U937 cells expressed GSMDE (FIG. 9H); after co-culture with Ex ILC2s, the GSDME was clearly cleaved in MOLM13 cells but only slightly cleaved in THP1 cells (FIG. 2G and FIG. 2J), while the caspase 3 was cleaved in these three AML cell lines, compared to co-culture without Ex ILC2s (FIGS. 2H-2J). These data suggest that: 1) Ex ILC2s can induce the GSDME-mediated pyroptosis of AML cells that express GSDME (e.g., MOLM13) and the caspase 3-mediated apoptosis of AML cells without GSDME expression (e.g., U937); 2) the ILC2-mediated caspase 3 cleavage was independent of the expression of GSDME. When we used GZMB KD Ex ILC2s instead of the wildtype (WT) Ex ILC2s to co-cultured with MOLM13 and U937 cells, the cleavage of GSDME in MOLM13 cells (FIG. 2K) and the cleavage of caspase 3 in MOLM13 (FIG. 2K) and U937 (FIG. 2L) cells disappeared, indicating that Ex ILC2-produced GZMB contributes to mediate the pyroptosis in GSDME-expressing AML cells and the apoptosis in non-GSDME-expressing AML cells.

To further determine the effect of GSDME on ILC2-mediated AML cell death, we used CRISPR-Cas9 system to knock down GSDME in MOLM13 cells. Knockdown of GSDME did not alter the expression of caspase 3 in MOLM13 (FIG. 2M). We next co-cultured Ex ILC2s with WT MOLM13 or GSDME KD MOLM13 cells and found knockdown of GSDME in MOLM13 reduced ILC2-induced cell death compared to WT MOLM13 cells, as measured by Annexin V/DAPI staining (FIG. 2N), suggesting that GSDME in AML cells mediates and is in part responsible for ILC2-induced AML cell death in those AML cells expressing GSDME. None of the necroptosis inhibitor necrostatin-1s or the ferroptosis inhibitors ferrostatin-1 suppressed, but caspase-3 inhibitor zDEVD-fmk that suppresses caspase 3 dependent apoptosis and pyroptosis did, ILC2s-mediated AML cell death (FIG. 9I), suggesting that necroptotic and ferroptotic forms of cell death were not involved here.

ILC2s Require Cell-Cell Contact with AML Cells to Induce GZMB Production Through DNAM-1 Interaction with its Ligands, CD112 and CD155

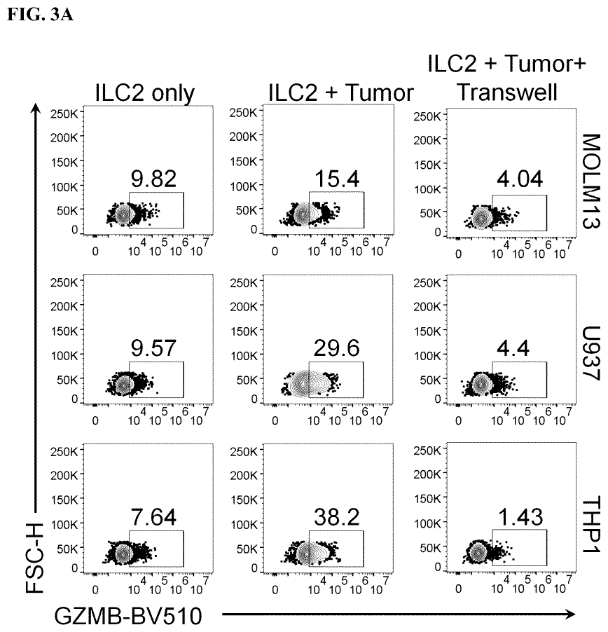

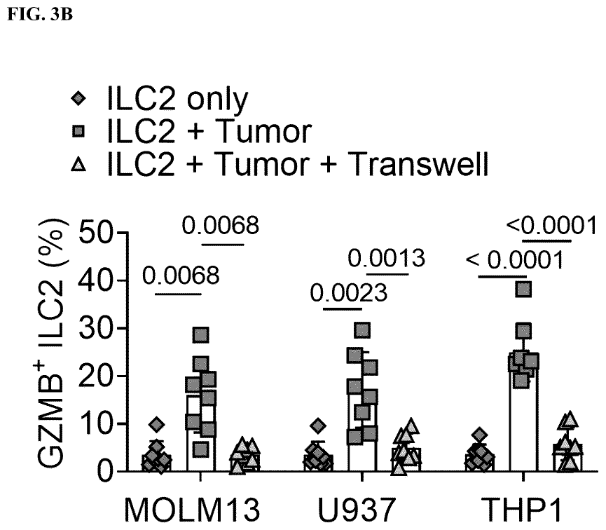

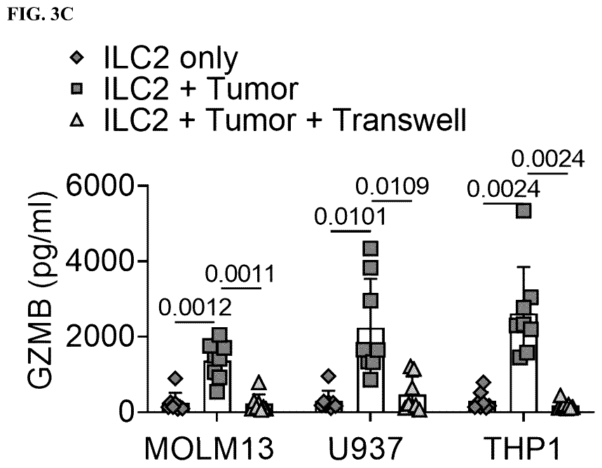

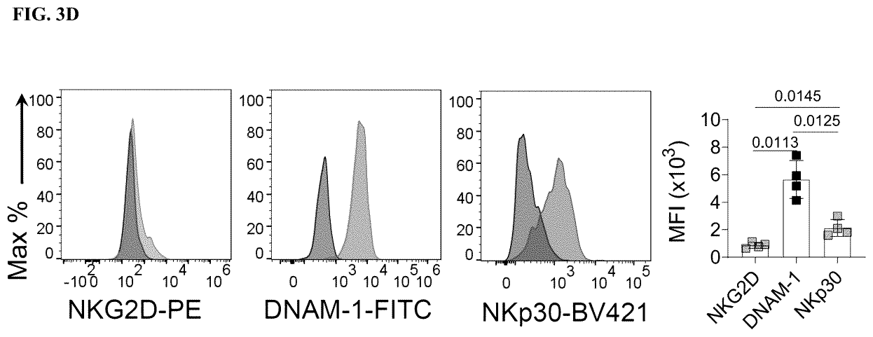

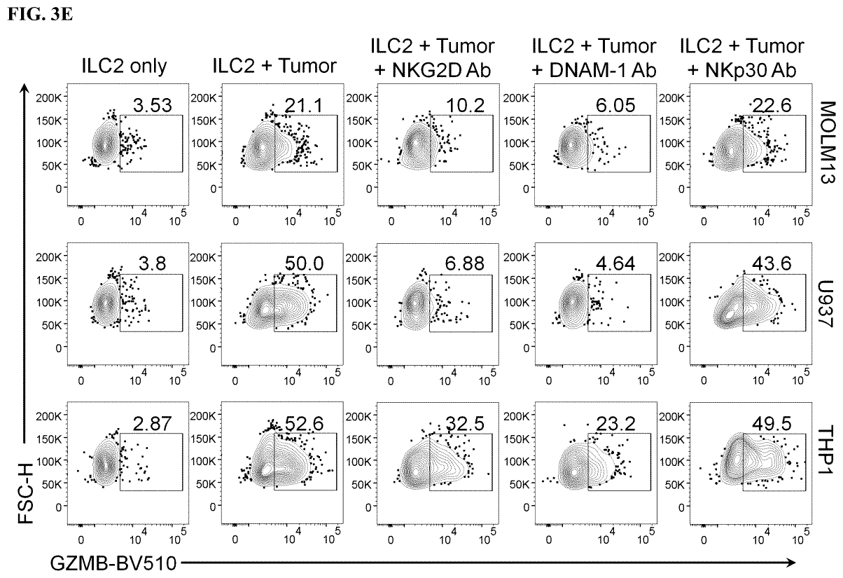

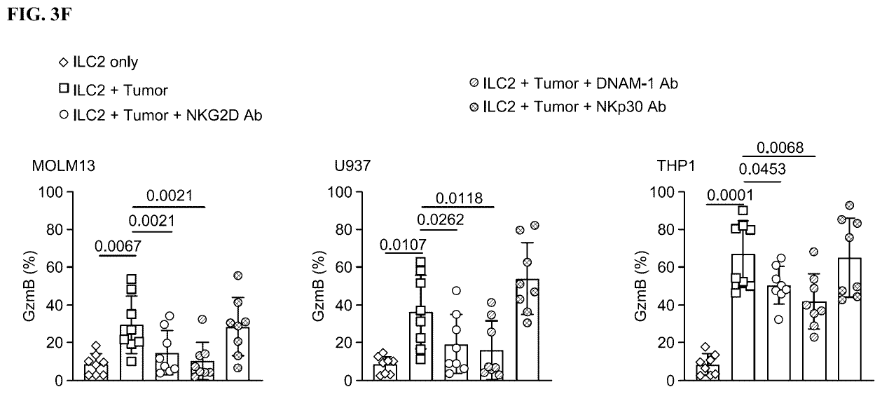

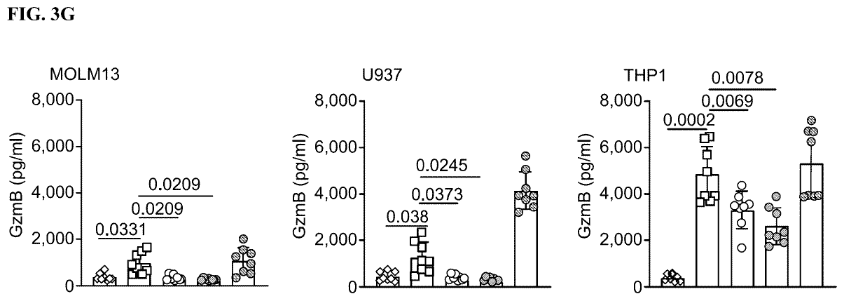

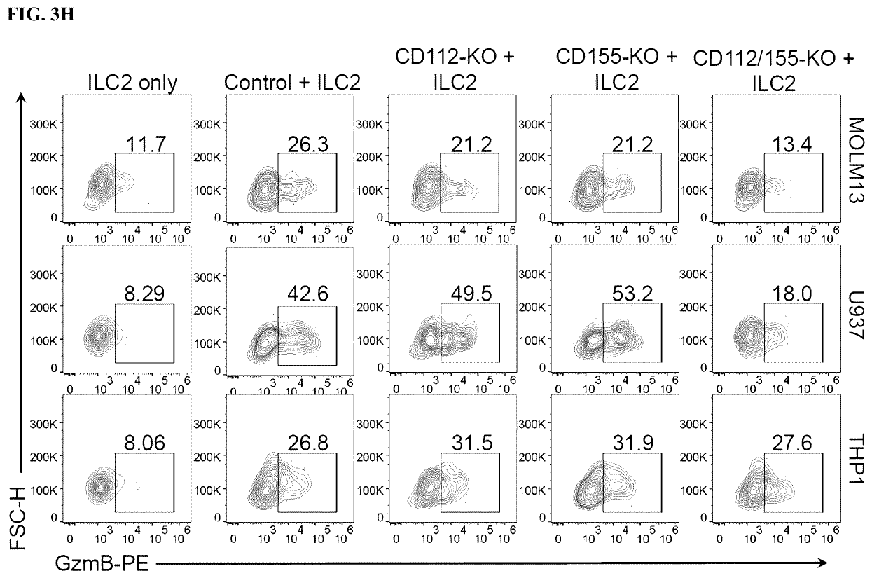

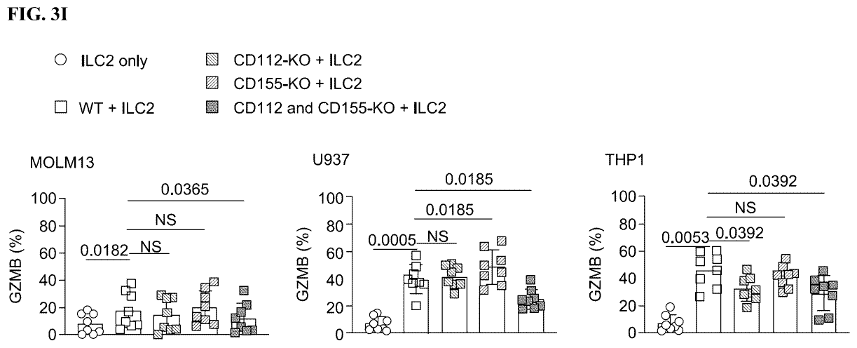

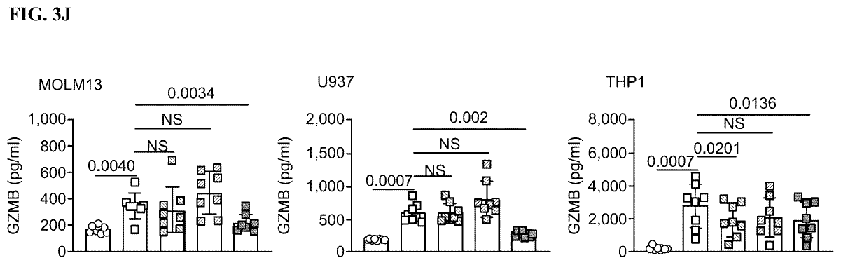

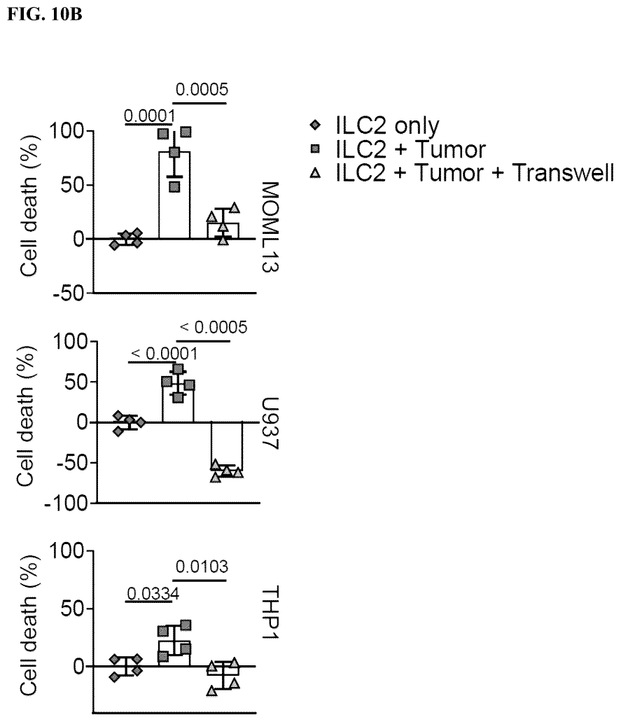

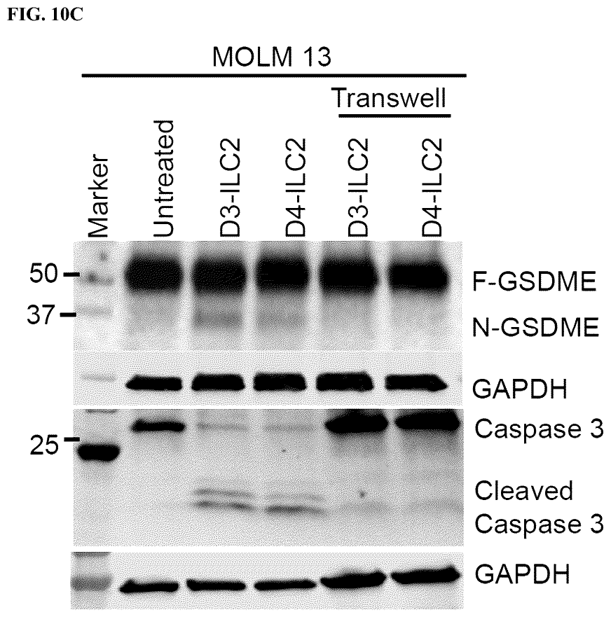

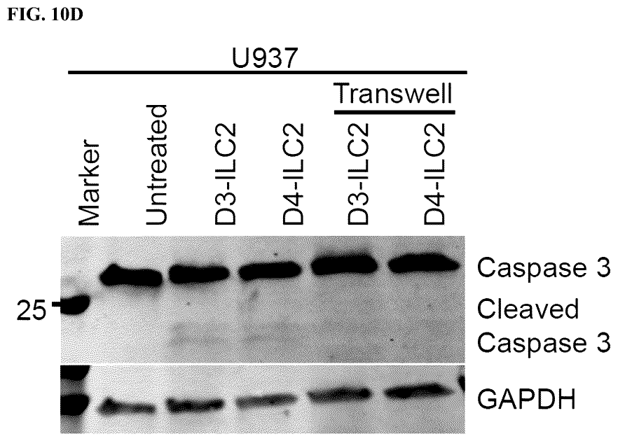

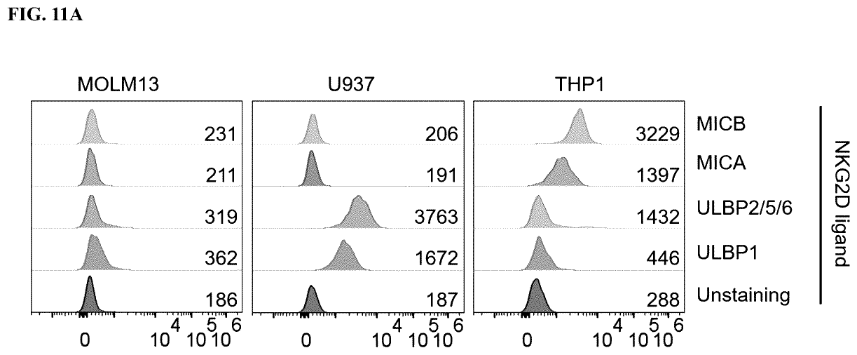

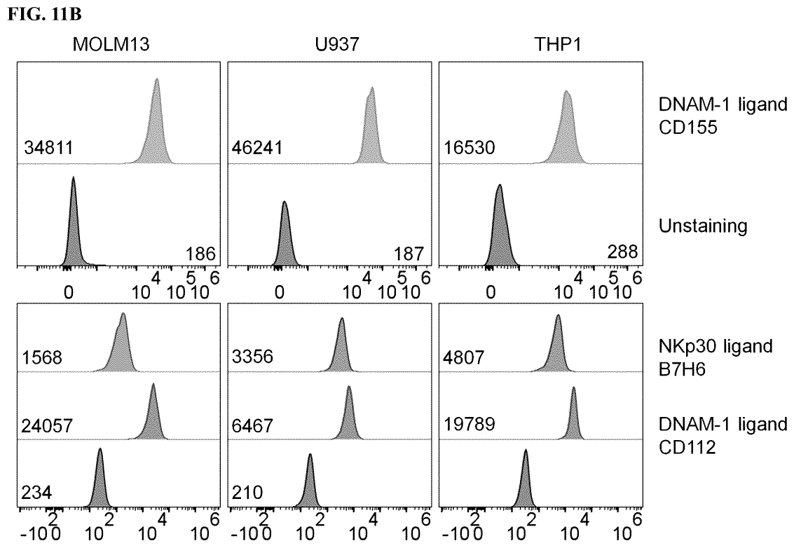

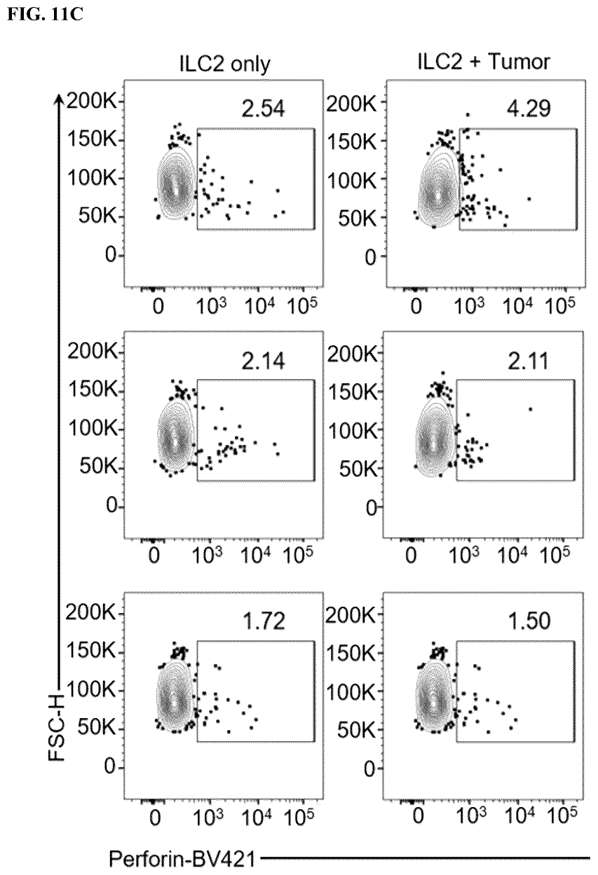

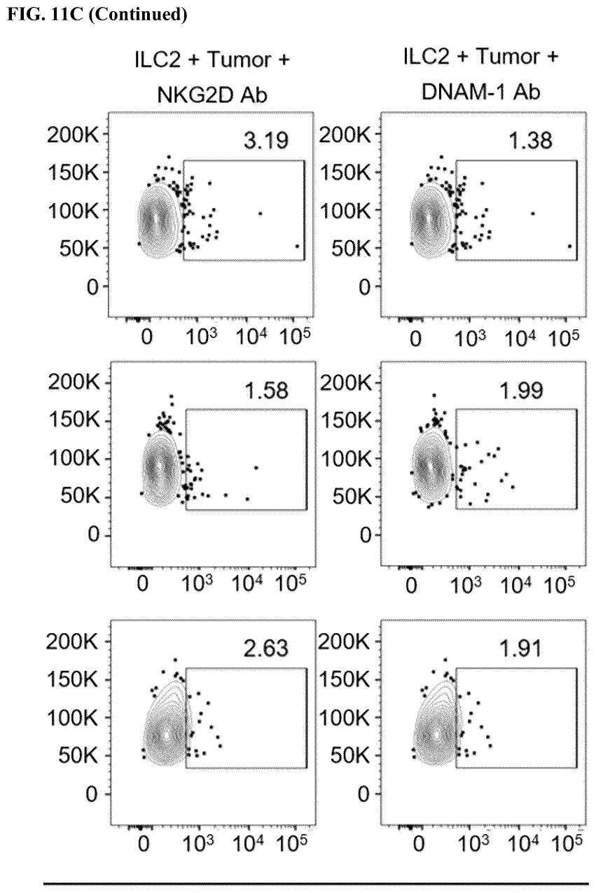

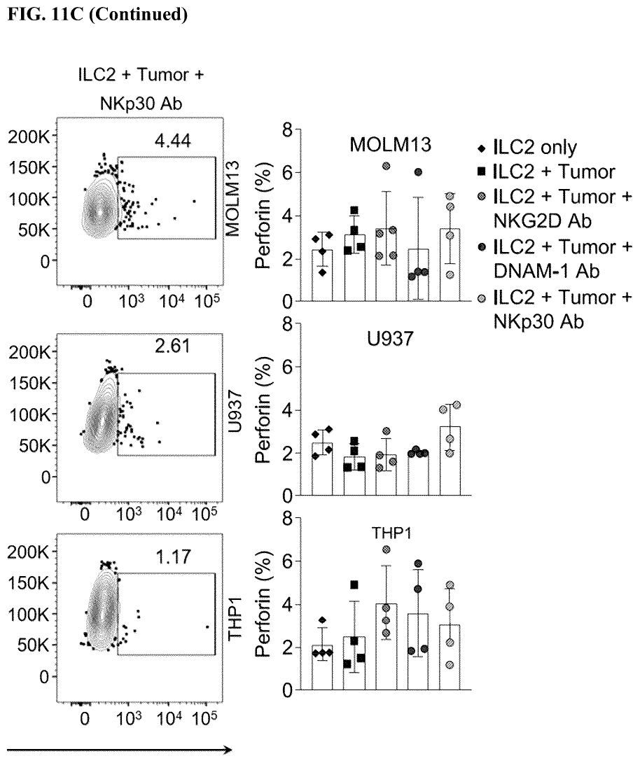

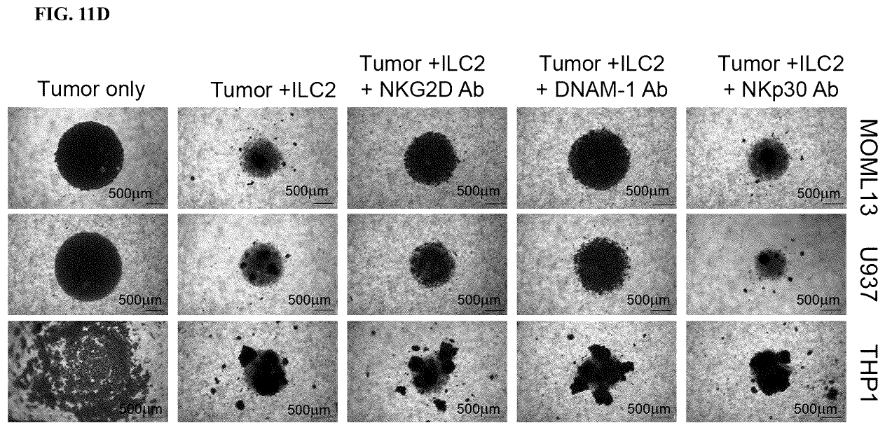

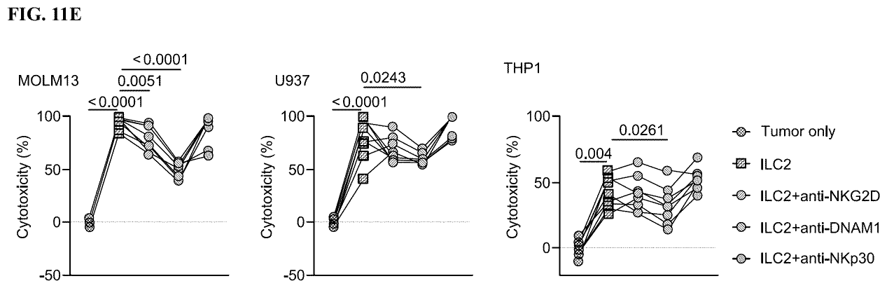

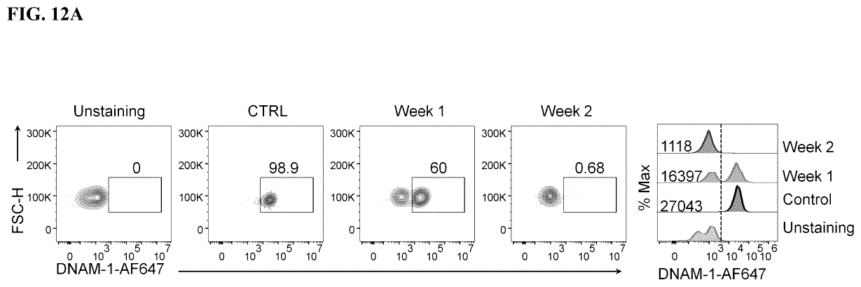

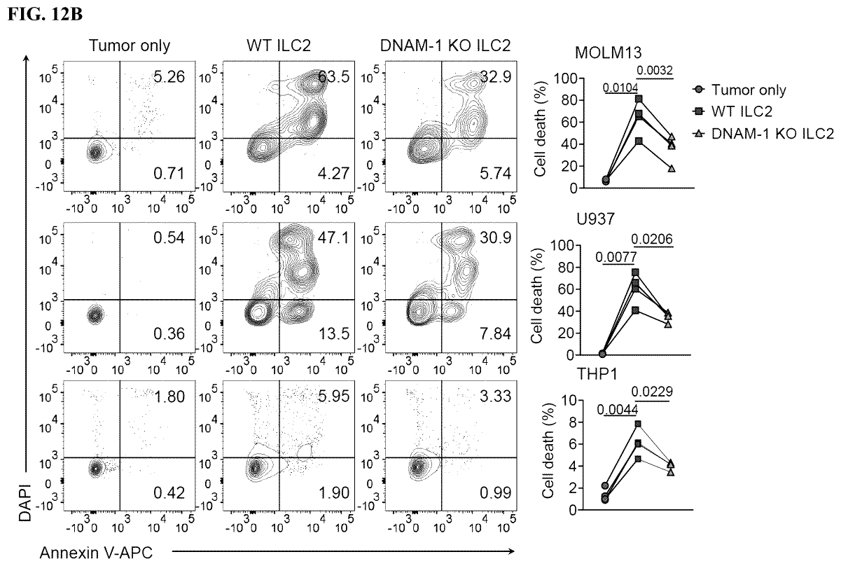

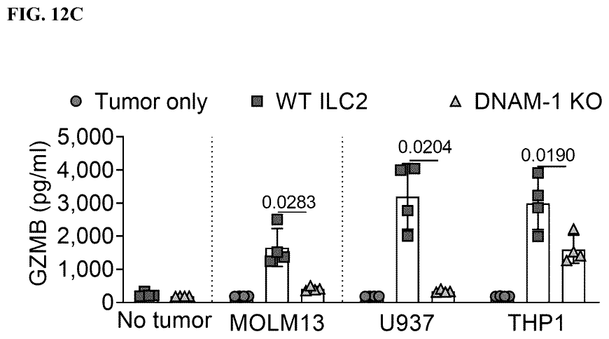

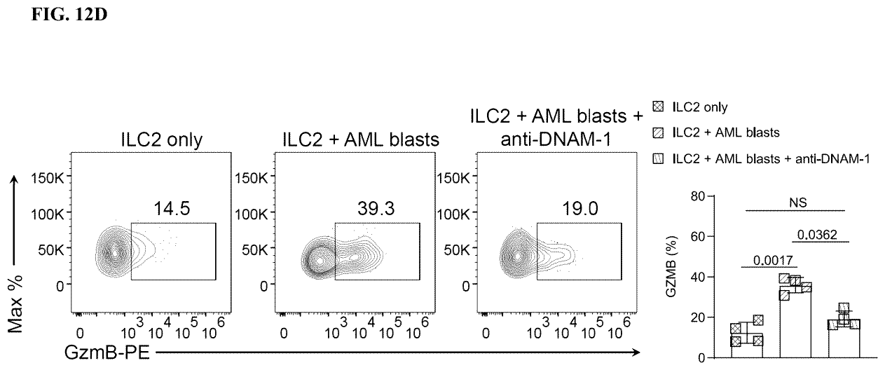

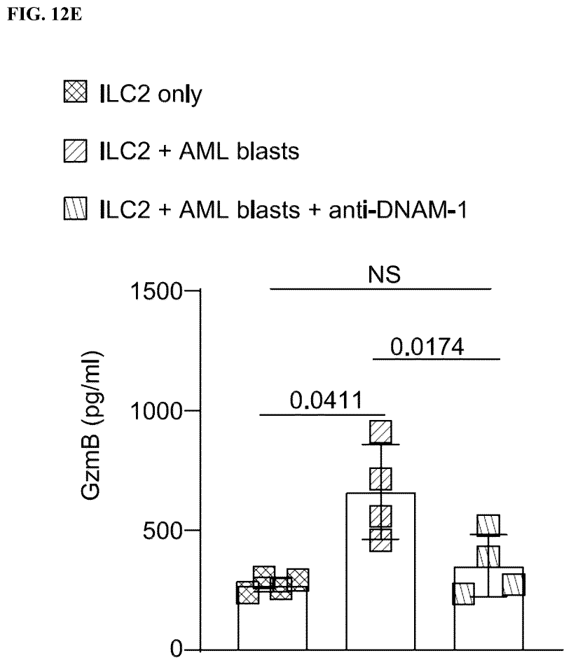

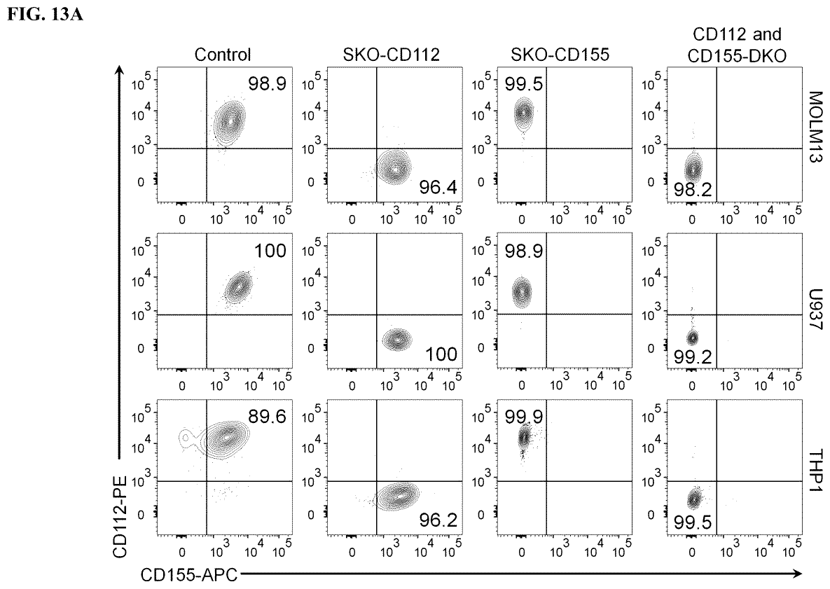

To determine the mechanism by which Ex ILC2s produced GZMB in the presence of AML cells (FIGS. 2D-2F), we co-cultured the two cell types separately in a transwell system and did not observe AML cell death using flow cytometry-based and luminescence-based assays (FIG. 10A and FIG. 10B). GZMB production by Ex ILC2s was significantly reduced in our transwell assay compared to without a transwell (FIGS. 3A-3C). The cleavage of GSDME in MOLM13 and the cleavage of caspase 3 in MOLM13 and U937 cells disappeared under conditions of transwell separation from Ex ILC2s (FIG. 10C and FIG. 10D). These results demonstrated that cell-cell contact is required to induce the production of GZMB by ILC2s and subsequent pyroptosis and/or apoptosis of AML cells. This observation prompted us to identify receptors and ligands necessary for recognition of target cells. The recognition and killing mediated by NK cells against tumor cells depends on signals triggered when ligands expressed on tumors are recognized by activating NK cell receptors³⁷. We observed that similar to NK cells, Ex ILC2s also expressed some activating receptors, such as the natural killer group 2 member D (NKG2D), DNAX Accessory Molecule-1 (DNAM-1 or CD226), and the natural cytotoxicity receptor 3 (NCR3 or NKp30) (FIG. 3D), with the highest level of expression for DNAM-1. It is known that AML cells express ligands for DNAM-1³⁸. Additionally, although NKG2D ligands MICA, MICB, and ULBP1/2/5/6) are at low levels in MOLM13, U937 has high expression of ULBP1/2/5/6 and THP1 has high expression of MICA and MICB (FIG. 11A). The ligands of DNAM-1 (CD155 and CD112) and the ligand of NKp30 (B7H6) were both highly expressed in all three of these AML cell lines (FIG. 11B). Thus, we wondered whether ILC2s recognize AML cells through NKG2D, DNAM-1, and/or NKp30. Assessment of GZMB and perforin by flow cytometry and ELISA demonstrated that antibodies that blocked binding of NKG2D or DNAM-1 significantly reduced Ex ILC2s production of GZMB but not perforin in the presence of MOLM13, U937, or THP1 AML cells. Blocking NKp30 had no significant effect (FIGS. 3E-3G; FIG. 11C). Consistent with this finding, the lysis of AML cells by Ex ILC2s was significantly reduced in the presence of antibodies that blocked binding of NKG2D or DNAM-1. Blocking NKp30 had no significant effect (FIG. 11D and FIG. 11E). The reduction of ILC2-mediated lysis of AML, however, was moderately more pronounced in the presence of antibody blocking the binding of DNAM-1 compared to that blocking the binding of NKG2D (FIG. 11D and FIG. 11E). Therefore, we primarily focused our attention on the function of DNAM-1 in ILC2-mediated lysis of AML. We performed knock out of DNAM-1 on ILC2s (DNAM-1-KO ILC2s) using the CRISPR-Cas9 system (FIG. 12A) and then co-cultured these cells with MOLM13, U937, or THP1 AML cells. We observed that compared to WT ILC2s, DNAM-1 KO ILC2s showed a significant decrease in their ability to lyse AML cells and to produce GZMB (FIG. 12B and FIG. 12C). Finally, we discovered that, similar to co-culturing with AML cell lines, GZMB production was also elevated when healthy Ex ILC2s were co-cultured with primary AML blasts isolated from the blood of patients with AML compared to without primary AML blasts (FIG. 12D and FIG. 12E). The elevation of GZMB was suppressed in Ex ILC2s co-cultured with primary AML blasts in the presence of DNAM-1 blockade antibody compared to in the absence of DNAM-1 blockade antibody (FIG. 12D and FIG. 12E).

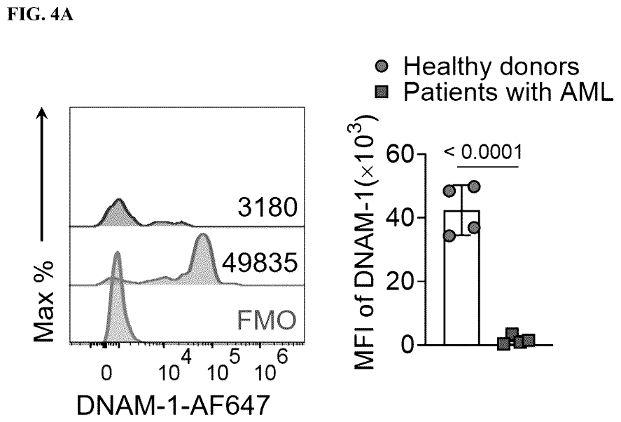

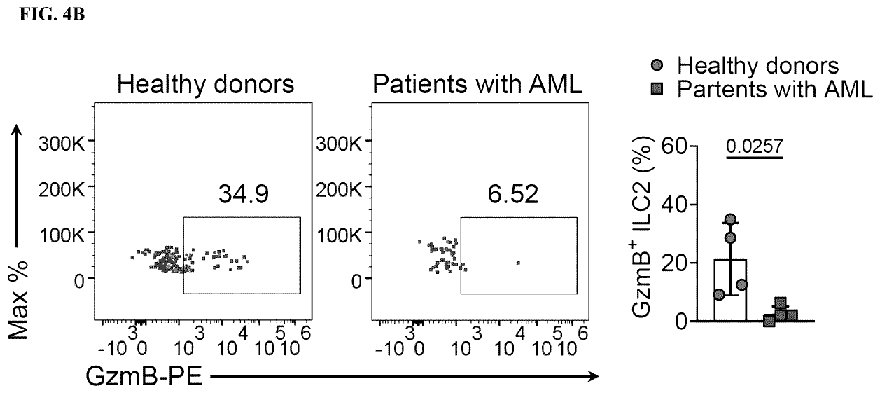

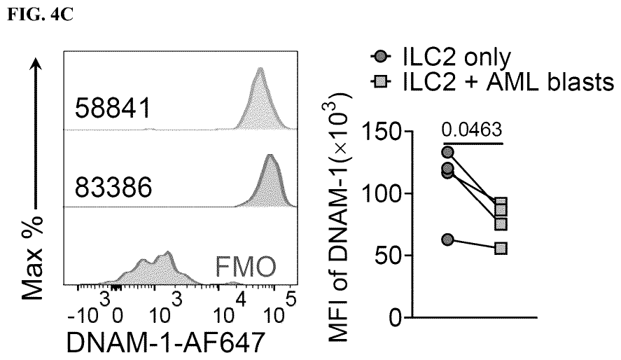

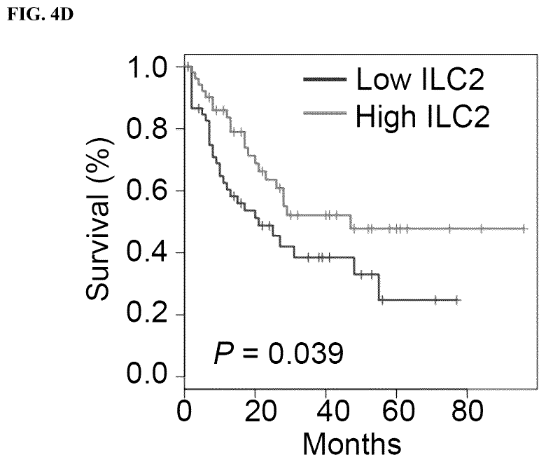

Separation of HD ILC2s and primary AML blasts in vitro culture using transwell rescued the primary AML blasts induced the downregulated DNAM-1 expression on HD ILC2s, suggesting the reduction of DNAM-1 on ILC2s of patients with AML requires the interaction between ILC2s and primary AML blasts. We therefore hypothesized that CD112 and/or CD155 expressed on primary AML blasts resulted in decreased expression of DNAM-1 on ILC2s. Indeed, CD155 single knockout or CD112 and CD155 double knockout rather than CD112 single knockout on primary AML blasts didn't result in the decreased DNAM-1 expression on HD ILC2s after in vitro culture, suggesting that CD155 but not CD112 contributes to the suppression of DNAM-1 on ILC2s. Thus, considering the relevance of DNAM-1 on ILC2s recognition and lysis of leukemic cells, the reduced expression of DNAM-1 on ILC2s from patients with AML may represent an additional mechanism of tumor escape or immune evasion. Analysis of 53 AML cases from TCGA showed that AML patients with high ILC2s gene signatures, as defined by CD117, PTGDR2, GATA3, IL9, IL13, HPGDS, S1PR1, TLE4, IL1RL1, IL17RB, and ICOS had a significantly prolonged overall survival compared to AML patients with low ILC2s gene signatures (FIG. 4D). Taken together, these data show that the functional roles of human ILC2s become dysregulated in the context of AML, and a high ILC2s gene signature correlates with more favorable clinical outcomes in AML.

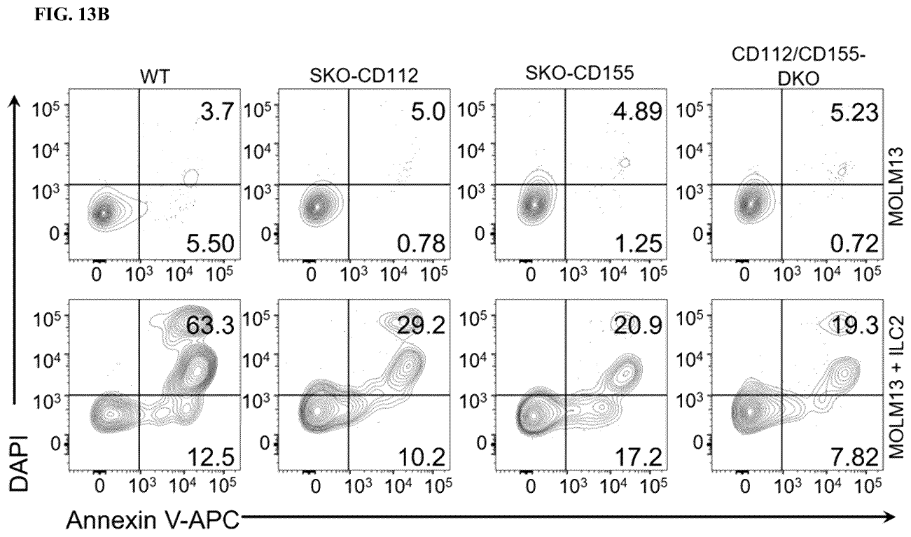

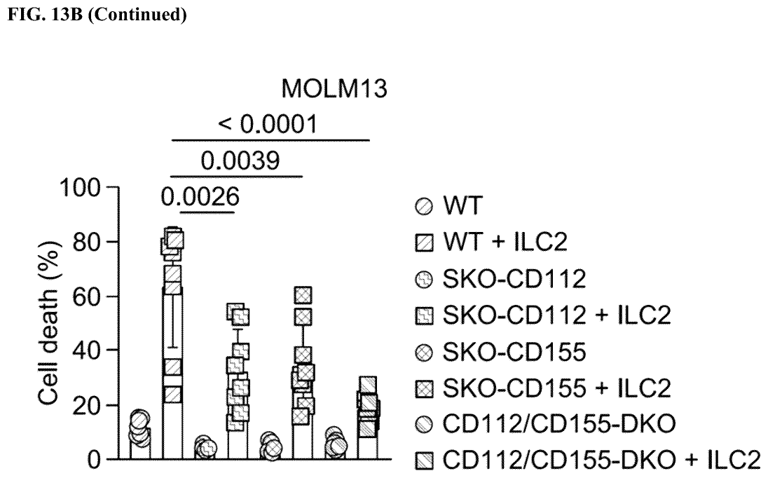

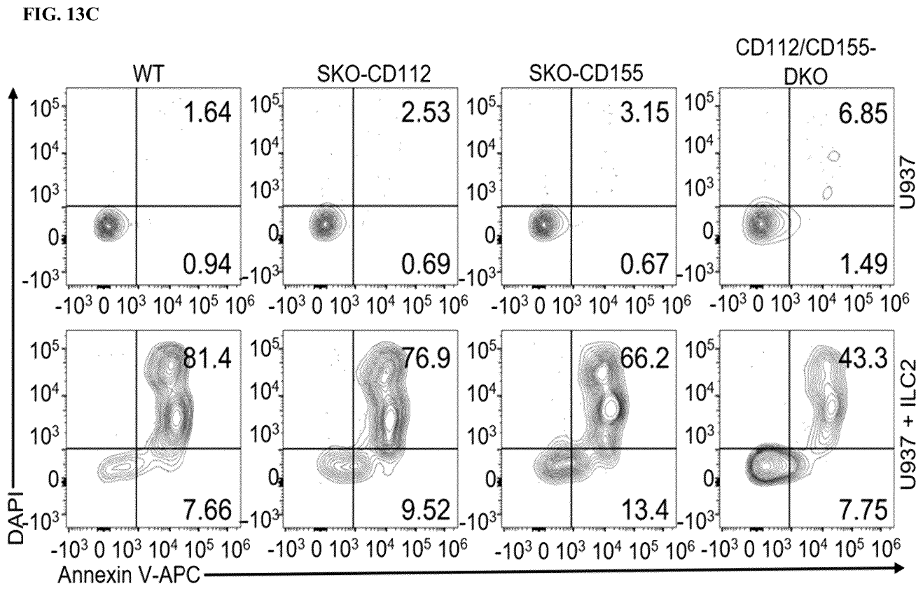

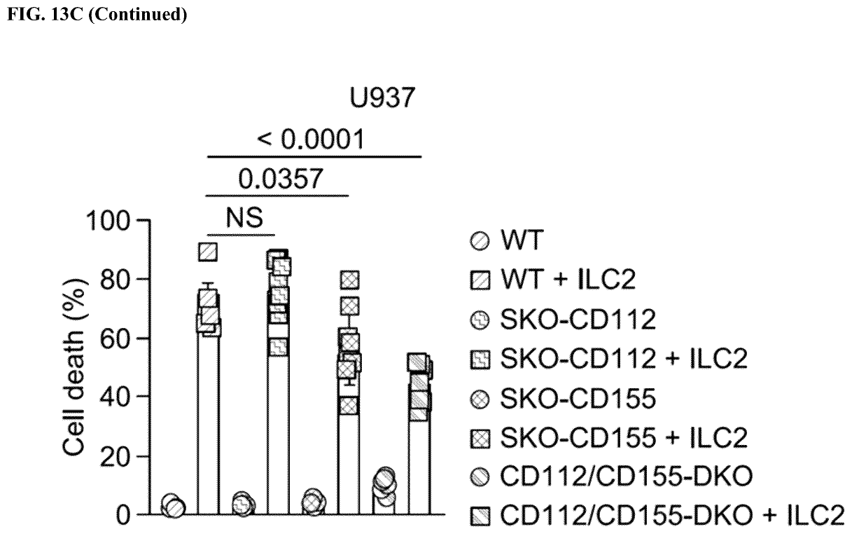

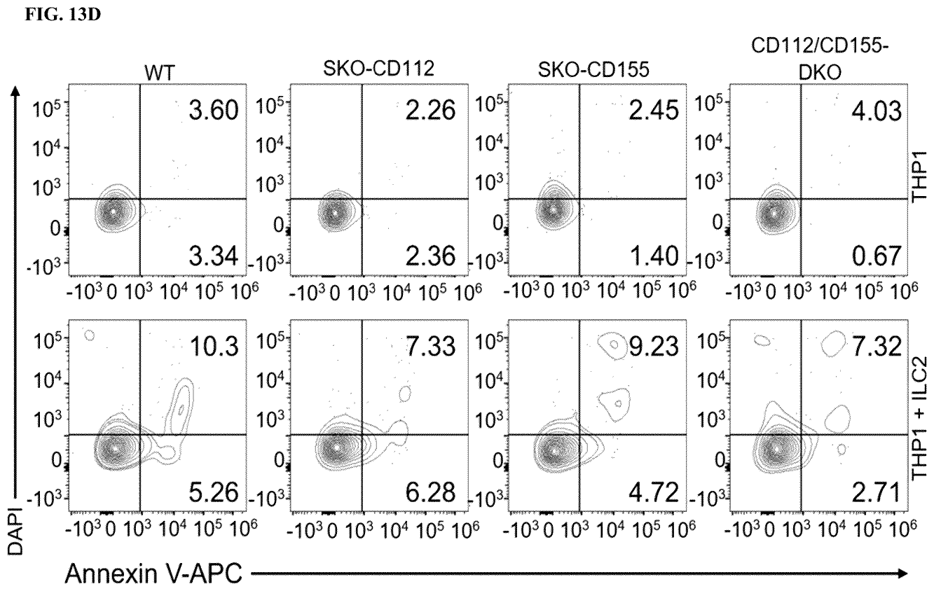

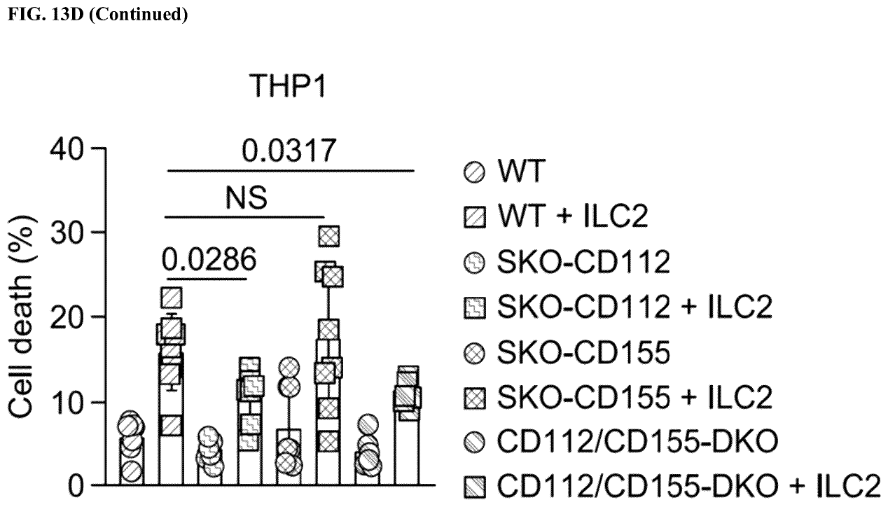

Using the CRISPR-Cas9 system, we additionally performed single-knockout of CD112 (SKO-CD112), CD155 (SKO-CD155), and double knockout of CD112 and CD155 (DKO) on MOLM13, U937, and THP1 (FIG. 13A). We then co-cultured these AML cells with Ex ILC2s. Flow cytometry and ELISA showed that compared to co-culture with WT AML cells, the production of GZMB in Ex ILC2s co-cultured with single SKO-112 or SKO-155 AML cells was not obviously altered, but significantly decreased in co-cultured DKO AML cells (FIGS. 3H-3I). Further, compared to WT AML cells, DKO AML cells co-cultured with Ex ILC2s reduced the lysis of target AML cells by Ex ILC2s (FIGS. 13B-13D). Together, these data suggest that 1) the production of GZMB by ILC2s is mediated through DNAM-1(+) ILC2s interacting with CD112 and CD155 expressed on AML cells; and 2) CD112 and CD155 expressed on AML cells appear to have a role in inducing the subsequent lysis of AML cells by ILC2s.

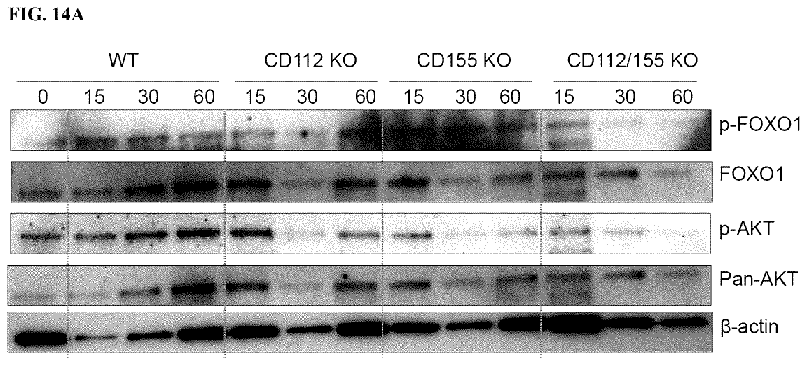

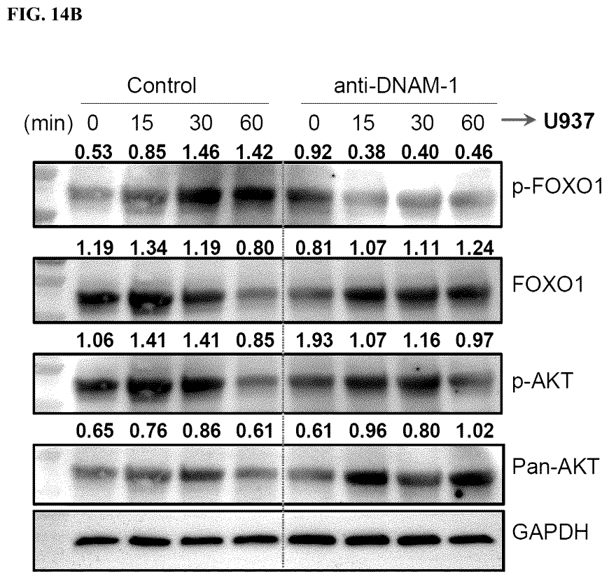

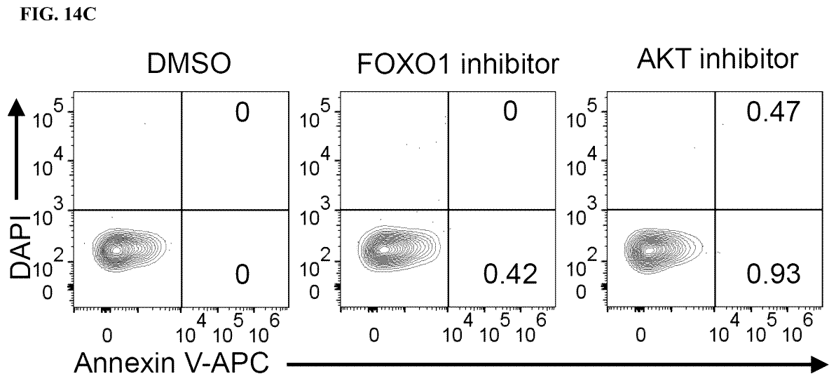

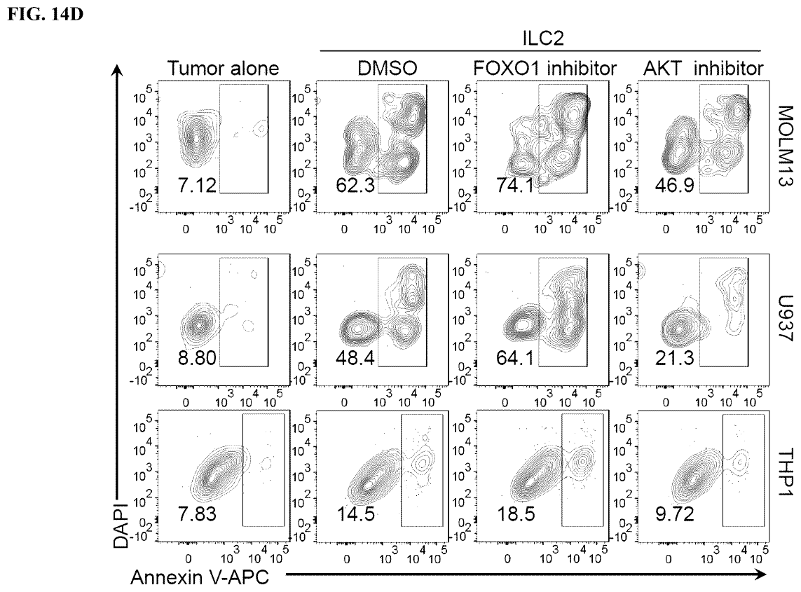

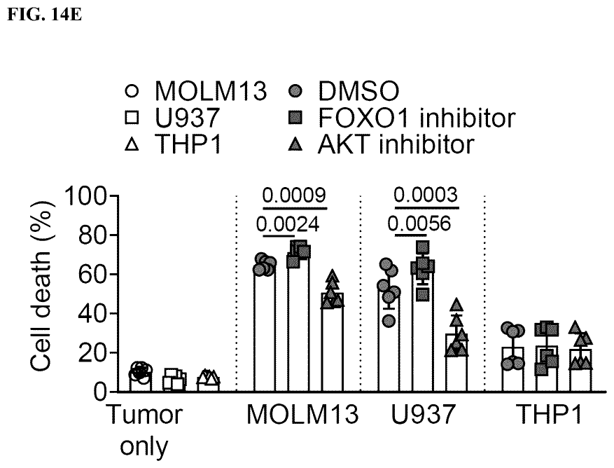

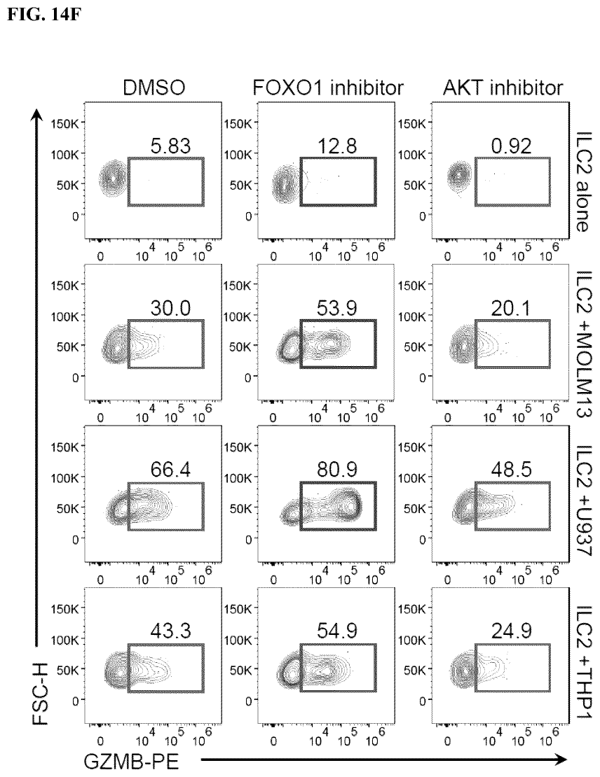

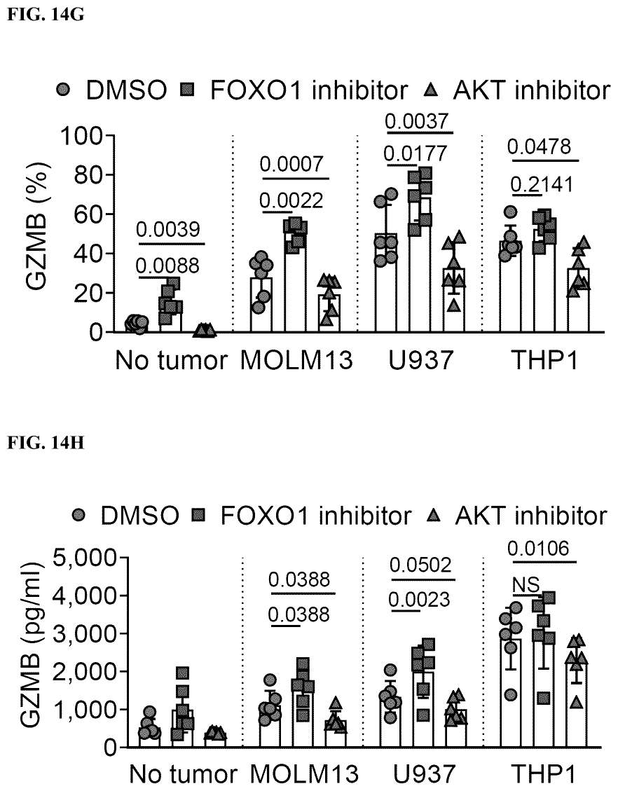

DNAM-1 activation requires engagement with its ligands CD112 and/or CD155, which has been confirmed by performing in vitro lysis assays using Ex ILC2s as effector cells (FIGS. 11D, 11E, 12B-12E). It was previously demonstrated that NK cell DNAM-1's engagement with its ligands results in inactivation of its negative regulatory control over mouse NK cell effector function through the phosphorylation of transcription factor forkhead box 01 (FOXO1)³⁹. To assess whether DNAM-1-mediated cytotoxic function in Ex ILC2s was dependent on signaling through FOXO1, we co-cultured Ex ILC2s with WT, SKO-CD112, SKO-CD155, and DKO U937 cells at various times (FIG. 14A). We found that co-culture of Ex ILC2s with WT U937 cells increased the phosphorylation of FOXO1 (p-FOXO1) in Ex ILC2s at 30 and 60 min, while the p-FOXO1 was obviously decreased in only Ex ILC2s co-cultured with DKO U937 cells at 30 and 60 min (FIG. 14A), suggesting that 1) the phosphorylation of FOXO1 may also be involved in inactivating the negative regulatory control over human ILC2s cell effector function; and 2) CD112 and/or CD155 contact with DNAM-1 results in inducing the phosphorylation of FOXO1 in Ex ILC2s. Incubation of ILC2s with WT U937 in the presence of a DNAM-1 blockade antibody resulted in the suppression of p-FOXO1 in Ex ILC2s compared to cultures with U937 cells in the absence of a DNAM-1 blockade antibody at 30 and 60 min (FIG. 14B). Similar results were found when examining the phosphorylation of AKT (FIG. 14A and FIG. 14B). Using AKT inhibitor (afuresertib) canceled the U937 cell-induced p-FOXO1, suggesting that FOXO1 is a direct substrate of phosphorylated AKT, and that DNAM-1 triggers AKT phosphorylation, which is consistent with the previous reports⁴⁰. The lysis of AML cells (FIGS. 14C-14E) and the production of GZMB (FIGS. 14F-14H) by Ex ILC2s was increased in the presence of a FOXO1 inhibitor (AS1842856, which can inactivate FOXO1) and reduced in the presence of an AKT inhibitor (which can suppress the AKT-FOXO1 signaling pathway), validating that DNAM-1-mediated phosphorylation of FOXO1 is required for the enhancement of ILC2s effector activity. Collectively, these data demonstrate that DNAM-1 engagement in Ex ILC2s triggers downstream inactivation of FOXO1, which then enhances the function of Ex ILC2s.

### ILC2s and NK Cells Exhibit Comparable Antitumor Efficacy Function In Vitro and In Vivo

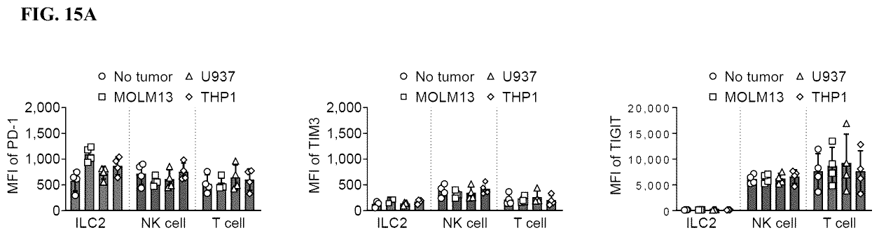

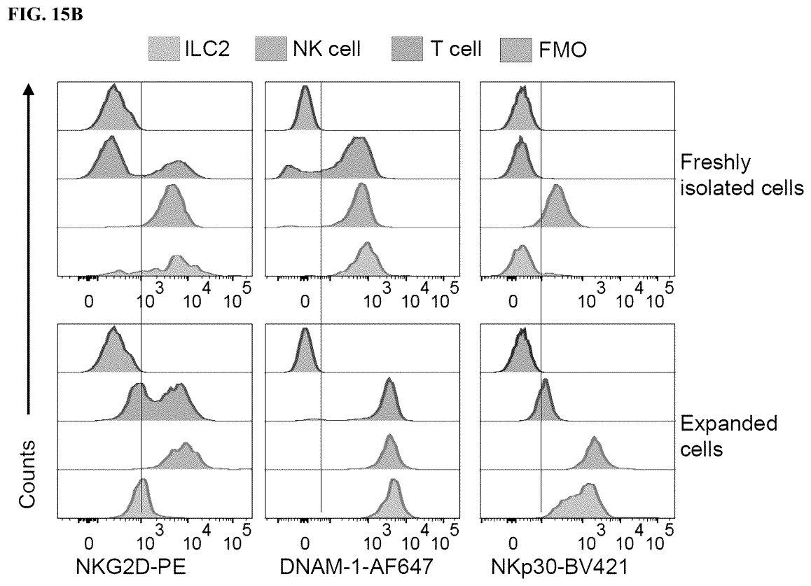

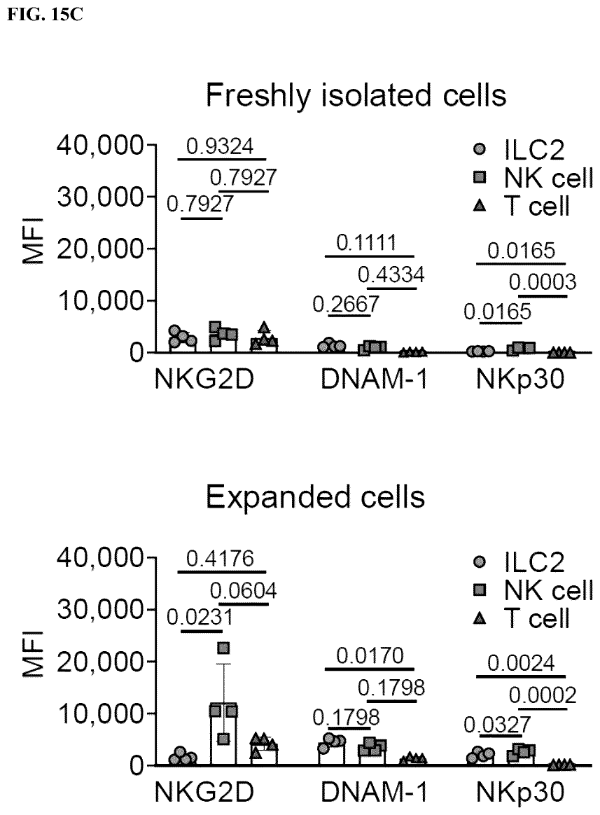

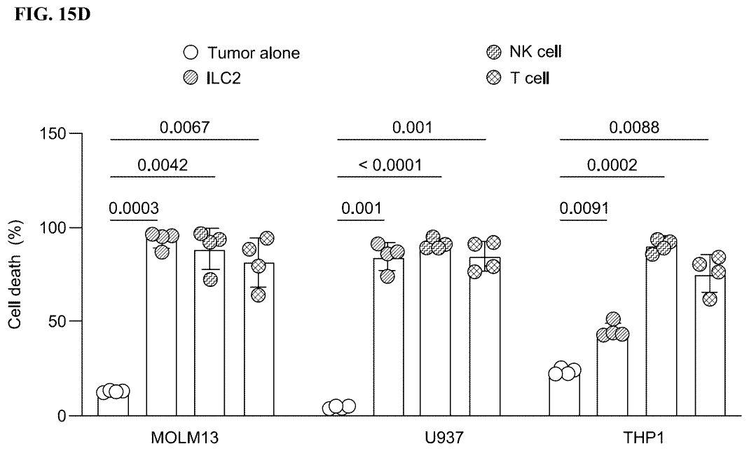

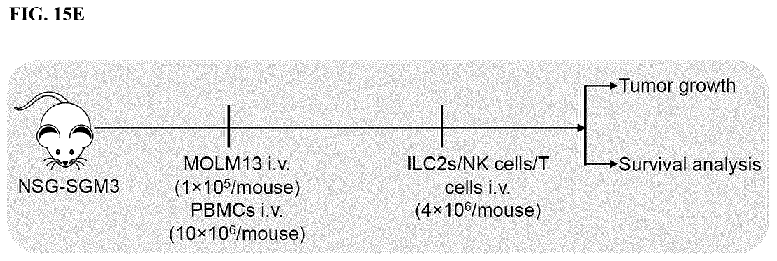

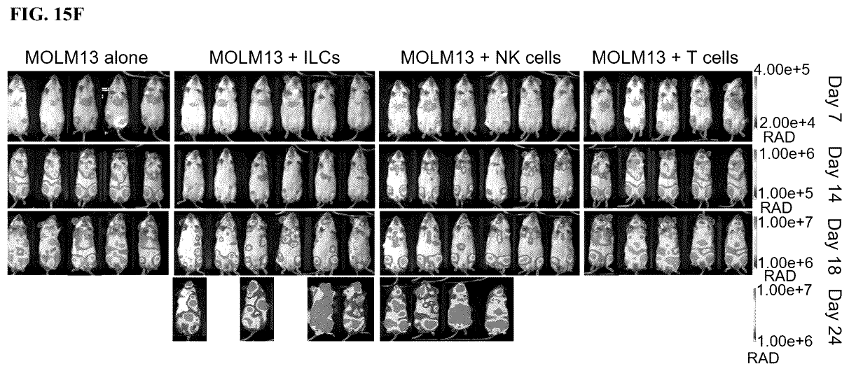

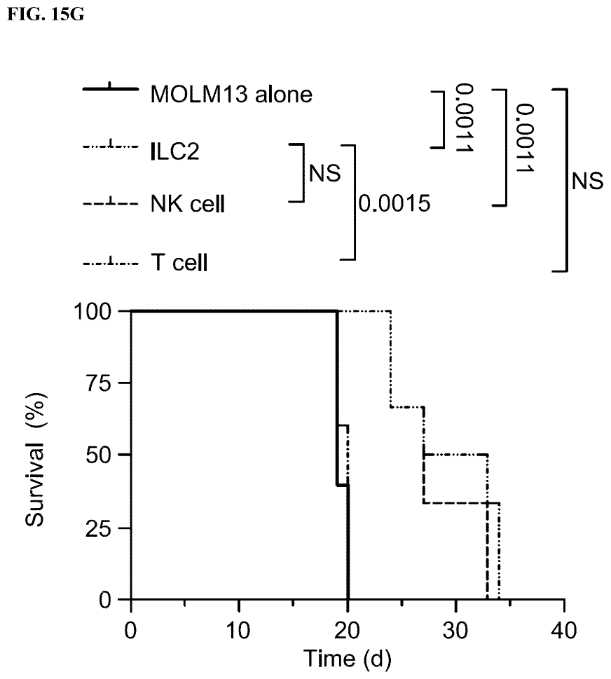

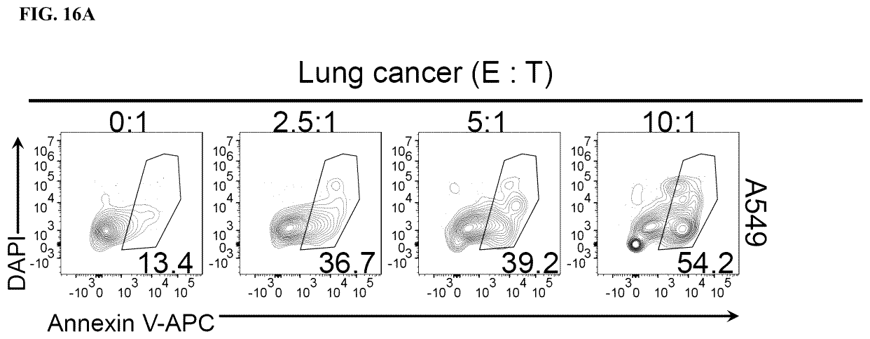

ILC2s, like NK cells and T cells, express an inhibitor receptor and activation receptor and the loss of the balance of activation signals and inhibitory signals leads to suppressed effector function of these immune cells^{22,24}. We compared the expression of inhibitor receptors in Ex ILC2s with expanded NK cells (Ex NK cells) and expanded pan T cells (Ex T cells) in the absence or presence of tumor cells for 48 h in vitro and observed that ILC2s, NK cells, and T cells have comparable PD-1 expression but low TIM3 and almost no TIGIT expression regardless of co-culture with or without tumor cells (FIG. 15A), suggesting ILC2s exhibit low exhausted characteristics partially. In an examination of activation receptors, we found freshly isolated ILC2s, NK cells, and T cells have similar expressions of NKG2D and DNAM-1. However, Ex ILC2s reduced NKG2D expression compared to Ex NK cells and Ex T cells and have comparable DNAM-1 expression with Ex NK cells but higher than Ex T cells (FIG. 15B and FIG. 15C). NK cells maintained higher NKp30 expression than ILC2s and T cells regardless of the freshly isolated or expanded cell (FIG. 15B and FIG. 15C). In a comparison of the ability of these three cells to lyse AML cells, we found Ex ILC2s, Ex NK cells, and Ex T cells exhibited the similar cytotoxic roles against MOLM13 and U937 cells but not THP1 cells in vitro (FIG. 15D). We next employed an AML model of i.v. injected firefly FFLuc-labeled MOML13 cells and PBMCs into NSG-SGM3 mice, followed by i.v. infusion of Ex ILC2s, Ex NK cells, and Ex T cells (FIG. 15E). Single dose infusion of Ex ILC2s and Ex NK cells improved tumor control and significantly prolonged mouse survival compared to the control group without Ex ILC2s treatment and the group with T cell infusion (FIG. 15E and FIG. 15F). Together, these results indicate that ILC2s exhibit antitumor efficacy function in vitro and in vivo similar with those of NK cells.

### Immune Evasion of AML by Inhibiting DNAM-1 Surface Expression on ILC2s

We had demonstrated that ILC2s require cell-cell contact with AML cells through ILC2s expression of DNAM-1 to produce GZMB (FIGS. 3A-3C). This led us to examine the expression levels of DNAM-1 on ILC2s in the blood of AML patients (AML ILC2s). DNAM-1 expression on ILC2s in AML patients was profoundly reduced compared to expression levels on healthy donor ILC2s (HD ILC2s; FIG. 4A). Likewise, the production of GZMB in ILC2s from AML patients was significantly decreased relative to ILC2s from HDs (FIG. 4B). Consistent with this, co-culture of HD ILC2s with primary AML blasts reduced DNAM-1 expression (FIG. 4C), suggesting that primary AML blasts could produce signals to reduce DNAM-1 expression on AML ILC2s in vivo. The analysis of DNAM-1 ligands showed a high expression of CD112 and CD155 on primary AML blasts (FIG. 4D). These two ligands suppressed the DNAM-1 expression on NK cells and T cells^{41,42}. An inverse correlation between CD112/CD155 expression and DNAM-1 expression in patients with AML was also found using the TCGA database (FIG. 4C).

### ILC2s Protect Against Solid Tumor Progression

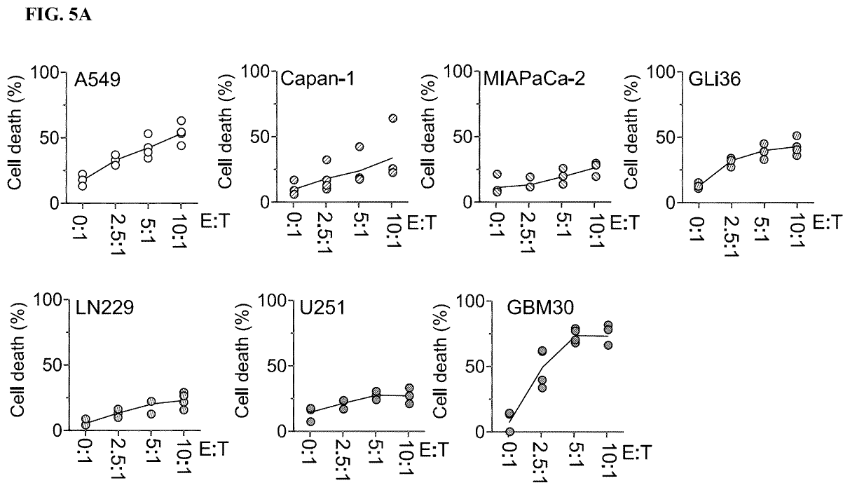

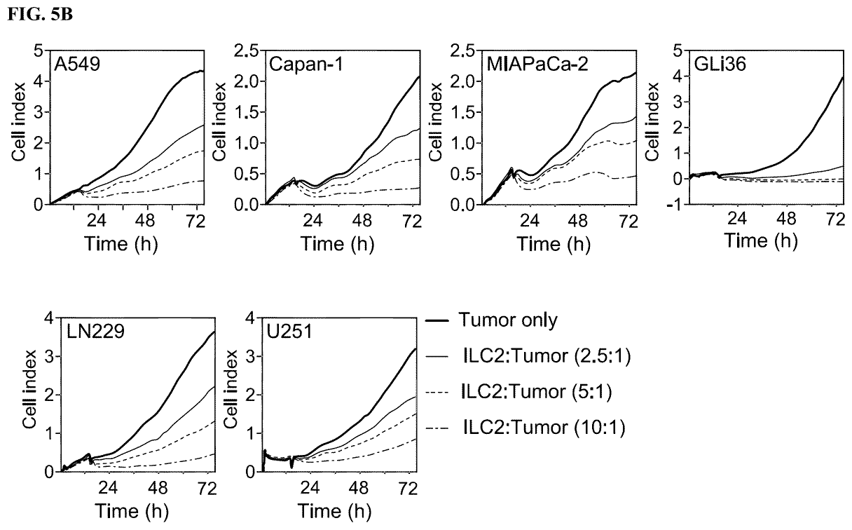

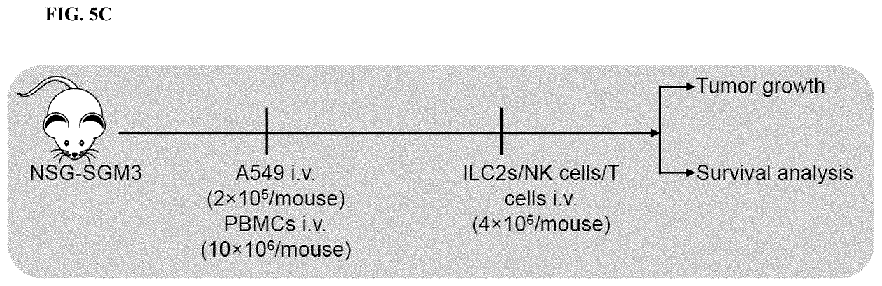

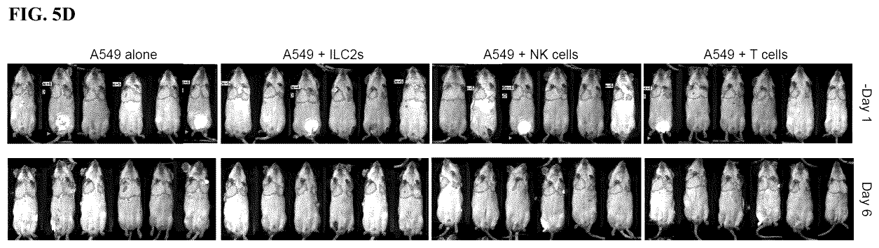

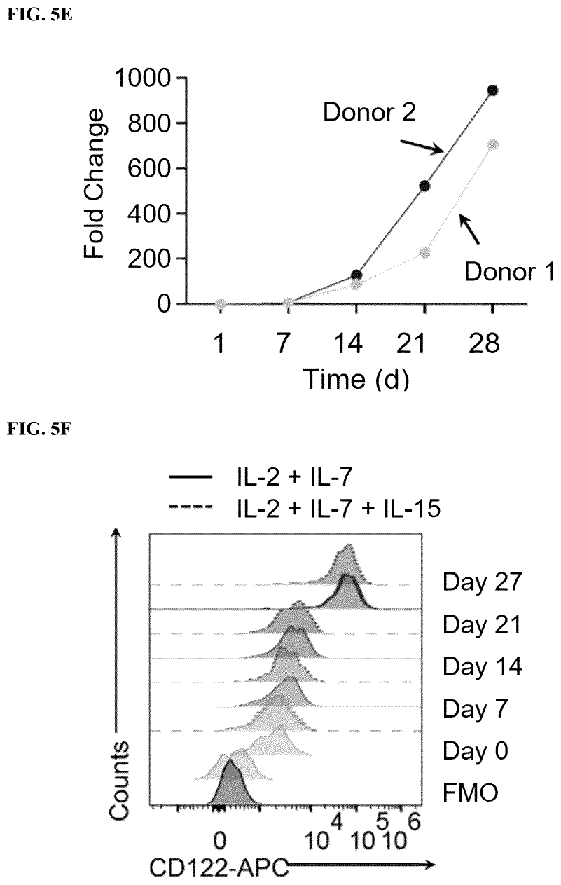

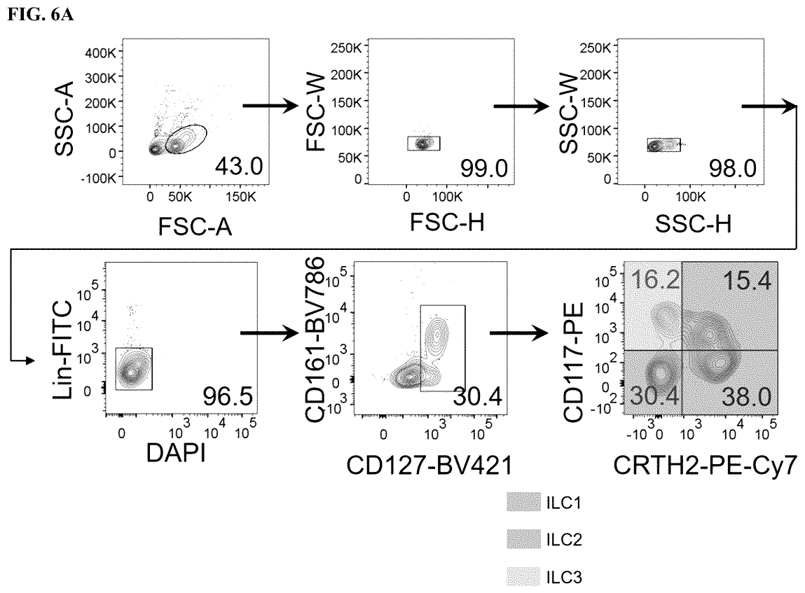

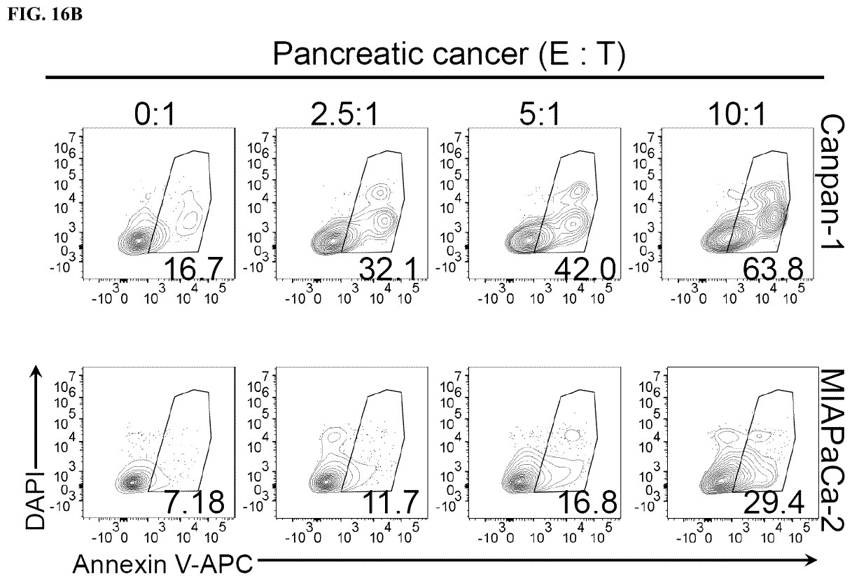

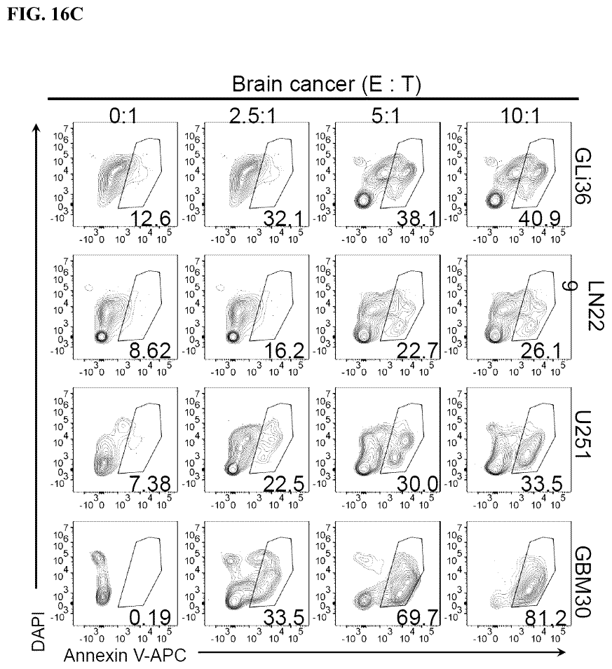

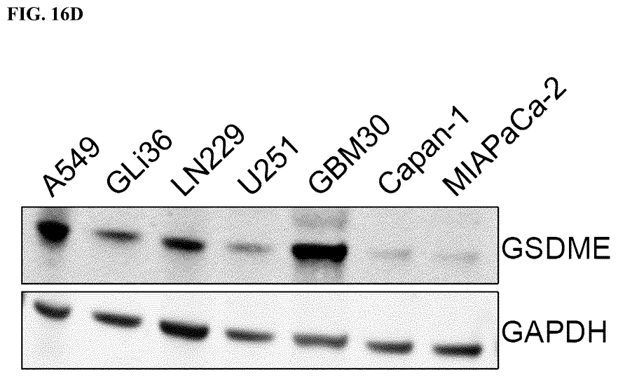

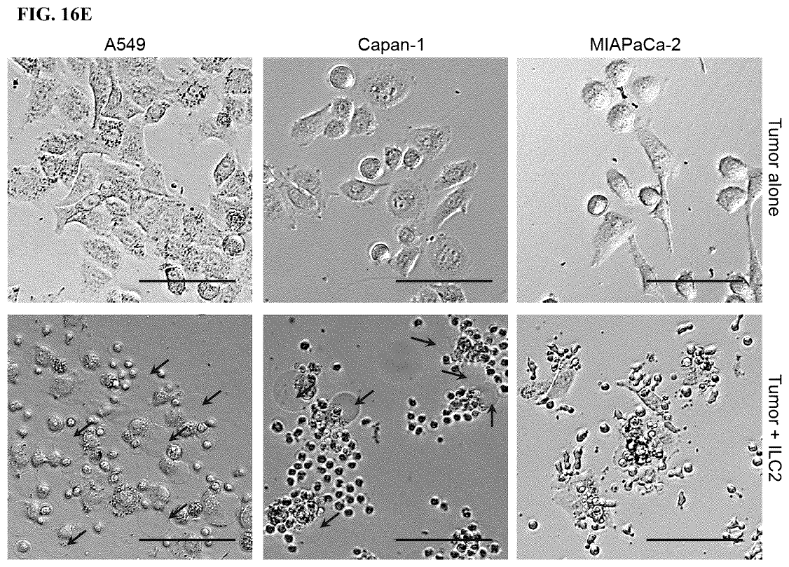

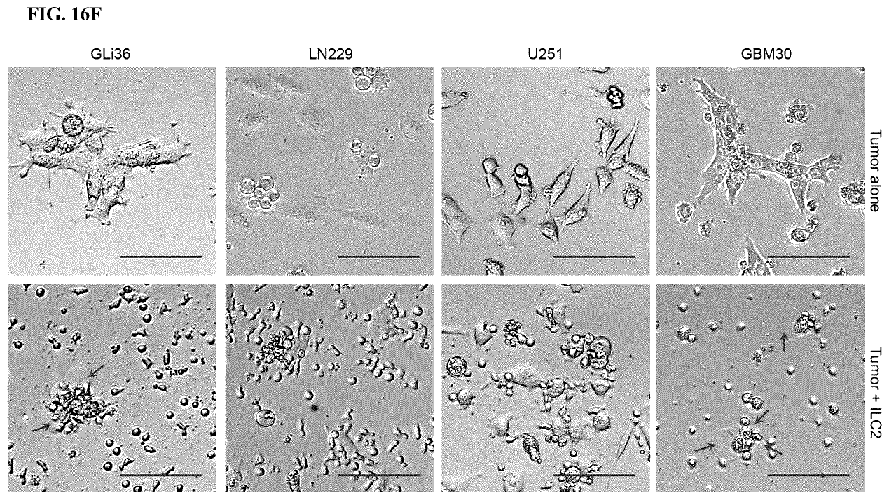

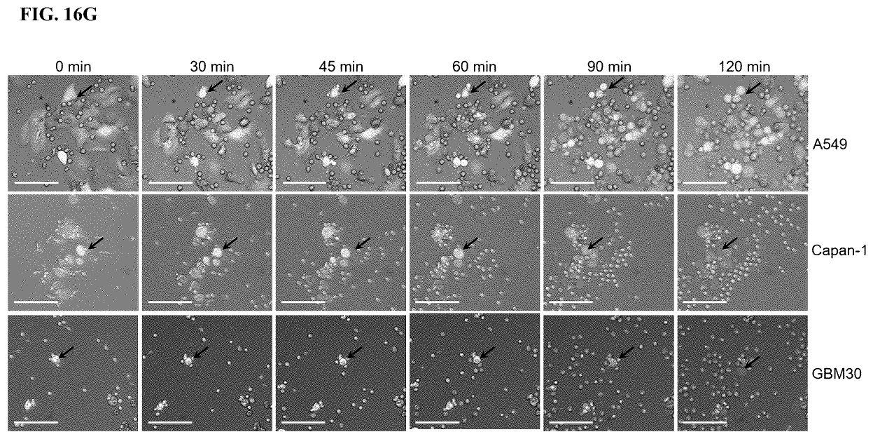

Finally, we investigated the role of ILC2s in several models of solid tumors. We used three different human solid tumor cell lines (lung, pancreatic, and brain cancer) in co-culture assays with Ex ILC2s. These tumor lines were selected because ILC2s have been described in these organs of healthy donors and/or patients with cancer^{22,43-45}, though data regarding the function of ILC2s in cancer patients is lacking. Following co-culture, we observed that Ex ILC2s can lyse solid tumor cell lines, as measured by Annexin V/DAPI staining and real-time cell analysis (RTCA) assay (FIGS. 5A-5C and FIG. 16A and FIG. 16B). We also observed that these solid tumor cells expressed GSDME (FIG. 16C). Some of them (e.g., A549, Capan-1, and GBM30) showed a pyroptosis morphology in co-culture with ILC2s (FIG. 16D). Using time-lapse microscopy, we further confirmed that A549, Capan-1, and GBM30 underwent increasing pyroptotic membrane ballooning, suggesting the pyroptosis of solid tumor cells was induced by ILC2s. We next used the lung cancer cell line A549, pancreatic cell line Capan-1, and brain cancer cell line GBM30 to establish in vivo animal models. As we did for liquid tumors, to best replicate physiologic conditions, we infused PBMCs into NSG-SGM3 mice before implantation of FFLuc-labeled solid tumor cells and Ex ILC2s. Tumor growth was monitored by measuring changes in tumor bioluminescence over time. We hypothesized that Ex ILC2s would protect against solid tumor progression in vivo. Indeed, infusion of Ex ILC2s suppressed observed tumor growth, reduced tumor burden, and prolonged mouse survival in these highly aggressive mouse models compared to control groups without Ex ILC2s treatment (FIGS. 5A-5C). Taken together, using the in vitro and in vivo models, we demonstrated that Ex ILC2s have the potential to control solid tumor progression.

### Discussion

ILC2s play a critical role in mediating immune responses and regulating tissue repair, metabolic homeostasis, and inflammation^{22,46-53}. However, their role in preventing cancer or its progression remains unclear. In the current report, we demonstrate that ILC2s isolated from human PBMCs can be reliably expanded up to 1200-fold and studied ex vivo. Using three different human AML cell lines, we discovered that human ILC2s isolated from HDs have the ability to control the development of AML in vitro and in vivo. Mechanistically, the lysis of AML cells by ILC2s is mediated by the release of GZMB following cell-cell interaction with AML cells through DNAM-1, which subsequently induces apoptosis in non-GSDME-expressing AML cells and pyroptosis and apoptosis in GSDME-expressing AML cells. DNAM-1 expressed by ILC2s engages with its ligands on AML cells and triggers downstream phosphorylation of FOXO1, which enhances their production of GZMB, thereby enabling the antitumor function of ILC2s. In patients with AML, the expression of DNAM-1 in ILC2s is profoundly diminished, and ILC2s fail to produce GZMB, creating an opportunity for immune evasion. The use of three different types of solid tumors (lung cancer, pancreatic cancer, and brain cancer) confirms that ILC2s can lyse also solid tumor cells through apoptosis and/or pyroptosis, facilitating antitumor immunity in vitro and in vivo. Collectively, these data show that ILC2s have the potential to act as a novel adoptive cell strategy for cancer immunotherapy.

Cells can die through distinct biochemical pathways with different morphological and physiological outcomes, including apoptosis and pyroptosis⁵⁴. Apoptosis is the most widely recognized programmed cell death and is mechanistically defined by the requirement for particular cysteine-dependent aspartate-specific proteases, or caspases, which produce an orchestrated disassembly of the cell⁵⁴. Pyroptosis, the inflammatory form of cell death, was defined as GSDM-mediated programmed death⁵⁵. Unlike apoptosis, pyroptotic tumor cells release several pro-inflammatory factors involved in the recruitment of cytotoxic lymphocytes to tumors⁵⁵. The GZMB-mediated cleavage of GSDM proteins dependent or independent caspases in cancer cells amplifies inflammation signals in the tumor microenvironment, thus recruiting more immune cells and further promoting antitumor immunity, which completes a positive-feedback loop of antitumor immunity.³⁶⁻⁵⁴. For example, GZMA cleaves GSDMB at the linker, which unleashes its pore-forming activity and results in pyroptotic cell death of GSDMB-expressing cancer cells⁵⁶. The GZMB produced by NK cells or CAR T cell directly cleaves GSDME at the same site as caspase 3, liberating cytotoxic N-terminus to form pore in membrane^{36,57}. In the present study, we show that GZMB released from ILC2s from HDs cleaves GSDME in GSDME (+) AML cells, providing mechanistic insight as to how ILC2s can exert anti-tumor immunity through pyroptosis.

Inhibitory receptors expressed as checkpoint molecules on the surface of immune cells have been targeted in some cases for successful tumor immunotherapy. Still, the success also depends on activating receptors on effector CD8⁺ T cells and NK cells recognizing their cognate ligands expressed on tumor cells⁵⁸. As one such activating receptor, DNAM-1 plays a critical role in anti-tumor immunity. Its ligands, CD112 and CD155, are highly expressed on tumor cells, and their engagement promotes DNAM-1(+) CD8⁺ T cell and NK cell-mediated recognition and lysis of tumor cells⁵⁹. DNAM-1-deficient mice show impaired clearance of CD155-expressing sarcoma cells, increased tumor development, and significantly worse mortality than their WT counterparts⁶⁰. Like CD8⁺ T cells and NK cells, ILC2s highly express DNAM-1. However, the mechanism by which this critical activation receptor regulates ILC2s effector function has not been elucidated. Our results show that AML cells or primary AML-derived blasts interact with healthy Ex ILC2s through DNAM-1 to elevate the GZMB production, which can induce AML cell pyroptosis and/or apoptosis, thereby enhancing antitumor immunity. However, the mechanism of the DNAM-1 in controlling GZMB production in ILC2s remains unknown. Our findings demonstrate that a DNAM-1-CD112/CD155 receptor-ligand interaction results in downstream phosphorylation and degradation of the inhibitor FOXO1, thereby enhancing the production of GZMB in ILC2s.

During tumor progression, cancer cells adopt a variety of features to escape from immune attack, such as the promotion of antiapoptotic factors, loss of tumor antigen expression, secretion of local immunosuppressive factors in the tumor microenvironment, the potentiation of immunosuppressive cells (e.g., T regulatory cells) and tumor-associated macrophages, and the expression of inhibitory molecules that can block the action of immune cells⁶¹. All these mechanisms work together to help the tumor avoid being detected and eliminated by the immune system. ILC2s become dysregulated in AML patients with a profound decrease in DNAM-1 expression. This may lead to a reduced recognition of cancer cells by ILC2s, which allows cancer cells to evade the surveillance and immune response of ILC2s. Indeed, healthy Ex ILC2s placed in contact with primary AML-derived blasts lose expression of DNAM-1 through an unknown mechanism that will be pursued in future studies. This loss could be due to CD155 expressed on AML cells in interaction with DNAM-1 expressed on ILC2s, resulting in the internalization and proteasome degradation of DNAM-1 as recently report⁴¹. This may in turn facilitate immune escape and support cancer progression as well as therapy resistance. AML cells or primary AML-derived blasts may also secrete some inhibitory factors to suppress the functions of ILC2s, creating an opportunity for cancer cells to escape immune surveillance and response. For example, TGF-β can suppress NK cell antitumor immunity by significantly downregulating the expression of NKp30, NKp46, NKG2D, and DNAM-1⁶². It can also decrease the expression of NKG2D ligands on tumor cells⁶³. Like NK cells, the decrease in DNAM-1 expression on ILC2s may result from the suppressive role of TGF-β. Should this prove to be the case, the combination of expanded healthy ILC2s with a neutralizing anti-TGF-β antibody may have a positive impact on prolonging the survival of individuals with AML. Regardless, our results provide the rationale that infusion of healthy Ex ILC2s has the potential for cancer therapy.

Our findings demonstrate that ILC2s, like NK cells, have the ability to lyse tumor cells directly in vitro and exert antitumor immunity in a pre-clinical humanized model of native organs in vivo. As ILC2s, NK cells, and T cells share some immune modulatory molecules, further investigations of a broader array of checkpoints that collectively target ILC2s for cancer immunotherapy are therefore warranted. The data presented herein underscore ILC2s as a new member of the cytotoxic effector cell family and may serve as a novel allogeneic therapy for cancer.

Autologous CAR T cell therapy has led to remarkable improvements in patients with aggressive B cell malignancies. However, challenges remain, such as CAR T cell-related CRS, neurotoxicity, and T cell receptor (TCR)-induced risk of GVHD, sometimes requiring intensive care unit support⁶⁶. NK cell therapy has also delivered promising results, showing encouraging efficacy and remarkable safety, but important questions remain open. For example, the methods to elucidate the key parameters that determine NK cell potency and persistence and the development and implementation of optimal methods for expansion and cryopreservation of NK cells to ensure high product quality likely require further development. Also, thus far there are only limited data showing the efficacy of modified NK cells in treating cancer patients. Like NK cell infusions, ILC2s infusions did not give rise to GVHD, tissue destruction, or toxicity in halting cancer progression in our tumor models. Although we observe that ILC2s exhibit low exhausted characteristics compared to NK cells, ILC2s and NK cells have similar anti-AML function in vivo. The phenomenon may reflect that downregulated the exhaustion of ILC2s in AML may not improve anti-AML efficacy. Further investigations of the exhaustion of ILC2s in other tumor models or how to improve the anti-AML function of ILC2s will be warranted. Therefore, reprogramming autologous ILC2s in vivo, administration of ex vivo expanded allogeneic ILC2s without or with CARs, or expanded ILC2s combination with an FDA-approved anti-tumor agent drug is all future opportunities worth exploring.

In summary, we present a system for the expansion of human ILC2s and identify previously unknown antitumor functions of ILC2s. We demonstrate that ILC2s, when expanded ex vivo, can play an important role in controlling the development of both hematological and solid tumors in our animal models. These studies provide us with an additional strategy to improve outcomes in both hematological and solid tumors by defining the role of ILC2s and exploring their therapeutic potential as a new member of the cell therapy family.

### Example 3: Methods

### Human Samples

AML specimens were collected from patients with AML registered at City of Hope National Medical Center who consented to an Institutional Review Board approved protocol (IRB #18067); healthy donor specimens were collected from patients who were consented to IRB #06229. Mononuclear cells were isolated using Ficoll separation. ILCs were isolated using EasySep™ Human Pan-TLC Enrichment Kit (STEMCELL) or were sorted using a BD FACSAria™ Fusion.

### Cells and Cell Culture

OP9-mDL1 and OP9-mDL4 were maintained in DMEM GlutaMAX media (Gibco) supplemented with 20% FBS (Gibco) and 50 M β-mercaptoethanol. The human AML cell lines MOLM13, U937, and THP1 were cultured in RPMI 1640 (Gibco) with 10% FBS. All AML cell lines expressed luciferase. The human lung cancer cell line A549, pancreatic cancer cell lines Capan-1 and MIA PaCa-2, and brain cancer cell lines GLi36, LN229, and U251 were cultured in DMEM GlutaMAX media with 10% FBS. GBM30 spheroid cells derived from a patient with GBM and modified to express luciferase were maintained with neurobasal media (DMEM/F12) supplemented with 2% B27 (Gibco), human epidermal growth factor (StemCell), basic fibroblast growth factor (StemCell), heparin (StemCell), and Glutamax (Gibco) in low-attachment cell culture flasks. These cell lines were either purchased from the American Type Culture Collection or obtained from Dr. E. Antonio Chiocca's laboratory at Harvard University (Cambridge, MA). All cell lines were routinely tested for the absence of Mycoplasma using the MycoAlert Plus Mycoplasma Detection Kit (Lonza). All cell culture media are supplemented with penicillin (100 U/mL) and streptomycin (100 mg/mL). Cultures were incubated at 37° C. in a humidified atmosphere of 5% CO2. Penicillin and streptomycin were from Thermo Fisher Scientific.

### Isolation and Expansion of Human ILC2s, NK Cells, and Pan T Cells

To isolate ILC2s, NK cells, and T cells from human peripheral blood, we diluted blood cone samples 1:1 with phosphate-buffered saline (PBS). We layered the blood on the top of Ficoll-Paque (GE Healthcare), and centrifuged it according to the manufacturer's instructions. The mononuclear cell fraction was aspirated and washed with PBS, and then the red blood cells were lysed. NK cells were enriched using the RosetteSep™ Human NK cell enrichment kit (StemCell). Total ILCs were enriched from the enriched NK cells using EasySep™ Human Pan-ILC enrichment kit (StemCell). T cells were isolated from the mononuclear cell fraction using Pan T cell isolation kit (Miltenyi Biotec). Human ILCs and NK cells were cultured in the MEM GlutaMAX media (Gibco). The medium was supplemented with 10% human AB serum (Sigma-Aldrich), IL-2 (500 IU/ml), IL-7 (20 ng/ml), and IL-15 (20 ng/ml). T cells were cultured in the X-VIVO 15 media (Lonza) supplemented with 10% human AB serum, IL-2 (50 IU/ml) and IL-15 (0.5 ng/ml). Medium and cytokines were refreshed every two days by replacing half of the media containing 1× concentration of cytokines. Every four days, ILC2s and NK cells were re-cultured onto new plates with fresh OP9. Two weeks later, the ILC2s stained with lineage (anti-CD3, anti-CD4, anti-CD8, anti-CD14, anti-CD15, anti-CD16, anti-CD19, anti-CD20, anti-CD33, anti-CD34, anti-CD203c, anti-FceRI, and anti-CD56), anti-CD161, anti-CRTH2, and anti-c-Kit (anti-CD117) antibodies, NK cells stained with anti-CD3 and anti-CD56 antibodies, and T cells stained with anti-CD3 antibody were sorted using a BD FACSAria™ Fusion (BD Biosciences). FACS-sorted ILC2s and NK cells were plated and cultured onto fresh OP9 cells in the fresh MEM GlutaMAX media with cytokines. FACS-sorted T cells were plated and cultured in the fresh X-VIVO 15 media with cytokines. All cytokines were provided by the National Institutes of Health.

### Flow Cytometry

ILC2s from human peripheral blood were identified using surface staining with a live/dead cell viability cell staining kit (Invitrogen) and the following monoclonal antibodies: lineage (FITC-conjugated anti-CD3, anti-CD4, anti-CD8, anti-CD14, anti-CD15, anti-CD16, anti-CD19, anti-CD20, anti-CD33, anti-CD34, anti-CD203c, anti-FceRI), CD56 (FITC, BV711 or BV421-conjugated anti-CD56), CD127 (BV421 or APC-conjugated anti-CD127), CRTH2 (PE-Cy7-conjugated anti-CRTH2), and c-Kit (CD117, PE, PECF594 or BV711-conjugated anti-c-Kit). NK cells and T cells from human peripheral blood were identified using surface staining with a live/dead cell viability cell staining kit and the following monoclonal antibodies: anti-CD3 and anti-CD56 (for NK cells) and anti-CD3 (for T cells), respectively. The expression of CD155 and CD112 on the MOLM13, U937, and THP1 was identified by APC-conjugated anti-CD155 and PE-conjugated anti-CD112, respectively. The expression of IL-33R on human ILC2s was identified by APC conjugated anti-IL-33R. The expression of DNAM-1, NKG2D, and NKp30 on human ILC2s, NK cells, and T cells was identified by FITC or AF647-conjugated anti-DNAM-1, PE-conjugated anti-NKG2D, and BV421 conjugated anti-NKp30, respectively. Human primary AML blasts were gated by Lin-CD45^{dim}. To examine intracellular cytokine production, we stimulated human freshly isolated ILC2s and expanded ILC2s with Leukocyte Activation Cocktail in the presence of BD GolgiPlug™ for 4 h. In some experiments, the expanded ILC2s were co-cultured with or without tumor cells for 48 h without stimulating the Leukocyte Activation Cocktail. Intracellular staining for granzyme B (GZMB) and perforin was performed using a Fix/Perm kit (BD Biosciences), followed by staining with a BV510 or PE-conjugated GZMB and a BV421-conjugated perforin, respectively. Intracellular staining for caspase 3, IL-4, IL-5, IL-9, IL-13, IFNγ, or TNF was performed using a Fix/Perm kit, followed by staining with a V450-conjugated anti-caspase 3, a BV510-conjugated anti-IL-4, a BV421-conjugated anti-IL-5, a PE-conjugated anti-IL-9, an APC-conjugated anti-IL-13, a PE-Cy7-conjugated anti-IFNγ or an APC-conjugated anti-TNF antibody, respectively. Intracellular staining for transcription factors GATA3, RORγt, EOMES, or T-BET was performed using a Fix/Perm kit (ThermoFisher), followed by staining with a PE-CF594-conjugated anti-GATA3, an AF647-conjugated anti-RORγt, a BUV395-conjugated anti-EOMES, or a BV421-conjugated anti-T-BET.

To examine cell death, the tumor cells were co-cultured with or without ILC2s for 48 h. Cells were then stained with PE or APC-conjugated Annexin V and with 7-amino-actinomycin D (7-AAD, BD Biosciences) or 4′,6-diamidino-2-phenylindole (DAPI), followed by flow cytometric analysis. All human antibodies were used at 1:50, except for CD34, CRTH2, and CD117, which were used at 1:100. All analyses were performed on a Fortessa X-20 flow cytometer (BD Biosciences), and sorting was performed using a BD FACSAria™ Fusion (BD Biosciences). Flow Cytometry data were analyzed by FlowJo V10 (Treestar).

### In Vitro Co-Culture Assay of ILC2s and Tumor Cells

For human ILC2s co-culture with AML cells in cell-cell contact manner assays, AML cells were co-cultured with various numbers of freshly isolated ILC2s or expanded ILC2s that were isolated from peripheral blood of healthy donors for 48 h. For the co-culture of AML cells and ILC2s in the Transwell co-culture system, AML cells were seeded in the lower chamber of a 24-well Transwell plate, while ILC2s were seeded in the upper chamber for 48 h. The ratio of ILC2s: AML cells were 2:1. For co-culture assays with antibodies, ILC2s were co-cultured with anti-DNAM-1 (10 μg/ml) antibody, anti-NKG2D (10 μg/ml) antibody, or anti-NKp30 antibody (10 μg/ml) for 2 h, followed by adding AML cells at the ratio of ILC2s: AML cells were 2:1.

For the co-culture assay of human ILC2s and solid tumor cells, solid tumor cells were co-cultured with various numbers of expanded ILC2s that were isolated from peripheral blood of healthy donors for 48 h. For all co-culture assays, cells were harvested after 2 days and analyzed using flow cytometry. Annexin V and 7-AAD or DAPI were used to identify dead cells following the manufacturer's instructions. Cell images or videos were taken by microscope (Zeiss AxioCam 702).

### In Vivo Tumor Cell Transplantation Assay

For the human AML cell engraftment experiment, 5×10⁴ MOLM13 or U937 or 0.1×10⁶ THP1 cells were transplanted via tail vein injection into 6-8-week-old NOD.Cg-Prkdc^{scid} Il2rg^{tmlWjl}/SzJ (NSG) purchased from the Jackson Laboratory, and followed by i.v. injection of 2×10⁶ expanded human ILC2s into the mice that were implanted with MOLM13 or U937 cells or i.v. injection of 4×10⁶ expanded human ILC2s into the mice implanted with THP1 cells. Recombinant human IL-2 (500 IU/mouse) was intraperitoneally injected into recipient mice daily for 7 days. For Human AML cell engraftment experiment with human PBMCs. 5×10⁴ MOLM13 and 10×10⁶ PBMCs were co-transplanted via tail vein injection into 6-8-week-old NOD.Cg-Prkdc^{scid} Il2rg^{tmlWjl}Tg(CMV-IL3, CSF2, KITLG)lEav/MloySzJ (NSG-SGM3) mice purchased from the Jackson Laboratory, and followed by i.v. injection of 2×10⁶ expanded human ILC2s, expanded NK cells, or expanded T cells into the mice that were implanted with MOLM13.

For the human lung cancer cell implantation experiment, 0.2×10⁶ A549 cells and 10×10⁶ PBMCs were co-transplanted via tail vein injection into 6-8-week-old NSG-SGM3. One day later, 4×10⁶ expanded human ILC2s, expanded NK cells, or expanded T cells were i.v. injected into the mice that were implanted with A549 cells.

For the human pancreatic cancer cell implantation experiment, 0.2×10⁶ Capan-1 cells were injected via intraperitoneal injection (i.p.) into 6-8-week-old NSG-SGM3. One day later, 4×10⁶ expanded human ILC2s, expanded NK cells, or expanded T cells were i.p. injected into the mice that were implanted with Capan-1 cells.

For the human brain cancer cell implantation experiment (orthotopic GBM models), 0.1×10⁶ luciferase-expressing GBM30 cells into the right frontal lobe of the brain (2-mm lateral and 1-mm anterior to bregma at a depth of 3-mm). One days later, 4×10⁶ expanded human ILC2s, expanded NK cells, or expanded T cells were intratumorally injected into the same location as above.

All mice were subsequently monitored frequently for AML, lung cancer, pancreatic cancer, or brain cancer disease progression and imaged to check tumor growth at different time points. Tumor burden was assessed via in vivo bioluminescence measurements using the IVIS Imaging System at the City of Hope Imaging Center. For luciferase detection imaging, 200 μL of 15 mg/mL D-luciferin (Caliper Life Sciences) in PBS was injected i.p. before imaging. The observers were blinded to the group allocation.

### Caspase 3/7 Activity Assay

ILC2s were co-cultured with AML cells at a ratio of 2:1 for 12 h. Next, 100 μl of Caspase-Glo 3/7 reagent was added to each well. Plates were then shaken at 300 rpm for 1 min, incubated for 60 min at room temperature, and then read on a luminometer (Promega, Glomax). Background luminescence was determined with 100 μl of culture medium without cells and subtracted before fold changes were calculated.

Luminescence-based in vitro killing assay

Freshly isolated or expanded ILC2s were cultured at indicated ratios with AML cells. After 48 h, 100 μL of the mixture was transferred to a 96-well white luminometer plate. Next, 10 μl of the substrate (Promega) was added, and luminescence (RLU) was immediately determined. The results are reported as percent killing based on luciferase activity in the wells with tumor cells but no ILC2s [% killing=100−[(RLU) from well with effector and target cell co-culture)/(RLU from well with target cells)×100)].

### Gene Expression Analyses

For regular PCR analyses, RNA was isolated from 50,000 cells using a miRNeasy mini kit (QIAGEN) and reverse transcribed using a PrimeScript RT reagent kit with gDNA Eraser (TAKARA). PCR reactions were run on a ProFlex PCR System (Applied Biosystems) using 2×MyTaq Red Mix (Meridian Bioscience). RT-PCR analysis was conducted to assess the expression of human IL-33 (Forward: 5′-GTGACGGTGTTGATGGTAAGAT-3′ (SEQ ID NO:1); reverse: 5′-AGCTCCACAGAGTGTTCCTTG-3′ (SEQ ID NO:2)), human IL-25 (forward:5′-CAGGTGGTTGCATTCTTGGC (SEQ ID NO:3); reverse: 5′-GAGCCGGTTCAAGTCTCTGT-3′ (SEQ ID NO:4)), human TSLP (forward: 5′-ATGTTCGCCATGAAAACTAAGGC-3′ (SEQ ID NO:5); reverse: 5′-GCGACGCCACAATCCTTGTA-3′ (SEQ ID NO:6)), PGD2 (forward: 5′-GGCGTTGTCCATGTGCAAG-3′ (SEQ ID NO:7); reverse: 5′-GGACTCCGGTAGCTGTAGGA-3′ (SEQ ID NO:8)), and 18S rRNA (forward: 5′-GTAACCCGTTGAACCCCATT-3′ (SEQ ID NO:9); reverse: 5′-CCATCCAATCGGTAGTAGCG-3′ (SEQ ID NO:10)). The primers were purchased from Integrated DNA Technologies.

RNA was extracted using TRIzol reagent according to the manufacturer's instructions and was subject to reverse transcription using the SuperScript III system (Invitrogen). Gsdme expression was assayed by qRT-PCR using SsoFast Supermix (Bio-Rad). Breast cancer (BRCA) and colon cancer (COAD) RNA-sequence expression data were obtained from TCGA using the University of California Santa Cruz (UCSC) Xena bioinformatic tool21. The log 2 difference between GSDME and GAPDH expression was calculated for both tumor and normal tissue and plotted using Prism software.

### Confocal Microscopy

FACS-sorted the expanded ILC2s were stimulated with Leukocyte Activation Cocktail in the presence of BD GolgiPlug™ for 4 h. 1×10⁵ ILC2s were cytospin on the glass microscope slides. Cells were fixed in 4% formaldehyde for 10 min, permeabilized using Triton X-100 for 20 min, and washed three times using PBS, followed by a blocking step with PBS supplemented with 5% BSA for 60 min. The cells were then stained with granzyme B recombinant rabbit monoclonal antibody (ThermoFisher) or perforin polyclonal antibody (ThermoFisher) diluted at 1:100, counter-stained with DAPI overnight at 4° C. The cells were stained with Alexa Fluor 647-conjugated AffiniPure Goat Anti-Rabbit IgG (H+L) secondary antibody (Jackson ImmunoResearch Laboratories INC). Cells were washed, and images were acquired using a 63×/1.40 Plan-Apochromat oil immersion objective on an LSM 880 confocal microscope (Carl Zeiss AG).

### Elisa

ILC2s were co-cultured with AML cells at a ratio of 2:1 for 48 h in the MEM GlutaMAX media supplemented with IL-2, IL-7, and IL-15. Cell supernatants were collected and analyzed for GZMB content by ELISA according to the manufacturer's protocols. Levels of GZMB and perforin production in culture supernatants were measured using the Human Granzyme B DuoSet ELISA Kit (Cat #DY2906-05, R&D) and Perforin Human ELISA Kit (Cat #BMS2306, ThermoFisher).

### Immunoblot

ILC2s labeled with 5 mM CellTrace Violet (CTV; Thermo Fisher Scientific) were co-cultured with AML cells at a ratio of 2:1 for indicated times. AML cells or ILC2s were sorted and lysed in RIPA lysis and extraction buffer (ThermoFisher). Cell lysate in SDS loading buffer (ThermoFisher) was boiled and analyzed using 4-20% Mini-PROTEAN® TGX™ Precast Protein Gels (Bio-Rad). The gels were transferred to polyvinylidene fluoride (PVDF) membranes (Sigma-Aldrich) and were then blocked with 2% milk in TBST buffer (Bio-Rad) for 60 min before incubation with primary antibodies at 4° C. overnight. The membranes were washed 3 times and incubated with the appropriate fluorescent secondary antibody, or Rabbit IgG HRP-conjugated Antibody, or Mouse IgG HRP-conjugated Antibody for 60 min at room temperature. The immunoreactive proteins were detected using the Odyssey DLX Imaging System (LI-COR) or FluorChem E System (ProteinSimple). Antibodies were anti-GSDME (ab215191, Abcam), anti-caspase 3 (9662S, Cell Signaling Technology), anti-GAPDH (60004-1-Ig, Proteintech), anti-Phospho-FoxO1 (Thr24)/FoxO3a (Thr32)/FoxO4 (Thr28) (2599S, Cell Signaling Technology), anti-FoxO1 (14952S, Cell Signaling Technology), anti-Phospho-Akt (Ser473) (4060S, Cell Signaling Technology), and anti-Akt (pan; 2920S, Cell Signaling Technology).

### CRISPR-Cas9 Knockout ILC2s Using Electroporation

GZMB sgRNA1: 5′-GGCCCACAATATCAAAGAAC-3′ (SEQ ID NO:11); GZMB sgRNA2: GCTACCTAGCAACAAGGCCC (SEQ ID NO:12). DNAM-1sgRNA1:Electroporation was performed after ILC2s expansion for two weeks. Cas9-gRNA RNP complex was made by mixing 2.1 μl PBS, 1.2 μl GZMB gRNA1 and GZMB gRNA2 (100 PM), and 1.7 μL Cas9 Nuclease V3 (10 mg/ml, IDT, Cat #1081059), followed by incubating the complex at room temperature for 15 min. 1.0×10⁶ ILC2s were washed with PBS two times and resuspended in 20 μL of P3 buffer according to the instruction of Amaxa™ P3 Primary Cell 4D-Nucleofector™ X Kit (Lonza, Cat #V4XP-3032), followed by mixing with the Cas9-gRNA RNP complex (5 μL). Cells in the electroporation buffer were then added and moved into electroporation cuvettes. Programme E0115 in a 4D-Nucleofector device was chosen for electroporation. After electroporation, the ILC2s was immediately supplemented with a prewarmed medium, transferred out of the electroporation cuvettes, and then cultured at 37° C. with 5% CO2.

### CRISPR-Cas9 Knockout AML Cell Lines by Lentivirus

CD112 sgRNA (5′-CGAGTTTGCCACCTTCCCCA-3′ (SEQ ID NO:13) and 5′-ACCTGCGAACCACCAGAATG-3′ (SEQ ID NO:14)), CD155 sgRNA (5′-CCAGCTATTCGGAGTCCAAA-3′ (SEQ ID NO:15) and 5′-CACGGAGTCGCCCAAGAAGC-3′ (SEQ ID NO:16)), and GSDME sgRNA (5′-GAGTACATCGCCAAGGGTGA-3′ (SEQ ID NO:17) and 5′-AAGTTTGCAAACCACGTGAG-3′ (SEQ ID NO:18)) were cloned into eSpCas9-2A-GFP (PX458, Genscript) as previously described⁶⁷. The resulting plasmids were transfected into HEK293T cells with pSPAX2 and pCMV-VSVG at a 1/1/2 ratio. Supernatants from HEK293T cells transfected with plasmids that express human CD112, CD155, and GSDME guide RNAs were collected 48 h later. The supernatants were transfected with plasmids expressing human CD112 and CD155 guide RNAs to transduce human MOLM13, U937, and THP1 cell lines. The supernatants were transfected with plasmids expressing GSDME guide RNAs to transduce human MOLM13. Five days later, cells were stained with CD112 and CD155 antibodies. CD112-CD155⁺GFP⁺, CD112⁺CD155⁻GFP⁺, and CD112⁻CD155⁻GFP⁺ cells were sorted using a BD FACSAria™ Fusion and were then cultured into 24-well plates. For GSDME knockdown MOML13 cell screening, GFP⁺ cells were sorted for GSDME expression by immunoblot.

### Representative Real-Time Cell Analysis (RTCA)-Based In Vitro Killing Assay

Solid tumor cells were used as target cells. Cell culture medium (50 μl) was added to each well of an E-plate (Cat #300601010, Agilent). The E-plate is a standard 96-well plate with a glass-bottom coated with gold microelectrodes covering approximately 75% of the well area. The E-plate was then connected to the system to check for proper electrical contacts and to obtain background impedance readings in the absence of cells. Target tumor cells (5000 cells in 50 μl of media) were plated into the E-plate and cultured overnight in the RTCA system installed in the CO2 incubator. Expanded ILC2s in 100 μl media were added into the E-plate and co-cultured for at least an additional 40 h in the RTCA system. The growth and cell index of target cells were measured following the manufacturer's instructions.

### Statistical Analysis

For continuous endpoints, Student's t test was used to compare two independent conditions and one-way ANOVA models were used to compare three or more independent conditions. For survival data, survival functions were estimated by the Kaplan-Meier method and compared by log-rank tests. All tests were two-sided. P values were adjusted for multiple comparisons by Holm's procedure. Data are presented as mean±SD. Prism software v.9 (GraphPad, CA, USA) and SAS v.9.4 (SAS Institute. NC, USA) were used to perform statistical analyses. The p-values are represented as: * <0.05, ** <0.01, *** <0.001, and **** <0.0001.

TABLE 1

| Gene | | No | No | No | No |
|---|---|---|---|---|---|
| Name | (pg/ml) | ILC2-1 | ILC2-2 | ILC2-3 | ILC2-4 |
| CXCL13 | BLC | 0 | 0 | 0 | 0 |
| BCA1 | | | | | |
| BLC | | | | | |
| SCYB13 | | | | | |
| CCL11 | Eotaxin | 0 | 0 | 0 | 0 |
| SCYA11 | | | | | |
| CCL24 | Eotaxin- | 0 | 0.8513292 | 0 | 0.2328498 |
| MPIF2 | 2 | | | | |
| SCYA24 | | | | | |
| CSF3 | G-CSF | 0 | 0 | 0 | 0 |
| C17ORF33 | | | | | |
| GCSF | | | | | |
| CSF2 | GM- | 1083.5733 | 768.36643 | 862.10116 | 1040.4885 |
| GMCSF | CSF | | | | |
| CCL1 | I-309 | 41.05141 | 158.80127 | 167.51351 | 144.92951 |
| SCYA1 | | | | | |
| ICAM1 | ICAM-1 | 0 | 0 | 0 | 0 |
| IFNG | IFNg | 0 | 0 | 0 | 0 |
| IL1A | IL-1a | 5.7696919 | 4.0557223 | 0 | 5.1646568 |
| IL1F1 | | | | | |
| IL1B | IL-1b | 0.1901428 | 0.8339777 | 0 | 0.2541729 |
| IL1F2 | | | | | |
| IL1RN | IL-1ra | 8.3966385 | 11.416146 | 0 | 2.5658272 |
| IL1F3 | | | | | |
| IL1RA | | | | | |
| IL2 | IL-2 | 0 | 4.424549 | 4.744447 | 4.5905476 |
| IL4 | IL-4 | 0 | 0.1218164 | 1.7893141 | 0 |
| IL5 | IL-5 | 2.4184291 | 5.2452785 | 3.0845056 | 4.150891 |
| IL6 | IL-6 | 0 | 0 | 0.4980327 | 0.9937111 |
| IFNB2 | | | | | |
| IL6R | IL-6R | 1236.1993 | 1461.471 | 2459.5851 | 2553.4422 |
| IL7 | IL-7 | 0 | 0 | 9.7435595 | 14.917493 |
| CXCL8 | IL-8 | 0.16351 | 0.07396 | 0.51837 | 0.31858 |
| IL8 | | 45 | 81 | 18 | 87 |
| IL10 | IL-10 | 0 | 0 | 0 | 0 |
| IL11 | IL-11 | 4.8682305 | 46.380836 | 4.1072028 | 35.226441 |
| IL12B | IL- | 0 | 0 | 0 | 0 |
| NKSF2 | 12p40 | | | | |
| IL12A/ | IL- | 0 | 0 | 0 | 0 |
| IL1 2B | 12p70 | | | | |
| IL13 | IL-13 | 0 | 0 | 0 | 0 |
| NC30 | | | | | |
| IL15 | IL-15 | 0.6713409 | 0.575251 | 0.1956419 | 0.9537838 |
| IL16 | IL-16 | 4.1056483 | 17.912712 | 14.738087 | 20.836126 |
| IL17A | IL-17 | 7.8498863 | 13.214211 | 1.6768317 | 0 |
| CTLA8 | | | | | |
| IL17 | | | | | |
| CCL2 | MCP-1 | 1.3250874 | 3.4430837 | 8.1756747 | 13.894581 |
| MCP1 | | | | | |
| SCYA2 | | | | | |
| CSF1 | MCSF | 0 | 0 | 0 | 0 |
| CXCL9 | MIG | 0 | 5.4360114 | 0 | 0.6232887 |
| CMK | | | | | |
| MIG | | | | | |
| SCYB9 | | | | | |
| CCL3 | MIP-1a | 5.249719 | 8.1999775 | 26.177342 | 8.4884526 |
| G0S19-1 | | | | | |
| MIP1A | | | | | |
| SCYA3 | | | | | |
| CCL4 | MIP-1b | 0 | 0.0225601 | 1.5111981 | 0.3255586 |
| LAG1 | | | | | |
| MIP1B | | | | | |
| SCYA4 | | | | | |
| CCL15 | MIP-1d | 0 | 0 | 0 | 0 |
| MIP5 | | | | | |
| NCC3 | | | | | |
| SCYA15 | | | | | |
| PDGFB | PDGF- | 7.6469789 | 23.819905 | 19.711945 | 31.743798 |
| PDGF2 | BB | | | | |
| SIS | | | | | |
| CCL5 | RANTES | 403.40815 | 254.91613 | 708.24606 | 962.13853 |
| D17S136E | | | | | |
| SCYA5 | | | | | |
| TIMP1 | TIMP-1 | 2690.0289 | 1978.4817 | 3035.5217 | 2871.9845 |
| CLGI | | | | | |
| TIMP | | | | | |
| TIMP2 | TIMP-2 | 968.27117 | 392.99288 | 4633.8892 | 2283.233 |
| TNF | TNFa | 0 | 0 | 0 | 0 |
| TNFA | | | | | |
| TNFSF2 | | | | | |
| LTA | TNFb | 0 | 0 | 0 | 0 |
| TNFB | | | | | |
| TNFSF1 | | | | | |
| TNFRSF1A | TNF RI | 62.090834 | 32.193063 | 0 | 33.747751 |
| TNFAR | | | | | |
| TNFR1 | | | | | |
| TNFRSF1B | TNF | 1123.7809 | 1820.095 | 3601.0372 | 4111.9278 |
| TNFBR | RII | | | | |
| TNFR2 | | | | | |
| Gene | | | | | |
| Name | (pg/ml) | ILC2-1 | ILC2-2 | ILC2-3 | ILC2-4 |
| CXCL13 | BLC | 1.7214181 | 77.001908 | 14.351866 | 0 |
| BCA1 | | | | | |
| BLC | | | | | |
| SCYB13 | | | | | |
| CCL11 | Eotaxin | 0 | 16.464006 | 18.426059 | 0 |
| SCYA11 | | | | | |
| CCL24 | Eotaxin- | 2.3335309 | 6.2955526 | 10.776617 | 13.933195 |
| MPIF2 | 2 | | | | |
| SCYA24 | | | | | |
| CSF3 | G-CSF | 0 | 0 | 0 | 0 |
| C17ORF33 | | | | | |
| GCSF | | | | | |
| CSF2 | GM- | 1032.8839 | 1390.9093 | 1181.803 | 1769.6845 |
| GMCSF | CSF | | | | |
| CCL1 | I-309 | 164.74555 | 171.29741 | 255.29894 | 3628.5252 |
| SCYA1 | | | | | |
| ICAM1 | ICAM-1 | 75.108047 | 2035.7818 | 3298.5315 | 1200.7799 |
| IFNG | IFNg | 7.0250797 | 25.905544 | 3.2233552 | 0 |
| IL1A | IL-1a | 7.6949511 | 10.385909 | 11.585139 | 4.8768431 |
| IL1F1 | | | | | |
| IL1B | IL-1b | 1.0859456 | 1.3575059 | 3.6742975 | 6.1348875 |
| IL1F2 | | | | | |
| IL1RN | IL-1ra | 6.4134467 | 25.588277 | 15.867034 | 43.158241 |
| IL1F3 | | | | | |
| IL1RA | | | | | |
| IL2 | IL-2 | 7.5902709 | 2.1340986 | 0 | 1.9439263 |
| IL4 | IL-4 | 5.1454618 | 12.867642 | 26.937007 | 1.6753689 |
| IL5 | IL-5 | 4.3611519 | 3.7871138 | 3.4966203 | 2.8928404 |
| IL6 | IL-6 | 2.0975104 | 0.6463239 | 0.8779061 | 0.3164867 |
| IFNB2 | | | | | |
| IL6R | IL-6R | 2949.7764 | 3690.0075 | 3702.2947 | 5861.458 |
| IL7 | IL-7 | 18.642195 | 2.8838106 | 3.271791 | 1.3296753 |
| CXCL8 | IL-8 | 0.7865284 | 1.269848 | 1.5752212 | 10.748693 |
| IL8 | | | | | |
| IL10 | IL-10 | 8.8644822 | 2.6326902 | 0 | 0 |
| IL11 | IL-11 | 105.80656 | 57.90928 | 48.462761 | 66.674567 |
| IL12B | IL- | 0 | 0 | 0 | 0 |
| NKSF2 | 12p40 | | | | |
| IL12A/ | IL- | 0 | 0 | 0 | 0 |
| IL1 2B | 12p70 | | | | |
| IL13 | IL-13 | 0 | 0 | 0 | 0 |
| NC30 | | | | | |
| IL15 | IL-15 | 1.2099544 | 2.0527153 | 1.4628772 | 0.7285471 |
| IL16 | IL-16 | 34.877721 | 204.98094 | 211.24329 | 740.06838 |
| IL17A | IL-17 | 12.054719 | 0 | 7.1504019 | 19.652987 |
| CTLA8 | | | | | |
| IL17 | | | | | |
| CCL2 | MCP-1 | 7.9649727 | 5.0439696 | 19.120496 | 60.951 |
| MCP1 | | | | | |
| SCYA2 | | | | | |
| CSF1 | MCSF | 0 | 11.57738 | 2.3865038 | 0 |
| CXCL9 | MIG | 2.9878097 | 8.3914093 | 0.5801 | 0 |
| CMK | | | | | |
| MIG | | | | | |
| SCYB9 | | | | | |
| CCL3 | MIP-1a | 31.463249 | 66.842487 | 30.769666 | 146.64101 |
| G0S19-1 | | | | | |
| MIP1A | | | | | |
| SCYA3 | | | | | |
| CCL4 | MIP-1b | 1.7009962 | 28.146861 | 10.78658 | 34.98126 |
| LAG1 | | | | | |
| MIP1B | | | | | |
| SCYA4 | | | | | |
| CCL15 | MIP-1d | 0 | 0.0629 | 0 | 0.2059757 |
| MIP5 | | | | | |
| NCC3 | | | | | |
| SCYA15 | | | | | |
| PDGFB | PDGF- | 30.568574 | 5.6995787 | 5.633306 | 8.2769243 |
| PDGF2 | BB | | | | |
| SIS | | | | | |
| CCL5 | RANTES | 406.58501 | 919.90993 | 1016.3103 | 4347.2508 |
| D17S136E | | | | | |
| SCYA5 | | | | | |
| TIMP1 | TIMP-1 | 3447.6892 | 7645.6632 | 7598.3269 | 8105.9893 |
| CLGI | | | | | |
| TIMP | | | | | |
| TIMP2 | TIMP-2 | 3085.1609 | 1477.7529 | 3088.4862 | 11439.254 |
| TNF | TNFa | 0 | 0 | 0 | 0 |
| TNFA | | | | | |
| TNFSF2 | | | | | |
| LTA | TNFb | 29.707442 | 21.926063 | 58.33262 | 0 |
| TNFB | | | | | |
| TNFSF1 | | | | | |
| TNFRSF1A | TNF RI | 90.085801 | 458.39178 | 951.85694 | 2256.2829 |
| TNFAR | | | | | |
| TNFR1 | | | | | |
| TNFRSF1B | TNF | 12211.853 | 18552.768 | 13107.165 | 15585.236 |
| TNFBR | RII | | | | |
| TNFR2 | | | | | |

### REFERENCES

1 Larson, R. C. & Maus, M. V. Recent advances and discoveries in the mechanisms and functions of CAR T cells. Nat Rev Cancer 21, 145-161, doi:10.1038/s41568-020-00323-z (2021).
2 Laskowski, T. J., Biederstddt, A. & Rezvani, K. Natural killer cells in antitumour adoptive cell immunotherapy. Nature Reviews Cancer 22, 557-575, doi:10.1038/s41568-022-00491-0 (2022).
3 Klichinsky, M. et al. Human chimeric antigen receptor macrophages for cancer immunotherapy. Nat Biotechnol 38, 947-953, doi:10.1038/s41587-020-0462-y (2020).
4 June, C. H. & Sadelain, M. Chimeric Antigen Receptor Therapy. N Engl J Med 379, 64-73, doi:10.1056/NEJMra1706169 (2018).
5 Papadopoulou, A. et al. Activity of broad-spectrum T cells as treatment for AdV, EBV, CMV, BKV, and HHV6 infections after HSCT. Sci Transl Med 6, 242ra283, doi:10.1126/scitranslmed.3008825 (2014).
6 Jackson, H. J., Rafiq, S. & Brentjens, R. J. Driving CAR T-cells forward. Nat Rev Clin Oncol 13, 370-383, doi:10.1038/nrclinonc.2016.36 (2016).
7 Spits, H. & Mjösberg, J. Heterogeneity of type 2 innate lymphoid cells. Nat Rev Immunol, 1-12, doi:10.1038/s41577-022-00704-5 (2022).
8 Huang, Y. et al. IL-25-responsive, lineage-negative KLRG1(hi) cells are multipotential ‘inflammatory’ type 2 innate lymphoid cells. Nat Immunol 16, 161-169, doi:10.1038/ni.3078 (2015).
9 Koyasu, S. Inflammatory ILC2s cells: disguising themselves as progenitors?Nature immunology 16, 133-134, doi:10.1038/ni.3080 (2015).
10 Monticelli, L. A. et al. Innate lymphoid cells promote lung-tissue homeostasis after infection with influenza virus. Nature immunology 12, 1045-1054, doi:10.1031/ni.2131 (2011).
11 Mjösberg, J. M. et al. Human IL-25- and IL-33-responsive type 2 innate lymphoid cells are defined by expression of CRTH2 and CD161. Nat Immunol 12, 1055-1062, doi:10.1038/ni.2104 (2011).
12 van der Ploeg, E. K., Carreras Mascaro, A., Huylebroeck, D., Hendriks, R. W. & Stadhouders, R. Group 2 Innate Lymphoid Cells in Human Respiratory Disorders. Journal of innate immunity 12, 47-62, doi:10.1159/000496212 (2020).
13 Vivier, E. et al. Innate Lymphoid Cells: 10 Years On. Cell 174, 1054-1066, doi:10.1016/j.cell.2018.07.017 (2018).
14 Xu, X. et al. Group-2 Innate Lymphoid Cells Promote HCC Progression Through CXCL2-Neutrophil-Induced Immunosuppression. Hepatology 74, 2526-2543, doi:10.1002/hep.31855 (2021).
15 Revia, S. et al. Histone H3K27 demethylase KDM6A is an epigenetic gatekeeper of mTORC1 signalling in cancer. Gut 71, 1613-1628, doi:10.1136/gutjnl-2021-325405 (2022).
16 Jacquelot, N. et al. Blockade of the co-inhibitory molecule PD-1 unleashes ILC2-dependent antitumor immunity in melanoma. Nat Immunol 22, 851-864, doi:10.1038/s41590-021-00943-z (2021).
17 Qi, J. et al. Single-cell transcriptomic landscape reveals tumor specific innate lymphoid cells associated with colorectal cancer progression. Cell Rep Med 2, 100353, doi:10.1016/j.xcrm.2021.100353 (2021).
18 Trabanelli, S. et al. Tumour-derived PGD2 and NKp30-B7H6 engagement drives an immunosuppressive ILC2-MDSC axis. Nat Commun 8, 593, doi:10.1038/s41467-017-00678-2 (2017).
19 Busser, B., Sancey, L., Brambilla, E., Coll, J. L. & Hurbin, A. The multiple roles of amphiregulin in human cancer. Biochimica et biophysica acta 1816, 119-131, doi:10.1016/j.bbcan.2011.05.003 (2011).
20 Chevalier, M. F. et al. ILC2-modulated T cell-to-MDSC balance is associated with bladder cancer recurrence. J Clin Invest 127, 2916-2929, doi:10.1172/jci89717 (2017).
21 Schuijs, M. J. et al. ILC2-driven innate immune checkpoint mechanism antagonizes NK cell antimetastatic function in the lung. Nat Immunol 21, 998-1009, doi:10.1038/s41590-020-0745-y (2020).
22 Moral, J. A. et al. ILC2s amplify PD-1 blockade by activating tissue-specific cancer immunity. Nature 579, 130-135, doi:10.1038/s41586-020-2015-4 (2020).
23 Saranchova, I. et al. Type 2 Innate Lymphocytes Actuate Immunity Against Tumours and Limit Cancer Metastasis. Scientific reports 8, 2924, doi:10.1038/s41598-018-20608-6 (2018).
24 Jacquelot, N. et al. Blockade of the co-inhibitory molecule PD-1 unleashes ILC2-dependent antitumor immunity in melanoma. Nature Immunology 22, 851-864, doi:10.1038/s41590-021-00943-z (2021).
25 Huang, Q. et al. Type 2 Innate Lymphoid Cells Protect against Colorectal Cancer Progression and Predict Improved Patient Survival. Cancers 13, doi:10.3390/cancers13030559 (2021).
26 Xue, H. H. et al. IL-2 negatively regulates IL-7 receptor alpha chain expression in activated T lymphocytes. Proc Natl Acad Sci USA 99, 13759-13764, doi:10.1073/pnas.212214999 (2002).
27 Vranjkovic, A., Crawley, A. M., Gee, K., Kumar, A. & Angel, J. B. IL-7 decreases IL-7 receptor alpha (CD127) expression and induces the shedding of CD127 by human CD8+ T cells. Int Immunol 19, 1329-1339, doi:10.1093/intimm/dxm102 (2007).
28 Hernández, D. C. et al. An in vitro platform supports generation of human innate lymphoid cells from CD34(+) hematopoietic progenitors that recapitulate ex vivo identity. Immunity 54, 2417-2432.e2415, doi:10.1016/j.immuni.2021.07.019 (2021).
29 Mjösberg, J. et al. The transcription factor GATA3 is essential for the function of human type 2 innate lymphoid cells. Immunity 37, 649-659, doi:10.1016/j.immuni.2012.08.015 (2012).
30 Salimi, M. et al. Group 2 Innate Lymphoid Cells Express Functional NKp30 Receptor Inducing Type 2 Cytokine Production. J Immunol 196, 45-54, doi:10.4049/jimmunol.1501102 (2016).
31 Spits, H. & Mjösberg, J. Heterogeneity of type 2 innate lymphoid cells. Nat Rev Immunol 22, 701-712, doi:10.1038/s41577-022-00704-5 (2022).
32 Brandwein, J. M. et al. Outcomes of patients with relapsed or refractory acute myeloid leukemia: a population-based real-world study. Am J Blood Res 10, 124-133 (2020).
33 Bal, S. M., Golebski, K. & Spits, H. Plasticity of innate lymphoid cell subsets. Nat Rev Immunol 20, 552-565, doi:10.1038/s41577-020-0282-9 (2020).
34 Camelo, A. et al. IL-33, IL-25, and TSLP induce a distinct phenotypic and activation profile in human type 2 innate lymphoid cells. Blood Adv 1, 577-589, doi:10.1182/bloodadvances.2016002352 (2017).
35 Xue, L. et al. Prostaglandin D2 activates group 2 innate lymphoid cells through chemoattractant receptor-homologous molecule expressed on TH2 cells. J Allergy Clin Immunol 133, 1184-1194, doi:10.1016/j.jaci.2013.10.056 (2014).
36 Zhang, Z. et al. Gasdermin E suppresses tumour growth by activating anti-tumour immunity. Nature 579, 415-420, doi:10.1038/s41586-020-2071-9 (2020).
37 Wolf, N. K., Kissiov, D. U. & Raulet, D. H. Roles of natural killer cells in immunity to cancer, and applications to immunotherapy. Nature Reviews Immunology, doi:10.1038/s41577-022-00732-1 (2022).
38 Kaito, Y. et al. DNAM-1/Tigit/CD155/CD112 Axis Is a Novel Target of NK-Cell Therapy in Acute Myeloid Leukemia. Blood 140, 4514-4516, doi:10.1182/blood-2022-164435 (2022).
39 Du, X. et al. CD226 regulates natural killer cell antitumor responses via phosphorylation-mediated inactivation of transcription factor FOXO1. Proc Natl Acad Sci USA 115, E11731-e11740, doi:10.1073/pnas.1814052115 (2018).
40 Deng, Y. et al. Transcription factor Foxol is a negative regulator of natural killer cell maturation and function. Immunity 42, 457-470, doi:10.1016/j.immuni.2015.02.006 (2015).
41 Braun, M. et al. CD155 on Tumor Cells Drives Resistance to Immunotherapy by Inducing the Degradation of the Activating Receptor CD226 in CD8(+) T Cells. Immunity 53, 805-823.e815, doi:10.1016/j.immuni.2020.09.010 (2020).
42 Briukhovetska, D. et al. T cell-derived interleukin-22 drives the expression of CD155 by cancer cells to suppress NK cell function and promote metastasis. Immunity 56, 143-161.eI11, doi:10.1016/j.immuni.2022.12.010 (2023).
43 Fung, I. T. H. et al. Activation of group 2 innate lymphoid cells alleviates aging-associated cognitive decline. J Exp Med 217, doi:10.1084/jem.20190915 (2020).
44 Shen, C. et al. PD-1 Affects the Immunosuppressive Function of Group 2 Innate Lymphoid Cells in Human Non-Small Cell Lung Cancer. Front Immunol 12, 680055, doi:10.3389/fimmu.2021.680055 (2021).
45 Alam, A. et al. Fungal mycobiome drives IL-33 secretion and type 2 immunity in pancreatic cancer. Cancer Cell 40, 153-167.e111, doi:https://doi.org/10.1016/j.ccell.2022.01.003 (2022).
46 Cortez, V. S., Robinette, M. L. & Colonna, M. Innate lymphoid cells: new insights into function and development. Current opinion in immunology 32, 71-77, doi:10.1016/j.coi.2015.01.004 (2015).
47 de Weerdt, I. et al. Innate lymphoid cells are expanded and functionally altered in chronic lymphocytic leukemia. Haematologica 101, e461-e464, doi:10.3324/haematol.2016.144725 (2016).
48 Tsou, A. M. et al. Neuropeptide regulation of non-redundant ILC2s responses at barrier surfaces. Nature 611, 787-793, doi:10.1038/s41586-022-05297-6 (2022).
49 Wallrapp, A. et al. The neuropeptide NMU amplifies ILC2-driven allergic lung inflammation. Nature 549, 351-356, doi:10.1038/nature24029 (2017).
50 Jarick, K. J. et al. Non-redundant functions of group 2 innate lymphoid cells. Nature 611, 794-800, doi:10.1038/s41586-022-05395-5 (2022).
51 Arifuzzaman, M. et al. Inulin fibre promotes microbita-derived bile acids and type 2 inflammation. Nature 611, 578-584, doi:10.1038/s41586-022-05380-y (2022).
52 Cardoso, F. et al. Neuro-mesenchymal units control ILC2s and obesity via a brain-adipose circuit. Nature 597, 410-414, doi:10.1038/s41586-021-03830-7 (2021).
53 Cardoso, V. et al. Neuronal regulation of type 2 innate lymphoid cells via neuromedin U. Nature 549, 277-281, doi:10.1038/nature23469 (2017).
54 Bergsbaken, T., Fink, S. L. & Cookson, B. T. Pyroptosis: host cell death and inflammation. Nat Rev Microbiol 7, 99-109, doi:10.1038/nrmicro2070 (2009).
55 Wu, D., Wei, C., Li, Y., Yang, X. & Zhou, S. Pyroptosis, a New Breakthrough in Cancer Treatment. Frontiers in oncology 11, 698811, doi:10.3389/fonc.2021.698811 (2021).
56 Zhou, Z. et al. Granzyme A from cytotoxic lymphocytes cleaves GSDMB to trigger pyroptosis in target cells. Science 368, doi:10.1126/science.aaz7548 (2020).
57 Liu, Y. et al. Gasdermin E-mediated target cell pyroptosis by CAR T cells triggers cytokine release syndrome. Sci Immunol 5, doi:10.1126/sciimmunol.aax7969 (2020).
58 Cao, Y. et al. Immune checkpoint molecules in natural killer cells as potential targets for cancer immunotherapy. Signal Transduct Target Ther 5, 250, doi:10.1038/s41392-020-00348-8 (2020).
59 Huntington, N. D., Cursons, J. & Rautela, J. The cancer-natural killer cell immunity cycle. Nature Reviews Cancer 20, 437-454, doi:10.1038/s41568-020-0272-z (2020).
60 Iguchi-Manaka, A. et al. Accelerated tumor growth in mice deficient in DNAM-1 receptor. J Exp Med 205, 2959-2964, doi:10.1084/jem.20081611 (2008).
61 Wellenstein, M. D. & de Visser, K. E. Cancer-Cell-Intrinsic Mechanisms Shaping the Tumor Immune Landscape. Immunity 48, 399-416, doi:10.1016/j.immuni.2018.03.004 (2018).
62 Han, B. et al. Altered NKp30, NKp46, NKG2D, and DNAM-1 Expression on Circulating NK Cells Is Associated with Tumor Progression in Human Gastric Cancer. J Immunol Res 2018, 6248590, doi:10.1155/2018/6248590 (2018).
63 Lazarova, M. & Steinle, A. Impairment of NKG2D-Mediated Tumor Immunity by TGF-β. Front Immunol 10, 2689, doi:10.3389/fimmu.2019.02689 (2019).
64 Jacquelot, N., Seillet, C., Vivier, E. & Belz, G. T. Innate lymphoid cells and cancer. Nature Immunology 23, 371-379, doi:10.1038/s41590-022-01127-z (2022).
65 Spits, H. & Mjasberg, J. Heterogeneity of type 2 innate lymphoid cells. Nature Reviews Immunology 22, 701-712, doi:10.1038/s41577-022-00704-5 (2022).
66 Neelapu, S. S. et al. Chimeric antigen receptor T-cell therapy assessment and management of toxicities. Nature Reviews Clinical Oncology 15, 47-62, doi:10.1038/nrclinonc.2017.148 (2018).
67 Sanjana, N. E., Shalem, O. & Zhang, F. Improved vectors and genome-wide libraries for CRISPR screening. Nature Methods 11, 783-784, doi:10.1038/nmeth.3047 (2014).

1. A method of expanding a population of human group 2 innate lymphoid cells (TLC2), comprising contacting the population of human TLC2s with IL-2, TL-7, and IL-15, thereby forming a population of expanded human TLC2.

2. The method of claim 1, wherein the population of human TLC2s is contacted with about 10 IU to about 10000 IU of the IL-2, about 0.1 ng/mL to about 500 ng/mL of the IL-7, and about 0.1 ng/mL to about 500 ng/mL of the IL-15.

3. (canceled)

4. The method of claim 1, wherein the population of human ILC2s is contacted with the IL-2, the IL-7, and the IL-15 for about 5 to about 60 days.

5. The method of claim 4, wherein the population of human TLC2s is contacted with the IL-2, the TL-7, and the IL-15 for about 28 days.

6. The method of claim 5, wherein the contacting comprises:
a) contacting the population of human ILC2s with the IL-2, the IL-7, and the IL-15 for 14 days;
b) isolating the population of human ILC2s from non-TLC2s cells; and
c) contacting the population of human TLC2s with the IL-2, the IL-7, and the IL-15 for a subsequent 14 days.

7. (canceled)

8. The method of claim 1, wherein the population of expanded human TLC2s comprises at least 30% CD161⁺ ILC2.

9.-11. (canceled)

12. The method of claim 8, wherein the CD161 ILC2s comprise at least 30% CRTH2⁺CD117⁺ ILC2.

13.-15. (canceled)

16. The method of claim 1, wherein the population of expanded human ILC2s comprises at least 30% CD122⁺ ILC2.

17.-20. (canceled)

21. The method of claim 1, wherein the population of expanded human ILC2s comprises DNAX Accessory Molecule-1 (DNAM-1) expressing ILC2.

22. The method of claim 1, wherein the population of expanded human ILC2s comprises granzyme B (GZMB) expressing ILC2.

23. The method of claim 1, wherein the population of expanded human ILC2s expresses IL-4, IL-5, IL-9, IL-13, or a combination thereof.

24. The method claim 1, wherein the population of expanded human ILC2s comprises IL-33R expressing ILC2, NKp30 expressing ILC2, or a combination thereof.

25. The method of claim 1, wherein the population of human ILC2s is expanded about 500-fold to about 3000-fold.

26. (canceled)

27. The method of claim 1, wherein the population of expanded human ILC2s does not comprise a substantial number of human group 1 innate lymphoid cells (ILC1), cytotoxic natural killer (NK) cells, or human group 3 innate lymphoid cells (ILC3).

28. A population of expanded human group 2 innate lymphoid cells (ILC2), comprising at least 30% ILC2.

29.-39. (canceled)

40. A method of treating cancer in a subject in need thereof, comprising administering to the subject an effective amount of the population of expanded human ILC2s of claim 28.

41.-44. (canceled)

45. A genetically modified human group 2 innate lymphoid cell (ILC2) comprising a chimeric antigen receptor (CAR), wherein the CAR comprises:
i) an antibody region; and
ii) a transmembrane domain.

46.-54. (canceled)

55. A method of treating cancer in a subject in need thereof, comprising administering to the subject an effective amount of the genetically modified human ILC2s of claim 45.

56.-59. (canceled)

60. A population of expanded human ILC2, made by the method of claim 1.

61. A method of treating cancer in a subject in need thereof, comprising administering to the subject an effective amount of the population of expanded human ILC2 of claim 60.
